(19) United States
(12) Patent Application Publication
(10) Pub. No.: US 2026/0151510 A1
(43) Pub. Date: Jun. 4, 2026

(54) RECOMBINANT AAVS WITH IMPROVED TROPISM AND SPECIFICITY

(71) Applicant: AFFINIA THERAPEUTICS INC., Waltham, MA (US)

(72) Inventors: Charles Francis ALBRIGHT, Boston, MA (US); Xiaohong CAO, Southborough, MA (US); Lin-Ya HUANG, Cambridge, MA (US)

(21) Appl. No.: 19/122,709

(22) PCT Filed: Oct. 19, 2023
(86) PCT No.: PCT/US2023/077340

§ 371 Date: Apr. 18, 2025

Related U.S. Application Data
(60) Provisional application No. 63/380,170, filed on Oct. 19, 2022.
(60) Provisional application No. 63/482,874, filed on Feb. 2, 2023.
(60) Provisional application No. 63/502,871, filed on May 17, 2023.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2750/14145

(57) ABSTRACT

The present disclosure provides a modified AAV capsid protein comprising a targeting peptide in variable region VIII (VR VIII). The modified AAV capsid protein can form an rAAV, which has a preferred tropism, specificity or biodistribution in vivo or in vitro. The rAAV of the present disclosure can be used for gene therapies targeted at a specific tissue.

### 1. CROSS-REFERENCE TO RELATED APPLICATIONS

This application claims the benefit of U.S. Provisional Application Nos. 63/380,170 filed on Oct. 19, 2022, 63/482,874 filed on Feb. 2, 2023, and 63/502,871 filed on May 17, 2023, each of which is hereby incorporated in its entirety by reference.

### 2. SEQUENCE LISTING

The instant application contains a Sequence Listing with 55,921 sequences, which has been submitted via Patent Center and is hereby incorporated by reference in its entirety. Said XML copy, created on Oct. 16, 2023 is named 53023WO_CRF_sequencelisting.xml, and is 48,736,195 bytes in size.

The instant application also incorporates by reference in their entireties the Appendices, titled “Appendix A” and “Appendix B” and the sequences described therein. Appendix A and Appendix B are filed concurrently herewith.

### 3. BACKGROUND

Adeno-associated virus (AAV) has become the vector system of choice for in vivo gene therapy. A growing variety of recombinant AAVs (rAAVs) engineered to deliver therapeutic nucleic acids have been developed and tested in nonhuman primates and humans, and the FDA has recently approved two rAAV gene therapy products for commercialization.

Although AAV vectors are safer and less inflammatory than other viruses, toxicities have occurred following administration of high doses of rAAVs for gene therapy. Thus, local administration of rAAVs to a target tissue or organ has been used to improve targeting and reduce systemic toxicity. Further, various natural and synthetic AAV variants have been tested to develop an AAV vector with desired tropism and specificity.

In general, the capsid is thought to be the primary determinant of infectivity and host-vector related properties such as adaptive immune responses, tropism, specificity, potency, and bio-distribution. Indeed, several of these properties are known to vary between natural serotypes and engineered AAV variants. Over the last decade, novel synthetic AAV variants have been developed by using a variety of capsid engineering techniques, one of which is the insertion of small, peptide into an exposed loop of the capsid protein, called variable region VIII (VRVIII). In some circumstances, the insertion of a novel peptide into a wild type capsid changes the tropism of the variant.

To date, however, there is little understanding as to how changes on the AAV capsid alter their biological properties and AAV vectors with a desired tropism and specificity to therapeutic targets, such as the central nervous system (CNS), have not yet been available. Species-specific differences in AAV tropism, for example between mice and nonhuman primates (NHP), has made it difficult to develop AAV vectors that have a desired tropism in humans.

Treatment of diseases of the CNS remains an intractable problem. Currently, therapeutics for CNS diseases are limited because many of them, when delivered intravenously, do not cross the blood-brain barrier, or, when delivered directly to the brain, are not widely distributed. Thus, there is a need for an AAV vector having a preferred and specific tropism to the CNS for treatment of such CNS diseases.

### 4. SUMMARY OF THE INVENTION

The present disclosure provides a modified AAV capsid protein that can form an rAAV having a preferred tropism and specificity to a therapeutic target. In some embodiments, a modified adeno-associated virus (AAV) capsid protein with the preferred tropism comprises (i) a targeting peptide at a site within variable region VIII (VR VIII); wherein the targeting peptide has a sequence of X₁X₂X₃X₄X₅X₆X₇X₈X₉, wherein X₁, X₂, X₃, X₄, X₅, X₆, X₇, X₈, and X₉ are each independently selected from any amino acid residue. In some embodiments, the modified capsid protein comprising the targeting peptide having a sequence of X₁X₂X₃X₄X₅X₆X₇X₈X₉ at a site within variable region VIII (VR VIII) includes a deletion of amino acids residues (e.g., A587 and Q588 in AAV9 capsid).

Applicant previously demonstrated that a single injection of Anc80L65, a rationally designed synthetic vector (vector described in WO2015/054653, which is incorporated by reference in its entirety herein), into the CSF of adult cynomolgus monkeys leads to efficient transduction of broad regions of the CNS and strikingly outperformed the capabilities of AAV9 to target the cortex and deep brain nuclei (PCT Application No. PCT/US2022/024262, which is incorporated by reference in its entirety herein).

Applicant now reports a modified AAV capsid protein (e.g., Anc80L65, AAV9, and other AAV capsid proteins) comprising a targeting peptide within variable region VIII (VR VIII)) provides synergistic effects to the specific targeting of an rAAV to a target tissue (e.g., the CNS). Therefore, the modified AAV capsid proteins (e.g., modified AAV capsid proteins comprising the targeting peptides described herein inserted at the insertion sites described herein) of the present disclosure can change the tropism, specificity and/or bio-distribution of an AAV comprising the modified AAV capsid protein.

Overall, the rAAVs containing the modified AAV capsid protein comprising a targeting peptide at a site within variable region VIII (VR VIII) demonstrate better targeting with more specific expression of a transgene in the target tissue, e.g., brain compared to a AAV capsid protein not comprising the targeting peptide at a site within variable region VIII.

In another aspect, this disclosure features a modified adeno-associated virus (AAV) capsid protein, comprising: a targeting peptide within variable region VIII (VR VIII), wherein the targeting peptide has a sequence of X₁X₂X₃X₄X₅X₆X₇X₈X₉ and X₁, X₂, X₃, X₄, X₅, X₆, X₇, X₈, and X₉ are each independently selected from any amino acid residue. In some embodiments,
(i) X₁ is independently selected from a proline (P) and a glycine (G);
(ii) X₂ is independently selected from a lysine (L), a threonine (T), a serine (S), an alanine (A), a valine (V), and an isoleucine (I);
(iii) X₃ is independently selected from an asparagine (N), a glutamine (Q), and a proline (P);
(iv) X₄ is independently selected from a glycine (G) and an alanine (A);
(v) X₅ is independently selected from an alanine (A), a threonine (T), a serine (S), a valine (V), and a glycine (G);
(vi) X₆ is independently selected from a valine (V), a leucine (L), an alanine (A), an isoleucine (I), a glycine (G), a serine (S), and a threonine (T);
(vii) X₇ is independently selected from a histidine (H), an arginine (R), and a lysine (K);
(viii) X₈ is independently selected from a leucine (L) and a valine (V); and
(ix) X₉ is independently selected from a tyrosine (Y), an arginine (R), a histidine (H), a lysine (K), and a phenylalanine (F).

In some embodiments, a targeting peptide within VR VIII has a sequence selected from SEQ ID NO: 620-55819.

In some embodiments, the targeting peptide has a sequence of PX₂X₃GAVX₇LY (SEQ ID NO: 2) and X₂, X₃, and X₇ are independently selected from any amino acid residue.

In some embodiments,
(i) X₂ is independently selected from a lysine (L), an isoleucine (I), a valine (V), and an alanine (A);
(ii) X₃ is an asparagine (N) or a glutamine (Q); and
(iii) X₇ is independently selected from a histidine (H), an arginine (R) and a lysine (K).

In some embodiments, the targeting peptide is:

```
(SEQ ID NO: 3)
(i) PLQGAVHLY;
(SEQ ID NO: 4)
(ii) PLQGAVRLY;
  (SEQ ID NO: 5)
(iii) PLQGAVKLY;
  (SEQ ID NO: 6)
(iv) PINGAVHLY;
  (SEQ ID NO: 7)
(v) PVNGAVHLY;
  (SEQ ID NO: 8)
(vi) PANGAVHLY;
or
(SEQ ID NO: 9)
(vii) PLNGAVHLY.
```

In some embodiments, the targeting peptide is inserted between S586 and A589 of an AAV9 capsid protein, thereby replacing A587 and Q588 of the AAV9 capsid protein.

In some embodiments, the modified AAV capsid protein comprises a sequence having at least 90%, 95%, 96%, 97%, 98% or 99% sequence identity to an AAV9 capsid protein.

In some embodiments, the targeting peptide is inserted
(i) between S586 and T589 of an Anc80L65 capsid protein, thereby replacing A587 and N588 of the Anc80L65 capsid protein;
(ii) between Q585 and N588 of an Anc80L65 capsid protein, thereby replacing S586 and A587 of the Anc80L65 capsid protein;
(iii) between L584 and A587 of an Anc80L65 capsid protein, thereby replacing Q585 and S586 of the Anc80L65 capsid protein;
(iv) between A587 and A590 of an Anc80L65 capsid protein, thereby replacing N588 and T589 of the Anc80L65 capsid protein; or
(v) between S586 and A587 of an Anc80L65 capsid protein.

In some embodiments, the targeting peptide comprises: PLNGAVHLY (SEQ ID NO: 9).

In some embodiments, the modified AAV capsid protein comprises a sequence having at least 90%, 95%, 96%, 97%, 98% or 99% sequence identity to an Anc80L65 capsid protein.

In some embodiments, the targeting peptide has a sequence of PX₂X₃GX₅X₆X₇LY (SEQ ID NO: 10) and X₂, X₃, X₅, X₆, and X₇ are independently selected from any amino acid residue. In some embodiments,
(i) X₂ is independently selected from a leucine (L), a threonine (T), or a serine (S);
(ii) X₃ is independently selected from an asparagine (N) and a glutamine (Q);
(iii) X₅ is independently selected from an alanine (A) and a threonine (T);
(iv) X₆ is independently selected from a valine (V) and a leucine (L); and
(v) X₇ is independently selected from a histidine (H), an arginine (R), and a lysine (K).

In some embodiments, the targeting peptide is:

```
  (SEQ ID NO: 11)
(i) PTNGTVRLY;
  (SEQ ID NO: 12)
(ii) PTNGTVHLY;
  (SEQ ID NO: 13)
(iii) PTNGTVKLY;
  (SEQ ID NO: 14)
(iv) PSNGTLRLY;
  (SEQ ID NO: 15)
(v) PSNGTLHLY;
  (SEQ ID NO: 16)
(vi) PSNGTLKLY;
  (SEQ ID NO: 17)
(vii) PTNGTLRLY;
  (SEQ ID NO: 18)
(viii) PTNGTLHLY;
or
(SEQ ID NO: 19)
(ix) PTNGTLKLY.
```

In some embodiments, the targeting peptide has a sequence of PX₂X₃GAVX₇X₈X₉ (SEQ ID NO: 20) and X₂, X₃, X₅, X₆, and X₇ are independently selected from any amino acid residue. In some embodiments,
(i) X₂ is independently selected from a leucine (L), a threonine (T), or a serine (S);
(ii) X₃ is independently selected from an asparagine (N) and a glutamine (Q);
(iii) X₇ is independently selected from a histidine (H) and a threonine (T);
(iv) X₈ is independently selected from a valine (V) and a leucine (L); and (v) X₉ is independently selected from a tyrosine (Y) and an arginine (R).

In some embodiments, the targeting peptide is:

```
  (SEQ ID NO: 21)
(i) PTQGAVTVR;
  (SEQ ID NO: 22)
(ii) PLQGAVTVR;
  (SEQ ID NO: 23)
(iii) PLQGAVHVR;
  (SEQ ID NO: 24)
(iv) PLQGAVHVY;
  (SEQ ID NO: 25)
(v) PSQGAVTLR;
  (SEQ ID NO: 26)
(vi) PLQGAVTLR;
  (SEQ ID NO: 27)
(vii) PLQGAVHLR;
or
(SEQ ID NO: 28)
(viii) PTQGAVTLR.
```

In some embodiments, the targeting peptide does not comprise PLNGAVHLY (SEQ ID NO: 9).

In some embodiments, the targeting peptide is inserted between S586 and A589 of an AAV9 capsid protein, thereby replacing A587 and Q588 of the AAV9 capsid protein.

In another aspect, this disclosure features a modified adeno-associated virus (AAV) capsid protein, comprising: a targeting peptide within VR VIII wherein the targeting peptide has a sequence selected from SEQ ID NO: 160-619.

In some embodiments, the modified AAV capsid protein comprises a sequence having at least 90%, 91%, 92%, 93%, 94%, 95%, 96%, 97%, 98%, 99% or 99.5% sequence identity to the sequence of a reference AAV capsid protein.

In some embodiments, the reference AAV capsid protein is selected from VP1, VP2 and VP3.

In some embodiments, the reference AAV capsid protein is a capsid protein of an AAV selected from the group consisting of: AAV9; Anc80L65; Anc80-55, Anc80-129, Anc80-156, Anc80-751, Anc80-1029, Anc80-1712, AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.1l-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

In some embodiments, the reference AAV capsid protein is a capsid protein having a sequence selected from SEQ ID Nos: 54-158, or a fragment thereof.

In some embodiments, the modified AAV capsid protein has one or more modifications comprising amino acid insertions, deletions, substitutions or combinations thereof as compared to a reference AAV capsid protein.

In some embodiments, the reference AAV capsid protein is a capsid protein having a sequence of SEQ ID NO: 61 or a fragment thereof.

In some embodiments, the reference AAV capsid protein is a capsid protein having a sequence of SEQ ID NO: 142 or a fragment thereof.

In some embodiments, the targeting peptide is positioned between 576 and 601 within VR VIII of the modified AAV capsid protein.

In some embodiments, the modified AAV capsid protein further comprises an N-terminal flanking region on the N-terminal end of the targeting peptide.

In some embodiments, the N-terminal flanking region comprises at least four consecutive amino acid residues from amino acids 576-585 of the reference AAV capsid protein, wherein the amino acid residues are numbered according to the amino acid sequence of the reference AAV capsid protein.

In some embodiments, the N-terminal flanking region has the sequence of B₁YGB₂VA′TNB₃QS (SEQ ID NO: 55849), and B₁, B₂, and B₃ are each independently selected from any amino acid residue. In some embodiments, B₁ is selected from an glutamate (E) or a serine (S), B₂ is selected from a threonine (T) or a glutamine (Q), and B₃ is selected from a leucine (L) or a histidine (H).

In some embodiments, the N-terminal flanking region has the sequence of SYGQVATNHQS (SEQ ID NO: 55848).

In some embodiments, the N-terminal flanking region replaces N-terminal reference sequence of the reference AAV capsid protein, wherein the N-terminal reference sequence has at least 60% sequence identity to the N-terminal flanking region and positioned at N-terminal end of an insertion site of the targeting peptide within the reference AAV capsid protein. In some embodiments, the N-terminal flanking region has the sequence of SYGQVATNHQS (SEQ ID NO: 55848).

In some embodiments, the modified AAV capsid protein further comprises a C-terminal flanking region on the C-terminal end of the targeting peptide.

In some embodiments, the C-terminal flanking region comprises at least four consecutive amino acids from amino acid residues 589-602 of the reference AAV capsid protein, wherein the amino acid residues are numbered according to the amino acid sequence of the reference AAV capsid protein.

In some embodiments, the C-terminal flanking region has the sequence of AQAQTGZ₁VZ₂Z₃QGZ₄ (SEQ ID NO: 55851), and Z₁, Z₂, Z₃, and Z₄ are each independently selected from any amino acid residue. In some embodiments, Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

In some embodiments, the C-terminal flanking region has the sequence of AQAQTGWVQNQGI (SEQ ID NO: 55850).

In some embodiments, the C-terminal flanking region replaces C-terminal reference sequence of the reference AAV capsid protein, wherein the C-terminal reference sequence has at least 60% sequence identity to the C-terminal flanking region and positioned at C-terminal end of an insertion site of the targeting peptide within the reference AAV capsid protein. In some embodiments, the C-terminal reference sequence has the sequence of AQAQTGZ₁VZ₂Z₃QGZ₄ (SEQ ID NO: 55851).

In some embodiments, the modified AAV capsid protein further comprises: a N-terminal flanking region having the sequence of B₁YGB₂VATNB₃QS (SEQ ID NO: 55849, where B₁, B₂, and B₃ are each independently selected from any amino acid residue; and a C-terminal flanking region having the sequence of AQAQTGZ₁VZ₂Z₃QGZ₄ (SEQ ID NO: 55851), where Z₁, Z₂, Z₃, and Z₄ are each independently selected from any amino acid residue.

T In some embodiments, the modified AAV capsid protein further comprises: a N-terminal flanking region having the sequence of B₁YGB₂VATNB₃QS (SEQ ID NO: 55860) and B₁ is selected from a glutamate (E) or a serine (S), B₂ is selected from a threonine (T) or a glutamine (Q), and B₃ is selected from a leucine (L) or a histidine (H); and a C-terminal flanking region having the sequence of AQAQTGZ₁VZ₂Z₃QGZ₄ (SEQ ID NO: 55861) and Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

In some embodiments, the modified AAV capsid protein further comprises:
a N-terminal flanking region having the sequence of SYGQVATNHQS (SEQ ID NO: 55848), and
a C-terminal flanking region having the sequence of AQAQTGWVQNQGI (SEQ ID NO: 55850).

In some embodiments, the modified AAV capsid protein comprises an amino acid sequence of B₁YGB₂VATNB₃QSPLMGAVHLYAQAQTGZ₁VZ₂Z₃QGZ₄ (SEQ ID NO: 55852), where B₁, B₂, B₃, Z₁, Z₂, Z₃, and Z₄ are each independently selected from any amino acid residue. In some embodiments, B₁ is selected from a glutamate (E) or a serine (S), B₂ is selected from a threonine (T) or a glutamine (Q), B₃ is selected from a leucine (L) or a histidine (H), Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

In some embodiments, the targeting peptide is a peptide having 1, 2, 3, 4, 5, 6, 7, 8, 9, 10, or more amino acid residues different to a sequence of SEQ ID NO: 29, wherein the different amino acid comprise an insertion, a deletion, or a substitution.

In some embodiments, the targeting peptide is SEQ ID NO: 29.

In some embodiments, the one or more modifications comprises an amino acid insertion, deletion, substitution, or a combination thereof to introduce the targeting peptide into VR VIII of the reference AAV capsid protein.

In some embodiments, the one or more modifications comprises an amino acid modification outside of VR VIII of the reference AAV capsid protein.

In some embodiments, the one or more modifications outside of VR VIII of the reference AAV capsid protein comprise one or more modifications in VR I, VR II, VR III, VR IV, VR V, VR VI, or VR VII.

In some embodiments, the one or more modifications outside of VR VIII of the reference AAV capsid protein comprise one or more modifications in VR IV and VR V.

In some embodiments, the one or more modifications in VR IV result in introduction of a sequence of SEQ ID NO: 30.

In some embodiments, the one or more modifications in VR V result in introduction of a sequence of SEQ ID NO: 31.

In some modifications, the one or more modifications comprises one or more amino acid deletions within VR VIII.

In some modifications, the one or more amino acid deletions comprise a deletion of one, two, three, four, or five or more amino acid residues immediately adjacent to the N terminal end of the targeting peptide within VR VIII.

In some modifications, the one or more amino acid deletions comprise a deletion of at least one amino acid residue at a position selected from 584, 585, 586, 587, or 588, or a combination thereof, relative to a reference sequence numbered according to the amino acid sequence of the reference AAV capsid protein. In some modifications, the one or more amino acid deletions comprises a deletion of the amino acid residue at position 587, relative to a reference sequence numbered according to the amino acid sequence of the reference AAV capsid protein. In some modifications, the one or more amino acid deletions comprises a deletion of the amino acid residue at position 588, relative to a reference sequence numbered according to the amino acid sequence of the reference AAV capsid protein.

In some modifications, the amino acid deletion comprises deletion of one, two, three, four, or five or more amino acid residues immediately adjacent to the C terminal end of the targeting peptide within VR VIII. In some modifications, the amino acid deletion comprises deletion of an amino acid residue at position 589, 590, or 591, or a combination thereof, relative to a reference sequence numbered according to the amino acid sequence of the reference AAV capsid protein.

In some modifications, the amino acid insertion comprises insertion of one, two, three, four, or five or more amino acid residues immediately adjacent to C terminal end of the targeting peptide within VR VIII.

In some modifications, the inserted amino acid residue is independently selected from any amino acid residue.

In some modifications, the inserted amino acid residue(s) is identical to the amino acid residue deleted adjacent to the N terminal end or C terminal end of the targeting peptide within VR VIII.

In some modifications, the inserted amino acid residue is an alanine (A) or an asparagine (N). In some modifications, the inserted amino acids are an alanine (A) at the position immediately adjacent to the C terminal end of the targeting peptide and an asparagine (N) at the next subsequent position, thereby having an amino acid sequence of AN immediately adjacent to the C terminal end of the targeting peptide.

In some modifications, the targeting peptide is:

(SEQ ID NO: 32)
(i) PLNGAVHLYN;
or
(SEQ ID NO: 33)
(ii) PLNGAVHLYAN.

In some embodiments,
(i) the reference AAV capsid protein is a capsid protein of AAV1 or a modification thereof and the targeting peptide is between S586 and T589 of the reference AAV capsid protein, thereby replacing residues S587 and S588 of the reference capsid protein;
(ii) the reference AAV capsid protein is a capsid protein of AAV2 or a modification thereof and the targeting peptide is between R585 and R588 of the reference AAV capsid protein, thereby replacing residues G586 and N587 of the reference capsid protein;
(iii) the reference AAV capsid protein is a capsid protein of AAV3 or a modification thereof and the targeting peptide is between S586 and T589 of the reference AAV capsid protein, thereby replacing residues S587 and N588 of the reference capsid protein;
(iv) the reference AAV capsid protein is a capsid protein of AAV4 or a modification thereof and the targeting peptide is between D582 and N585 of the reference AAV capsid protein, thereby replacing residues Q583 and S584 of the reference capsid protein;
(v) the reference AAV capsid protein is a capsid protein of AAV5 or a modification thereof and the targeting peptide is between S575 and T578 of the reference AAV capsid protein, thereby replacing residues S576 and S577 of the reference capsid protein;
(vi) the reference AAV capsid protein is a capsid protein of AAV6 or a modification thereof and the targeting peptide is between S586 and T589 of the reference AAV capsid protein, thereby replacing residues S587 and S588 of the reference capsid protein;
(vii) the reference AAV capsid protein is a capsid protein of AAV7 or a modification thereof and the targeting peptide is between A587 and T590 of the reference AAV capsid protein, thereby replacing residues A588 and N589 of the reference capsid protein;
(viii) the reference AAV capsid protein is a capsid protein of AAV8 or a modification thereof and the targeting peptide is between Q588 and A591 of the modified AAV capsid protein thereby replacing residues Q589 and N590 of the reference capsid protein;
(ix) the reference AAV capsid protein is a capsid protein of AAV9 or a modification thereof and the targeting peptide is between S586 and A589 of the reference AAV capsid protein thereby replacing residues A587 and Q588 of the reference capsid protein;
(x) the reference AAV capsid protein is a capsid protein of AAVrh10 or a modification thereof and the targeting peptide is between Q588 and A591 of the reference AAV capsid protein thereby replacing residues Q589 and N590 of the reference capsid protein;
(xi) the reference AAV capsid protein is a capsid protein of AAVpo.1 or a modification thereof and the targeting peptide is between N565 and S568 of the reference AAV capsid protein thereby replacing residues Q566 and N567 of the reference capsid protein;
(xii) the reference AAV capsid protein is a capsid protein of AAV12 or a modification thereof and the targeting peptide is between N590 and A593 of the reference AAV capsid protein thereby replacing residues Q591 and N592 of the reference capsid protein;
(xiii) the reference AAV capsid protein is a capsid protein of Anc80 or a modification thereof and the targeting peptide is between S586 and T589 of the reference AAV capsid protein thereby replacing residues S587 and N588 of the reference capsid protein;
(xiv) the reference AAV capsid protein is a capsid protein of Anc80-55 or a modification thereof and the targeting peptide is between S586 and T589 of the reference AAV capsid protein thereby replacing residues S587 and N588 of the reference capsid protein;
(xv) the reference AAV capsid protein is a capsid protein of Anc80-129 or a modification thereof and the targeting peptide is between S586 and T589 of the reference AAV capsid protein thereby replacing residues A587 and N588 of the reference capsid protein;
(xvi) the reference AAV capsid protein is a capsid protein of Anc80-156 or a modification thereof and the targeting peptide is between S586 and T589 of the reference AAV capsid protein thereby replacing residues A587 and N588 of the reference capsid protein;
(xvii) the reference AAV capsid protein is a capsid protein of Anc80-751 or a modification thereof and the targeting peptide is between S586 and T589 of the reference AAV capsid protein thereby replacing residues A587 and N588 of the reference capsid protein;
(xviii) the reference AAV capsid protein is a capsid protein of Anc80-1029 or a modification thereof and the targeting peptide is between S586 and T589 of the reference AAV capsid protein thereby replacing residues A587 and N588 of the reference capsid protein; or
(xix) the reference AAV capsid protein is a capsid protein of Anc80-1712 or a modification thereof and the targeting peptide is between S586 and T589 of the reference AAV capsid protein thereby replacing residues A587 and N588 of the reference capsid protein.

In some embodiments,
(i) the reference AAV capsid protein is a capsid protein of AAV1 or a modification thereof and the targeting peptide is between D590 and P591 or between S588 and T589 of the reference AAV capsid protein;
(ii) the reference AAV capsid protein is a capsid protein of AAV2 or a modification thereof and the targeting peptide is between R588 and Q589 or between N587 and R588 of the reference AAV capsid protein;
(iii) the reference AAV capsid protein is a capsid protein of AAV3 or a modification thereof and the targeting peptide is between S586 and S587 or between N588 and T589 of the reference AAV capsid protein;
(iv) the reference AAV capsid protein is a capsid protein of AAV4 or a modification thereof and the targeting peptide is between S584 and N585 or between S586 and N587 of the reference AAV capsid protein;
(v) the reference AAV capsid protein is a capsid protein of AAV5 or a modification thereof and the targeting peptide is between S575 and S576 or between T577 and T578 of the reference AAV capsid protein;
(vi) the reference AAV capsid protein is a capsid protein of AAV6 or a modification thereof and the targeting peptide is between D590 and P591 or S588 and T589 of the reference AAV capsid protein;
(vii) the reference AAV capsid protein is a capsid protein of AAV7 or a modification thereof and the targeting peptide is between N589 and T590 of the reference AAV capsid protein;
(viii) the reference AAV capsid protein is a capsid protein of AAV8 or a modification thereof and the targeting peptide is between N590 and T591 of the modified AAV capsid protein;
(ix) the reference AAV capsid protein is a capsid protein of AAV9 or a modification thereof and the targeting peptide is between Q588 and A589 of the reference AAV capsid protein;
(x) the reference AAV capsid protein is a capsid protein of AAVrh10 or a modification thereof and the targeting peptide is between N590 and A591 of the reference AAV capsid protein;
(xi) the reference AAV capsid protein is a capsid protein of AAVpo.1 or a modification thereof and the targeting peptide is between N567 and S568 or between N569 and T570 of the reference AAV capsid protein;
(xii) the reference AAV capsid protein is a capsid protein of AAV12 or a modification thereof and the targeting peptide is between N592 and A593 or between T594 and T595 of the reference AAV capsid protein;
(xiii) the reference AAV capsid protein is a capsid protein of Anc80 or a modification thereof and the targeting peptide is between T589 and A590, between N588 and T589, or between N587 and T588 of the reference AAV capsid protein;
(xiv) the reference AAV capsid protein is a capsid protein of Anc80-55 or a modification thereof and the targeting peptide is between T589 and A590 or between N587 and T588 of the reference AAV capsid protein;
(xv) the reference AAV capsid protein is a capsid protein of Anc80-129 or a modification thereof and the targeting peptide is between T589 and A590 or between N587 and T588 of the reference AAV capsid protein;
(xvi). the reference AAV capsid protein is a capsid protein of Anc80-156 or a modification thereof and the targeting peptide is between T589 and A590 or between N587 and T588 of the reference AAV capsid protein;
(xvii) the reference AAV capsid protein is a capsid protein of Anc80-751 or a modification thereof and the targeting peptide is between T589 and A590 or between N587 and T588 of the reference AAV capsid protein;
(xviii) the reference AAV capsid protein is a capsid protein of Anc80-1029 or a modification thereof and the targeting peptide is between T589 and A590 or between N587 and T588 of the reference AAV capsid protein; or
(xix) the reference AAV capsid protein is a capsid protein of Anc80-1712 or a modification thereof and the targeting peptide is between T589 and A590 or between N587 and T588 of the reference AAV capsid protein.

In another aspect, this disclosure features a polynucleotide encoding any of the modified AAV capsid proteins described herein.

In another aspect, this disclosure features a vector comprising any of the polynucleotides described herein. In some embodiments, the vector includes a promoter operably linked to the polynucleotide.

In another aspect, this disclosure features a host cell comprising any of the modified AAV capsid proteins described herein, any of the polynucleotides described herein, or any of the vectors described herein.

In another aspect, this disclosure features a recombinant AAV virion (rAAV) comprising any of the modified AAV capsid proteins described herein.

In some embodiments, the rAAV further comprises an exogenous polynucleotide. In some embodiments, the exogenous polynucleotide comprises a template for homology directed repair. In some embodiments, the exogenous polynucleotide comprises an expressible polynucleotide encoding a therapeutic tRNA, miRNA, gene editing guide RNA, or RNA-editing guide RNA. In some embodiments, the exogenous polynucleotide comprises an expressible polynucleotide encoding a therapeutic protein. In some embodiments, the therapeutic protein is used for treating and/or preventing a disease of the central nervous system (CNS).

In some embodiments, the AAV virion, when administered in a therapeutically effective amount to a subject, has increased specificity to a central nervous system (CNS) tissue, relative to a reference AAV virion comprising a reference AAV capsid protein without the targeting peptide.

In some embodiments, the AAV virion, when administered in a therapeutically effective amount to a subject, has increased transduction efficiency in the CNS, relative to a reference AAV virion comprising a reference AAV capsid protein without the targeting peptide.

In some embodiments, the AAV virion, when administered in a therapeutically effective amount to a subject, has increased blood brain barrier penetration in the subject, relative to a reference AAV virion comprising a reference capsid protein without the targeting peptide.

In another aspect, this disclosure features a pharmaceutical composition comprising any of the AAV virions described herein.

In another aspect, this disclosure features a method for treating or ameliorating or preventing a disease or condition in a subject, comprising administering a therapeutically effective amount of any of the AAV virions described herein or any of the pharmaceutical compositions described herein.

In some embodiments, the disease is a disease of the central nervous system (CNS).

In some embodiments, the CNS disease is a lysosomal storage disease (LSD).

In some embodiments, the CNS disease is a leukodystrophy.

In some embodiments, the CNS disease is metachromatic leukodystrophy (MLD).

In some embodiments, the CNS disease is Krabbe.

In some embodiments, the CNS disease is cancer. In some embodiments, the CNS disease is metastatic breast cancer.

In some embodiments, any of the modified adeno-associated virus (AAV) capsid proteins described herein are used for treating and/or preventing a disease of the central nervous system (CNS).

In another aspect, this disclosure features an AAV virion comprising any of the modified AAV capsid proteins described herein or any of the AAV virions described herein used for treating and/or preventing a disease of the central nervous system (CNS).

In another aspect, this disclosure features any of the pharmaceutical compositions comprising any of the modified AAV capsid proteins described herein, and/or any of the AAV virions described herein used for treating and/or preventing a disease of the central nervous system (CNS).

In another aspect, this disclosure features a method of transferring an exogenous polynucleotide to the central nervous system (CNS), comprising the step of administering any of the AAV virions described herein to a subject. In some embodiments, the administration results in transfer of the exogenous polynucleotide in the CNS, at a CNS:liver infection ratio of greater than 1 when measured by genome copies of the AAV virion. In some embodiments, the administration results in expression of the exogenous polynucleotide in CNS, at a CNS:liver expression ratio of greater than 10. In some embodiments, the CNS:liver expression ratio of greater than 10 when measured by protein expression.

In another aspect, this disclosure features use of any of the AAV capsid proteins described herein, and/or any of the AAV virions described herein for transferring an exogenous polynucleotide to the central nervous system. In some embodiments, the use is a non-therapeutic use.

It is expected that the modified rAAVs of the disclosure will provide improved therapeutics indices due to specific tropism to the target tissues and greater expression of a therapeutic protein as compared to a control rAAV having an unmodified capsid protein.

### 5. BRIEF DESCRIPTION OF THE SEVERAL VIEWS OF THE DRAWINGS

These and other features, aspects, and advantages of the present invention will become better understood with regard to the following description, and accompanying drawings where:

FIG. 1 summarizes the NHP study design described in the Examples.

FIGS. 2A-2D are immunohistochemistry (IHC) images of brain sections, obtained from NHPs administered with (i) Anc80L65-CAG-GFP or (ii) AAV9-CAG-GFP by intracisternal magna injection (ICM) or lumbar-puncture (LP). Brown stain=GFP expression (arrows). Inset in Anc80L65-LP (FIG. 2B) shows mostly neuronal staining. FIG. 2A shows GFP expression after administration of Anc80L65 via ICM injection. FIG. 2B shows GFP expression after administration of Anc80L65 via LP. FIG. 2C shows GFP expression after administration of AAV9 via ICM injection. FIG. 2D shows GFP expression after administration of AAV via LP.

FIGS. 3A-3C are IHC images of brain sections including cortex, obtained from a NHP administered with vehicle (FIG. 3A), Anc80L65-CAG-GFP (FIG. 3B), or AAV9-CAG-GFP (FIG. 3C). Brown stain=GFP expression.

FIGS. 4A-4B are IHC images of a brain section including ependyma and caudate nucleus, obtained from a NHP administered Anc80L65-CAG-GFP by ICM injection. FIG. 4B is an enlarged image of a portion of FIG. 4A. Brown stain=GFP expression.

FIGS. 5A-5B are IHC images of a brain section including caudate nucleus, obtained from a NHP administered with Anc80L65-CAG-GFP by ICM injection. FIG. 5B is an enlarged image of a portion of FIG. 5A. Brown stain=GFP expression.

FIG. 6 is an IHC image of a brain section including substantia nigra, obtained from a NHP administered with Anc80L65-CAG-GFP by ICM injection. Brown stain=GFP expression.

FIGS. 7A and 7B are IHC images of a brain section including perivascular cells, obtained from a NHP administered with Anc80L65-CAG-GFP by ICM injection. FIG. 7B is an enlarged image of a portion of FIG. 7A. Brown stain=GFP expression.

FIGS. 8A and 8B are IHC images of a brain section including cortex, obtained from a NHP administered with Anc80L65-CAG-GFP by ICM injection. FIG. 8B is an enlarged image of a portion of FIG. 8A. Brown stain=GFP expression.

FIG. 9 is an IHC image of a brain section including cortex, obtained from a NHP administered with Anc80L65-CAG-GFP by lumbar puncture (LP). Brown stain=GFP expression.

FIGS. 10A-10C provide one-way analysis of transgene expression determined by measurement of mRNA transcript of eGFP calculated according to the equation: % eGFP expression=(eGFP cp/uL÷RPP30 cp/uL)×100, in various brain regions in animals administered with AAV9-CAG-GFP by ICM injection or with Anc80L65-CAG-GFP by LP.

FIG. 10A provides data for the frontal cortex; FIG. 10B provides data for the motor cortex; and FIG. 10C provides data for the parietal lobe of the cortex.

FIGS. 11A-11B provide one-way analysis of transgene expression determined by measurement of mRNA transcript of eGFP calculated according to the equation: % eGFP expression=(eGFP cp/uL÷RPP30 cp/uL)×100, in various brain regions administered with AAV9-CAG-GFP by ICM injection or with Anc80L65-CAG-GFP by LP. FIG. 11A provides data for the caudate nucleus; and FIG. 11B provides data for the globus pallidus.

FIGS. 12A-12B provide one-way analysis of transgene expression determined by measurement of mRNA transcript of eGFP calculated according to the equation: % eGFP expression=(eGFP cp/uL÷RPP30 cp/uL)×100, in various brain regions administered with AAV9-CAG-GFP by ICM injection or with Anc80L65-CAG-GFP by LP. FIG. 12A provides data for the putamen; and FIG. 12B provides data for the substantia nigra.

FIGS. 13A-17 provide one-way analysis of viral genome (DNA) copy per diploid genome (VGC/DG) determined by measurement of the genome copy numbers using ddPCR and calculation of (VGC/DG) values using the equation: VGC/DG=(eGFP cp/uL÷RPP30 cp/uL)×2. Each figure provides data for a different brain region or liver, including cerebellar cortex (FIG. 13A), dorsal root ganglia, cervical (FIG. 13B), dorsal root ganglia, lumbar (FIG. 14A), frontal cortex (FIG. 14B), liver (FIG. 15A), motor cortex (FIG. 15B), spinal cord, cervical (FIG. 16A), spinal cord, lumbar (FIG. 16B), and sciatic nerve (FIG. 17).

FIGS. 18A, 18B, 19A, 19B, 20A, 20B and 21 provide one-way analysis of transgene expression determined by measurement of mRNA transcript of eGFP calculated according to the equation: % eGFP expression=(eGFP cp/uL÷RPP30 cp/uL)×100. Each figure provides data for a different brain region, including caudate nucleus (FIG. 18A), frontal cortex (FIG. 18B), globus pallidus (FIG. 19A), motor cortex (FIG. 19B), parietal cortex (FIG. 20A), putamen (FIG. 20B), and substantia nigra (FIG. 21).

FIGS. 22A-22D are immunohistochemistry (IHC) images of brain sections, obtained from NHPs administered with Anc80L65-CAG-GFP or AAV9-CAG-GFP by intracisternal magna injection. Brown stain=GFP expression. FIG. 22A shows GFP expression in the cortex after administration of Anc80L65-CAG-GFP. FIG. 22B shows GFP expression in the caudate nucleus after administration of Anc80L65-CAG-GFP. FIG. 22C shows GFP expression in the cortex after administration of AAV9-CAG-GFP. FIG. 22D shows GFP expression in the caudate nucleus after administration of AAV9-CAG-GFP.

FIGS. 23 and 24 illustrate the GFP mRNA expression measured by ddPCR in the NHP brain and spinal cord 2 weeks after ICM or LP delivery of AAV9-CAG-GFP or Anc80L65-CAG-GFP. FIG. 23 provides % GFP expression in the frontal cortex, motor cortex, and parietal cortex. FIG. 24 Provides % GFP expression in the caudate nucleus, globus palidus, putamen, and substantia nigra.

FIG. 25 illustrates the vector genome copy analysis via qPCR. VGCs per cell (presented as mean vector genome copies per diploid genome VGC/DG) in NHPs injected with Anc80L65-CAG-GFP and AAV9-CAG-GFP by LP or ICM injection are provided.

FIGS. 26A-26F are double immunofluorescence (IF) staining images of brain sections administered with Anc80L65-CAG-GFP (FIGS. 26A, 26B and 26C) or AAV9-CAG-GFP (FIGS. 26D, 26E and 26F). The transgene expression from the AAVs was detected by staining against GFP and cell types were detected by staining against cell-type specific markers, including NeuN for neurons (FIG. 26A and FIG. 26D), GFAP for astrocytes (FIG. 26B and FIG. 26E), and Iba1 for microglial cells (FIG. 26C and FIG. 26F). Examples were imaged from the motor cortex. In all cases, GFP+ cells are shown in red, the cell specific marker is shown in green, and the merged images are shown with double-labeled cells in yellow/orange (arrows for double-labeled cells).

FIGS. 27A-27F are double immunofluorescence (IF) staining images of brain sections from NHP administered with Anc80L65-CAG-GFP via LP (FIGS. 27A, 27B and 27C) or via ICM (FIGS. 27D, 27E and 27F). Examples were imaged from the motor cortex. The transgene expression from Anc80L65 was detected by staining against GFP and oligodendrocyte cells were detected by staining against oligodendrocyte specific marker OLIG2, shown in green (FIG. 27A and FIG. 27D). GFP+ cells are shown (FIG. 27B and FIG. 27E). The merged images are shown with arrows pointing to double-labeled cells (FIG. 27C and FIG. 27F).

FIG. 28 illustrates the structure of an AAV VP1 protein with certain variable regions (VR I, VR IV, VR V, and VR VIII) highlighted. The location of the targeting peptide insertion site in VR VIII is indicated.

FIGS. 29A-29C provide the sequence alignment of VP1 sequences of certain AAV variants using AAV2 VP1 as a reference. The location of the insertion site of a targeting peptide (FIG. 29B) is indicated. FIG. 29 discloses SEQ ID NOS 55, 54, 58, 56, 64, 59, 60, 89, 111, 61, 63, 62, and 57, respectively, in order of appearance.

FIGS. 30A-30D provide the sequence alignment of VP1 sequences of ancestral AAVs using AAV2 as a reference. The location of the insertion site of a targeting peptide (FIG. 30C) is indicated. One or more representative member sequences for each of the Anc80, Anc81, Anc82, Anc83, Anc84, Anc94, Ac110, Anc113, Anc 126 and Anc127 libraries were used for the alignment. FIGS. 30A-30D disclose SEQ ID NOS 55, 55911, 143, 147, 144, 146, 145, 148, 149-150, 142, and 55912-55921, respectively, in order of appearance.

FIG. 31 provides sequences for a window of VR VIII of modified AAV capsid proteins (e.g., Anc80L65) with targeting peptides at various positions in VR VIII. Underlined sequences represent amino acid residues of the targeting peptide. FIG. 31 discloses SEQ ID NOS 55875-55881, respectively, in order of appearance.

FIG. 32A illustrates the structure of an AAV VP1 capsid protein focusing on the variable region (VR) IV, VR V, and VR VIII.

FIG. 32B provides partial sequence information for three AAV capsid protein variant having various combinations of VR IV, VR V, and VR VIII regions of an Anc80L65 or AAV9 capsid protein.

FIG. 32C provides a sequence alignment of amino acid residues 540-640 of SEQ ID NOs: 61 (AAV9 capsid protein), 142 (Anc80L65 capsid protein), and 55853. Box indicates amino acids positions 576-601 corresponding to positions in AAV9 without a targeting peptide inserted. FIG. 32C discloses SEQ ID NOS 55853, and 55882-55883, respectively, in order of appearance.

FIG. 32D provides a sequence alignment of amino acid residues 540-640 of AAV9 (SEQ ID NO: 61), AAV9 (SEQ ID NO: 61) and targeting peptide (SEQ ID NO: 9) inserted in between 588 and 589 (with 586 and 587 removed), and AFT-6 has the Anc80L65 capsid protein backbone with an N-terminal flanking region from AAV9 (SEQ ID NO: 55848), a targeting peptide of SEQ ID NO: 9, and a C-terminal flanking region from AAV9 (SEQ ID NO: 55850). Box indicates amino acids positions 559-635. FIG. 32D discloses SEQ ID NOS 55884-55885, 55884, and 55886, respectively, in order of appearance.

FIG. 33 provides sequences for a window of VR VIII of modified AAV capsid proteins (e.g., AAV9) with targeting peptides shown as underlined amino acid residues and blank spaces at positions 587 and 588 corresponding to deletion of A587 and Q588 from the modified AAV9 capsid protein. Each of AFT-9 to AFT-31 are inserted as described into an AAV9 capsid protein. FIG. 33 discloses SEQ ID NOS 55875, and 55887-55909, respectively, in order of appearance.

FIG. 34 shows a plot of gene transfer efficacy data (RNA logMN_Fold Change) averaged across all brain tissues for each of the rAAV in the AAV^{mini} library. ** denotes technical replicate of AFT-6 (SEQ ID NO: 55820).

FIG. 35 shows a plot of tissue enrichment scores (log 10 scale of expression) for the indicated tissues. Capsids tested included AAV9 and an Anc80L65 capsid comprising an N2 targeting peptide (SEQ ID NO: 9) located in VPR VIII (full capsid sequence is referred to as AFT-6 (SEQ ID NO: 55820)). “On-target” CNS tissues analyzed included frontal lobe, motor cortex, parietal lobe, occipital lobe, temporal lobe, cerebellum, putamen, thalamus, globus pallidus, caudate, and substantia nigra. “Off-target” tissues analyzed included dorsal root ganglion; such as cervical, lumbar, thoracic; and liver. Higher tissue enrichment score values represent greater tropism.

FIG. 36 shows a plot of tissue enrichment scores (log 10 scale of expression) for the indicated tissues. Capsids tested included AAV9, an AAV9 comprising an N3 targeting peptide (PLNGSVHLY (SEQ ID NO: 3603 in Appendix B)) positioned in VR VIII between amino acid residues 586 and 589, replacing amino acids A587 and Q588, and an AAV9 comprising an N4 targeting peptide (PLNGTVHLY (SEQ ID NO: 1232 in Appendix B)) positioned in VR VIII between amino acid residues 586 and 589, replacing amino acids A587 and Q588. CNS tissues analyzed included frontal cortex, temporal lobe, putamen, thalamus, and globus pallidus. Higher tissue enrichment score values represent greater tropism.

FIG. 37 shows a plot of tissue enrichment scores (log10 scale of expression) for the indicated tissues. Capsids tested included an AAV9 comprising an N1 targeting peptide (SEQ ID NO: 9) located in VR VIII of AAV9, an Anc80L65 capsid comprising an N2 targeting peptide (SEQ ID NO: 9) located in VR VIII (full capsid sequence is referred to as AFT-6 (SEQ ID NO: 55820)); an AAV9 comprising an N3 targeting peptide (PLNGSVHLY (SEQ ID NO: 3603)) positioned in VR VIII between amino acid residues 586 and 589, replacing amino acids A587 and Q588, and an AAV9 comprising an N4 targeting peptide (PLNGTVHLY (SEQ ID NO: 1232)) positioned in VR VIII between amino acid residues 586 and 589, replacing amino acids A587 and Q588. Tissues analyzed included frontal lobe, motor cortex, parietal lobe, occipital lobe, cerebellum, putamen, thalamus, globus pallidus, caudate, substantia nigra, liver, DRG-cervical, DRG-lumbar, and DRG-thoracic. Higher tissue enrichment score values represent greater tropism.

FIG. 38 provides sequences for a window of VR VIII of modified AAV capsid library having targeting peptides (e.g., X₁X₂X₃X₄X₅X₆X₇X₈X₉) where a residue at X₁ to X₉ is selected from the amino acid residues in the corresponding column. In the library, blank spaces at positions 587 and 588 correspond to deletion of A587 and Q588 from the modified AAV capsid protein. FIG. 38 discloses SEQ ID NOS 55875 and 55910, respectively, in order of appearance.

### 6. DETAILED DESCRIPTION OF THE INVENTION

### 6.1. Definitions

“AAV” is adeno-associated virus and may be used to refer to the virus itself or derivatives thereof. The term covers all subtypes, serotypes and pseudotypes, and both naturally occurring and recombinant forms, except where required otherwise.

The term “AAV capsid protein” or simply “capsid protein” refers to a VP1, VP2, or VP3 capsid protein of AAV. In some embodiments, the AAV capsid protein is a wild type or modified capsid protein of AAV9; AAV2; AAV1; AAV6; AAV3; AAV LK03; AAV7; AAV8; AAV hu.37; AAV rh 10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc8l; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; and Anc80DI.

The term “modified AAV capsid protein” or simply “modified capsid protein” refers to a capsid protein that is modified as compared to such naturally occurring or synthetic/artificial capsid protein, which is referred to as the “reference AAV capsid protein” or “reference capsid protein.” The reference AAV capsid protein as used herein may be a VP1, VP2, or VP3 capsid protein of a naturally occurring AAV variant or a non-naturally occurring VP1, VP2, or VP3 capsid protein that is known in the art.

The term “targeting peptide” as used herein refers to an amino acid sequence ranging from 5 to 16 amino acids in length within the variable region VIII (VRVIII) of a modified AAV capsid protein introduced by one or more modifications described herein. AAVs comprising a modified capsid protein with a targeting peptide can have localization and distribution in a target cell, tissue or organ different from the AAV with a capsid protein without the target peptide.

The term “amino acid position” within an AAV capsid protein as here herein refers to a position of an amino acid residue in an AAV VP1 protein sequence, counted from the first amino acid in the N terminal. As used herein, the term “amino acid” comprises naturally occurring L- and D-amino acids and artificial, i.e. non-naturally occurring, α-amino acids. Preferably, the amino acid is a naturally occurring amino acid. In preferred embodiments, the amino acid is a naturally occurring L-α-amino acid.

For the avoidance of doubt, as used herein, the indication that an insertion site is at amino acid position X means that the targeting peptide is inserted between amino acids X and X+1, i.e., the targeting peptide is inserted after the amino acid at position X and before the amino acid at position X+1.

The term “inverted terminal repeat” (or “ITR”) refers to a polynucleotide sequence found at the ends of AAV genomes that form a hairpin, which contributes to the genome's ability to self-prime (allowing for primase-independent synthesis of the complementary second DNA strand) and provides for encapsidation of the genome into an AAV particle. An ITR can be a wild-type ITR or a variant thereof.

The term “modification” when in conjunction with an amino acid residue, amino acid residues, or a modified sequence, refers to insertion(s), deletion(s), substitution(s) or a combination thereof.

The terms “operably linked” and “operatively linked” refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA.

The term “parenteral” administration of a composition includes, e.g., subcutaneous (s.c.), intravenous (i.v.), intramuscular (i.m.), or intrasternal injection, or infusion techniques.

The terms “peptide”, “polypeptide” and “protein” are used interchangeably herein to refer to a polymer of amino acid residues.

The term “pharmaceutically acceptable carrier” includes any of the standard pharmaceutical carriers, excipients, stabilizers and adjuvants. For examples of carriers, excipients, stabilizers and adjuvants, see Remington: The Science and Practice of Pharmacy, 22nd Revised Ed., Pharmaceutical Press, 2012.

The abbreviation “rAAV” refers to a recombinant adeno-associated viral particle composed of at least one AAV capsid protein and an encapsidated polynucleotide, sometimes referred to herein as a “genome”. rAAV can include a genome that comprises a heterologous polynucleotide (i.e., a polynucleotide other than a wild-type AAV genome), such as a heterologous polynucleotide encoding a gene delivered to a mammalian cell such as sequence encoding a therapeutic protein.

The terms “percent sequence identity” (% sequence identity), “percent identical” (% identical) and the like refer to percent sequence identity between two nucleotide sequences or between two amino acid sequences calculated by aligning the two sequences, determining the number of matches of nucleotides or amino acid residues between the two sequences, dividing the number of matches by the length of the aligned region (i.e., the number of aligned nucleotides or amino acid residues), and multiplying by 100 to arrive at a percent sequence identity value. For calculation of the percent sequence identity (% sequence identity), two or more sequences are aligned using the EMBOSS Needle Pairwise Sequence Alignment software tool based on the Needleman and Wunsch algorithm (available at www.ebi.ac.uk/Tools/psa/emboss_needle) with the following parameters: Matrix: BLOSUM62 (for protein sequences) or DNAfull (for DNA sequences); Gap Open: 10; Gap Extend: 0.5; End Gap Penalty: false; End Gap Open: 10; and End Gap Extend: 0.5.

Methods of alignment of nucleotide and amino acid sequences for comparison are well known in the art. The local homology algorithm (BESTFIT) of Smith and Waterman (1981) Adv. Appl. Math 2:482, may permit optimal alignment of compared sequences; by the homology alignment algorithm (GAP) of Needleman and Wunsch (1970) J. Mol. Biol. 48:443-453; by the search for similarity method (Tfasta and Fasta) of Pearson and Lipman (1988) Proc. Natl. Acad. Sci. USA 85:2444; by computerized implementations of these algorithms, including, but not limited to: CLUSTAL in the PC/Gene program by Intelligenetics, Mountain View, Calif, GAP, BESTFIT, BLAST, FASTA and TFASTA in the Wisconsin Genetics Software Package, Version 8 (available from Genetics Computer Group (GCG™ programs (Accelrys, Inc., San Diego, Calif.)). The CLUSTAL program is well described by Higgins and Sharp (1988) Gene 73:237-244; Higgins and Sharp (1989) CABIOS 5: 151-153; Corpet, et al. (1988) Nucleic Acids Res. 16: 10881-10890; Huang, et al. (1992) Computer Applications in the Biosciences 8: 155-165; and Pearson, et al. (1994) Meth. Mol. Biol. 24:307-331. An example of a good program to use for optimal global alignment of multiple sequences is PileUp (Feng and Doolittle (1987) J. Mol. Evol. 25:351-260, which is similar to the method described by Higgins and Sharp (1989) CABIOS 5: 151-153 (and is hereby incorporated by reference). The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., eds., Greene Publishing and Wiley-Interscience, New York (1995). An updated version of the BLAST family of programs includes the BLAST+ suite. (Camacho, C., et al. (2009 Dec. 15) BLAST+: architecture and applications. BMC Bioinformatics 10:421).

The term “tissue-specific” promoter or expression regulatory element (ERE) as used herein refers to a nucleotide sequence which, when operably linked with a polynucleotide encodes or specified by a gene, causes the gene product to be produced in a cell substantially only if the cell is a cell of the tissue type corresponding to the promoter.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “vector”, “AAV vector” and “rAAV vector” refer to an rAAV that comprises a heterologous polynucleotide, e.g., a transgene.

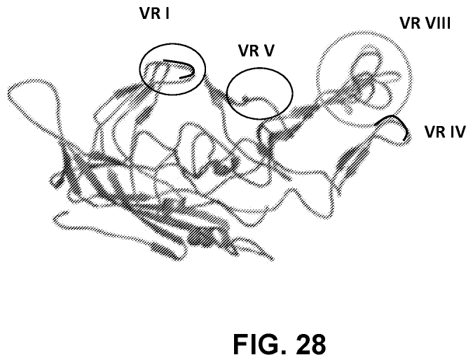

The terms “variable region” or “VR” as used herein refer to one or more of nine sequence variable regions (e.g., VRI to VRIX) in an AAV capsid protein previously defined by comparison and alignment of various AAV capsid proteins. See e.g., Govindasamy et al., Structurally mapping the diverse phenotype of adeno-associated virus serotype 4, J. Virol. (2006); Meyer et al. Structure of the gene therapy vector, adeno-associated virus with its cell receptor, AAVR, eLife (2019). The VRs are known to contain amino acids that contribute to slight differences in surface topologies and distinct functional phenotypes, such as in receptor binding, transduction efficiency, and antigenic re-activity. The relative positions of the VR 1, VR IV, VR V, and VIIII are illustrated in FIG. 28, but the specific positions of the variable regions within a capsid protein can vary depending on the capsid protein and/or the sequence alignment method.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### 6.2. Modified AAV Capsid Proteins

One aspect of the present disclosure provides a modified adeno-associated virus (AAV) capsid protein comprising: a targeting peptide within variable region VIII (VR VIII), wherein the targeting peptide has a sequence of X₁X₂X₃X₄X₅X₆X₇X₈X₉ and X₁, X₂, X₃, X₄, X₅, X₆, X₇, X₈, and X₉ are each independently selected from any amino acid residue.

In some embodiments, the modified adeno-associated virus (AAV) capsid protein comprises: a targeting peptide within VR VIII wherein the targeting peptide has a sequence selected from SEQ ID NOs: 620-55819.

In some embodiments, the modified adeno-associated virus (AAV) capsid protein comprises: a targeting peptide within variable region VIII (VR VIII), wherein the targeting peptide has a sequence of PX₂X₃GAVX₇LY (SEQ ID NO: 2) and X₂, X₃, and X₇ are independently selected from any amino acid residue.

In some embodiments, the modified adeno-associated virus (AAV) capsid protein comprises: a targeting peptide within variable region VIII (VR VIII), wherein the targeting peptide has a sequence of PX₂X₃GX₅X₆X₇LY (SEQ ID NO: 10) and X₂, X₃, X₅, X₆, and X₇ are independently selected from any amino acid residue.

In some embodiments, the modified adeno-associated virus (AAV) capsid protein comprises: a targeting peptide within variable region VIII (VR VIII), wherein the targeting peptide has a sequence of PX₂X₃GAVX₇X₈X₉ (SEQ ID NO: 20) and X₂, X₃, X₅, X₆, and X₇ are independently selected from any amino acid residue.

In some embodiments, the modified AAV capsid protein has one or more amino acid insertions, deletions, substitutions, or combinations thereof as compared to a reference AAV capsid protein. Some or all of the amino acid insertions, deletions, substitutions, or combinations thereof are for introduction of the targeting peptide into the reference AAV capsid protein. In some embodiments, all the differences between the modified AAV capsid protein and the reference AAV capsid protein are within variable region VIII (VR VIII). In some embodiments, all the differences between the modified AAV capsid protein and the reference AAV capsid protein are within the targeting peptide.

In some embodiments, the modified AAV capsid protein further comprises one or more modifications comprising an amino acid insertion, deletion, substitution, or a combination thereof outside of the introduction of the targeting peptide within VR VIII of the reference AAV capsid protein.

In some embodiments, the modified capsid protein further comprises one or more modifications outside of the VR VIII of the reference AAV capsid protein.

In some embodiments, the modified AAV capsid protein includes one or more modifications comprising an amino acid insertion, deletion, substitution, or a combination thereof to introduce the targeting peptide within VR VIII (e.g., wherein VR VIII corresponds to amino acids between positions 565 and 595 of the reference AAV capsid protein).

In some embodiments, the reference AAV capsid protein is an AAV9 capsid protein. In some embodiments, the modified AAV capsid protein has a sequence having at least 90%, 95%, 96%, 97%, 98% or 99% sequence identity to an AAV9 capsid protein. In some embodiments, the modified AAV capsid protein has a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from an AAV9 capsid protein. In some embodiments, the modified AAV capsid protein has a sequence having at least 90%, 95%, 96%, 97%, 98% or 99% sequence identity to an AAV9 capsid protein having a targeting peptide (e.g., any of the targeting peptides described herein) in VR VIII. In some embodiments, the modified AAV capsid protein has a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from an AAV9 capsid protein having a targeting peptide (e.g., any of the targeting peptides described herein) in VR VIII.

In some embodiments, the reference AAV capsid protein is an Anc80L65 capsid protein. In some embodiments, the modified AAV capsid protein has a sequence having at least 90%, 95%, 96%, 97%, 98% or 99% sequence identity to an Anc80L65 capsid protein. In some embodiments, the modified AAV capsid protein has a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from an Anc80L65 capsid protein. In some embodiments, the modified AAV capsid protein has a sequence having at least 90%, 95%, 96%, 97%, 98% or 99% sequence identity to an Anc80L65 capsid protein having a targeting peptide (e.g., any of the targeting peptides described herein) in VR VIII. In some embodiments, the modified AAV capsid protein has a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from an Anc80L65 capsid protein having a targeting peptide (e.g., any of the targeting peptides described herein) in VR VIII.

In some embodiments, the modified AAV capsid protein has a sequence having at least 90%, 95%, 96%, 97%, 98% or 99% sequence identity to a sequence selected from SEQ ID NOs: 34-38 or 55820-55847. In some embodiments, the modified AAV capsid protein has a sequence selected from SEQ ID NOs: 34-38 or 55820-55847. In some embodiments, the modified AAV capsid protein has a sequence selected from SEQ ID NOs: 34-39.

In some embodiments, the AAV virion comprising the modified AAV capsid protein, when administered in a therapeutically effective amount to a subject, has increased specificity to a central nervous system (CNS) tissue, relative to an AAV virion comprising a reference capsid protein without the targeting peptide.

In some embodiments, the AAV virion comprising the modified AAV capsid protein, when administered in a therapeutically effective amount to a subject, has increased transduction efficiency in the CNS, relative to an AAV virion comprising a reference capsid protein without the targeting peptide.

In some embodiments, the AAV virion comprising the modified AAV capsid protein, when administered in a therapeutically effective amount to a subject, has increased blood brain barrier penetration in the subject, relative to an AAV virion comprising a reference capsid protein without the targeting peptide.

In some embodiments, the AAV virion comprising the modified AAV capsid protein, when administered in a therapeutically effective amount to a subject, has decreased specificity to a central nervous system (CNS) tissue, relative to an AAV virion comprising a reference capsid protein without the targeting peptide.

In some embodiments, the AAV virion comprising the modified AAV capsid protein, when administered in a therapeutically effective amount to a subject, has decreased transduction efficiency in the CNS, relative to an AAV virion comprising a reference capsid protein without the targeting peptide.

In some embodiments, the AAV virion comprising the modified AAV capsid protein, when administered in a therapeutically effective amount to a subject, has decreased blood brain barrier penetration in the subject, relative to an AAV virion comprising a reference capsid protein without the targeting peptide.

### 6.2.1. Targeting Peptide

In some embodiments, the targeting peptide within variable region VR VIII has a sequence of X₁X₂X₃X₄X₅X₆X₇X₈X₉ and X₁, X₂, X₃, X₄, X₅, X₆, X₇, X₈, and X₉ are each independently selected from any amino acid residue. In some embodiments, the modified capsid protein comprises the targeting peptide having a sequence of X₁X₂X₃X₄X₅X₆X₇X₈X₉ at a site within variable region VIII (VR VIII), wherein the targeting peptide is inserted between S586 and A589 of an AAV9 capsid protein, thereby replacing A587 and Q588 of the AAV9 capsid protein. In some embodiments,
(i) X₁ is independently selected from a proline (P) and a glycine (G);
(ii) X₂ is independently selected from a lysine (L), a threonine (T), a serine (S), an alanine (A), a valine (V), and an isoleucine (I);
(iii) X₃ is independently selected from an asparagine (N), a glutamine (Q), and a proline (P);
(iv) X₄ is independently selected from a glycine (G) and an alanine (A);
(v) X₅ is independently selected from an alanine (A), a threonine (T), a serine (S), a valine (V), and a glycine (G);
(vi) X₆ is independently selected from a valine (V), a leucine (L), an alanine (A), an isoleucine (I), a glycine (G), a serine (S), and a threonine (T);
(vii) X₇ is independently selected from a histidine (H), an arginine (R), and a lysine (K);
(viii) X₈ is independently selected from a leucine (L) and a valine (V); and
(ix) X₉ is independently selected from a tyrosine (Y), an arginine (R), a histidine (H), a lysine (K), and a phenylalanine (F).

In some embodiments, the modified AAV capsid protein comprises a targeting peptide having at least 90%, at least 95%, at least 98%, at least 99%, or at least 100% sequence identity to an amino acid sequence selected from SEQ ID NOs.: 620-55819 that is introduced into VRVIII. In some embodiments, the modified AAV capsid protein comprises a targeting peptide where the peptide has a sequence that differs from a comparative peptide by substitution of 0, 1, 2, 3 or 4 residues to an amino acid sequence selected from SEQ ID NOs.: 620-55819 that is introduced into VR VIII.

In some embodiments, the targeting peptide has a sequence of PX₂X₃GAVX₇LY (SEQ ID NO: 2) and X₂, X₃, and X₇ are independently selected from any amino acid residue. In some embodiments, the modified capsid protein comprises the targeting peptide having the sequence of PX₂X₃GAVX₇LY (SEQ ID NO: 2), wherein insertion of the targeting peptide replaces amino acids residues A587 and Q588. In some embodiments,
(i) X₂ is independently selected from a lysine (L), an isoleucine (I), a valine (V), and an alanine (A);
(ii) X₃ is an asparagine (N) or a glutamine (Q); and
(iii) X₇ is independently selected from a histidine (H), an arginine (R) and a lysine (K).

In some embodiments, the targeting peptide is selected from:

```
  (SEQ ID NO: 3)
(i) PLQGAVHLY;
  (SEQ ID NO: 4)
(ii) PLQGAVRLY;
  (SEQ ID NO: 5)
(iii) PLQGAVKLY;
  (SEQ ID NO: 6)
(iv) PINGAVHLY;
  (SEQ ID NO: 7)
(v) PVNGAVHLY;
  (SEQ ID NO: 8)
(vi) PANGAVHLY;
or
(SEQ ID NO: 9)
(vii) PLNGAVHLY.
```

In some embodiments, the targeting peptide is PLNGAVHLY (SEQ ID NO: 9). In some embodiments, the targeting peptide is not PLNGAVHLY (SEQ ID NO: 9).

In some embodiments, the targeting peptide the targeting peptide has a sequence selected from SEQ ID NOs: 620-55819.

In some embodiments, the targeting peptide has a sequence of PX₂X₃GX₅X₆X₇LY (SEQ ID NO: 10) and X₂, X₃, X₅, X₆, and X₇ are independently selected from any amino acid residue. In some embodiments, the modified capsid protein comprising the targeting peptide having the sequence of PX₂X₃GX₅X₆X₇LY (SEQ ID NO: 10), wherein insertion of the targeting peptide replaces amino acids residues A587 and Q588. In some embodiments,
(i) X₂ is independently selected from a leucine (L), a threonine (T), or a serine (S);
(ii) X₃ is independently selected from an asparagine (N) and a glutamine (Q);
(iii) X₅ is independently selected from an alanine (A) and a threonine (T);
(iv) X₆ is independently selected from a valine (V) and a leucine (L); and
(v) X₇ is independently selected from a histidine (H), an arginine (R), and a lysine (K).

In some embodiments, the targeting peptide is selected from:

```
  (SEQ ID NO: 11)
(i) PTNGTVRLY;
  (SEQ ID NO: 12)
(ii) PTNGTVHLY;
  (SEQ ID NO: 13)
(iii) PTNGTVKLY;
  (SEQ ID NO: 14)
(iv) PSNGTLRLY;
  (SEQ ID NO: 15)
(v) PSNGTLHLY;
  (SEQ ID NO: 16)
(vi) PSNGTLKLY;
(SEQ ID NO: 17)
(vii) PTNGTLRLY;
  (SEQ ID NO: 18)
(viii) PTNGTLHLY;
or
(SEQ ID NO: 19)
(ix) PTNGTLKLY.
```

In some embodiments, the targeting peptide has a sequence of PX₂X₃GAVX₇X₈X₉ (SEQ ID NO: 20) and X₂, X₃, X₅, X₆, and X₇ are independently selected from any amino acid residue. In some embodiments, the modified capsid protein comprising the targeting peptide having the sequence of PX₂X₃GAVX₇X₈X₉ (SEQ ID NO: 20), wherein insertion of the targeting peptide replaces amino acids residues A587 and Q588. In some embodiments,
(i) X₂ is independently selected from a leucine (L), a threonine (T), or a serine (S);
(ii) X₃ is independently selected from an asparagine (N) and a glutamine (Q);
(iii) X₇ is independently selected from a histidine (H) and a threonine (T);
(iv) X₈ is independently selected from a valine (V) and a leucine (L); and
(v) X₉ is independently selected from a tyrosine (Y) and an arginine (R).

In some embodiments, the targeting peptide is selected from:

```
  (SEQ ID NO: 21)
(i) PTQGAVTVR;
  (SEQ ID NO: 22)
(ii) PLQGAVTVR;
  (SEQ ID NO: 23)
(iii) PLQGAVHVR;
  (SEQ ID NO: 24)
(iv) PLQGAVHVY;
  (SEQ ID NO: 25)
(v) PSQGAVTLR;
  (SEQ ID NO: 26)
(vi) PLQGAVTLR;
  (SEQ ID NO: 27)
(vii) PLQGAVHLR;
or
(SEQ ID NO: 28)
(viii) PTQGAVTLR.
```

In some embodiments, the targeting peptide is PLNGSVHLY (SEQ ID NO: 3603).

In some embodiments, the modified AAV capsid protein comprises a modified AAV9 capsid protein comprising a targeting peptide having the sequence of PLNGSVHLY (SEQ ID NO: 3603) positioned in VR VIII between amino acid residues 586 and 589, replacing amino acids A587 and Q588.

In some embodiments, the targeting peptide is PLNGTVHLY (SEQ ID NO: 1232).

In some embodiments, the modified AAV capsid protein comprises a modified AAV9 capsid protein comprising a targeting peptide having the sequence of PLNGTVHLY (SEQ ID NO: 1232) positioned in VR VIII between amino acid residues 586 and 589, replacing amino acids A587 and Q588.

In some embodiments, the targeting peptide is anyone selected from Appendix B.

In some embodiments, the targeting peptide does not comprise a sequence of SEQ ID NO: 9.

In some embodiments, the targeting peptide within VR VIII wherein the targeting peptide has a sequence selected from SEQ ID NO: 160-619. In some embodiments, the targeting peptide is inserted into an AAV9 or Anc80L65 backbone.

In some embodiments, the modified AAV capsid protein comprises a targeting peptide having at least 90%, at least 95%, at least 98%, at least 99%, or at least 100% sequence identity to the amino acid sequence selected from SEQ ID NOs.: 160-619 that is introduced into the VRVIII. In some embodiments, the modified AAV capsid protein comprises a targeting peptide where the peptide has a sequence that differs from a comparative peptide by substitution of 0, 1, 2, 3 or 4 residues to an amino acid sequence selected from SEQ ID NOs.: 160-619 that is introduced into VR VIII.

In some embodiments, the modified AAV capsid protein comprises a targeting peptide having a sequence selected from SEQ ID NOs: 518, 396, 393, 186, 368, 493, 179, 217, 614, 475, 523, 411, 443, 192, 403, 266, 416, 579, 619, 589, 247, 367, 263, 410, 615, and 597. In some embodiments, the modified AAV capsid protein comprises a targeting peptide having a sequence selected from SEQ ID NOs: 518, 396, 393, 186, 368, 493, 179, 217, 614, 475, 523, 411, 443, 192, 403, 266 and 416. In some embodiments, the modified AAV capsid protein comprises a targeting peptide having a sequence selected from SEQ ID NOs: 518, 396, 393, 186 and 368. In some embodiments, the modified AAV capsid protein comprises a targeting peptide having a sequence selected from Appendix A.

In some embodiments, the targeting peptide is the targeting peptide disclosed in US2017/0166926, incorporated by reference in its entirety herein.

In some embodiments, the targeting peptide is not a targeting peptide disclosed in WO 2020/210655; WO 2021/222831; WO 2021/202651; WO 2021/211753; WO 2021/226167; WO 2021/230987; or WO 2022/040527, incorporated by reference in their entireties herein.

In some embodiments, the targeting peptide does not have a sequence identified in the variable region VR VIII of a reference AAV capsid protein.

### 6.2.2. Targeting Peptide Site

A modified AAV capsid protein of the present disclosure comprises a targeting peptide within VR VIII of the reference AAV capsid protein (FIG. 28).

Preferably, the targeting peptide is at a site exposed to the exterior of the capsid, preferably based on structure predictions and/or experimental data. More preferably, the targeting peptide is at a site exposed to the exterior of the AAV capsid in a manner that does not interfere with the activity of said protein in capsid assembly.

In some embodiments, the one or more modifications comprises an amino acid insertion, deletion, substitution, or a combination thereof to introduce the targeting peptide into VR VIII of the reference AAV capsid protein.

In some embodiments, the one or more modifications comprises one or more amino acid deletions within VR VIII. In some embodiments, the one or more amino acid deletions comprise a deletion of one, two, three, four, or five or more amino acid residue immediately adjacent to the N terminal end of the targeting peptide within VR VIII. In some embodiments, the one or more amino acid deletions comprise a deletion of one amino acid residue immediately adjacent to the N terminal end of the targeting peptide within VR VIII. In some embodiments, the one or more amino acid deletions comprise a deletion of two amino acid residue immediately adjacent to the N terminal end of the targeting peptide within VR VIII. In some embodiments, the one or more amino acid deletions comprise a deletion of three amino acid residue immediately adjacent to the N terminal end of the targeting peptide within VR VIII. In some embodiments, the one or more amino acid deletions comprise a deletion of four amino acid residue immediately adjacent to the N terminal end of the targeting peptide within VR VIII.

In some embodiments, the one or more amino acid deletions comprise a deletion of at least one amino acid residue at a position selected from 584, 585, 586, 587, or 588, or a combination thereof, relative to a reference sequence numbered according to the amino acid sequence of the reference AAV capsid protein. In some embodiments, the one or more amino acid deletions comprise a deletion of an amino acid residue at position 587, relative to a reference sequence numbered according to the amino acid sequence of the reference AAV capsid protein. In some embodiments, the one or more amino acid deletions comprise a deletion of an amino acid residue at position 588, relative to a reference sequence numbered according to the amino acid sequence of the reference AAV capsid protein. In some embodiments, the one or more amino acid deletions comprise a deletion of amino acid residues at positions 587 and 588, relative to a reference sequence numbered according to the amino acid sequence of the reference AAV capsid protein. In some embodiments, the one or more amino acid deletions comprise a deletion of amino acid residues at positions 586, 587 and 588, relative to a reference sequence numbered according to the amino acid sequence of the reference AAV capsid protein. In some embodiments, the one or more amino acid deletions comprise a deletion of amino acid residues at positions 585, 586, 587 and 588, relative to a reference sequence numbered according to the amino acid sequence of the reference AAV capsid protein.

In some embodiments, the one or more amino acid deletions comprises deletion of one, two, three, four, or five or more amino acid residues immediately adjacent to the C terminal end of the targeting peptide within VR VIII. In some embodiments, the one or more amino acid deletions comprise a deletion of one amino acid residue immediately adjacent to the C terminal end of the targeting peptide within VR VIII. In some embodiments, the one or more amino acid deletions comprise a deletion of two amino acid residue immediately adjacent to the C terminal end of the targeting peptide within VR VIII. In some embodiments, the one or more amino acid deletions comprise a deletion of three amino acid residue immediately adjacent to the C terminal end of the targeting peptide within VR VIII. In some embodiments, the one or more amino acid deletions comprise a deletion of four amino acid residue immediately adjacent to the C terminal end of the targeting peptide within VR VIII.

In some embodiments, the amino acid deletion comprises deletion of an amino acid residue at position 589, 590, or 591, or a combination thereof, relative to a reference sequence numbered according to the amino acid sequence of the reference AAV capsid protein. In some embodiments, the amino acid deletion comprises deletion of an amino acid residue at position 589, relative to a reference sequence numbered according to the amino acid sequence of the reference AAV capsid protein. In some embodiments, the amino acid deletion comprises deletion of an amino acid residue at position 590, relative to a reference sequence numbered according to the amino acid sequence of the reference AAV capsid protein. In some embodiments, the amino acid deletion comprises deletion of an amino acid residue at position 591, relative to a reference sequence numbered according to the amino acid sequence of the reference AAV capsid protein. In some embodiments, the amino acid deletion comprises deletion of an amino acid residues at positions 589 and 590, relative to a reference sequence numbered according to the amino acid sequence of the reference AAV capsid protein. In some embodiments, the amino acid deletion comprises deletion of an amino acid residues at positions 590 and 591, relative to a reference sequence numbered according to the amino acid sequence of the reference AAV capsid protein. In some embodiments, the amino acid deletion comprises deletion of an amino acid residues at positions 589, 590, and 591, relative to a reference sequence numbered according to the amino acid sequence of the reference AAV capsid protein.

In some embodiments, the one or more modifications comprises an amino acid insertion to introduce the targeting peptide into VR VIII of the reference AAV capsid protein. In some embodiments, the amino acid insertion comprises insertion of one, two, three, four, or five or more amino acid residues immediately adjacent to C terminal end of the targeting peptide within VR VIII. In some embodiments, the one or more amino acid insertions comprise an insertion of one amino acid residue immediately adjacent to the C terminal end of the targeting peptide within VR VIII. In some embodiments, the one or more amino acid insertions comprise an insertion of two amino acid residue immediately adjacent to the C terminal end of the targeting peptide within VR VIII. In some embodiments, the one or more amino acid insertions comprise an insertion of three amino acid residue immediately adjacent to the C terminal end of the targeting peptide within VR VIII. In some embodiments, the one or more amino acid insertions comprise an insertion of four amino acid residue immediately adjacent to the C terminal end of the targeting peptide within VR VIII. In some embodiments, the one or more amino acid insertions comprise an insertion of five amino acid residue immediately adjacent to the C terminal end of the targeting peptide within VR VIII.

In some embodiments, the one or more modifications comprises an amino acid insertion to introduce the targeting peptide into VR VIII of the reference AAV capsid protein. In some embodiments, the amino acid insertion comprises insertion of one, two, three, four, or five or more amino acid residues immediately adjacent to N terminal end of the targeting peptide within VR VIII. In some embodiments, the one or more amino acid insertions comprise an insertion of one amino acid residue immediately adjacent to the N terminal end of the targeting peptide within VR VIII. In some embodiments, the one or more amino acid insertions comprise an insertion of two amino acid residue immediately adjacent to the N terminal end of the targeting peptide within VR VIII. In some embodiments, the one or more amino acid insertions comprise an insertion of three amino acid residue immediately adjacent to the N terminal end of the targeting peptide within VR VIII. In some embodiments, the one or more amino acid insertions comprise an insertion of four amino acid residue immediately adjacent to the N terminal end of the targeting peptide within VR VIII. In some embodiments, the one or more amino acid insertions comprise an insertion of five amino acid residue immediately adjacent to the N terminal end of the targeting peptide within VR VIII.

In some embodiments, the inserted amino acid residue is independently selected from any amino acid residue. In some embodiments where VR VIII comprises an insertion and a deletion relative to the amino acid sequence of the reference AAV capsid protein, the inserted amino acid residue(s) is identical to the amino acid residue deleted adjacent to the N terminal end or C terminal end of the targeting peptide within VR VIII. In some embodiments, the inserted amino acid residue is an alanine (A) or an asparagine (N). In some embodiments, the inserted amino acid residue is an alanine (A). In some embodiments, the inserted amino acid residue is or an asparagine (N). In some embodiments, the inserted amino acids are an alanine (A) at the position immediately adjacent to the C terminal end of the targeting peptide and an asparagine (N) at the next subsequent position, thereby having an amino acid sequence of AN immediately adjacent to the C terminal end of the targeting peptide.

In some embodiments where the modified capsid protein includes an amino acid insertion in VR VIII, the targeting peptide comprises: (i) PLNGAVHLYN (SEQ ID NO: 32); or (ii) PLNGAVHLYAN (SEQ ID NO: 33).

In some embodiments, the one or more modifications comprises an amino acid insertion, an amino acid deletion and/or an amino acid substitution to introduce the targeting peptide into VR VIII of the reference AAV capsid protein. In one embodiment, a modified capsid protein includes insertion of two amino acid residues immediately adjacent to the C-terminal end of the targeting peptide and a deletion of four amino acid residues immediately adjacent to the N-terminal end of the targeting peptide. In another embodiment, a modified capsid protein includes insertion of one amino acid residue immediately adjacent to the C-terminal end of the targeting peptide and a deletion of three amino acid residues immediately adjacent to the N-terminal end of the targeting peptide. In yet another embodiment, a modified capsid protein includes insertion of two amino acid residues immediately adjacent to the C-terminal end of the targeting peptide and a deletion of two amino acid residues immediately adjacent to the N-terminal end of the targeting peptide. In yet another embodiment, a modified capsid protein includes no insertion of amino acid residues immediately adjacent to the C-terminal end of the targeting peptide and a deletion of one amino acid residues immediately adjacent to the N-terminal end of the targeting peptide.

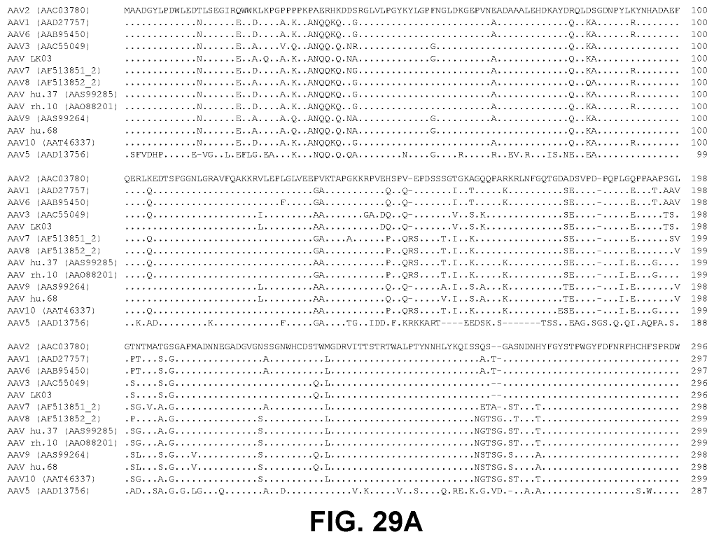

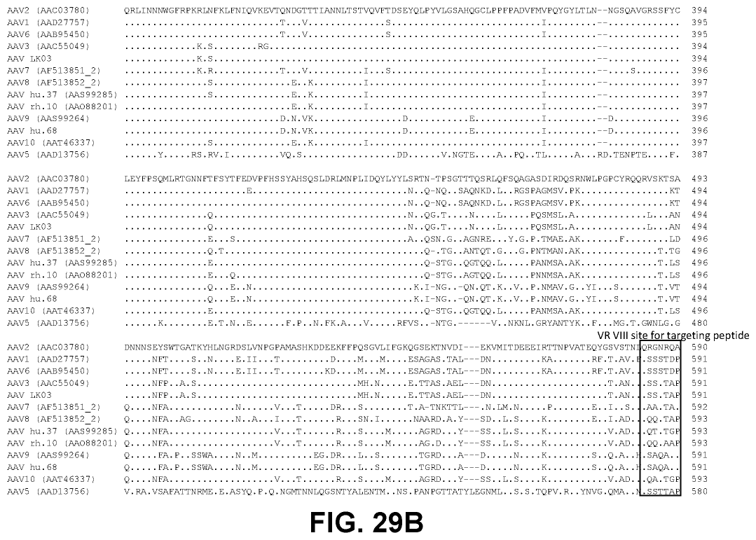

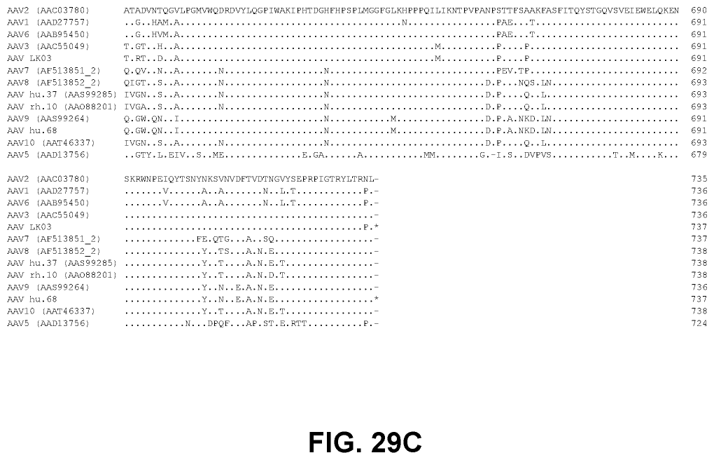

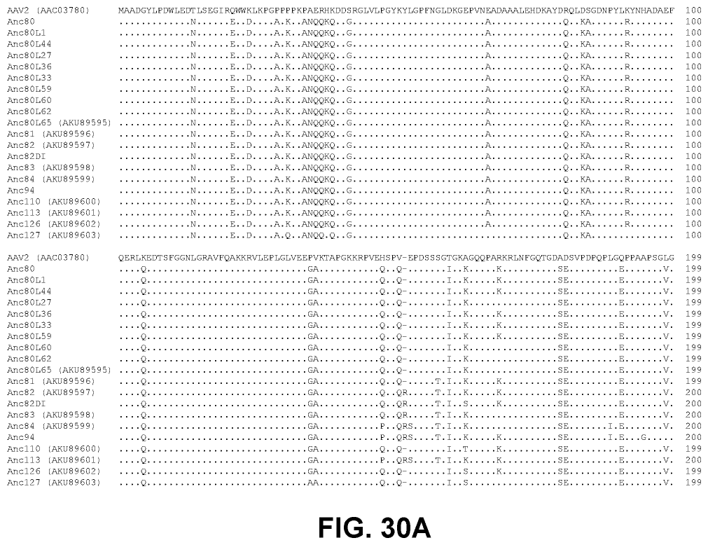

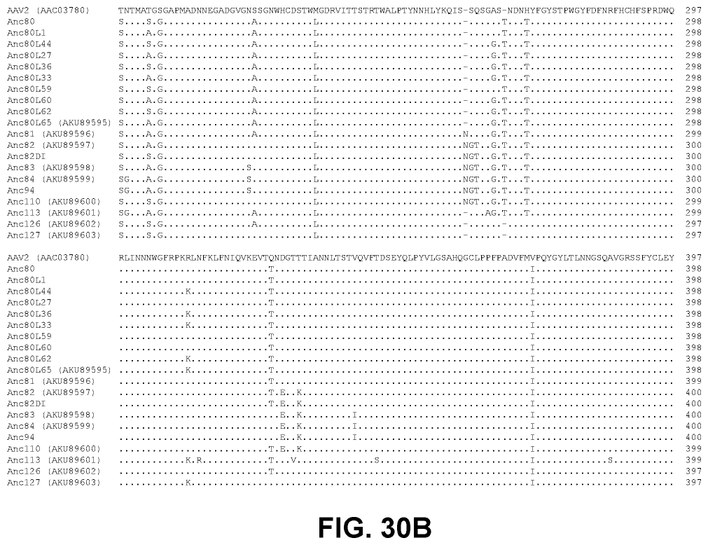

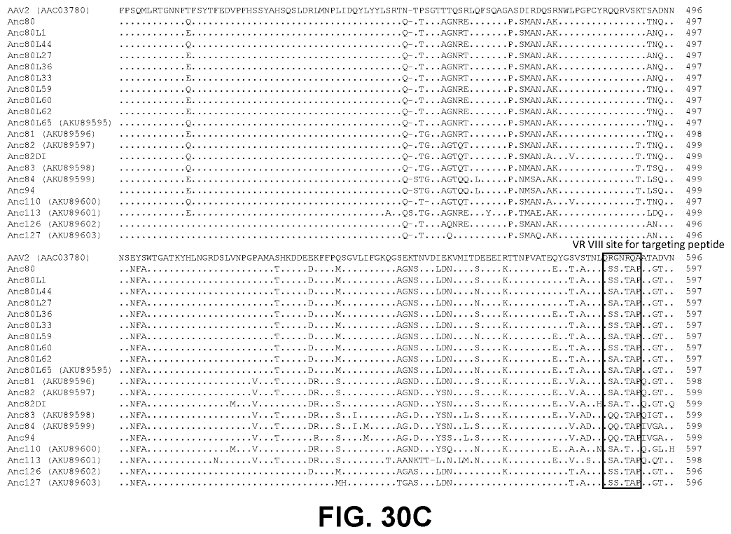

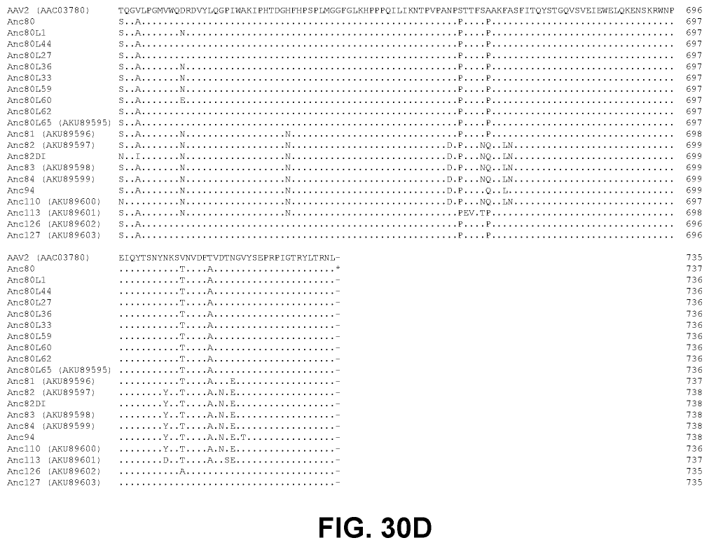

In some embodiments, insertion sites for the targeting peptides are provided in FIGS. 29A-29C and FIGS. 30AE-30D. In some embodiments, insertions sites for the targeting peptides are provided in FIG. 29B. In some embodiments, the insertion sites for the targeting peptides are provided in FIG. 30C.

In some embodiments, insertion sites for the targeting peptides are provided in Table 1. In Table 1, the preferred sites are indicated by a “-” relative to wild type VP₁ capsid polypeptide. In some embodiments, Table 1 includes exemplary insertion sites for targeting peptides selected from SEQ ID NO: 620-55819. In some embodiments, Table 1 includes exemplary insertion sites for targeting peptides selected from SEQ ID NO: 160-619.

TABLE 1
Exemplary insertion sites

| | Insertion sites 1 | | Insertion sites 2 | | Insertion sites 3 | |
|---|---|---|---|---|---|---|
| AAV1 | D590 | STD- | S588 | SSS- | S586 | S[ ][ ]-T |
| | P591 | PAT | T589 | TDP | T589 | |
| AAV2 | R588 | GNR- | N587 | RGN- | R585 | R[ ][ ]-R |
| | Q589 | QAA | R588 | RQA | R588 | |
| AAV3b | S586 | LQS- | N588 | SSN- | S586 | S[ ][ ]-T |
| | S587 | SNT | T589 | TAP | T589 | |
| AAV4 | S584 | DQS- | S586 | SNS- | D582 | D[ ][ ]-N |
| | N585 | NSN | N587 | NLP | N585 | |
| AAV5 | S575 | NQS- | T577 | SST- | S575 | S[ ][ ]-T |
| | S576 | STT | T578 | TAP | T578 | |
| AAV6 | D590 | STD- | S588 | SSS- | S586 | S[ ][ ]-T |
| | P591 | PAT | T589 | TDP | T589 | |
| AAV7 | N589 | AAN- | | | A587 | A[ ][ ]-T |
| | T590 | TAA | | | T590 | |
| AAV8 | N590 | QQN- | | | Q588 | Q[ ][ ]-T |
| | T591 | TAP | | | T591 | |
| AAV9 | Q588 | SAQ- | | | S586 | S[ ][ ]-A |
| | A589 | AQA | | | A589 | |
| AAVrh10 | N590 | QQN- | | | Q588 | Q[ ][ ]-A |
| | A591 | AAP | | | A591 | |
| AAVpo.1 | N567 | NON- | N569 | NSN- | N565 | N[ ][ ]-S |
| | S568 | SNT | T570 | THP | S568 | |
| AAV12 | N592 | NQN- | T594 | NAT- | N590 | N[ ][ ]-A |
| | A593 | ATT | T595 | TAP | A593 | |
| Anc80 | N588 | QSXN-T | | | S586 | S[ ][ ]-T |
| | T589 | | | | T589 | |
| Anc80L65 | N588 | QSAN-T | | | S586 | S[ ][ ]-T |
| | T589 | | | | T589 | |
| Anc80-55 | N588 | QSSN-T | | | S586 | S[ ][ ]-T |
| | T589 | | | | T589 | |
| Anc80-129 | N588 | QSAN-T | | | S586 | S[ ][ ]-T |
| | T589 | | | | T589 | |
| Anc80-156 | N588 | QSAN-T | | | S586 | S[ ][ ]-T |
| | T589 | | | | T589 | |
| Anc80-751 | N588 | QSAN-T | | | S586 | S[ ][ ]-T |
| | T589 | | | | T589 | |
| Anc80-1029 | N588 | QSAN-T | | | S586 | S[ ][ ]-T |
| | T589 | | | | T589 | |
| Anc80-1712 | N588 | QSAN-T | | | S586 | S[ ][ ]-T |
| | T589 | | | | T589 | |

Three exemplary insertion sites are indicated in Table 1. For insertion sites 1 and 2, the targeting peptide is inserted between 2 amino acid positions, where the insertion site is denoted as “-”. For example, for AAV1 capsid protein insertion site 1, the targeting peptide is inserted between positions D590 and P591. In some embodiments, the AAV capsid protein is modified by way of mutation or substitution of one or more amino acids, followed by insertion of the targeting peptide. For example, for insertion site 3 of Table 1, two amino acids are deleted prior to insertion of the targeting peptide. In such cases, “[ ]” represents a deleted amino acid residue/position. For example, for AAV9 capsid protein insertion site 3, the targeting peptide is inserted between position S586 and A589 after the amino acids “AQ” positioned between A586 and A589 (A587 and Q588) are deleted, denoted as “S[ ][ ]-A”.

In some embodiments, the targeting peptide is at between 560 and 610 within the VR VIII of the modified AAV capsid protein. In some embodiments, the targeting peptide is at between 565 and 605 within the VR VIII of the modified AAV capsid protein. In some embodiments, the targeting peptide is at between 570 and 600 within the VR VIII of the modified AAV capsid protein. In some embodiments, the targeting peptide is at between 575 and 595 within the VR VIII of the modified AAV capsid protein. In some embodiments, the targeting peptide is at between 580 and 590 within the VR VIII of the modified AAV capsid protein.

In some embodiments, the reference AAV capsid protein is a capsid protein of AAV1 or a modification thereof and the targeting peptide is between D590 and P591 or between S588 and T589 of the modified AAV capsid protein. In some embodiments, the reference AAV capsid protein is a capsid protein of AAV1 or a modification thereof and the targeting peptide is between positions 587 and 594 or between positions 585 and 592 of the modified AAV capsid protein. In some embodiments, the reference AAV capsid protein is a capsid protein of AAV1 or a modification thereof and the targeting peptide is between S586 and T589 of the reference AAV capsid protein and amino acid residues S587 and S588 are deleted.

In some embodiments, the reference AAV capsid protein is a capsid protein of AAV2 or a modification thereof and the targeting peptide is between R585 and Q589 or between N587 and R588 of the modified AAV capsid protein. In some embodiments, the reference AAV capsid protein is a capsid protein of AAV2 or a modification thereof and the targeting peptide is between positions 582 and 592 or between positions 585 and 591 of the modified AAV capsid protein. In some embodiments, the reference AAV capsid protein is a capsid protein of AAV2 or a modification thereof and the targeting peptide is between R585 and R588 thereby replacing amino acid residues G586 and N587.

In some embodiments, the reference AAV capsid protein is a capsid protein of AAV3 or a modification thereof and the targeting peptide is between S586 and S587 or between N588 and T589 of the modified AAV capsid protein. In some embodiments, the reference AAV capsid protein is a capsid protein of AAV3 or a modification thereof and the targeting peptide is between positions 583 and 590 or between positions 585 and 592 of the modified AAV capsid protein. In some embodiments, the reference AAV capsid protein is a capsid protein of AAV3 or a modification thereof and the targeting peptide is between S586 and T589 of the reference AAV capsid protein, thereby replacing amino acid residues S587 and N588.

In some embodiments, the reference AAV capsid protein is a capsid protein of AAV4 and the targeting peptide is between S584 and N585 or between S586 and N587 of the modified AAV capsid protein. In some embodiments, the reference AAV capsid protein is a capsid protein of AAV4 or a modification thereof and the targeting peptide is between positions 581 and 586 or between positions 583 and 590 of the modified AAV capsid protein. In some embodiments, the reference AAV capsid protein is a capsid protein of AAV4 or a modification thereof and the targeting peptide is between D582 and N585 of the reference AAV capsid protein, thereby replacing amino acid residues Q583 and S584.

In some embodiments, the reference AAV capsid protein is a capsid protein of AAV5 or a modification thereof and the targeting peptide is between S575 and S576 or between T577 and T578 of the capsid protein. In some embodiments, the reference AAV capsid protein is a capsid protein of AAV5 or a modification thereof and the targeting peptide is between positions 572 and 579 or between positions 574 and 581 of the capsid protein. In some embodiments, the reference AAV capsid protein is a capsid protein of AAV5 and the targeting peptide is between S575 and T578 of the reference AAV capsid protein, thereby replacing S576 and S577.

In some embodiments, the reference AAV capsid protein is a capsid protein of AAV6 or a modification thereof and the targeting peptide is between D590 and P591 or S588 and T589 of the modified AAV capsid protein. In some embodiments, the reference AAV capsid protein is a capsid protein of AAV6 or a modification thereof and the targeting peptide is between positions 587 and 594 or positions 585 and 592 of the modified AAV capsid protein. In some embodiments, the reference AAV capsid protein is a capsid protein of AAV6 or a modification thereof and the targeting peptide is between S586 and T589 of the reference AAV capsid protein, thereby replacing amino acid residues S587 and S588.

In some embodiments, the reference AAV capsid protein is a capsid protein of AAV7 or a modification thereof and the targeting peptide is between N589 and T590 of the modified AAV capsid protein. In some embodiments, the reference AAV capsid protein is a capsid protein of AAV7 or a modification thereof and the targeting peptide is between positions 586 and 593 of the modified AAV capsid protein. In some embodiments, the reference AAV capsid protein is a capsid protein of AAV7 or a modification thereof and the targeting peptide is between A587 and T590 of the reference AAV capsid protein, thereby replacing amino acid residues A588 and N589.

In some embodiments, the reference AAV capsid protein is a capsid protein of AAV8 and the targeting peptide is between N590 and T591 of the modified AAV capsid protein. In some embodiments, the reference AAV capsid protein is a capsid protein of AAV8 or a modification thereof and the targeting peptide is between positions 587 and 594 of the modified AAV capsid protein. In some embodiments, the reference AAV capsid protein is a capsid protein of AAV8 or a modification thereof and the targeting peptide is between Q588 and T591 of the modified AAV capsid protein, thereby replacing amino acid residues Q589 and N590.

In some embodiments, the reference AAV capsid protein is a capsid protein of AAV9 and the targeting peptide is between Q588 and A589 of the modified AAV capsid protein. In some embodiments, the reference AAV capsid protein is a capsid protein of AAV9 or a modification thereof and the targeting peptide is between positions 585 and 592 of the modified AAV capsid protein. In some embodiments, the reference AAV capsid protein is a capsid protein of AAV9 or a modification thereof and the targeting peptide is between S586 and A589 of the reference AAV capsid protein, thereby replacing amino acid residues A587 and Q588.

In some embodiments, the reference AAV capsid protein is a capsid protein of AAVrh10 or a modification thereof and the targeting peptide is between N590 and A591 of the modified AAV capsid protein. In some embodiments, the reference AAV capsid protein is a capsid protein of AAVrh10 or a modification thereof and the targeting peptide is between positions 587 and 594 of the modified AAV capsid protein. In some embodiments, the reference AAV capsid protein is a capsid protein of AAVrh10 or a modification thereof and the targeting peptide is between Q588 and A591 of the reference AAV capsid protein, thereby replacing amino acid residues Q589 and N590.

In some embodiments, the reference AAV capsid protein is a capsid protein of AAVpo.1 or a modification thereof and the targeting peptide is between N567 and S568 or between N569 and T570 of the modified AAV capsid protein. In some embodiments, the reference AAV capsid protein is a capsid protein of AAVpo.1 or a modification thereof and the targeting peptide is between positions 570 and 571 or between positions 566 and 573 of the modified AAV capsid protein. In some embodiments, the reference AAV capsid protein is a capsid protein of AAVpo.1 or a modification thereof and the targeting peptide is between N565 and S568 of the reference AAV capsid protein, thereby replacing amino acid residues Q566 and N567.

In some embodiments, the reference AAV capsid protein is a capsid protein of AAV12 or a modification thereof and the targeting peptide is between N592 and A593 or between T594 and T595 of the modified AAV capsid protein. In some embodiments, the reference AAV capsid protein is a capsid protein of AAV12 or a modification thereof and the targeting peptide is between positions 589 and 596 or between positions 591 and 598 of the modified AAV capsid protein. In some embodiments, the reference AAV capsid protein is a capsid protein of AAV12 or a modification thereof and the targeting peptide is between N590 and A593 of the reference AAV capsid protein, thereby replacing amino acid residues Q591 and N592.

In some embodiments, the reference AAV capsid protein is a capsid protein of Anc80 or a modification thereof and the targeting peptide is between T589 and A590, between N588 and T589, or between S587 and N588 of the modified AAV capsid protein. In some embodiments, the reference AAV capsid protein is a capsid protein of Anc80 or a modification thereof and the targeting peptide is between positions 586 and 593 or between positions 584 and 591 of the modified AAV capsid protein. In some embodiments, the reference AAV capsid protein is a capsid protein of Anc80 or a modification thereof and the targeting peptide is between S586 and T589 of the reference AAV capsid protein, thereby replacing amino acid residues S587 and N588.

In some embodiments, the reference AAV capsid protein is a capsid protein of Anc80L65 or a modification thereof and the targeting peptide is between T589 and A590, between N588 and T589 or between A587 and N588 of the modified AAV capsid protein. In some embodiments, the reference AAV capsid protein is a capsid protein of Anc80L65 or a modification thereof and the targeting peptide is between positions 586 and 593 or between positions 584 and 591 of the modified AAV capsid protein.

In some embodiments, the reference capsid protein is a capsid protein of Anc80L65 or a modification thereof and the targeting peptide is (i) between S586 and T589 of an Anc80L65 capsid protein, thereby replacing A587 and N588 of the Anc80L65 capsid protein; (ii) between Q585 and N588 of an Anc80L65 capsid protein, thereby replacing S586 and A587 of the Anc80L65 capsid protein; (iii) between L584 and A587 of an Anc80L65 capsid protein, thereby replacing Q585 and S586 of the Anc80L65 capsid protein; (iv) between A587 and A590 of an Anc80L65 capsid protein, thereby replacing N588 and T589 of the Anc80L65 capsid protein; or (v) between S586 and A587 of an Anc80L65 capsid protein.

In some embodiments, the reference AAV capsid protein is a capsid protein of Anc80-55 or a modification thereof and the targeting peptide is between T589 and A590 or between S587 and N588 of the modified AAV capsid protein. In some embodiments, the reference AAV capsid protein is a capsid protein of Anc80-55 or a modification thereof and the targeting peptide is between positions 586 and 593 or between positions 584 and 591 of the modified AAV capsid protein. In some embodiments, the reference AAV capsid protein is a capsid protein of Anc80-55 or a modification thereof and the targeting peptide is between S586 and T589 of the reference AAV capsid protein, thereby replacing amino acid residues S587 and N588.

In some embodiments, the reference AAV capsid protein is a capsid protein of Anc80-129 or a modification thereof and the targeting peptide is between T589 and A590 or between A587 and N588 of the modified AAV capsid protein. In some embodiments, the reference AAV capsid protein is a capsid protein of Anc80-129 or a modification thereof and the targeting peptide is between positions 586 and 593 or between positions 584 and 591 of the modified AAV capsid protein. In some embodiments, the reference AAV capsid protein is a capsid protein of Anc80-129 or a modification thereof and the targeting peptide is between S586 and T589 of the reference AAV capsid protein, thereby replacing amino acid residues A587 and N588.

In some embodiments, the reference AAV capsid protein is a capsid protein of Anc80-156 or a modification thereof and the targeting peptide is between T589 and A590 or between A587 and N588 of the modified AAV capsid protein. In some embodiments, the reference AAV capsid protein is a capsid protein of Anc80-156 or a modification thereof and the targeting peptide is between positions 586 and 593 or between positions 584 and 591 of the modified AAV capsid protein. In some embodiments, the reference AAV capsid protein is a capsid protein of Anc80-156 or a modification thereof and the targeting peptide is between S586 and T589 of the reference AAV capsid protein, thereby replacing amino acid residues A587 and N588.

In some embodiments, the reference AAV capsid protein is a capsid protein of Anc80-751 or a modification thereof and the targeting peptide is between T589 and A590 or between A587 and N588 of the modified AAV capsid protein. In some embodiments, the reference AAV capsid protein is a capsid protein of Anc80-751 or a modification thereof and the targeting peptide is between positions 586 and 593 or between positions 584 and 591 of the modified AAV capsid protein. In some embodiments, the reference AAV capsid protein is a capsid protein of Anc80-751 or a modification thereof and the targeting peptide is between S586 and T589 of the reference AAV capsid protein, thereby replacing amino acid residues A587 and N588.

In some embodiments, the reference AAV capsid protein is a capsid protein of Anc80-1029 or a modification thereof and the targeting peptide is between T589 and A590 or between A587 and N588 of the modified AAV capsid protein. In some embodiments, the reference AAV capsid protein is a capsid protein of Anc80-1029 or a modification thereof and the targeting peptide is between positions 586 and 593 or between positions 584 and 591 of the modified AAV capsid protein. In some embodiments, the reference AAV capsid protein is a capsid protein of Anc80-1029 or a modification thereof and the targeting peptide is between S586 and T589 of the reference AAV capsid protein, thereby replacing amino acid residues A587 and N588.

In some embodiments, the reference AAV capsid protein is a capsid protein of Anc80-1712 or a modification thereof and the targeting peptide is between T589 and A590 or between A587 and T588 of the modified AAV capsid protein. In some embodiments, the reference AAV capsid protein is a capsid protein of Anc80-1712 or a modification thereof and the targeting peptide is between positions 586 and 593 or between positions 584 and 591 of the modified AAV capsid protein. In some embodiments, the reference AAV capsid protein is a capsid protein of Anc80-1712 or a modification thereof and the targeting peptide is between S586 and T589 of the reference AAV capsid protein, thereby replacing amino acid residues A587 and N588.

### 6.2.3. Modified AAV Capsid Proteins Having Flanking Regions

In some embodiments, the modified AAV capsid protein further comprises a N-terminal flanking region on the N-terminal end of the targeting peptide, a C-terminal flanking region on the C-terminal end of the targeting peptide, or both.

In some embodiments, the N-terminal flanking region comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, amino acid residues. In a non-limiting example, the N-terminal flanking region comprises 11 amino acids, where the amino acids are selected from amino acid residues 578-588 of AAV9, 577-587 of AAV9, or 576-586 of AAV9.

In some embodiments, the N-terminal flanking region comprises at least four (e.g., at least five, at least six, at least seven, at least eight, at least nine, or at least ten) consecutive amino acid residues from amino acid 558-589 (e.g., 558-588, 558-587, 558-586, and 558-585) of the reference AAV capsid protein, wherein the amino acid residues are numbered according to the amino acid sequence of the reference AAV capsid protein. In some embodiments, the N-terminal flanking region comprises a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from amino acids of 558-589 of the reference AAV capsid protein, wherein the amino acid residues are numbered according to the amino acid sequence of the reference AAV capsid protein.

In some embodiments, the N-terminal flanking region comprises at least four (e.g., at least five, at least six, at least seven, at least eight, at least nine, or at least ten) consecutive amino acid residues from amino acids 576-585 of the reference AAV capsid protein, wherein the amino acid residues are numbered according to the amino acid sequence of the reference AAV capsid protein. In some embodiments, the N-terminal flanking region comprises at least four (e.g., at least five, at least six, at least seven, at least eight, at least nine, or at least ten) consecutive amino acid residues from amino acids 576-586 of the reference AAV capsid protein, wherein the amino acid residues are numbered according to the amino acid sequence of the reference AAV capsid protein. In some embodiments, the N-terminal flanking region comprises at least four (e.g., at least five, at least six, at least seven, at least eight, at least nine, or at least ten) consecutive amino acid residues from amino acids 576-587 of the reference AAV capsid protein, wherein the amino acid residues are numbered according to the amino acid sequence of the reference AAV capsid protein. In some embodiments, the N-terminal flanking region comprises at least four (e.g., at least five, at least six, at least seven, at least eight, at least nine, or at least ten) consecutive amino acid residues from amino acids of the reference AAV capsid protein, wherein the amino acid residues are numbered according to the amino acid sequence of the reference AAV capsid protein. In some embodiments, the N-terminal flanking region comprises at least four (e.g., at least five, at least six, at least seven, at least eight, at least nine, or at least ten) consecutive amino acid residues from amino acids 576-589 of the reference AAV capsid protein, wherein the amino acid residues are numbered according to the amino acid sequence of the reference AAV capsid protein.

In some embodiments, the N-terminal flanking region has the sequence of B₁YGB₂VATNB₃QS (SEQ ID NO: 55849), and B₁, B₂, and B₃ are each independently selected from any amino acid residue. In some embodiments, the N-terminal flanking region has the sequence of B₁YGB₂VATNB₃QS (SEQ ID NO: 55849) where B₁ is selected from a glutamate (E) or a serine (S), B₂ is selected from a threonine (T) or a glutamine (Q), and B₃ is selected from a leucine (L) or a histidine (H).

In some embodiments, the N-terminal flanking region has the sequence of SYGQVATNHQS (SEQ ID NO: 55848). In some embodiments, the N-terminal flanking region comprises a sequence having 1, 2, 3, 4, 5, or more amino acid residues different (e.g., insertion, deletion, or substitution) to the sequence of SYGQVATNHQS (SEQ ID NO: 55848).

In some embodiments, the N-terminal flanking region replaces N-terminal reference sequence of the reference AAV capsid protein, wherein the N-terminal reference sequence has at least 60% (e.g., at least 70%, at least 80%, at least 90% or at least 95%) sequence identity to the N-terminal flanking region and positioned at N-terminal end of an insertion site of the targeting peptide within the reference AAV capsid protein. The modified AAV capsid protein of claim 34, wherein the N-terminal reference sequence has the sequence of B₁YGB₂VATNB₃QS (SEQ ID NO: 55849).

In some embodiments, the modified AAV capsid protein further comprises a C-terminal flanking region on the C-terminal end of the targeting peptide.

In some embodiments, the C-terminal flanking region has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, amino acid residues. In a non-limiting example, the C-terminal flanking region comprises 13 amino acids, where the amino acids are selected from amino acid residues 589-602 of AAV9, 588-601 of AAV9, or 587-602 of AAV9.

In some embodiments, the C-terminal flanking region comprises at least four (e.g., at least five, at least six, at least seven, at least eight, at least nine, or at least ten) consecutive amino acid residues from amino acids 589-635 of the reference AAV capsid protein, wherein the amino acid residues are numbered according to the amino acid sequence of the reference AAV capsid protein. In some embodiments, the C-terminal flanking region comprises a sequence having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) from amino acid residues of 589-635, where residues 589-635 relative to a reference sequence numbered according to the amino acid sequence of the reference AAV capsid protein.

In some embodiments, the C-terminal flanking region comprises at least four (e.g., at least five, at least six, at least seven, at least eight, at least nine, or at least ten) consecutive amino acid residues from amino acids 589-601 of the reference AAV capsid protein, wherein the amino acid residues are numbered according to the amino acid sequence of the reference AAV capsid protein. In some embodiments, the C-terminal flanking region comprises at least four (e.g., at least five, at least six, at least seven, at least eight, at least nine, or at least ten) consecutive amino acid residues from amino acids 590-601 of the reference AAV capsid protein, wherein the amino acid residues are numbered according to the amino acid sequence of the reference AAV capsid protein. In some embodiments, the C-terminal flanking region comprises at least four (e.g., at least five, at least six, at least seven, at least eight, at least nine, or at least ten) consecutive amino acid residues from amino acids 591-601 of the reference AAV capsid protein, wherein the amino acid residues are numbered according to the amino acid sequence of the reference AAV capsid protein.

In some embodiments, the C-terminal flanking region has the sequence of AQAQTGZ₁VZ₂Z₃QGZ₄ (SEQ ID NO: 55851), where Z₁, Z₂, Z₃, and Z₄ are each independently selected from any amino acid residue. In some embodiments, the C-terminal flanking region has the sequence of AQAQTGZ₁VZ₂Z₃QGZ₄ (SEQ ID NO: 55861), where wherein Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

In some embodiments, the C-terminal flanking region has the sequence of AQAQTGWVQNQGI (SEQ ID NO: 55850). In some embodiments, the C-terminal flanking region comprises a sequence having 1, 2, 3, 4, 5, or more amino acid residues different (e.g., insertion, deletion, or substitution) to the sequence of AQAQTGWVQNQGI (SEQ ID NO: 55850).

In some embodiments, the C-terminal flanking region replaces C-terminal reference sequence of the reference AAV capsid protein, wherein the C-terminal reference sequence has at least 60% (e.g., at least 70%, at least 80%, at least 90% or at least 95%) sequence identity to the C-terminal flanking region and positioned at C-terminal end of an insertion site of the targeting peptide within the reference AAV capsid protein. In some embodiments, the C-terminal reference sequence has the sequence of AQAQTGZ₁VZ₂Z₃QGZ₄ (SEQ ID NO: 55851).

In some embodiments, the modified AAV capsid protein further comprises a N-terminal flanking region having the sequence of B₁YGB₂VATNB₃QS (SEQ ID NO: 55849, where B₁, B₂, and B₃ are each independently selected from any amino acid residue; and a C-terminal flanking region having the sequence of AQAQTGZ₁VZ₂Z₃QGZ₄ (SEQ ID NO: 55851), where Z₁, Z₂, Z₃, and Z₄ are each independently selected from any amino acid residues.

In some embodiments, the modified AAV capsid protein further comprises a N-terminal flanking region having the sequence of B₁YGB₂VATNB₃QS (SEQ ID NO: 55860) and B₁ is selected from a glutamate (E) or a serine (S), B₂ is selected from a threonine (T) or a glutamine (Q), and B₃ is selected from a leucine (L) or a histidine (H); and a C-terminal flanking region having the sequence of AQAQTGZ₁VZ₂Z₃QGZ₄ (SEQ ID NO: 55861) and Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

In one embodiment, the modified AAV capsid protein further comprises a N-terminal flanking region having the sequence of SYGQVATNHQS (SEQ ID NO: 55848), and a C-terminal flanking region having the sequence of AQAQTGWVQNQGI (SEQ ID NO: 55850).

In some embodiments, the modified AAV capsid protein further comprises an amino acid sequence of B₁YGB₂VATNB₃QSPLMGAVHLYAQAQTGZ₁VZ₂Z₃QGZ₄ (SEQ ID NO: 55852), where B₁, B₂, B₃, Z₁, Z₂, Z₃, and Z₄ are each independently selected from any amino acid residue. In some embodiments, the modified AAV capsid protein further comprises an amino acid sequence of B₁YGB₂VATNB₃QSPLMGAVHLYAQAQTGZ₁VZ₂Z₃QGZ₄ (SEQ ID NO: 55862), where B₁ is selected from a glutamate (E) or a serine (S), B₂ is selected from a threonine (T) or a glutamine (Q), B₃ is selected from a leucine (L) or a histidine (H), Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I)..

In one embodiments, the targeting peptide in VR VIII is SEQ ID NO: 29. In some embodiments, the targeting peptide is SEQ ID NO: 29, wherein upon insertion in VR VIII one or more residues are replaced by the insertion.

In some embodiments, the targeting peptide is a peptide having at least 90% (e.g., 92%, 94%, 96%, or 98%) sequence identity to the sequence of SEQ ID NO: 29. In some embodiments, the targeting peptide is a peptide having 1, 2, 3, 4, 5, 6, 7, 8, 9, 10, or more amino acid residues different (e.g., insertion, deletion, or substitution) to a sequence of SEQ ID NO: 29.

### 6.2.4. Additional Modifications in Modified AAV Capsid Protein

In some embodiments, the modified AAV capsid protein has one or more modifications comprising amino acid insertions, deletions, substitutions or combinations thereof as compared to a reference AAV capsid protein.

In some embodiments, the one or more modifications comprises an amino acid insertion, deletion, substitution, or a combination thereof to introduce the targeting peptide into VR VIII of the reference AAV capsid protein.

In some embodiments, the one or more modifications comprises an amino acid modification outside of VR VIII of the reference AAV capsid protein.

In some embodiments, the one or more modifications outside of VR VIII of the reference AAV capsid protein comprise one or more modifications in VR I, VR II, VR III, VR IV, VR V, VR VI, or VR VII.

In some embodiments, variable region I (VR I) corresponds to sequences between about position 259 to about position 275 in the AAV capsid protein (e.g., reference capsid or modified capsid). In some embodiments, VR I corresponds to sequence between about position 262 to about position 272 in the AAV capsid protein (e.g., reference capsid or modified capsid).

In some embodiments, variable region II (VR II) corresponds to sequences between about position 329 to about position 336 in the AAV capsid protein (e.g., reference capsid or modified capsid). In some embodiments, VR II corresponds to sequence between about position 330 to about position 335 in the AAV capsid protein (e.g., reference capsid or modified capsid).

In some embodiments, variable region III (VR III) corresponds to sequences between about position 378 to about position 400 in the AAV capsid protein (e.g., reference capsid or modified capsid). In some embodiments, VR III corresponds to sequence between about position 385 to about position 394 in the AAV capsid protein (e.g., reference capsid or modified capsid).

In some embodiments, variable region IV (VR IV) corresponds to sequences between about position 438 to about position 480 in the AAV capsid protein (e.g., reference capsid or modified capsid). I n some embodiments, VTR IV corresponds to sequence between about position 456 to about position 476 in the AAV capsid protein (e.g., reference capsid or modified capsid). In some embodiments, VR IV corresponds to sequence between about position 449 to about position 468 in the AAV capsid protein (e.g., reference capsid or modified capsid).

In some embodiments, variable region V (VR V) corresponds to sequences between about position 483 to about position 518 in the AAV capsid protein (e.g., reference capsid or modified capsid). In some embodiments, VR V corresponds to sequence between about position 494 to about position 512 in the AAV capsid protein (e.g., reference capsid or modified capsid). In some embodiments, VR V corresponds to sequence between about position 487 to about position 504 in the AAV capsid protein (e.g., reference capsid or modified capsid).

In some embodiments, variable region VI (VR VI) corresponds to sequences between about position 531 to about position 549 in the AAV capsid protein (e.g., reference capsid or modified capsid). In some embodiments, VR VI corresponds to sequence between about position 533 to about position 545 in the AAV capsid protein (e.g., reference capsid or modified capsid).

In some embodiments, variable region VII (VR VII) corresponds to sequences between about position 551 to about position 567 in the AAV capsid protein (e.g., reference capsid or modified capsid). In some embodiments, VR VII corresponds to sequence between about position 553 to about position 563 in the AAV capsid protein (e.g., reference capsid or modified capsid).

In some embodiments, variable region VIII (VR VIII) corresponds to sequences between about position 570 to about position 605 in the AAV capsid protein (e.g., reference capsid or modified capsid). In some embodiments, variable region VIII (VR VIII) corresponds to sequences between about position 576 to about position 601 in the AAV capsid protein (e.g., reference capsid or modified capsid).). In some embodiments, variable region VIII (VR VIII) corresponds to sequences between about position 576 to about position 608 in the AAV capsid protein (e.g., reference capsid or modified capsid). In some embodiments, variable region VIII (VR VIII) corresponds to sequences between about position 579 to about position 594 in the AAV capsid protein (e.g., reference capsid or modified capsid). In some embodiments, VR VIII corresponds to sequence between about position 585 to about position 591 in the AAV capsid protein (e.g., reference capsid or modified capsid).

In some embodiments, variable region IX (VR IX) corresponds to sequences between about position 709 to about position 736 in the AAV capsid protein (e.g., reference capsid or modified capsid). In some embodiments, VR IX corresponds to sequence between about position 714 to about position 721 in the AAV capsid protein (e.g., reference capsid or modified capsid).

In some embodiments, the variable regions in an AAV capsid are as defined in Padron et al., J. Virology, 79(8): 5047-5058 (2005), doi.org/10.1128/JVI.79.8.5047-5058.2005; Dimattia, et al., J. Virology, 86(12): 6947-6958 (2012), doi.org/10.1128/JVI.07232-11; Meyer et al., eLIFE; 8:e44707. DOI: doi.org/10.7554/eLife.44707 (2019); Goertsen et al., Nat Neurosci., 25(1):106-115. doi: 10.1038/s41593-021-00969 (2022), each of which is herein incorporated by reference in its entirety.

In some embodiments, the variable regions in an AAV capsid are defined by which amino acids are present on surface-exposed loops. In some embodiments, the variable regions in an AAV capsid are defined by which amino acids are present on surface-exposed loops and where the surface-exposed loop contributes to tropism (i.e., contributes to the AAV virion binding to a receptor). In some embodiments, the presence of a targeting peptide in the modified capsid protein may change the amino acid residues present on a surface exposed-loop.

In some embodiments, the one or more modifications outside of VR VIII of the reference AAV capsid protein comprise one or more modifications in VR IV and VR V.

In some embodiments, the one or more modifications in VR IV comprises modifications result in introduction of a sequence of SEQ ID NO: 30. In some embodiments, the one or more modifications in VR VI results in introduction of a sequence having at least 90% (e.g., 91%, 92%, 93%, 94%, 95%, 96%, 97%, 98%, 99% or 99.5%) sequence identity to the sequence of SEQ ID NO: 30. In some embodiments, the one or more modifications in VR VI results in introduction of a sequence having 1, 2, 3, 4, 5, 6, 7, 8, 9, 10, or more amino acid residues different (e.g., insertion, deletion, or substitution) to a sequence of SEQ ID NO: 30.

In some embodiments, the one or more modifications in VR V comprises modifications result in introduction of a sequence of SEQ ID NO: 31. In some embodiments, the one or more modifications in VR VI results in introduction of a sequence having at least 90% (e.g., 91%, 92%, 93%, 94%, 95%, 96%, 97%, 98%, 99% or 99.5%) sequence identity to the sequence of SEQ ID NO: 31. In some embodiments, the one or more modifications in VR VI results in introduction of a sequence having 1, 2, 3, 4, 5, 6, 7, 8, 9, 10, or more amino acid residues different (e.g., insertion, deletion, or substitution) to a sequence of SEQ ID NO: 31.

In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR I derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR II derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR III derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR IV derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR V derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR VI derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR VII derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR VIII derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises a VR IX derived from a second reference capsid protein (e.g., any of the reference capsid proteins described herein).

In some embodiments, a modified capsid protein has at least 90% (e.g., 91%, 92%, 93%, 94%, 95%, 96%, 97%, 98%, 99% or 99.5%) sequence identity to SEQ ID NO: 142 and comprises a VR I derived from AAV9. In some embodiments, a modified capsid protein has at least 90% (e.g., 91%, 92%, 93%, 94%, 95%, 96%, 97%, 98%, 99% or 99.5%) sequence identity to SEQ ID NO: 142 and comprises a VR IV derived from AAV9. In some embodiments, a modified capsid protein has at least 90% (e.g., 91%, 92%, 93%, 94%, 95%, 96%, 97%, 98%, 99% or 99.5%) sequence identity to SEQ ID NO: 142 and comprises a VR V derived from AAV9. In some embodiments, a modified capsid protein has at least 90% (e.g., 91%, 92%, 93%, 94%, 95%, 96%, 97%, 98%, 99% or 99.5%) sequence identity to SEQ ID NO: 142 and comprises a VR VIII derived from AAV9.

In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein) and comprises two or more variable regions (VR I, II, III, IV, V, VI, VII, VIII, or IX) derived from a second reference capsid protein (e.g., any of the reference capsid proteins described herein). In some embodiments, a modified capsid protein has at least 90% (e.g., 91%, 92%, 93%, 94%, 95%, 96%, 97%, 98%, 99% or 99.5%) sequence identity to the sequence of a reference AAV capsid protein (e.g., any of the reference capsid proteins described herein), and comprises a VR IV derived from a second reference capsid protein (e.g., any of the reference capsid proteins described herein), and a VR V derived from a second reference capsid protein (e.g., any of the reference capsid proteins described herein). In one embodiment, a modified capsid protein has at least 90% (e.g., 91%, 92%, 93%, 94%, 95%, 96%, 97%, 98%, 99% or 99.5%) sequence identity to SEQ ID NO: 142, and comprises a VR IV derived from AAV9, and a VR V derived from AAV9.

A modified AAV capsid protein of the present disclosure can change the tropism, specificity and/or bio-distribution of an AAV comprising the modified AAV capsid protein. In preferred embodiments, an AAV comprising the modified AAV capsid protein has increased targeting to a target cell, tissue or organ when administered to a subject. In some embodiments, an AAV comprising the modified AAV capsid protein has decreased distribution outside of a target cell, tissue or organ when administered to a subject. In some embodiments, tropism of a modified AAV capsid protein can be measured using an enrichment score. A non-limiting example of an enrichment score is based on the combination of amino acid residues present in the modified sequence within VR VIII. An exemplary enrichment formula is provided below:

$$Tissue Enrichment score = log 2 FoldChange = log 2 {MN}_{Tissue} - log 2 {MN}_{TestArticle}$$

The formula for the scaled log 2 fold change is provided below:

$$Scaled log 2 FoldChange = ( log 2 FoldChange - min ( log 2 FoldChange ) ) / ( max ( log 2 FoldChange ) - min ( log 2 FC ) )$$

### 6.2.5. Reference AAV Capsid Proteins

The reference AAV capsid protein used in various embodiments of the present disclosure is a VP1, VP2 or VP3 capsid protein of an AAV known in the art. It can be a VP1, VP2 or VP3 capsid protein of a naturally occurring or non-naturally occurring AAV variant. The reference AAV capsid protein does not comprise a targeting peptide disclosed herein in VR VIII.

The non-naturally occurring VP1, VP2, or VP3 capsid protein includes a capsid protein generated by biological or chemical alteration or in silico design, or variation of a naturally occurring AAV capsid protein. Accordingly, the reference AAV capsid protein includes, but is not limited to, a capsid protein of various AAV serotypes (e.g., AAV1, AAV2, AAV3B, AAV5, AAV6, AAV8, and AAV9) or a variant thereof. A non-naturally occurring VP1, VP2, or VP3 capsid protein further includes an artificial capsid protein created by in silico design or synthesis. An artificial capsid protein includes, but is not limited to, AAV capsid proteins disclosed in PCT/US2014/060163, U.S. Pat. No. 9,695,220, PCT/US2016/044819, PCT/US2018/032166, PCT/US2019/031851, and PCT/US2019/047546, which are incorporated herein by reference in their entireties.

In some embodiments, the reference AAV capsid protein is the capsid protein of AAV9 (Genbank Ace. No: AAS99264.1), AAV1 (Genbank Ace. No: AAD27757.1), AAV2 (Genbank Ace. No: AAC03780.1), AAV3 (Genbank Ace. No: AAC55049.1), AAV3b (Genbank Ace. No: AF028705.1), AAV4 (Genbank Ace. No: AAC58045.1), AAV5 (Genbank Ace. No: AAD13756.1), AAV6 (Genbank Ace. No: AF028704.1), AAV7 (Genbank Ace. No: AAN03855.1), AAV 8 (Genbank Ace. No: AAN03857.1), AAV10 (Genbank Ace. No: AAT46337.1), AAVrh10 (Genbank Ace. No: AY243015.1), AAV11 (Genbank Ace. No: AAT46339.1), AAV12 (Genbank Ace. No: ABI16639.1), or AAV13 (Genbank Ace. No: ABZ10812.1), AAVpol (Genbank Ace. No: FJ688147.1). In certain embodiments, the AAV capsid protein is the capsid protein of AAV9 (Genbank Ace. No: AAS99264.1).

The reference AAV capsid protein can be VP1 capsid protein having a sequence selected from: SEQ IID NO: 54 (AAV1 (AAD27757)), SEQ ID NO: 55 (AAV2 (AAC03780)), SEQ ID NO: 56 (AAV3 (AAC55049)), SEQ ID NO: 57 (AAV5 (AAD13756)), SEQ ID NO: 58 (AAV6 (AAB₉₅₄₅₀)), SEQ ID NO: 59 (AAV7 (AF513851_2)), SEQ ID NO: 60 (AAV8 (AF513852_2)), SEQ ID NO: 61 (AAV9 (AAS99264)), SEQ ID NO: 62 (AAV10 (AAT46337)), SEQ ID NO: 63 (AAV hu.68), SEQ ID NO: 64 (AAV LK03), SEQ ID NO: 65 (AAV hu.1 (AAS99260)), SEQ ID NO: 66 (AAV hu.2 (AAS99270)), SEQ ID NO: 67 (AAV hu.3 (AAS99280)), SEQ ID NO: 68 (AAV hu.4 (AAS99287)), SEQ ID NO: 69 (AAV hu.6 (AAS99306)), SEQ ID NO: 70 (AAV hu.7 (AAS99313)), SEQ ID NO: 71 (AAV hu.9 (AAS99314)), SEQ ID NO: 72 (AAV hu.10 (AAS99261)), SEQ ID NO: 73 (AAV hu.11 (AAS99262)), SEQ ID NO: 74 (AAV hu.15 (AAS99265)), SEQ ID NO: 75 (AAV hu.16 (AAS99266)), SEQ ID NO: 76 (AAV hu.17 (AAS99267)), SEQ ID NO: 77 (AAV hu.18 (AAS99268)), SEQ ID NO: 78 (AAV hu.20 (AAS99271)), SEQ ID NO: 79 (AAV hu.21 (AAS99272)), SEQ ID NO: 80 (AAV hu.22 (AAS99273)), SEQ ID NO: 81 (AAV hu.23 (AAS99274)), SEQ ID NO: 82 (AAV hu.25 (AAS99276)), SEQ ID NO: 83 (AAV hu.27 (AAS99277)), SEQ ID NO: 84 (AAV hu.28 (AAS99278)), SEQ ID NO: 85 (AAV hu.29 (AAS99279)), SEQ ID NO: 86 (AAV hu.31 (AAS99281)), SEQ ID NO: 87 (AAV hu.32 (AAS99282)), SEQ ID NO: 88 (AAV hu.34 (AAS99283)), SEQ ID NO: 89 (AAVhu.37 (AAS99285)), SEQ ID NO: 90 (AAV hu.39 (AAS99286)), SEQ ID NO: 91 (AAV hu.41 (AAS99289)), SEQ ID NO: 92 (AAV hu.42 (AAS99290)), SEQ ID NO: 93 (AAV hu.43 (AAS99291)), SEQ ID NO: 94 (AAV hu.44 (AAS99292)), SEQ ID NO: 95 (AAV hu.45 (AAS99293)), SEQ ID NO: 96 (AAV hu.46 (AAS99294)), SEQ ID NO: 97 (AAV hu.47 (AAS99295)), SEQ ID NO: 98 (AAV hu.48 (AAS99296)), SEQ ID NO: 99 (AAV hu.51 (AAS99298)), SEQ ID NO: 100 (AAV hu.52 (AAS99299)), SEQ ID NO: 101 (AAV hu.53 (AAS99300)), SEQ ID NO: 102 (AAV hu.54 (AAS99301)), SEQ ID NO: 103 (AAV hu.55 (AAS99302)), SEQ ID NO: 104 (AAV hu.56 (AAS99303)), SEQ ID NO: 105 (AAV hu.57 (AAS99304)), SEQ ID NO: 106 (AAV hu.60 (AAS99307)), SEQ ID NO: 107 (AAV hu.61 (AAS99308)), SEQ ID NO: 108 (AAV hu.63 (AAS99309)), SEQ ID NO: 109 (AAV hu.66 (AAS99311)), SEQ ID NO: 110 (AAV hu.67 (AAS99312)), SEQ ID NO: 111 (AAV rh.10 (AAO88201)), SEQ ID NO: 112 (AAV rh.13 (AAO88199)), SEQ ID NO: 113 (AAV rh.19 (AAO88194)), SEQ ID NO: 114 (AAV rh.22 (AAO88192)), SEQ ID NO: 115 (AAV rh.23 (AAO88191)), SEQ ID NO: 116 (AAV rh.24 (AAO88190)), SEQ ID NO: 117 (AAV rh.35 (AAO88186)), SEQ ID NO: 118 (AAV rh.43 (AAS99245)), SEQ ID NO: 119 (AAV rh.48 (AAS99246)), SEQ ID NO: 120 (AAV rh.49 (AAS99247)), SEQ ID NO: 121 (AAV rh.50 (AAS99248)), SEQ ID NO: 122 (AAV rh.51 (AAS99249)), SEQ ID NO: 123 (AAV rh.52 (AAS99250)), SEQ ID NO: 124 (AAV rh.53 (AAS99251)), SEQ ID NO: 125 (AAV rh.54 (AAS99252)), SEQ ID NO: 126 (AAV rh.55 (AA599253)), SEQ ID NO: 127 (AAV rh.57 (AA599254)), SEQ ID NO: 128 (AAV rh.58 (AAS99255)), SEQ ID NO: 129 (AAV rh.62 (AAS99258)), SEQ ID NO: 130 (AAV rh.64 (AAS99259)), SEQ ID NO: 131 (AAV rh.56 (JA400164)), SEQ ID NO: 143 (Anc80L1), SEQ ID NO: 144 (Anc80L27), SEQ ID NO: 145 (Anc80L33), SEQ ID NO: 146 (Anc80L36), SEQ ID NO: 147 (Anc80L44), SEQ ID NO: 148 (Anc80L59), SEQ ID NO: 149 (Anc80L60), SEQ ID NO: 150 (Anc80L62), SEQ ID NO: 151 (Anc82DI), SEQ ID NO: 152 (AAV rh.74); Anc80-55 (SEQ ID NO: 44885); Anc80-129 (SEQ ID NO: 44887); Anc80-156 (SEQ ID NO: 44889); Anc80-751 (SEQ ID NO: 44916); Anc80-1029 (SEQ ID NO: 44917); and Anc80-1712 (SEQ ID NO: 44893). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein.

The reference AAV capsid protein can be VP1 capsid protein having any member sequence of the ancestral AAV library selected from SEQ ID NO: 132 (Anc80), SEQ ID NO: 133 (Anc81 (AKU89596)), SEQ ID NO: 134 (Anc82 (AKU89597)), SEQ ID NO: 135 (Anc83 (AKU89598)), SEQ ID NO: 136 (Anc84 (AKU89599)), SEQ ID NO: 137 (Anc94) SEQ ID NO: 138 (Ancl 10 (AKU89600)), SEQ ID NO: 139 (Anc113 (AKU89601)), SEQ ID NO: 140 (Anc126 (AKU89602)), SEQ ID NO: 141 Anc127 (AKU89603), and SEQ ID NO: 142 (Anc80L65 (AKU89595)). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein. When a SEQ ID NO for a library sequence is used in this disclosure, it refers to a sequence of any one member of the library.

In some embodiments, the reference AAV capsid protein is a liver-toggle mutant described in WO2019/217911, which is incorporated by reference in its entirety herein.

In some embodiments, the reference AAV capsid protein is a capsid protein (VP1, VP2 or VP3) of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-13; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; rh74; hu.67-E; hu.17-E; hu.6-E; hu.66-E; rh.38-E; hu.32-F; AAV9/hu; hu.31-F; Anc80L27; Anc80L59; Anc80L60; Anc80L62; Anc80L65; Anc80L33; Anc80L36; Anc80L44; Anc80L1; Anc80-55, Anc80-129, Anc80-156, Anc80-751, Anc80-1029, Anc80-1712; Anc 10; and Anc80DI. In some embodiments, the reference AAV capsid protein is a capsid protein of any member protein of an ancestral AAV library selected from: Anc80; Anc81; Anc82; Anc83; Anc84; Anc94; Anc113; Anc126; and Anc127.

In some embodiments, the reference AAV capsid protein is a protein having at least 90%, 95%, 96%, 97%, 98% or 99% sequence identity to a sequence selected from SEQ ID NOs: 54-131 and 143-158. In some embodiments, the reference AAV capsid protein is a protein having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues different (e.g., insertion, deletion, or substitution) to a sequence selected from SEQ ID NOs: 54-131 and 143-158. In some embodiments, the reference AAV capsid protein is a protein having a sequence selected from SEQ ID NOs: 54-131 and 143-158. In some embodiments, the reference AAV capsid protein is a protein having a VP2 (corresponding to amino acids 138 to 736 of AAV9 VP1) or VP3 portion (corresponding to amino acids 138 to 736 of AAV9 VP1) of the protein having a sequence selected from SEQ ID NOs: 54-131 and 143-158.

### 6.2.6. Other Modifications

In some embodiments, the modified AAV capsid protein comprises one or more additional modifications compared to a reference AAV capsid protein. The one or more additional modifications can be an insertion, deletion, substitution or a combination thereof, and can be located in the VRVIII and/or outside of the VRVIII.

In some embodiments, the modified AAV capsid protein is different from the reference AAV capsid protein by having one or more amino acid substitutions at a variable region of the reference AAV capsid protein. In some embodiments, the one or more amino acid substitutions is at a variable region, VR I, of the reference AAV capsid protein (FIG. 28).

### 6.3. Polynucleotides Encoding Modified AAV Capsid Proteins; Vectors; Host Cells

In another aspect, the present disclosure provides a polynucleotide encoding a modified AAV capsid protein described herein. In some embodiments, the polynucleotide is codon optimized for expression in a bacterial or mammalian cell.

In some embodiments, the polynucleotide is inserted into an expression vector. In some embodiments, the polynucleotide is operably linked to a promoter or a sequence inducing expression of a protein from the polynucleotide. The present disclosure provides a vector including the polynucleotide encoding a modified AAV capsid protein. The vector can be used for generation of the modified AAV capsid protein. In some embodiments, the vector is used to generate an AAV virion comprising the modified AAV capsid protein. In some embodiments, the vector further comprises an AAV rep protein or a fragment thereof. In some embodiments, the reference capsid protein for the modified AAV capsid protein and the rep protein are originated from an AAV of the same clade. In some embodiments, the reference capsid protein for the modified AAV capsid protein and the rep protein are originated from an AAV of different clades.

In some embodiments, the polynucleotide is transfected to a host cell. The present disclosure provides a host cell comprising the polynucleotide encoding a modified AAV capsid protein. The host cell can be a prokaryotic cell or eukaryotic cell. In some embodiments, the host cell is a mammalian cell or a yeast cell.

In some embodiments, the host cell further comprises another polynucleotide encoding an AAV protein. In some embodiments, the host cell comprises a functional rep gene; a recombinant nucleic acid vector comprising AAV inverted terminal repeats (ITRs) and an expressible polynucleotide; and sufficient helper functions to permit packaging of the recombinant nucleic acid vector into the modified AAV capsid protein.

In some embodiments, the components required for the host cell to package a recombinant nucleic acid vector in a modified AAV capsid protein are provided to the host cell in trans. In some embodiments, any one or more of the required components (e.g., a recombinant nucleic acid vector, rep sequences, cap sequences, and/or helper functions) are provided by a stable host cell which has been engineered to contain one or more of the required components using methods known to those of skill in the art. In some embodiments, such a stable host cell contains the required component(s) under the control of an inducible promoter. In some embodiments, the required component(s) is under the control of a constitutive promoter.

### 6.4. Modified AAV Virions

The present disclosure further provides a modified recombinant AAV (rAAV) virion comprising a modified AAV capsid protein described herein. In some embodiments, the modified rAAV comprises a modified AAV capsid protein and a recombinant nucleic acid vector.

In some embodiments, the modified rAAV comprising a modified AAV capsid protein achieves higher infection of a target following administration to a mammalian subject as compared to an rAAV comprising a corresponding reference AAV capsid protein. In some embodiments, the modified rAAV achieves higher expression in a target of an expressible polynucleotide within the recombinant nucleic acid vector following administration to a mammalian subject when compared to expression of the expressible polynucleotide administered in an rAAV comprising a corresponding reference AAV capsid protein.

In some embodiments, the modified rAAV comprising a modified AAV capsid protein achieves lower infection of an off-target following administration to a mammalian subject as compared to an rAAV comprising a corresponding reference AAV capsid protein. In some embodiments, the modified rAAV achieves lower expression in an off-target of an expressible polynucleotide within the recombinant nucleic acid vector following administration to a mammalian subject as compared to expression of the expressible polynucleotide administered in an rAAV comprising a corresponding reference AAV capsid protein. In some embodiments, the corresponding reference AAV capsid protein is a capsid protein identical to the modified AAV capsid protein except that it does not include a targeting peptide described above.

In some embodiments, the target is brain, muscle, spinal cord, eye, or other organ. In some embodiments, the off-target tissue is muscle, liver, or other organ. In one embodiment, the target is CNS.

In some embodiments, the modified rAAV has less liver toxicity than an rAAV comprising a corresponding reference AAV capsid protein administered by the same route of administration and in the same dose. In some embodiments, the less liver toxicity is because of de-targeting of the modified rAAV to a liver.

### 6.5. Methods of Producing rAAV

The rAAV of the disclosure comprises a recombinant nucleic acid vector containing a heterologous polynucleotide. In some embodiments, the heterologous polynucleotide comprises an expressible polynucleotide operably linked to an ERE. The expressible polynucleotide and ERE optionally replace the AAV genomic coding region (e.g., replace the AAV rep and cap genes). The expressible polynucleotide and ERE are generally flanked on either side by AAV inverted terminal repeat (ITR) regions, although a single ITR may be sufficient to carry out the functions normally associated with configurations comprising two ITRs (see, for example, WO 94/13788), and vector constructs with only one ITR can thus be employed in conjunction with the rAAV of the present disclosure.

In some embodiments, the rAAV of the disclosure comprise a therapeutic protein coding sequence operably linked to an ERE. The therapeutic protein coding sequence and ERE optionally replace the AAV genomic coding region (e.g., replace the AAV rep and cap genes).

In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome.

The rAAV vector construct, and the complementary packaging gene constructs can be implemented in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., WO 95/13392; WO 98/23018; and U.S. Pat. No. 5,656,785). The AAV cap gene, providing the encapsidation proteins as described above, can be provided together with an AAV rep gene or separately (see, e.g., the above-referenced patent documents as well as WO 98/27204).

Thus, the rAAV of the disclosure can be assembled by, for example, expression of its components in a packaging host cell. The components of a virus particle (e.g., rep sequences, cap sequences, inverted terminal repeat (ITR) sequences) can be introduced into a packaging host cell using one or more viral vectors.

Once assembled, rAAV particles can be purified, if desired, using routine methods. As used herein, “purified” virus particles refer to virus particles that are removed from components in the mixture in which they were made such as, but not limited to, viral components (e.g., rep sequences, cap sequences), packaging host cells, and partially- or incompletely-assembled virus particles.

### 6.6. Pharmaceutical Composition Comprising Modified rAAV

In one aspect, the present disclosure provides a pharmaceutical composition comprising a modified AAV capsid protein or a modified rAAV of the present disclosure and a pharmaceutically acceptable carrier. The modified rAAV can comprise a modified AAV capsid protein as described herein and a recombinant nucleic acid vector containing an expressible polynucleotide.

In particular embodiments, the present disclosure provides a pharmaceutical composition comprising an rAAV whose genome comprises therapeutic protein used for treating and/or preventing a disease of the central nervous system where the coding sequence for the therapeutic protein is operably linked to an expression regulatory element (ERE).

The pharmaceutical composition can be formulated using one or more carriers, excipients, stabilizers and adjuvants to, for example: (1) increase stability; (2) increase cell transfection or transduction; (3) permit the sustained or delayed release; (4) alter the biodistribution (e.g., target the rAAV particle to specific tissues or cell types); (5) increase the translation of encoded protein in vivo; and/or (6) alter the release profile of encoded protein in vivo.

Formulations of the pharmaceutical compositions provided herein can include, without limitation, saline, which may be formulated with a variety of buffering solutions (e.g., phosphate buffered saline), lactose, sucrose, calcium phosphate, gelatin, dextran, agar, pectin, water, lipidoids, liposomes, lipid nanoparticles, polymers, lipoplexes, core-shell nanoparticles, peptides, proteins, nanoparticle mimics and combinations thereof.

Formulations of the pharmaceutical compositions described herein can be prepared by any method known or hereafter developed in the art of pharmacology. In general, such preparatory methods include the step of associating the active ingredient with a carrier and/or one or more other accessory ingredients (e.g., excipients, stabilizers and adjuvants).

A pharmaceutical composition in accordance with the present disclosure can be prepared, packaged, and/or sold in bulk, as a single unit dose, and/or as a plurality of single unit doses. As used herein, a unit dose refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g., rAAV), the pharmaceutically acceptable carrier, and/or any additional ingredients in a pharmaceutical composition in accordance with the present disclosure can vary, depending upon the identity, size, and/or condition of the subject being treated and further depending upon the route by which the composition is to be administered.

Various carriers, excipients, stabilizers and adjuvants for formulating pharmaceutical compositions and techniques for preparing the composition are known in the art (see Remington: The Science and Practice of Pharmacy, 22nd Revised Ed., Pharmaceutical Press, 2012; incorporated herein by reference in its entirety). The use of suitable conventional carriers, excipients, stabilizers and adjuvants is contemplated within the scope of the present disclosure.

In some embodiments, the pharmaceutical composition is in the form of a solution containing from about 1×10¹ to about 1×10¹⁶ genome copies (GCs)/ml of rAAV (e.g., a solution containing concentrations of from about 1×10³ to about 1×10¹⁴ GCs/ml).

### 6.6.1. Routes of Administration

The present disclosure provides a method of administering an rAAV to transfer a polynucleotide to the CNS. In some embodiments, the rAAV is administered locally or systematically.

In certain embodiments, the rAAV is administered locally to the CNS. In some embodiments, rAAV is administered to the cerebral spinal fluid (CSF) of said subject. In some embodiments, the rAAV is administered to the cisternae magna, intraventricular space, brain ventricle, subarachnoid space, intrathecal space and/or ependyma of the subject.

In some embodiments, rAAV is administered by intrathecal administration, intracranial administration, intracerebroventricular (ICV), or intraparenchymal administration or administration to the lateral ventricles of the brain.

In some embodiments, rAAV is administered by lumbar injection (e.g., into the lumbar cistern) and/or injection into the intra cisterna magna (ICM).

In some embodiments, rAAV is administered to the ventricular system. In some embodiments, rAAV is administered to the rostral lateral ventricle; and/or administered to the caudal lateral ventricle; and/or administered to the right lateral ventricle; and/or administered to the left lateral ventricle; and/or administered to the right rostral lateral ventricle; and/or administered to the left rostral lateral ventricle; and/or administered to the right caudal lateral ventricle; and/or administered to the left caudal lateral ventricle.

In some embodiments, rAAV is administered such that the rAAV contacts ependymal cells of said subject. Such ependymal cells express the encoded polypeptide and optionally the polypeptide is expressed by the cells.

In some embodiments, the polypeptide is expressed and/or is distributed in the lateral ventricle, CSF, and/or brain (e.g., striatum, thalamus, medulla, cerebellum, occipital cortex, and/or prefrontal cortex).

In some embodiments, rAAV is administered intravenously or systemically.

In some embodiments, rAAV is administered inter-digitally.

To deliver the rAAV specifically to a particular region of the CNS, especially to a particular region of the brain, it may be administered by stereotaxic microinjection. For example, on the day of surgery, patients can have the stereotaxic frame base fixed in place (screwed into the skull). The brain with stereotaxic frame base (MRI-compatible with fiduciary markings) can be imaged using high resolution MRI. The MRI images can then be transferred to a computer that runs stereotaxic software. A series of coronal, sagittal and axial images can be used to determine the target site of vector injection, and trajectory. The software directly translates the trajectory into 3-dimensional coordinates appropriate for the stereotaxic frame. Burr holes can be drilled above the entry site and the stereotaxic apparatus localized with the needle implanted at the given depth. The vector in a pharmaceutically acceptable carrier can then be injected. The AAV vector can be then administrated by direct injection to the primary target site and retrogradely transported to distal target sites via axons. Additional routes of administration can be used, e.g., superficial cortical application under direct visualization, or other non-stereotaxic application.

In some embodiments, rAAV is delivered by a pump. The pump may be implantable. Another convenient way to administer the rAAV is to use a cannula or a catheter.

In some embodiments, rAAV is administered by Convection-enhanced delivery (CED) (Nguyen et al., (2003) J. Neurosurg. 98:584-590), which has been used clinically in gene therapy (AAV2-hAADC) for Parkinson's disease (Fiandaca et al., (2008) Exp. Neurol. 209:51-57). The underlying principle of CED involves pumping infusate into brain parenchyma under sufficient pressure to overcome the hydrostatic pressure of interstitial fluid, thereby forcing the infused particles into close contact with the dense perivasculature of the brain. Pulsation of these vessels acts as a pump, distributing the particles over large distances throughout the parenchyma (Hadaczek et al., (2006) Hum. Gene Ther. 17:291-302). To increase the safety and efficacy of CED, a reflux-resistant cannula (Krauze et al., (2009) Methods Enzymol. 465:349-362) can be employed along with monitored delivery with real-time MRI. Monitored delivery allows for the quantification and control of aberrant events, such as cannula reflux and leakage of infusate into ventricles (Eberling et al., (2008) Neurology 70:1980-1983; Fiandaca et al., (2009) Neuroimage 47 Suppl. 2:T27-35; Saito et al., (2011) Journal of Neurosurgery Pediatrics 7:522-526). US20190111157A1 provides improved procedures to achieve widespread expression of AAV vectors in the cortex and/or striatum.

In some embodiments, the rAAV is administered to the striatum. In some embodiments, the rAAV is administered to at least the putamen and the caudate nucleus of the striatum. In some embodiments, the rAAV is administered to at least the putamen and the caudate nucleus of each hemisphere of the striatum. In some embodiments, the rAAV is administered to at least one site in the caudate nucleus and two sites in the putamen.

In some embodiments, rAAV is delivered by intraparenchymal administration to a specific area of the brain. In some embodiments, rAAV is delivered by intraparenchymal administration to putamen, striatum, basal forebrain region, substantia nigra and/or ventral tegmental area.

In some embodiments of the above aspects and embodiments, the rAAV is delivered by stereotactic delivery. In some embodiments, the rAAV is delivered by convection enhanced delivery (CED). In some embodiments, the rAAV is delivered using a CED delivery system. In some embodiments, the CED system comprises a cannula. In some embodiments, the cannula is a reflux-resistant cannula or a stepped cannula. In some embodiments, the CED system comprises a pump. In some embodiments, the pump is a manual pump. In some embodiments, the pump is an osmotic pump. In some embodiments, the pump is an infusion pump.

A modified rAAV of the present disclosure can be administered to a subject (e.g., a human or non-human mammal) in a suitable carrier. Suitable carriers include saline, which may be formulated with a variety of buffering solutions (e.g., phosphate buffered saline), lactose, sucrose, calcium phosphate, gelatin, dextran, agar, pectin, and water. A modified rAAV typically is administered in sufficient amounts to transduce or infect the desired cells and to provide sufficient levels of gene transfer and expression to provide a therapeutic benefit without undue adverse effects. Conventional and pharmaceutically acceptable routes of administration include, but are not limited to, direct delivery to an organ such as, for example, the muscle, liver or lung, orally, intranasally, intratracheally, intrathecally, intravenously, intramuscularly, intraocularly, subcutaneously, intradermally, or by other routes of administration. Routes of administration can be combined, if desired.

### 6.6.2. Subject

The present disclosure provides a method of transferring a polynucleotide to the central nervous system (CNS) of a subject, e.g., a mammal. In some embodiments, the subject is a human. In some embodiments, the subject has a CNS disease. In some embodiments, the subject has a genetic defect associated with CNS disease or disorder.

In some embodiments, the CNS disease or disorder is selected from Adrenoleukodystrophy, Alexander Disease, Alzheimer disease, Amyotrophic lateral sclerosis, Angelman syndrome, Ataxia telangiectasia, Canavan disease, Charcot-Marie-Tooth syndrome, Cockayne syndrome, Chronic inflammatory demyelinating polyneuropathy (CIDP), Deafness, Duchenne muscular dystrophy, Epilepsy, Essential tremor, Fragile X syndrome, Friedreich's ataxia, Gaucher disease, GM1 gangliosidosis, GM2 gangliosidoses, Huntington disease, Frontotemporal Degeneration (FTD), Lesch-Nyhan syndrome, Maple syrup urine disease, Menkes syndrome, Metachromatic leukodystrophy (MLD), Myotonic dystrophy, Multiple sclerosis, Narcolepsy, Neurofibromatosis, Niemann-Pick disease, Parkinson's disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

In some embodiments, the CNS disease or disorder is a demyelinating or white matter disease. In some embodiments, the subject has a monogenetic defect. In some embodiments, the subject has a genetic defect in a protein expressed in the CNS. In some embodiments, the subject has a monogenetic defect in a protein expressed in the CNS.

In some embodiments, the subject has a lysosomal storage disease (LDS). In some embodiments, the subject has a disease selected from: mucopolysaccharidosis type I e.g., Hurler syndrome and the variants Scheie syndrome and Hurler-Scheie syndrome; Hunter syndrome; mucopolysaccharidosis type III, e.g., Sanfilippo syndrome; mucopolysaccharidosis type IV, e.g., Morquio syndrome; mucopolysaccharidosis type VI, e.g., Maroteaux-Lamy syndrome; mucopolysaccharidosis type II; mucopolysaccharidosis type III; mucopolysaccharidosis type IV; mucopolysaccharidosis type VI; mucopolysaccharidosis type VII; mucopolysaccharidosis type VIII; mucopolysaccharidosis type IX; Tay-Sachs disease; Sandhoff disease; GM1 gangliosidosis; Fabry disease; Krabbe's disease; leukodystrophy; metachromatic leukodystrophy; Pompe disease; Fucosidosis deficiency; alpha-mannosidosis deficiency; beta-mannosidosis deficiency; Gaucher disease; Infantile Batten Disease; Classic Late Infantile Batten Disease; Juvenile Batten Disease; Batten, other forms Niemann-Pick disease; Niemann-Pick disease without sphingomyelinase deficiency; and Wolman disease.

In some embodiments, the subject has a brain cancer. In some embodiments, the subject has brain metastases of a cancer. In some embodiments, the subject has brain metastases of breast cancer. In some embodiments, the subject has brain metastases of HER2 positive breast cancer.

### 6.6.3. Dosages

The dose of a viral vector administered to a subject will depend primarily on factors such as the condition being treated, and the age, weight, and health of the subject. For example, a therapeutically effective dosage of a viral vector to be administered to a human subject generally is in the range of from about 0.1 ml to about 10 ml of a solution containing concentrations of from about IE1 to about 1E16 genome copies (GCs)/ml of viruses (e.g., a solution containing concentrations of from about 1E3 to about 1E14 GCs/ml). In some embodiments, the total dose of the rAAV administered to a subject is less than 3E14 GCs, e.g., 1E14 GCs or less, 5E13 GCs or less, 1E13 GCs or less, 5E12 GCs or less, or 1E12 GCs or less.

In another embodiment, a therapeutically effective dosage of a viral vector to be administered to a human subject generally is in the range of from about 0.1 ml to about 10 ml of a solution containing concentrations of from about 1E1 to 1E12 genome copies (GCs) of viruses (e.g., about 1E3 to 1E9 GCs). Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

In some embodiments, the effective dose is between 1E10 to 1E16 genome copy numbers (GC) of the rAAV per subject. In some embodiments, the effective dose for a human patient corresponds to a monkey dose of 1E12 to 1E15 GC of the rAAV. In some embodiments, the effective dose for a human patient corresponds to a monkey dose of 1E13 to 1E14 GC of the rAAV. In some embodiments, the effective dose for a human patient corresponds to a monkey dose of about 4E13 GC of the rAAV.

In some embodiments, the effective dose is 1E11 to 1E15 GC of the rAAV per a gram brain mass. In some embodiments, the effective dose is 1E11 to 1E13 GC of the rAAV per a gram brain mass. In some embodiments, the effective dose is 1E11 to 1E12 GC of the rAAV per a gram brain mass. In some embodiments, the effective dose is 1E12 to 1E14 GC of the rAAV per a gram brain mass. In some embodiments, the effective dose is about 5E11 GC of the rAAV per a gram brain mass. In some embodiments, the effective dose is about 2.5E11 GC of the rAAV per a gram brain mass. In some embodiments, the effective dose is about 5E10 GC of the rAAV per a gram brain mass. In some embodiments, the effective dose is about 2.5E10 GC of the rAAV per a gram brain mass.

In some embodiments, the effective dose is between 1E10-1E16 genome copy numbers (GC) of the rAAV per kg body weight. In some embodiments, the effective dose is between 1E11-1E15 genome copy numbers (GC) of the rAAV per kg body weight. In some embodiments, the effective dose is between 1E12-5E14 genome copy numbers (GC) of the rAAV per kg body weight. In some embodiments, the effective dose is between 0.5E13-2E14 genome copy numbers (GC) of the rAAV per kg body weight.

Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

In one aspect, the present invention provides a unit dose of rAAV provided herein. The unit dose comprises about 0.1 ml to about 10 ml of a solution containing concentrations of from about 1E9 to 1E17 genome copies (GCs) per ml of rAAV described herein. In some embodiments, the unit dose contains about 1E10 to 1E16 genome copies (GCs) per ml of rAAV described herein. In some embodiments, the unit dose contains about 1E11 to 1E15 genome copies (GCs) per ml of rAAV described herein. In some embodiments, the unit dose contains about 1E12 to 1E14 genome copies (GCs) per ml of rAAV described herein. In some embodiments, the unit dose contains about 2E13 genome copies (GCs) per ml of rAAV described herein.

In some embodiments, the unit dose contains about 1E10 to 1E16 genome copies (GCs) of rAAV described herein. In some embodiments, the unit dose contains about 1E11 to 1E15 genome copies (GCs) of rAAV described herein. In some embodiments, the unit dose contains about 1E12 to 1E15 genome copies (GCs) of rAAV described herein. In some embodiments, the unit dose contains about 1E13 to E15 genome copies (GCs) of rAAV described herein.

The unit dose further comprises a pharmaceutically acceptable excipient.

### 6.6.4. Targeting

The pharmaceutical composition can be used to deliver the recombinant nucleic acid vector to a target within a mammalian subject. When the pharmaceutical composition is administered, the modified rAAV can achieve a higher infection of target cells following administration to a mammalian subject as compared to an rAAV comprising a corresponding reference AAV capsid protein administered by the same route of administration and in the same dose. In some embodiments, the modified rAAV achieves higher expression in target cells of an expressible polynucleotide within the recombinant nucleic acid genome following administration to a mammalian subject as compared to the expressible polynucleotide administered in an rAAV comprising a corresponding reference AAV capsid protein administered by the same route of administration and in the same dose.

Targeting of rAAVs can be tested in an experimental animal by measuring rAAV infection or expression of a polynucleotide. In some embodiments, targeting is measured in a non-human primate (NHP), mice, rats, birds, rabbits, guinea pigs, hamsters, farm animals (including pigs and sheep), dogs, or cats.

Targeting of rAAVs can be measured after systemic or local administration of rAAVs. In some embodiments, targeting of rAAVs is measured after intravenous infusion of rAAVs or local administration to CNS. In certain embodiments, targeting is measured after administration to the CNS by lumbar puncture (LP) via injection into the lumbar cistern (e.g., approximately L3-L4) or intra cisterna magna (ICM) administration.

In some embodiments, targeting of modified rAAVs is measured by measuring the ratio between the copy numbers of the transgene transcripts and a housekeeping gene (e.g., RPP30, actin, GAPDH or ubiquitin) transcripts. In a particular embodiment, the transcripts are measured by RT-ddPCR. In some embodiments, the ratio is measured after a first administration into a mammal such as a primate, e.g., monkey (such as cynomolgus or rhesus macaque) or a mouse.

In some embodiments, rAAV of the present disclosure provides the ratio of infection (i.e., expression) in a brain (or target region of the brain) or other tissue (or non-target region of the brain) of at least 10, at least 20, at least 30, at least 40, at least 50, at least 60, at least 70, at least 80, at least 90, at least 100, at least 150, at least 200, at least 500, at least 1000 fold, compared to AAV9.

In some embodiments, a brain: comparative tissue infection ratio is measured by comparing the ratios between the copy numbers of the transgene transcripts and house keeping gene (e.g., RPP30) transcripts in the same organs (e.g., brain) or in the same tissues (e.g., caudate nucleus, frontal cortex, globus pallidum, motor cortex, parietal cortex, putamen, substantia nigra) in two individual or two groups of animals, each administered with a test rAAVₜₑₛₜ (e.g., rAAV comprising a modified AAV capsid protein) or AAV9.

$$rAAVtest : AAV 9 infection ratio = \frac{( \frac{tansgene transcripts}{housekeeping transctips} ) in rAAVtest}{( \frac{tansgene transcripts}{housekeeping transctips} ) in AAV 9}$$

In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 in the brain. In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 at one of the target tissues, caudate nucleus, frontal cortex, globus pallidum, motor cortex, parietal cortex, putamen, and substantia nigra.

In some embodiments, targeting of modified rAAVs is measured by measuring the ratio between the copy numbers of the transgene DNA genomes to copy numbers of host genes or genetic loci (e.g., RPP30). In a particular embodiment, the genomes are measured by RT-ddPCR. In some embodiments, the ratio is measured after a first administration into a mammal, e.g., a mouse, or a non-human primate such as a marmoset or rhesus macaque.

In some embodiments, a brain: comparative tissue infection ratio (DNA) is measured by comparing the ratios between the copy numbers of the transgene DNA genomes and housekeeping gene (e.g., RPP30) genomes in the same organ (e.g., brain) or in the same tissues (e.g., caudate nucleus, frontal cortex, globus pallidum, motor cortex, parietal cortex, putamen, substantia nigra) in two individual or two groups of animals, each administered with a test rAAVₜₑₛₜ (e.g., rAAV comprising a modified AAV capsid protein) or AAV9.

$$rAAV : test AAV 9 infection ratio ( DNA ) = \frac{( \frac{transgene DNA genomes}{houskeeping genomes} ) in rAAVtest}{( \frac{transgene DNA genomes}{houskeeping genomes} ) in AAV 9}$$

In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 in the brain. In some embodiments, the rAAVₜₑₛₜ achieves infection ratio of at least 2, at least 3, at least 4, at least 5, at least 6, at least 7, at least 8, at least, at least 10, at least 20, at least 30, at least 40, or at least 50 compared to AAV9 at one of the target tissues, caudate nucleus, frontal cortex, globus pallidum, motor cortex, parietal cortex, putamen, and substantia nigra.

In certain embodiments, when the (transgene genomes/housekeeping genomes) in the comparison tissue is zero or below detection limits, the brain:comparative tissue infection ratio is reported as >10,000 by convention.

In some embodiments, the modified rAAV of the present disclosure provides a brain: comparative tissue infection ratio (DNA) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1,000 or at least 10,000. In some embodiments, the muscle is triceps surae, biceps, heart or quadricep.

In some embodiments, modified rAAV of the present disclosure provides a brain: comparative tissue infection ratio (DNA) in the range of 0.5 to 1, 0.5 to 5, 0.5 to 10, 1 to 10, 1 to 100, 2 to 8, 5 to 10, 10 to 20, 20 to 80, 10 to 50, 10 to 100, 50 to 80, 100 to 500, 100 to 1000, or 500 to 1000. In some embodiments, the muscle is triceps surae, biceps, heart, or quadricep. In some embodiments, the modified rAAV achieves a brain: comparative tissue infection ratio (DNA) of at least 2, at least 5, at least 10, at least 20, at least 30, at least 40, at least 50, at least 60, at least 70, at least 80, at least 90, at least 100, at least 150, at least 200, at least 500, at least 1000. In some embodiments, the modified rAAV achieves a brain: comparative tissue infection ratio of 0.1 to 1, 1 to 5, 1 to 10, I to 20, 1 to 50, i to 100, 1 to 200, 1 to 300, 100 to 500, 250 to 750, or 500 to 1000.

### 6.6.4.1 RNA Data—Brain:Liver Infection Ratio

In some embodiments, targeting of modified rAAVs is measured by measuring the ratio between the copy numbers of the transgene transcripts and housekeeping gene (e.g., RPP30) transcripts. In a particular embodiment, the transcripts are measured by RT-ddPCR. In some embodiments, the ratio is measured after a first administration into a mammal, e.g., a mouse, or a non-human primate such as a marmoset or rhesus macaque.

In some embodiments, a brain:liver infection ratio (RNA) is measured by comparing the ratios between the copy numbers of the transgene transcripts and housekeeping gene (e.g., RPP30) transcripts in the two different organs (e.g., brain v. liver).

$$brain : liver infection ratio ( RNA ) = \frac{( \frac{tansgene transcripts}{housekeeping transctips} ) in brain}{( \frac{tansgene transcripts}{housekeeping transctips} ) in liver}$$

In some embodiments, modified rAAV of the present disclosure provides a (transgene transcripts/housekeeping transcripts) ratio in liver of less than 1000, less than 900, less than 800, less than 700, less than 600, less than 500, less than 400, less than 300, less than 200, less than 100, less than 90, less than 80, less than 70, less than 60, less than 50, less than 40, less than 30, less than 20, or less than 10.

In certain embodiments, when the (transgene transcripts/housekeeping transcripts) in the liver is zero or below detection limits, the brain:liver infection ratio is reported as >10,000 by convention.

In some embodiments, the modified rAAV of the present disclosure provides a brain:liver infection ratio (RNA) of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 500, at least 1000. In some embodiments, the muscle is caudate nucleus, frontal cortex, globus pallidum, motor cortex, parietal cortex, putamen, or substantia nigra.

In some embodiments, modified rAAV of the present disclosure provides a brain:liver infection ratio (RNA) of 1 to 10, 1 to 100, 10 to 20, 10 to 50, 10 to 80, 10 to 100, 20 to 100, 100 to 500, 100 to 1000, or 500 to 1000. In some embodiments, the muscle is caudate nucleus, frontal cortex, globus pallidum, motor cortex, parietal cortex, putamen, or substantia nigra.

### 6.6.4.2 DNA Data—Brain:Liver Infection Ratio

In some embodiments, targeting of modified rAAVs is measured by measuring the ratio between the copy numbers of the transgene DNA genomes to copy numbers of host genes or genetic loci (e.g., RPP30). In a particular embodiment, the genomes are measured by RT-ddPCR. In some embodiments, the ratio is measured after a first administration into a mammal, e.g., a mouse, or a non-human primate such as a marmoset or rhesus macaque.

In some embodiments, a brain:liver infection ratio (DNA) is measured by comparing the ratios between the copy numbers of the transgene DNA genomes and housekeeping gene (e.g., RPP30) genomes in the two different organs (e.g., brain v. liver).

$$brain : liver infection ratio ( DNA ) = \frac{( \frac{transgene DNA genomes}{houskeeping genomes} ) in brain}{( \frac{transgene DNA genomes}{houskeeping genomes} ) in liver}$$

In some embodiments, modified rAAV of the present disclosure provides a (transgene genomes/housekeeping genomes) ratio in liver of less than 1, or in a range from 1 to 10, 1 to 5, i to 2, 0.1 to 1, 0 to 1, 0.01 to 0.1, 0.01 to 0.5, or 0.01 to 0.05.

In certain embodiments, when the (transgene genomes/housekeeping genomes) in the liver is zero or below detection limits, the brain:liver infection ratio is reported as >10,000 by convention.

In some embodiments, the modified rAAV of the present disclosure provides a brain:liver infection ratio (DNA) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1,000 or at least 10,000. In some embodiments, the muscle is caudate nucleus, frontal cortex, globus pallidum, motor cortex, parietal cortex, putamen, or substantia nigra.

In some embodiments, modified rAAV of the present disclosure provides a brain:liver infection ratio (DNA) in the range of 0.5 to 1, 0.5 to 5, 0.5 to 10, I to 10, 1 to 100, 2 to 8, 5 to 10, 10 to 20, 20 to 80, 10 to 50, 10 to 100, 50 to 80, 100 to 500, 100 to 1000, or 500 to 1000. In some embodiments, the muscle is caudate nucleus, frontal cortex, globus pallidum, motor cortex, parietal cortex, putamen, or substantia nigra. In some embodiments, the modified rAAV achieves a brain:liver infection ratio (DNA) of at least 2, at least 5, at least 10, at least 20, at least 30, at least 40, at least 50, at least 60, at least 70, at least 80, at least 90, at least 100, at least 150, at least 200, at least 500, at least 1000. In some embodiments, the modified rAAV achieves a brain:liver infection ratio of 0.1 to 1, 1 to 5, I to 10, 1 to 20, 1 to 50, 1 to 100, 1 to 200, 1 to 300, 100 to 500, 250 to 750, or 500 to 1000.

### 6.6.4.3 IHC Data Brain:Liver Infection Ratio

In some embodiments, targeting of modified rAAVs is calculated using the % of cells that have been successfully transduced and express a transgene in a tissue (e.g., eGFP). In a particular embodiment, the transgene expression is measured by immunohistochemistry. In some embodiments, the ratio is measured after a first administration into a mammal, e.g., a mouse, or a non-human primate such as a marmoset or rhesus macaque.

In some embodiments, a brain:liver infection ratio (IHC) is measured by comparing the ratios between the transgene % GFP+ cells and housekeeping gene (e.g., RPP30) % GFP+ cells in the two different organs (e.g., brain v. liver).

$$brain : liver infection ratio ( IHC ) = \frac{( \frac{transgene \% GFP + cells}{housekeeping \% GFP + cells} ) in brain}{( \frac{transgene \% GFP + cells}{housekeeping \% GFP + cells} ) in liver}$$

In some embodiments, modified rAAV of the present disclosure provides a (transgene % GFP/housekeeping % GFP) ratio in liver of less than 1, less than 5, less than 10, or in a range from 1 to 10, 1 to 5, 1 to 2, 0.1 to 1, 0 to 1, 0.01 to 0.1, 0.01 to 0.5, or 0.01 to 0.05.

In certain embodiments, when the (transgene % GFP/housekeeping % GFP) in the liver is zero or below detection limits, the brain:liver infection ratio is reported as >10,000 by convention.

In some embodiments, the modified rAAV of the present disclosure provides a brain:liver infection ratio (IHC) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1000. In some embodiments, the muscle is caudate nucleus, frontal cortex, globus pallidum, motor cortex, parietal cortex, putamen, or substantia nigra.

In some embodiments, modified rAAV of the present disclosure provides a brain:liver infection ratio (IHC) of 1 to 5, 1 to 10, 1 to 100, 2 to 8, 10 to 20, 20 to 30, 10 to 50, 10 to 100, 20 to 80, 50 to 80, 100 to 500, 100 to 1000, or 500 to 1000. In some embodiments, the muscle is caudate nucleus, frontal cortex, globus pallidum, motor cortex, parietal cortex, putamen, or substantia nigra.

### 6.7. Method of Use

A modified rAAV as described herein can be used in research and/or therapeutic applications. In some embodiments, a modified rAAV is for genetically modifying a cell in vitro or in vivo. In some embodiments, a modified rAAV is used for gene therapy or for vaccination in a human or animal. More specifically, a modified rAAV can be used for gene addition, gene augmentation, genetic delivery of a polypeptide therapeutic, genetic vaccination, gene silencing, genome editing, gene therapy, RNAi delivery, cDNA delivery, mRNA delivery, miRNA delivery, miRNA sponging, genetic immunization, optogenetic gene therapy, transgenesis, DNA vaccination, or DNA immunization of liver cells or non-liver cells.

In some embodiments, a modified rAAV of the present disclosure is used for treatment of treating or ameliorating or preventing a disease or condition in a subject. In some embodiments, the disease is a disease of the central nervous system (CNS).

In some embodiments, a modified rAAV of the present disclosure is used for transferring an exogenous polynucleotide to the central nervous system (CNS). In some embodiment, transferring the exogenous polynucleotide to the CNS results in a CNS:liver infection ratio of greater than 1 when measured by genome copies of the AAV virion. In some embodiments, transferring the exogenous polynucleotide to the CNS results in expression of the exogenous polynucleotide in the CNS at a CNS:liver expression ratio of greater than 10. In some embodiments, transferring the exogenous polynucleotide to the CNS results in expression of the exogenous polynucleotide in the CNS at a CNS:liver expression ratio of greater than 10 when measured by protein expression.

A modified rAAV of the present disclosure can be administered to a subject in a suitable pharmaceutical carrier.

The rAAV of the disclosure are typically administered in sufficient amounts to transduce or infect the desired cells and to provide sufficient levels of gene transfer and expression to provide a therapeutic benefit to subjects suffering from a disease. In particular embodiments, the rAAV is administered in sufficient amounts to provide a therapeutic benefit to subjects suffering from a disease of the central nervous system (CNS).

Conventional and pharmaceutically acceptable routes of administration include, but are not limited to, direct delivery to an organ such as, for example, the brain, intra cisterna magna (ICM), inter-digitally, intravenously, orally, intranasally, intratracheally, intrathecally, intramuscularly, intraocularly, subcutaneously, intradermally, or by other routes of administration. Routes of administration can be combined, if desired.

Transduction and/or expression of the transgene can be monitored at various time points following administration by DNA, RNA, or protein assays.

Accordingly, the present disclosure provides a method of treating and/or preventing a muscular disease and/or muscle degeneration by administering a modified rAAV described herein.

### 7. EXAMPLES

### 7.1. Example 1: Assessment of CNS Tropism of Anc80L65 Compared to AAV9

Applicant evaluated distribution of AAV9 and Anc80L65 vectors encoding the EGFP reporter 14 days following injection by either lumbar puncture (LP) injection into the lumbar cistern (approximately L3-L4) or intra cisterna magna (ICM) injection (4E13gc/animal; 2E13 vg/ml) in adult cynomolgus macaques. Applicant demonstrated that a single injection of Anc80L65 into the CSF of adult cynomolgus monkeys led to the efficient transduction of broad regions of the CNS.

Following ICM injection, Anc80L65 distributes more broadly throughout the cortex and into deep brain nuclei compared to AAV9. Following LP injection, Anc80L65 distribution throughout the cortex was on par with ICM delivery and superior to that seen with AAV9 via ICM delivery. AAV9 showed limited transduction in the cortex following LP delivery. AAV9 and Anc80L65 efficiently transduced spinal cord ventral horn motor neurons with both routes of administration.

Specifically, Anc80L65 transducing both neurons and astrocytes. Rare oligodendrocyte transduction was also observed in cortical regions with Anc80L65, however no microglial cells were found to be transduced using the microglial marker Iba1. AAV9 showed a similar tropism in the nonhuman primate CNS to Anc80L65, transducing largely neurons and astrocytes. Similar to Anc80L65 no microglial double labeling was observed. Oligodendrocyte transduction was not observed with AAV9, however there was less transduction overall in the CNS compared to Anc80L65 making it a difficult comparison.

This work demonstrated the ability of Anc80L65 to target widespread regions of the CNS following CSF routes of delivery and outperforms the distribution of AAV9 in targeting cortical and deep brain regions. The ability of Anc80L65 to mediate efficient gene transfer and expression in neurons and astrocytes throughout the brain and spinal cord of NHPs supports use of Anc80L65 vector for treatment of a wide range of neurologic disorders.

### 7.1.1. Experimental Procedures

### 7.1.1.1 Lumbar Puncture (LP) Injection

The animal was injected with anesthesia and were placed in lateral recumbency. A 22-gauge Gerti Marx spinal needle was percutaneously inserted into the lumbar cistern (approximately L3-L4). Fluoroscopy was used for guidance if necessary. Once the needle was placed, the stylet was removed, and positive cerebral spinal fluid (CSF) flow confirmed, and predose CSF was collected. The test article syringe was then attached to the needle and the test article slowly infused by hand as a slow bolus over approximately 120±5 seconds. After completion of the injection, the needle was removed, and brief pressure was applied by hand over the injection site. Animal was then be placed in the Trendelenburg position (30°, head down) for a minimum of approximately 10 minutes. The animal was then allowed to recover naturally from anesthesia. Lumbar puncture is an intrathecal injection.

### 7.1.1.2 Intracisternal Magna (ICM) Injection

The animal was injected with anesthesia and placed in lateral recumbency. A 22-gauge spinal needle was advanced percutaneously into the cisterna magna, correct needle placement was verified by the presence of positive cerebral spinal fluid (CSF) flow, and predose CSF was collected. An appropriate Test Article syringe was then be connected to the spinal needle and the Test Article was administered by hand via a slow bolus injection (120±5 seconds). After completion of the injection, the syringe was removed, and pressure was applied briefly by hand. Animal was then placed in the Trendelenburg position (30°, head down) for a minimum of approximately 10 minutes. The animal was then be allowed to recover naturally from anesthesia.

### 7.1.1.3 Immunohistochemistry (IHC)

Two weeks after injection, tissue samples were collected and preserved in 10% neutral buffered formalin (NBF) for 48-72 hours, then transferred to 70% ethanol. The brain was placed into a pre-chilled brain matrix and sliced into 4 mm sections, then hemisected. Even-numbered hemisected slabs were preserved in 10% NBF and used for immunohistochemistry (IHC). Odd-numbered hemisected brain slabs were frozen on dry ice and stored at −60 to −90° C. until used for ddPCR analysis.

For detection of GFP expression, slides were incubated with antibodies against GFP (GeneTex, GTX₂₀₂₉₀) diluted 1:1,000 in Monet Blue Diluent (Biocare Medical, PD901). The slides were washed with Valent Wash Buffer (Biocare Medical, VLT8013MX) and incubated with anti-rabbit antibody conjugated with Farma HRP for 30 minutes (Biocare Medical, BRR4009). The slides were washed and then reacted with Betazoid DAB for 5 minutes (Biocare Medical, BDB2004) and counterstained with Mayer's Hematoxylin for 5 minutes (StatLab, HXMMHPT). After the reactions with Betazoid DAB or Mayer's Hematoxylin, the slides were washed with Aqua Rinse (Biocare Medical, VLT8012MX).

GFP staining by 3,3′-diaminobenzidine (DAB): Sections (3 per each 6-mm block: separation of 2 mm) were washed 3 times in PBST followed by treatment with 1% H2O2. Sections were stained with the primary anti-GFP antibody diluted 1:1000 in Da Vinci Green Diluent as previously described (Lluis Samaranch, Ernesto A. Salegio, Waldy San Sebastian, Adrian P. Kells, John R. Bringas, John Forsayeth, and Krystof S. Bankiewicz Human Gene Therapy. Volume: 24 Issue 5: Mar. 20, 2013, incorporated herein by reference).

For detection of Trastuzumab expression, slides were incubated with antibodies against IgG (Fc). IgG (Fc) can serve as a proxy for Trastuzumab expression.

### 7.1.1.4 Double-Immunofluorescence:

Fluorescence immunostaining of different cellular markers (NeuN, GFAP, Iba1, Olig2+) with GFP as previously described (San Sebastian et al., 2013).

### Sample Collection:

Tissue samples were collected and preserved in 10% neutral buffered formalin (NBF) for 48-72 hours, then transferred to 70% ethanol. The brain was placed into a pre-chilled brain matrix and sliced into 4 mm sections, then hemisected. Even-numbered hemisected slabs were preserved in 10% NBF and used for immunohistochemistry (IHC). Odd-numbered hemisected brain slabs were frozen on dry ice and stored at −60 to −90° C. until used for ddPCR analysis.

### Immunohistochemistry Protocol for GFP Expression:

Bake slides for 15 minutes at 55-65 Celsius to remove paraffin
Load slides onto Valent Staining Platform (Biocare Medical)
Val DePar 8 minutes (Biocare Medical, VLT800 I MM)
Lo pH1 AR at 98 Celsius for 60 minutes (Biocare Medical, VLT8004MM)
Peroxidazed 1 for 5 minutes (Biocare Medical, PX₉₆₈)
Background Punisher for 5 minutes (Biocare Medical, BP974)
GFP (GeneTex, GTX₂₀₂₉₀) 1:1,000 in Monet Blue Diluent (Biocare Medical, PD901)
Rabbit on Farma H1RP for 30 minutes (Biocare Medical, BRR4009)
Betazoid DAB for 5 minutes (Biocare Medical, BDB2004)
Counterstain with Mayer's Hematoxylin for 5 minutes (StatLab, HXMMHPT)

Valent Wash Buffer (Biocare Medical, VLT8013MX) was used after all steps expect for Betazoid DAB and Mayer's Hematoxylin. Aqua Rinse (Biocare Medical, VLT8012MX) was used after these reagents.

Dual Staining Methods for the IBA1, NeuN and GFAP with the GFP:

### Reagents:

GFP (GeneTex, GTX20290) 1:1,000, GFAP (Cell Signaling, 3670) 1:500 in Monet Blue Diluent (Biocare Medical, PD901)
GFP (GeneTex, GTX20290) 1:1,000, IBA1 (Millipore, MABN92) 1:250 in Monet Blue Diluent (Biocare Medical, PD901)
GFP (GeneTex, GTX20290) 1:1,000, NeuN (Abcam, ab104224) 1:250 in Monet Blue Diluent (Biocare Medical, PD901)

### Protocol:

Bake slides for 15 minutes at 55-65 Celsius to help remove paraffin
Load slides onto Valent Staining Platform (Biocare Medical)
Val DePar 8 minutes (Biocare Medical, VLT8001MM)
Lo pH AR at 98 Celsius for 60 minutes (Biocare Medical, VLT8004MM)
Peroxidazed 1 for 5 minutes (Biocare Medical, PX968)
Background Punisher for 10 minutes (Biocare Medical, BP974)
Primary Antibody Cocktail: Rabbit 594 nm (Invitrogen, A32740) 1:500, Mouse 488 nm (Invitrogen, A−21202) 1:500, cocktailed together in Da Vinci Green for 60 minutes (Biocare Medical, PD900)
Coverslip with Prolong Diamond Antifade Reagent with DAPI
Valent Wash Buffer (Biocare Medical, VLT8013MX) was used after all steps.

### 7.1.1.5 Ddpcr

After euthanasia and exsanguination, brains were placed into a pre-chilled brain matrix and sliced into 4 mm sections, then hemisected. Odd numbered hemisected slabs were frozen over dry ice, then stored at −60° C. to −90° C. until analyzed. Brain regions were isolated using 2 mm or 3 mm diameter tissue punches (Miltex, Cat. No.: 95039-098 and 98PUN6-4) prior to nucleic acid isolation.

Tissues were homogenized in a Qiagen Tissuelyser II (20rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. Prior to analysis, DNA and RNA concentration and quality were determined using a NanoDrop One, using the nucleic acid (DNA or RNA) program. DNA samples were analyzed for biodistribution of vector genomes using a duplexed ddPCR method targeting the transgene (eGFP) and a reference gene (RPP30). RNA samples were analyzed for expression of the eGFP transgene using a duplexed, one-step RT-ddPCR method) and a reference gene (RPP30).

### Dual Staining Method for Olig22 and GFP (Performed at StageBio):

### Reagents:

GFP (GeneTex, GTX₂₀₂₉₀) 1:1,000, Olig2 (Millipore, MABN50) 1:250 in Monet Blue Diluent (Biocare Medical, PD901)

### Protocol:

Bake slides for 15 minutes at 55-65 Celsius to help remove paraffin
Load slides onto Valent Staining Platform (Biocare Medical)
Val DePar 8 minutes (Biocare Medical, VLT8001MM)
Lo pH AR at 98 Celsius for 60 minutes (Biocare Medical, VLT8004MM)
Peroxidazed for 5 minutes (Biocare Medical, PX₉₆₈)
Background Punisher for 10 minutes (Biocare Medical, BP974)
Primary Antibody Cocktail: Biotinylated Mouse (Vector Laboratories, BA-9200) 1:500 in Da Vinci Green Diluent
Rabbit 594 nm (Invitrogen, A32740) 1:500, Streptavidin 488 nm (Invitrogen, S11223) 1:500, cocktailed together in Da Vinci Green for 60 minutes (Biocare Medical, PD900)
Coverslip with Prolong Diamond Antifade Reagent with DAPIValent Wash Buffer (Biocare Medical, VLT8013MX) was used after all steps.

### DNA Analysis:

For isolation of DNA, tissues were homogenized in a Qiagen Tissuelyser II (20rps for 2 min) in lysis buffer from the Qiagen DNeasy Blood and Tissue Kit (Part No. 69506), following the standard Qiagen protocol. Samples were eluted in 50 uL of AE buffer. Prior to analysis, DNA concentration and quality were determined using a NanoDrop One, using the nucleic acid (DNA) program.

DNA samples were analyzed for biodistribution of vector genomes using a duplexed ddPCR method targeting the transgene (eGFP or Trastuzumab) and a reference gene (RPP30). Specific primer probe sequences are listed in the table below.

| | | SEQ | |
|---|---|---|---|
| Name | Target | ID NO: | Sequence |
| pCAG.eGFP_DNA | eGFP | 55863 | GCTTCTGGCGTGTGACC |
| FWD Set 4 | | | |
| pCAG.eGFP_DNA | eGFP | 55864 | TGATGAGACAGCACAAT |
| REV Set 4 | | | AACCAG |
| pCAG.eGFP_DNA | eGFP | 55865 | FAM/TTTCCTACA/ZEN/ |
| PRB Set 4 | | | GCTCCTGGGCAACG/ |
| | | | 3IABKFQ |
| RPP30_NHP_DNA | RPP30 | 55866 | GAACCTGAAACTTCACA |
| FWD Set 3 | | | |
| RPP30_NHP_DNA | RPP30 | 55867 | CCATTTAAGGAGTGGTTAT |
| REV Set 3 | | | |
| RPP30_NHP_DNA | RPP30 | 55868 | HEX/TAAAGTCTA/ZEN/ |
| PRB Set 3 | | | CGCACTACCACTTAC/ |
| | | | 3IABKFQ |

The samples were analyzed following the standard Bio-Rad ddPCR protocol for probe-based analysis of DNA biodistribution. Briefly, reaction mixes containing the 2 primer probe sets, DNA samples and Bio-Rad ddPCR Supermix for Probes (no dUTP) (Part No. 186-3024) were prepared according to the recipe in the table below.

| Reagent | Vol/reaction |
|---|---|
| 2X ddPCR Supermix | 10 |
| 20X RPP30 PnP | 1 |
| 20X eGFP PnP or 20X Trastuzumab PnP | 1 |
| Water | 3 |
| Sample* | 3 |

*DNA samples were pre-diluted to 2 ng/μL (liver), 10 ng/μL (DRG, no dilution for the samples with the concentration <10 ng/μL) and 20 ng/μL (other samples) using nuclease-free water.

After droplet generation, reactions were amplified using the thermal cycling program indicated below.

| Parameter | Time | Temp ° C. | Cycles |
|---|---|---|---|
| Enzyme Activation | 10 min | 95° C. | 1 |
| Denaturation | 30 sec | 94° C. | 40 |
| Annealing | 30 sec | 54° C. | |
| Extension | 60 sec | 74° C. | |
| Enzyme deactivation | 10 min | 98° C. | 1 |
| Hold | ∞ (Press Cancel Run to end | 4° C. | 1 |
| | program) | | |

| | |
|---|---|
| Ramp Rate | 2° C./sec |
| Volume | 40 μL |
| Lid Temperature | 105° C. |

Data is reported in vector genomes copied per diploid genome (VGC/DG). The formula for calculating the output is VGC/DG=(eGFP cp/μL÷RPP30 cp/μL)×2 for eGFP or VGC/DG=(Trastuzumab cp/μL÷RPP30 cp/μL)×2 for Trastuzumab.

### RNA Analysis:

For isolation of mRNA, tissues were homogenized in a Qiagen Tissuelyser II (20rps for 1 min) in 1 ml of Qiazol from the Qiagen RNeasy Lipid Tissue Mini Kit (Part No. 74804), following the standard Qiagen protocol. Samples were eluted in 50 L of Nuclease-free water. Prior to analysis, RNA concentration and quality were determined using a NanoDrop One, using the nucleic acid (RNA) program.

DNA samples were analyzed for expression of the eGFP transgene or the Trastuzumab transgene using a duplexed, one-step RT-ddPCR method targeting the transgene (eGFP or Trastuzumab) and a reference gene (RPP30). Specific primer probe sequences are listed in the table below.

| | | SEQ ID | |
|---|---|---|---|
| Name | Target | NO: | Sequence |
| pCAG.eGFP | eGFP | 55869 | CACAGCTCCTGGGCAAC |
| RNA FWD Set 5 | | | |
| pCAG.eGFP | eGFP | 55870 | AGCTCGACCAGGATGGG |
| RNA REV Set 5 | | | |
| pCAG.eGFP | eGFP | 55871 | FAM/ATGGTGAGC/ZEN/ |
| RNA PRB Set 5 | | | AAGGGCGAGGA/ |
| | | | 3IABKFQ |
| RPP30 NHP R | RPP30 | 55872 | GCGGGTTCTGACCTGAAG |
| NA FWD Set 3 | | | |
| RPP30 NHP R | RPP30 | 55873 | TCCCTGTACAATCGGTAA |
| NA REV Set 3 | | | AGTTG |
| RPP30 NHP R | RPP30 | 55874 | HEX/CGGCTCACC/ZEN/ |
| NA PRB Set 3 | | | TTGGCTATTCAGTTGT/ |
| | | | 3IABkFQ |

The samples were analyzed following the standard Bio-Rad RT-ddPCR protocol for probe-based analysis of RNA expression. Briefly, reaction mixes containing the 2 primer probe sets, RNA samples and Bio-Rad One-Step RT-ddPCR Advanced Kit for Probes (Part No. 186-4021) were prepared according to the recipe in the table below.

| Reagent | Vol (uL)/reaction |
|---|---|
| Supermix | 5 |
| 300 mM DTT | 1 |
| Reverse Transcriptase | 2 |
| 20X RPP30 PnP | 1 |
| 20X eGFP PnP or 20x Trastuzumab PnP | 1 |
| Nuclease-free water | 5 |
| RNA Sample* | 5 |

*RNA samples were pre-diluted to 20 ng/μL using nuclease-free water.

After droplet generation, reactions were amplified using the thermal cycling program indicated below.

| Parameter | Time | Temp ° C. | Cycles |
|---|---|---|---|
| Reverse Transcription | 60 min | 48° C. | 1 |
| Enzyme Activation | 10 min | 95° C. | 1 |
| Denaturation | 30 sec | 94° C. | 40 |
| Annealing | 30 sec | 57 | |
| Extension | 60 sec | 74° C. | |
| Enzyme deactivation | 10 min | 98° C. | 1 |
| Hold | ∞ (Press Cancel Run | 4° C. | 1 |
| | to end program) | | |

| | |
|---|---|
| Ramp Rate | 2° C./sec |
| Volume | 40 μL |
| Lid Temperature | 105° C. |

Data is reported as 00 eGFP expression, which is calculated according to the formula, 00 eGFP expression=(eGFP cp/μL÷RPP30 cp/μL)×100 or 00 Trastuzumab expression=(Trastuzumab cp/μL÷RPP30 cp/μL)×100.

### 7.1.2. Broad CNS Penetration and Wide Distribution of Anc80L65 Compared to AAV9

The objective of this study is to determine the biodistribution and initial feasibility of Anc80L65 vector compared to AAV9 vector, when administered by a single lumbar puncture or intra-cisterna magna administration. The results confirm broad penetration and wide distribution of Anc80L65 compared to AAV9.

Two AAV constructs were used in the experiment: (i) Anc80L65-CAG-GFP, and (ii) AAV9-CAG-GFP, each including an AAV genome construct containing a coding sequence of GFP. GFP was used to detect distribution of AAVs and expression of the transgene. Cynomolgus monkeys were used as the subject animals.

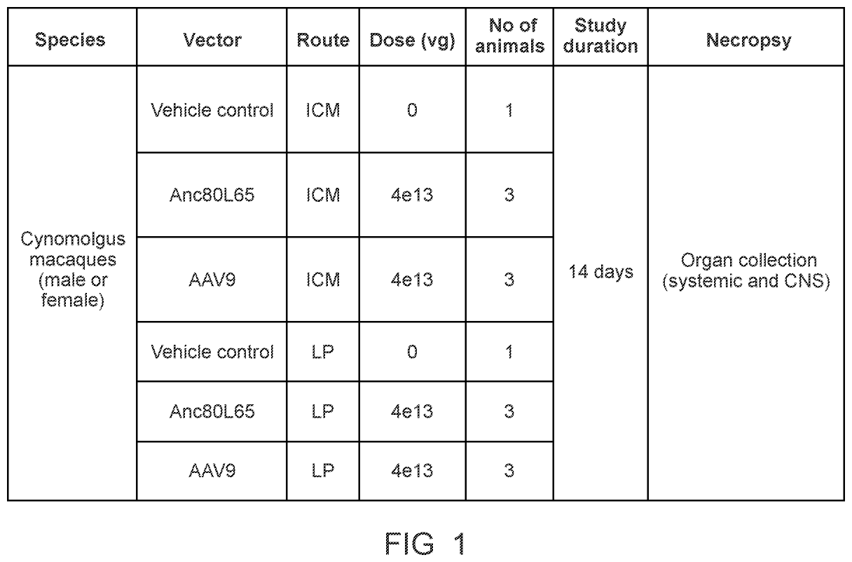

Total 14 animals were divided into 6 groups as summarized in the FIG. 1 and TABLE 2. Animals in Group 1 and 4 are control animals administered with vehicle. Animals in Group 2 and 5 were administered with 4E13vg (viral genome or GC) of Anc80L65, and animals in Group 3 and 6 were administered with 4E13vg of AAV9. Two routes of administration were tested—animals in Group 1-3 were administered by ICM, and animals in Group 4-6 were administered by LP. Animals were sacrificed on day 14 or 15 after the vehicle or AAV administration and their organ samples were collected for analysis.

TABLE 2
NHP Experiment Design

| | | | | Dose | Dose Con- | |
|---|---|---|---|---|---|---|
| Group | Test | Dose | Dose Level | Volume | centration | # of |
| No. | Material | Route | vg/animal | (mL) | (vg/mL) | animals |
| 1 | Vehicle | ICM | 0 | 2 | 0 | 1 |
| 2 | Anc80L65 | ICM | 4E13 | 2 | 2E13 | 3 |
| 3 | AAV9 | ICM | 4E13 | 2 | 2E13 | 3 |
| 4 | Vehicle | LP | 0 | 2 | 0 | 1 |
| 5 | Anc80L65 | LP | 4E13 | 2 | 2E13 | 3 |
| 6 | AAV9 | LP | 4E13 | 2 | 2E13 | 3 |

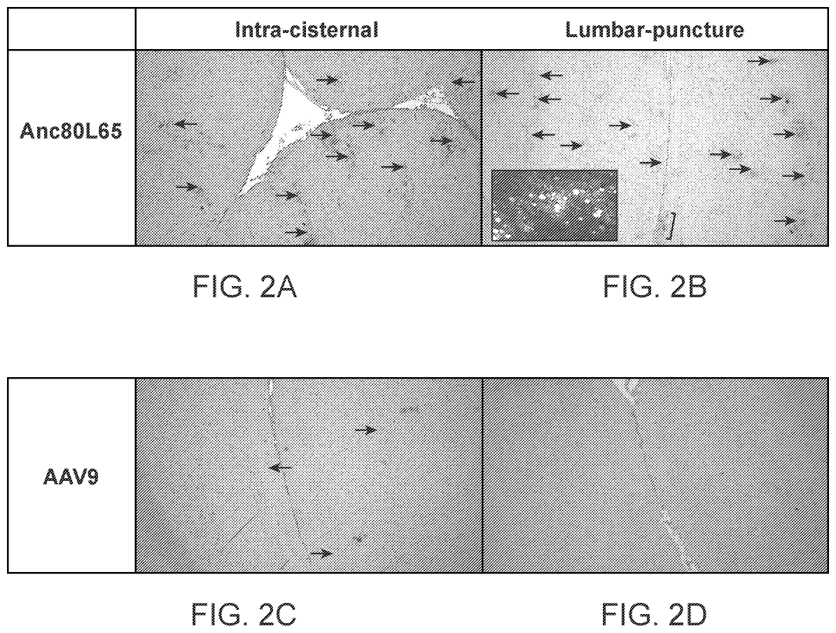

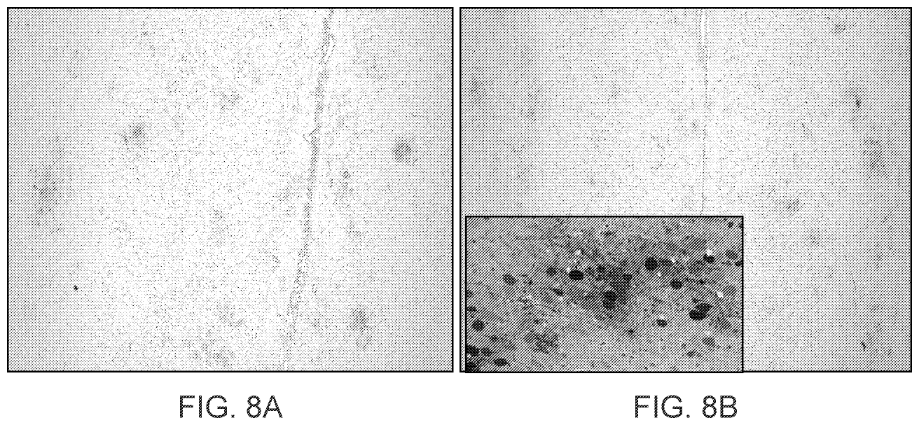

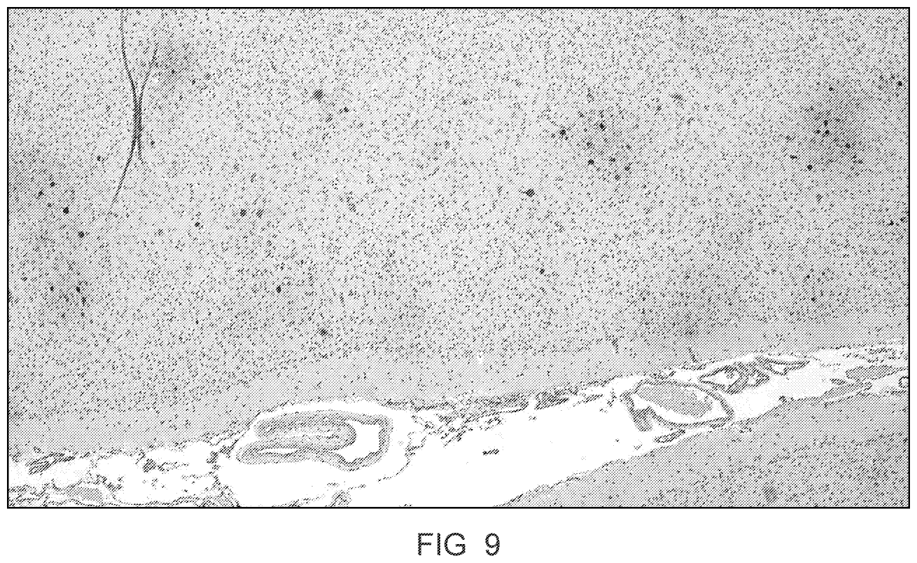

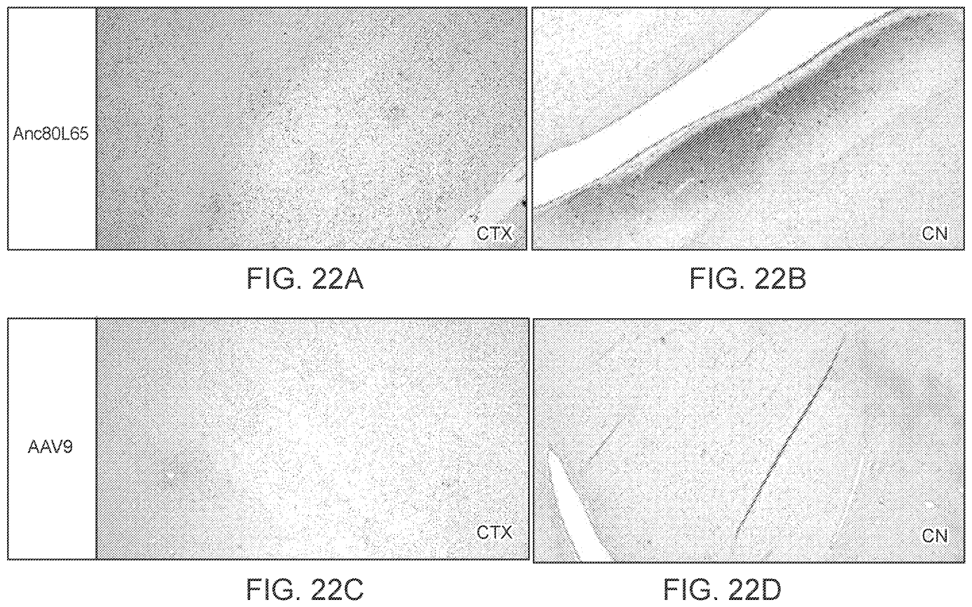

Collected samples were processed for IHC and stained with an antibody against GFP. Images of the IHC staining are provided in FIGS. 2A-9 and 22A-22D. FIGS. 2A-2D provide immunohistochemistry (IHC) images of cortical tissue from the brain sections obtained from NHPs administered with Anc80L65 or AAV9 by intracisternal magna injection or lumbar-puncture. FIGS. 22A-22D provide IHC images of brain sections of cortex and caudate nucleus obtained from NHPs administered with Anc80L65 or AAV9 by intracisternal magna injection.

These results show transgene (GFP) expression capabilities of Anc80L65 are superior compared to AAV9 both by ICM and LP administrations. More cells were stained for GFP expression in the cortex and caudate nucleus after administration of Anc80L65 compared to AAV9. FIGS. 2A-2D further show that ICM administration provides better results than LP administration with both vectors (i.e., Anc80L65 and AAV9) in terms of breadth of distribution within the brain.

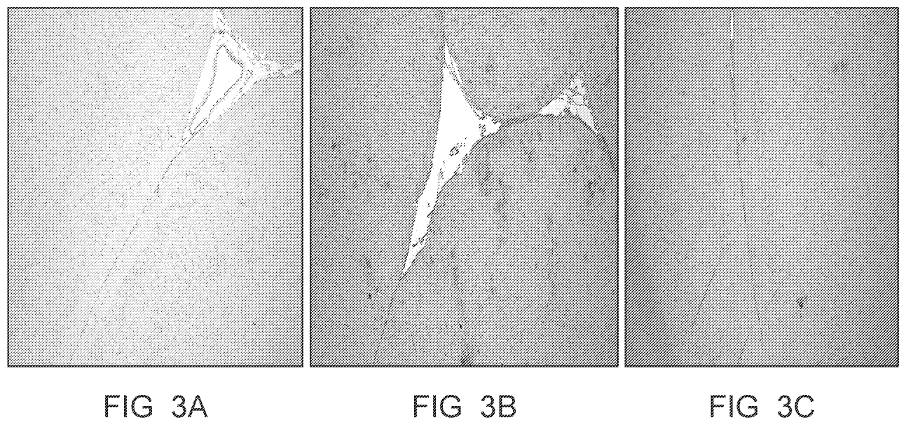

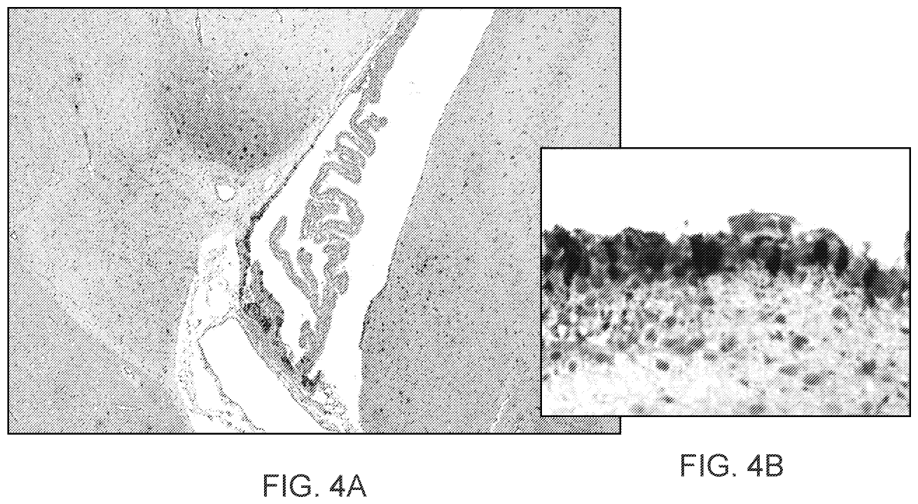

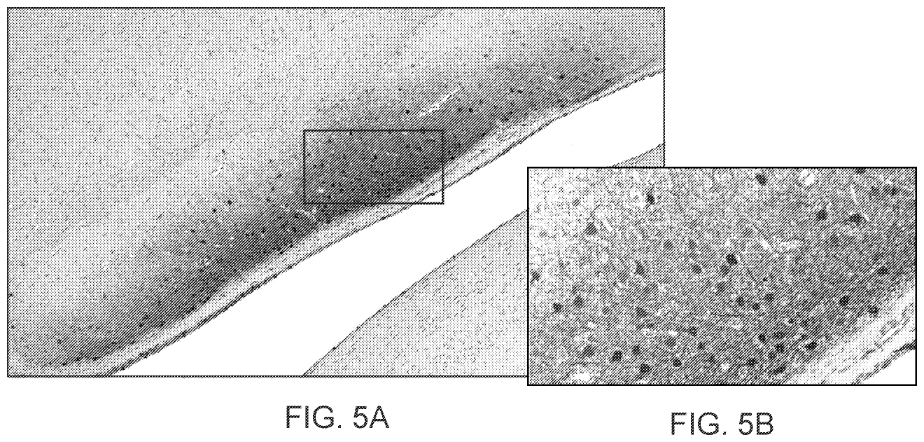

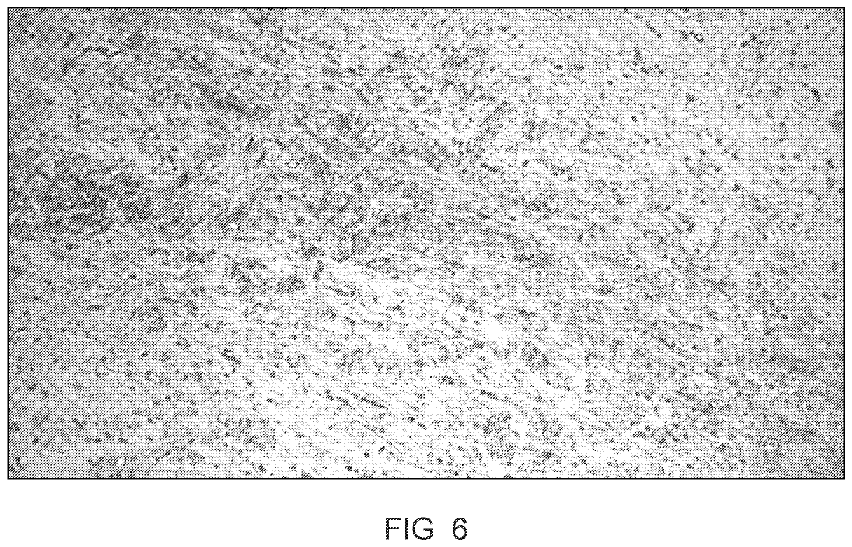

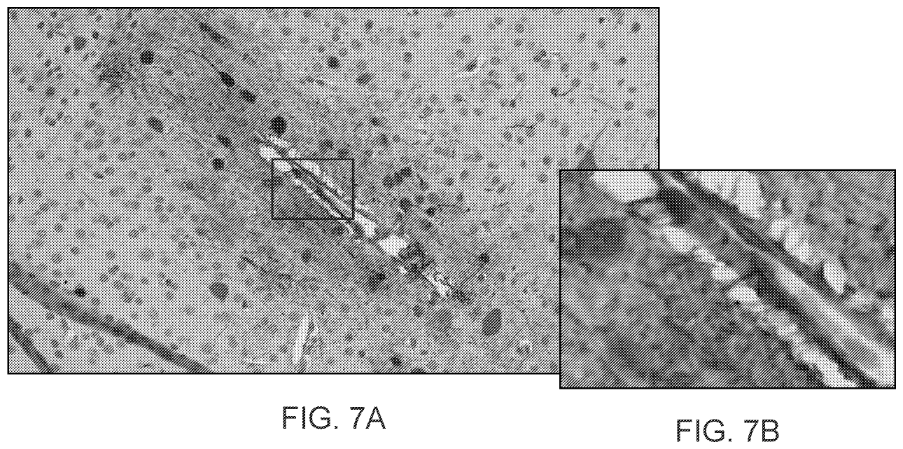

IHC results in other parts of the brain are also provided—specifically, in the cortex (FIGS. 3A-3C, 8A-8C and 9), ependyma and caudate nucleus (FIGS. 4A-4B), caudate nucleus (FIGS. 5A-5B), substantia nigra (FIG. 6), and perivascular cells (FIG. 7A-7B). The results show broad penetration and wide distribution of Anc80L65 compared to AAV9.

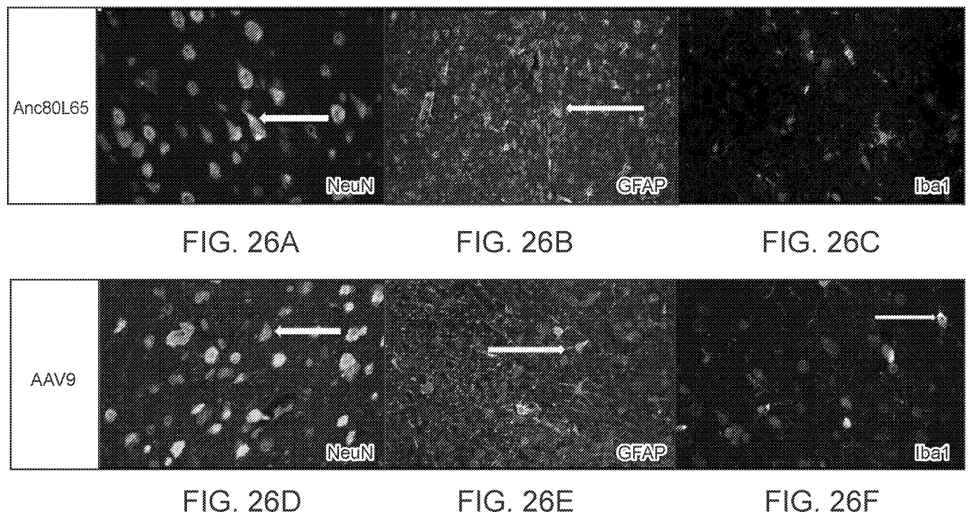

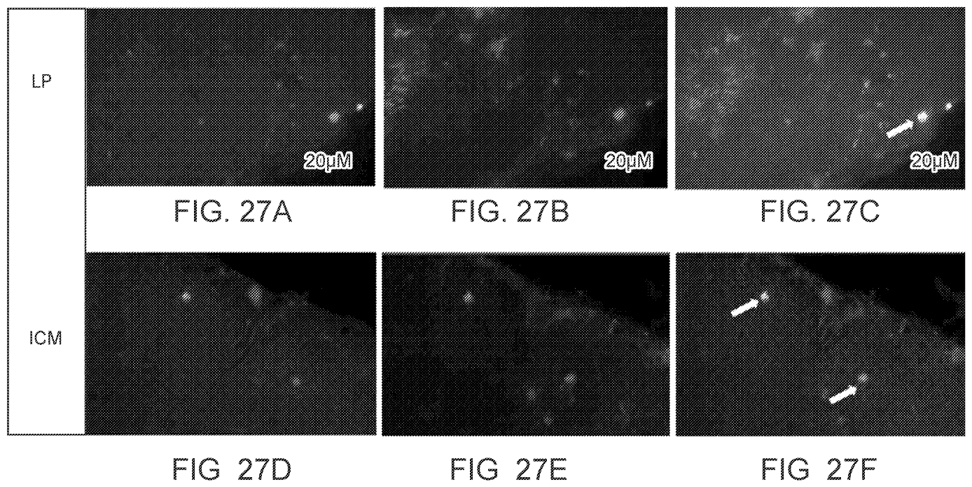

To characterize cell types expressing GFP after Anc80L65 or AAV9 administration, the NHP brain sections were double stained for GFP and a cell-type specific marker. FIGS. 26A-26F and FIGS. 27A-27F provide the images of the double staining —-against GFP and a marker for neurons (NeuN) (FIGS. 26A and 26D), against GFP and a marker for astrocytes (FIGS. 26B and 26E), against GFP and a marker for microglial cells (iba1), against GFP and a marker for oligodendrocyte (FIGS. 27A, 27B and 27C) in the motor cortex transfected with Anc80L65 or AAV9. In all cases, GFP+ cells are shown in red, the cell specific marker is shown in green, and the merged images are shown with double-labeled cells in yellow/orange (arrows). The staining results show that Anc80L65 can mediate efficient transgene expression in neurons, astrocytes and oligodendrocytes across large regions of the NHP brain following a single LP or ICM injection. This suggests that Anc80L65 can be used for clinical applications to treat a wide range of neurologic disorders, particularly using a relatively noninvasive route of administration such as LP.

Transgene transfer and expression capabilities of Anc80L65 and AAV9 administered by ICM or LP to NHPs were also tested with ddPCR, by measuring amounts of DNA and mRNA of the transgene (eGFP) in the NHP brain and spinal cord 2 weeks after ICM or LP delivery. DNA genome copies and mRNA transcript copies of the transgene (eGFP) were quantified in comparison to the amounts of DNA genome copies or mRNA transcript copies of a house keeping gene (RPP30), respectively. Specifically, DNA genome copies are reported as vector genomes copies per diploid genome (VGC/DG). The formula for calculating the output is VGC/DG=(eGFP cp/μL÷RPP30 cp/μL)×2. RNA transcript copies are reported as % eGFP expression, which is calculated according to the formula, % eGFP expression=(eGFP cp/μL÷RPP30 cp/μL)×100.

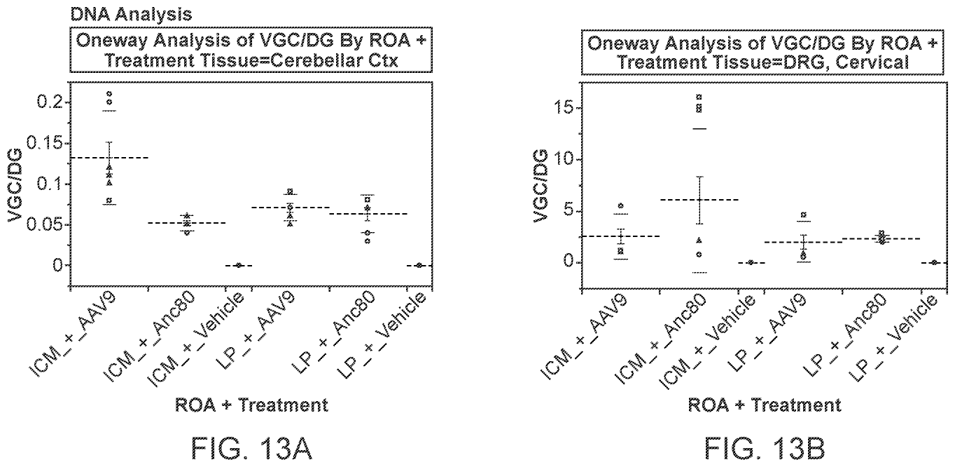

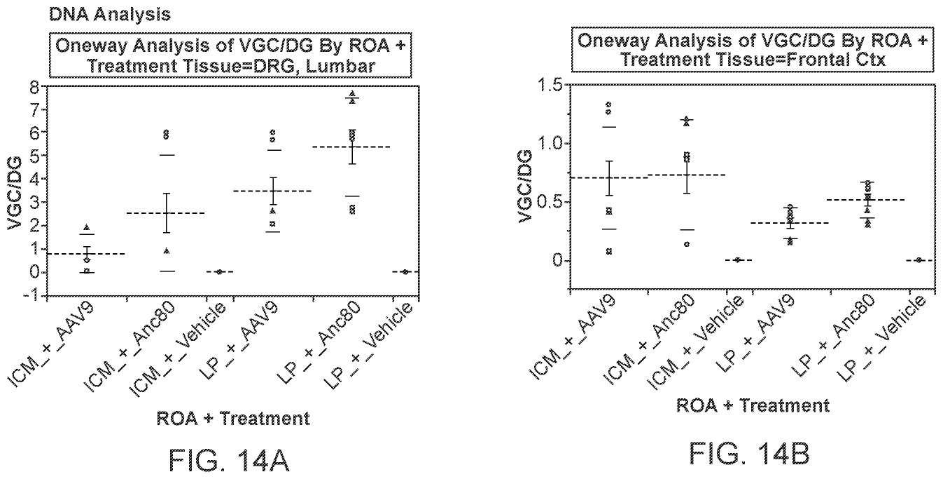

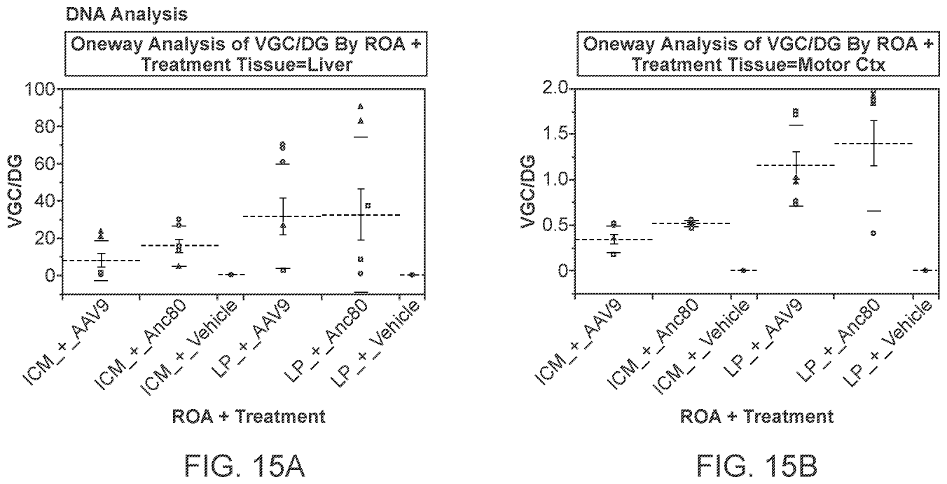

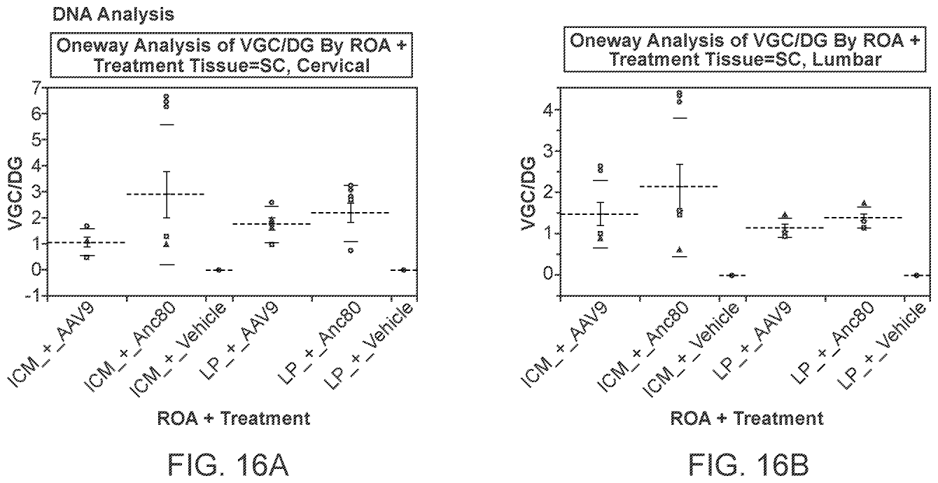

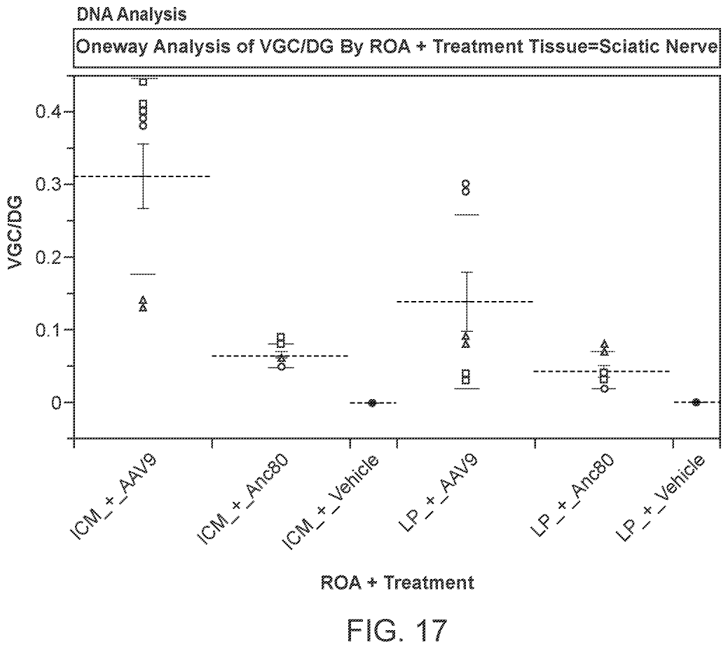

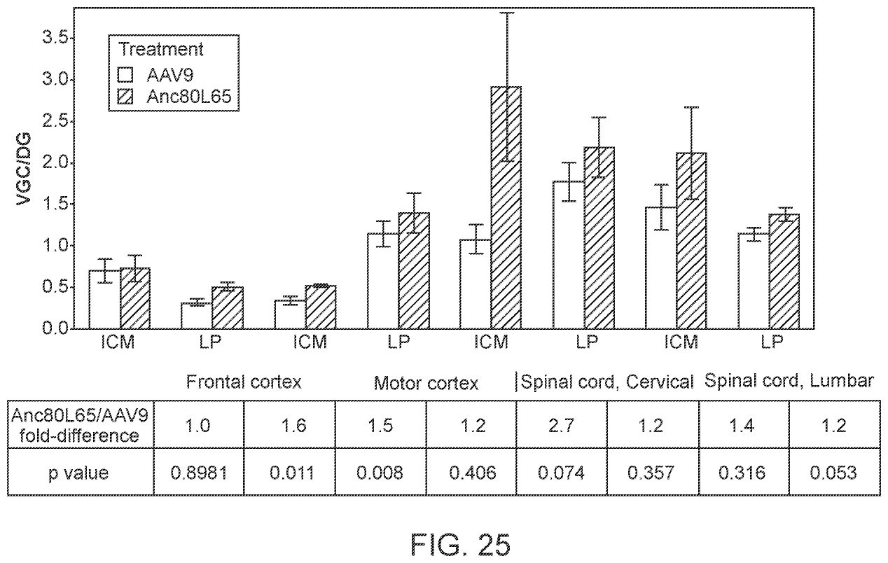

Viral DNA genome copies (VGCs) per diploid genome (i.e., VGCs per cell) measured in the experiment are provided in FIGS. 13A-17. Each figure provides data corresponding to different brain regions or liver, including cerebellar cortex (FIG. 13A), dorsal root ganglia, cervical (FIG. 13B), dorsal root ganglia, lumbar (FIG. 14A), frontal cortex (FIG. 14B), liver (FIG. 15A), motor cortex (FIG. 15B), spinal cord, cervical (FIG. 16A), spinal cord, lumbar (FIG. 16B), and sciatic nerve (FIG. 17). The VGCs data are further analyzed and summarized in FIG. 25.

The data show Anc80L65 led to more vector genome copies per cell in frontal cortex, motor cortex and spinal cord (cervical and lumbar) compared with AAV9, irrespective of injection route as shown in FIG. 25.

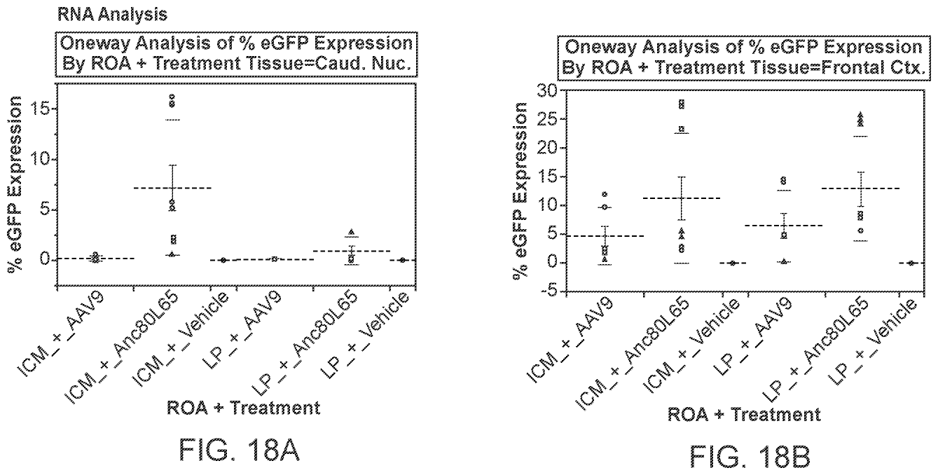

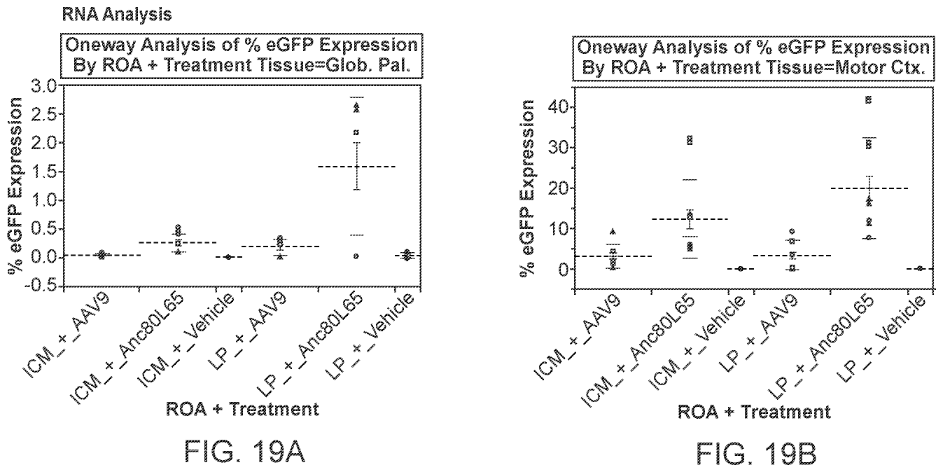

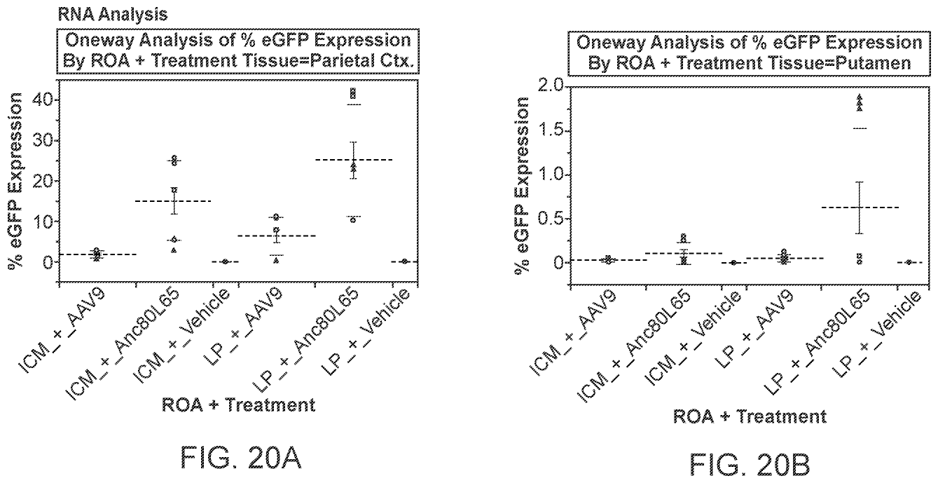

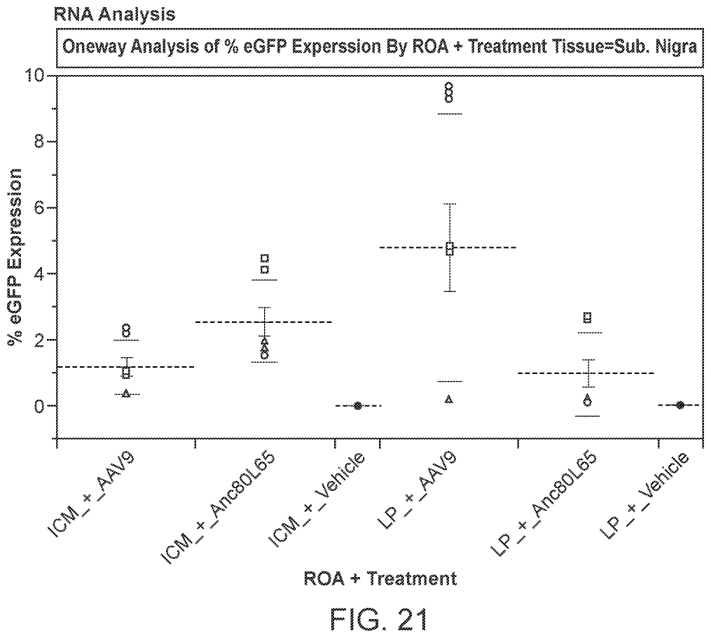

RNA transcripts measured from the experiment are provide in FIGS. 18A, 18B, 19A, 19B, 20A, 20B and 21. Each figure provides data corresponding to different brain regions, including caudate nucleus (FIG. 18A), frontal cortex (FIG. 18B), globus pallidus (FIG. 19A), motor cortex (FIG. 19B), parietal cortex (FIG. 20A), putamen (FIG. 20B), and substantia nigra (FIG. 21). Administration of Anc80L65 induced higher levels of GFP expression in several brain regions, including caudate nucleus after ICM administration, globus pallidus after LP administration, motor cortex after both ICM and LP administration, parietal cortex after both ICM and LP administration, and putamen after LP administration.

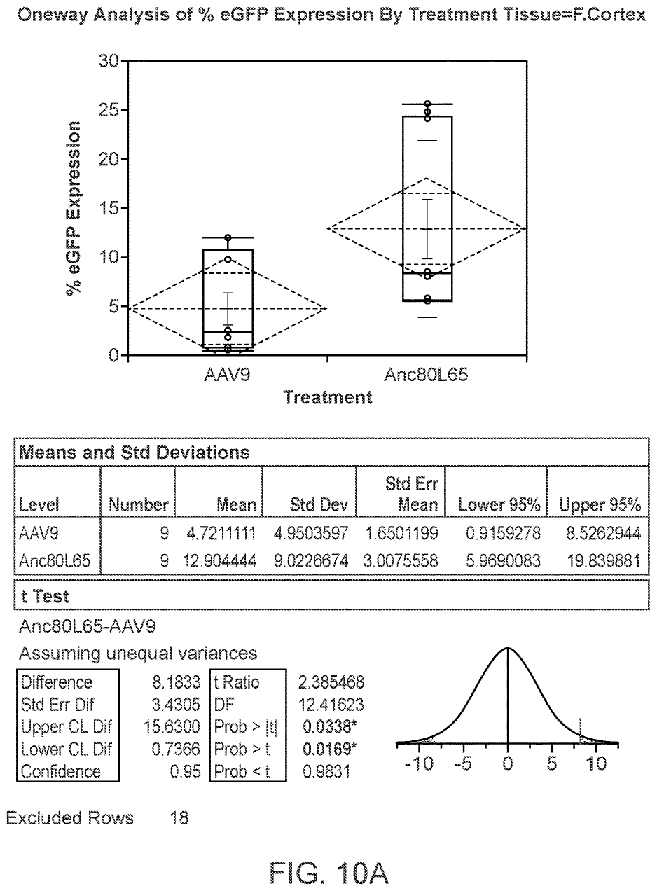

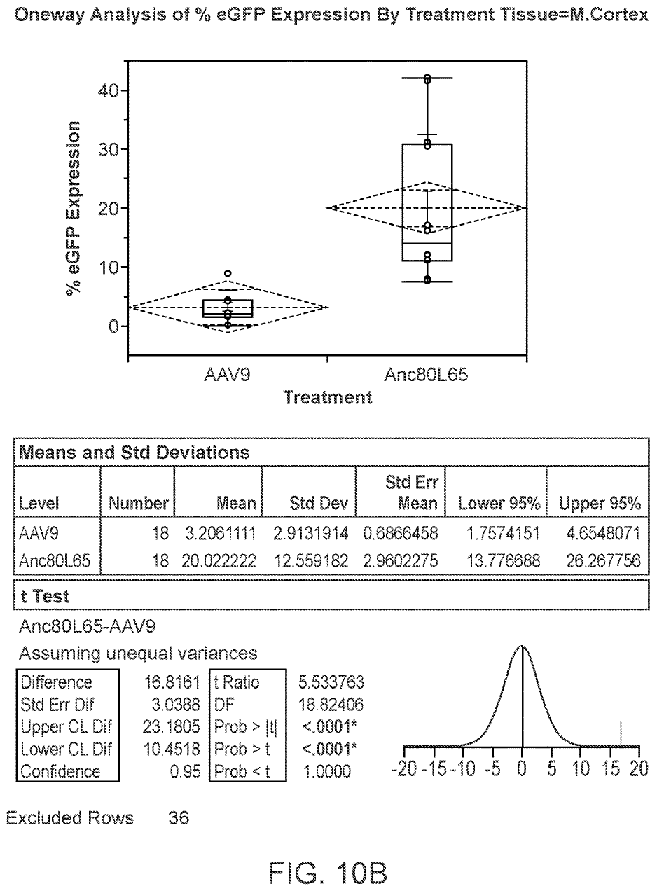

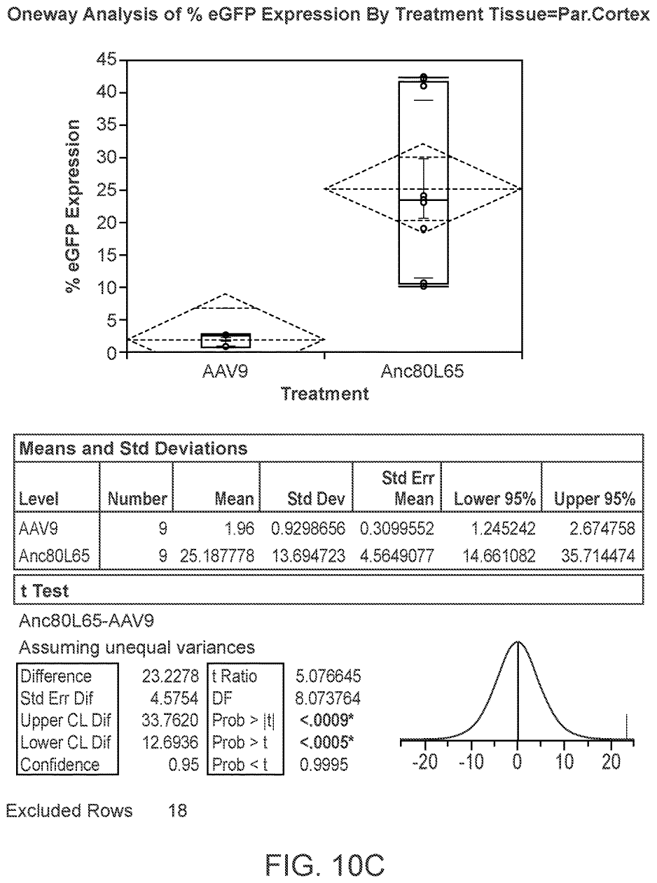

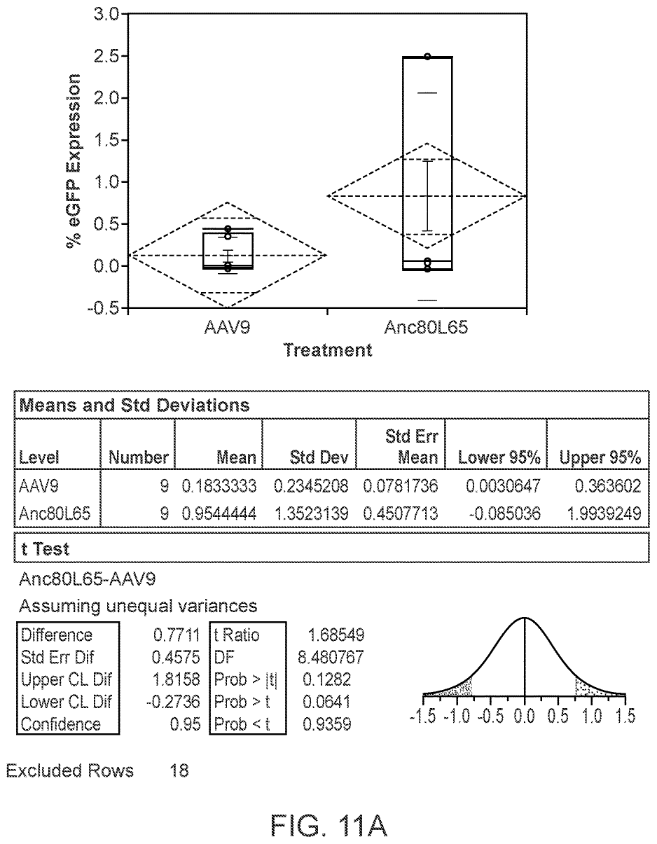

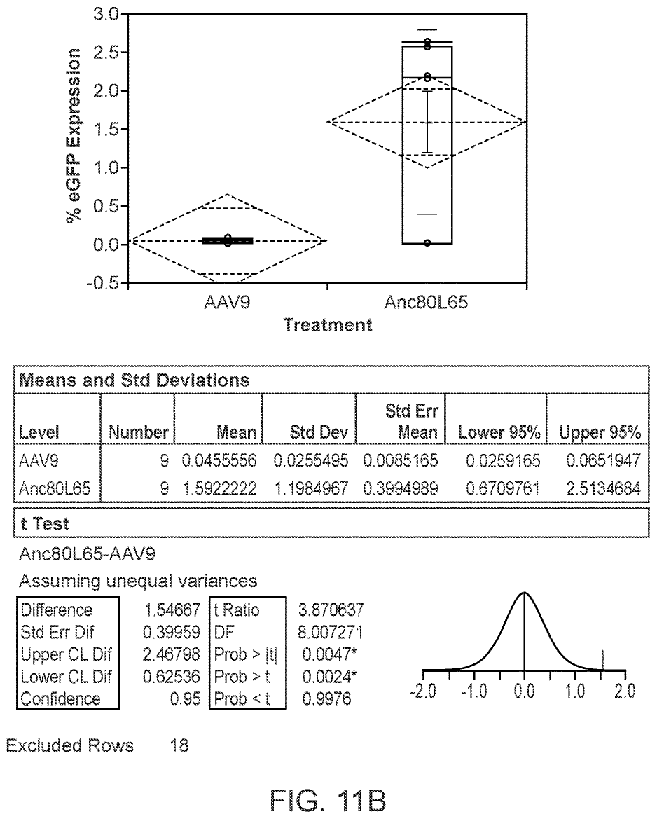

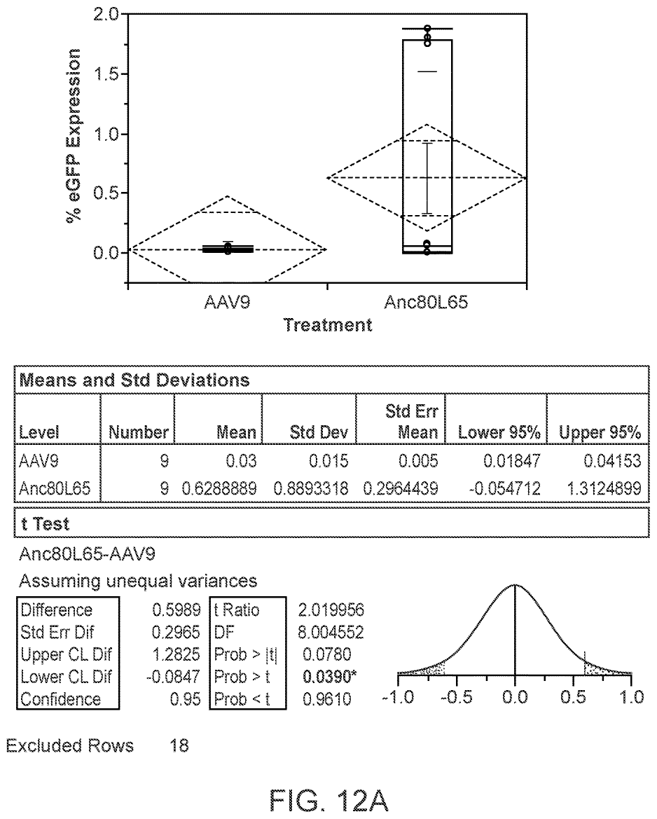

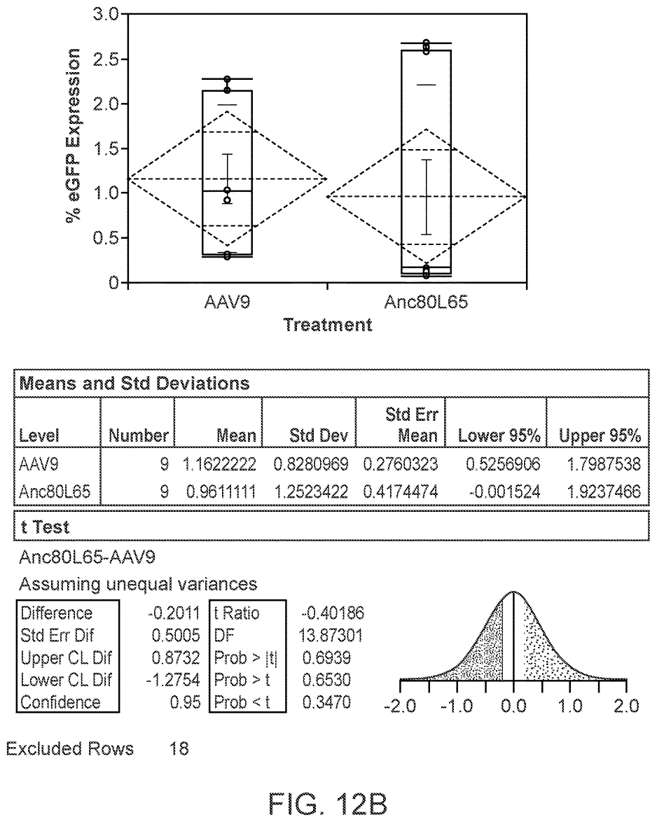

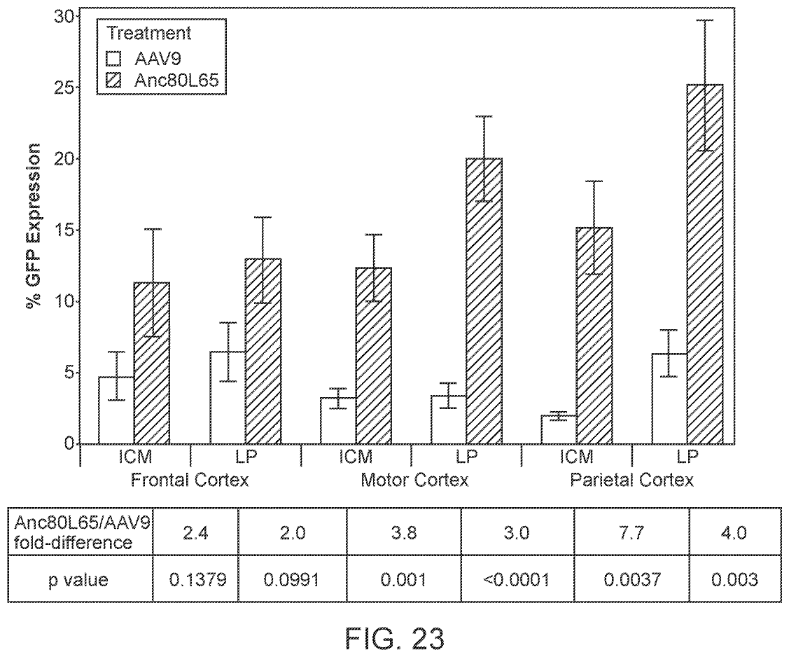

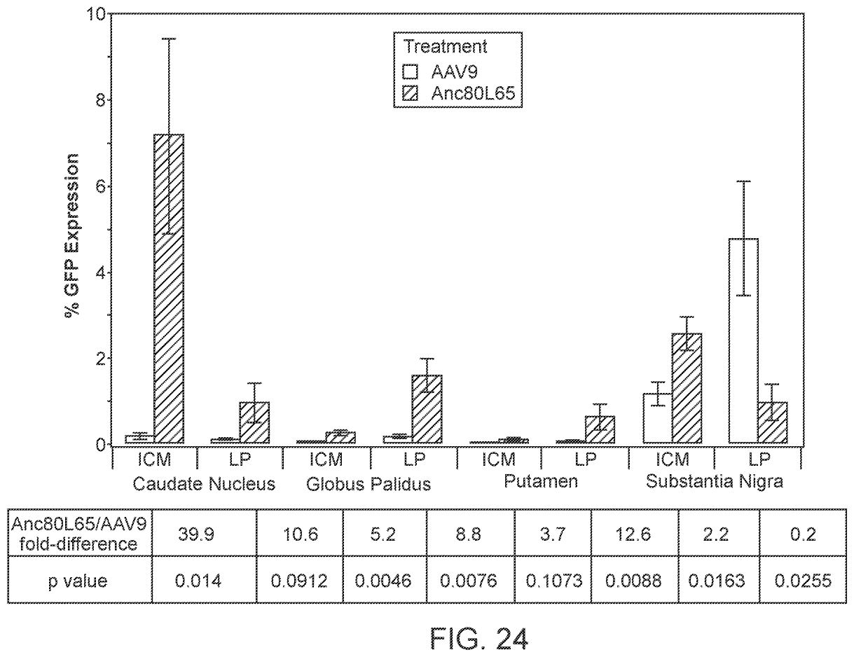

One-way statistical analysis of the expression data is provided in FIGS. 10A-FIG. 12B. The analysis results are also tabulated in FIG. 23 and FIG. 24. FIGS. 10A-10C and 23 provide analysis of the data from the frontal cortex (FIG. 10A, FIG. 23), motor cortex (FIG. 10B, FIG. 23); and parietal lobe of the cortex (FIG. 10C, FIG. 23). The data show significantly higher expression of GFP in the cortex of the animals injected with Anc80L65 by ICM or LP compared to AAV9 by ICM or LP. FIGS. 11A-11B, FIGS. 12A-12B and FIG. 24 show similar analysis in caudate nucleus (FIG. 11A, FIG. 24), globus pallidus (FIG. 11B, FIG. 24), putamen (FIG. 12A, FIG. 24) and substantia nigra (FIG. 12B, FIG. 24). These figures also show significantly higher GFP expression in the most brain areas of animals injected with Anc80L65 by ICM or LP compared to AAV9 by ICM or LP. These results suggest that both ICM and LP injections of Anc80L65 can be effective ways of delivering and expressing a transgene, superior to ICM administration of AAV9.

The statistical analysis of the ddPCR data is also provided below in TABLE 3. The table provides fold differences and p-value results from the Tukey-Kramer HSD test showing comparisons of GFP transcript (RNA) expression in various tissues between Anc80L65 (ICM) vs. AAV9 (ICM), Anc80L65 (LP) vs. AAV9 (ICM), and Anc80L65 (LP) vs. AAV9 (LP). Positive differences indicate the magnitude of expression advantage attributed to Anc80L65. Statistically significant p-Values are indicated in red (asterisk). The analysis shows that superiority of Anc80L65 is statistically significant compared to AAV9 in various brain regions.

TABLE 3

| | | | Anc80L65 Expression | |
|---|---|---|---|---|
| Tissue | Treatment 1 | Treatment 2 | Advantage | p-Value |
| Caud. Nuc. | ICM_+_Anc80L65 | ICM_+_AAV9 | +7.0 | 0.00* |
| Caud. Nuc. | LP_+_Anc80L65 | ICM_+_AAV9 | +0.8 | 0.96 |
| Caud. Nuc. | LP_+_Anc80L65 | LP_+_AAV9 | +0.9 | 0.95 |
| Frontal | ICM_+_Anc80L65 | ICM_+_AAV9 | +6.6 | 0.35 |
| Ctx. | | | | |
| Frontal | LP_+_Anc80L65 | ICM_+_AAV9 | +8.2 | 0.17 |
| Ctx. | | | | |
| Frontal | LP_+_Anc80L65 | LP_+_AAV9 | +6.4 | 0.36 |
| Ctx. | | | | |
| Glob. Pal. | ICM_+_Anc80L65 | ICM_+_AAV9 | +0.2 | 0.88 |
| Glob. Pal. | LP_+_Anc80L65 | ICM_+_AAV9 | +1.5 | <.0001* |
| Glob. Pal. | LP_+_Anc80L65 | LP_+_AAV9 | +1.4 | 0.0001* |
| Motor Ctx. | ICM_+_Anc80L65 | ICM_+_AAV9 | +9.1 | 0.01* |
| Motor Ctx. | LP_+_Anc80L65 | ICM_+_AAV9 | +16.8 | <.0001* |
| Motor Ctx. | LP_+_Anc80L65 | LP_+_AAV9 | +16.6 | <.0001* |
| Parietal | ICM_+_Anc80L65 | ICM_+_AAV9 | +13.2 | 0.02* |
| Ctx. | | | | |
| Parietal | LP_+_Anc80L65 | ICM_+_AAV9 | +23.2 | <.0001* |
| Ctx. | | | | |
| Parietal | LP_+_Anc80L65 | LP_+_AAV9 | +18.8 | 0.0004* |
| Ctx. | | | | |
| Putamen | ICM_+_Anc80L65 | ICM_+_AAV9 | +0.1 | 0.98 |
| Putamen | LP_+_Anc80L65 | ICM_+_AAV9 | +0.6 | 0.04* |
| Putamen | LP_+_Anc80L65 | LP_+_AAV9 | +0.6 | 0.05* |
| Sub. Nigra | ICM_+_Anc80L65 | ICM_+_AAV9 | +1.4 | 0.56 |
| Sub. Nigra | LP_+_Anc80L65 | ICM_+_AAV9 | −0.2 | 1.00 |
| Sub. Nigra | LP_+_Anc80L65 | LP_+_AAV9 | −3.8 | 0.01* |

### 7.2. Example 2: Analysis of AAV-^{Lib460} in Non-Human Primate to Identify Capsid Variants with Enhanced CNS Tropism

The objective of this study was to assess the 460 modified capsid proteins from the AAV-^{Lib460} in non-human primates (NHP) to identify capsids having VR VIII variants that enhance CNS tropism. The 460 targeting peptides included sequences of SEQ ID NO: 160-619. The targeting peptides were inserted between Q588 and A589 of the Anc80L65 VP1 capsid protein.

NHPs were administered the library containing 460 unique sequences of modified Anc80L65 VP1 capsid proteins (referred to herein as the AAV-^{Lib460} library) produced as described above. The targeting peptides were positioned between Q588 and A589 in VR VIII for each of the 460 modified Anc80L65 VP1 capsid variants. Controls include wild-type Anc80L65, wild-type AAV9, and wild-type AAV9-retro (as described in Tervo et al., Neuron, 92(2): 372-382 (2016), doi.org/10.1016/j.neuron.2016.09.021 and Lin et al., Molecular Brain, 13:138 (2020), doi.org/10.1186/s13041-020-00679-1).

### 7.2.1. Experimental Design

Three Cynomolgus macaques were treated as summarized in the Table 4 below. All animals received all 3 treatments with the indicated amounts or concentrations of AAV-^{Lib460} (comprising the 460 modified capsid proteins and controls (an Anc80L65 capsid protein, an AAV9 capsid protein, and an AAV9-Retro capsid protein)) (see Table 4) by ICM, IG, or IV.

TABLE 4
Experimental design for non-human primate study treated with AAV^{-medium}

| | | | | Study end |
|---|---|---|---|---|
| | | | | point |
| Treatment | | Dose | | (necropsy |
| No. | Treatment | Route | Dose (vg/animal or kg) | day 28) |
| 1 | Anc80L65- | ICM | Library-BC1(1-460): 4E+13 | 3 |
| | neurotropic peptide | | pLib-AAV9-BC1a: 8.7E+10 | |
| | (460 variants) and | | pLib-Anc80L65-BC1b: 8.7E+10 | |
| | controls | | pLib-AAV9-Retro-BC1c: 8.7E+10 | |
| 2 | Anc80L65- | IV | Library-BC2(1-460): 1.2E+14 | 3 |
| | neurotropic peptide | | pLib-AAV9-BC2a: 2.06E+11 | |
| | (460 variants) and | | pLib-Anc80L65-BC2b: 2.06E+11 | |
| | controls | | pLib-AAV9-Retro-BC2c: 2.06E+11 | |
| 3 | Anc80L65- | IG | Library-BC3(1-460): 1.4E+13 | 3 |
| | neurotropic peptide | (inter- | pLib-AAV9-BC3a: 3.06E+10 | |
| | (460 variants) and | digital) | pLib-Anc80L65-BC3b: 3.06E+10 | |
| | controls | | pLib-AAV9-Retro-BC3c: 3.06E+10 | |

### 7.2.2. Intracisternal Magna (ICM) Injection

ICM was performed as described in Example 1.

### 7.2.3. Inter-Digital Injection

The animal is injected with anesthesia and placed in lateral recumbency. A 25 gauge needle loaded with an appropriate Test Article syringe is advanced into the pad of the animals upper limb digits. In particular, the middle and index finger (which are both connected to the median nerve) on the right hand are injected with equal volumes of 0.2 mL of Test Article per site (total 0.4 mL).

### 7.2.4. Intravenous Injection

The animal was injected with anesthesia and placed in lateral recumbency. The animal was restrained in a position that allows access to the vein. The injection site was surgically prepared (shaved and cleaned aseptically). The vein was distended by compressing the vein closer to the heart than the catheter entry site. Compression was applied manually. The vein was visualized, and the catheter was inserted and advanced into the vein, the stylet was held stationary as the catheter was slowly advanced into the vessel until the hub reaches the skin puncture site. The dose was administered by a syringe through the injection cap. The catheter was flushed with sterile saline following the administration of the test article.

### 7.2.5. Analysis

Animals were sacrificed on day 28 after AAV vector administration and their tissue samples were collected for analysis. Tissue samples collected include CNS, liver, DRG, peripheral nerves, and spinal cord.

All analytical work was conducted by the Sponsor, using an analytical method developed and qualified by that laboratory.

DNA samples were analyzed for biodistribution of vector genomes in the CNS, liver, DRG, peripheral nerves, and spinal cord using NGS.

Gene transfer efficacy of each AAV vector was assessed by measuring mRNA transcript in the CNS, liver, DRG, peripheral nerves, and spinal cord using NGS.

Appendix A provides a rank order list of the 64 targeting peptides selected among 460 peptides based on their CNS targeting capability when incorporated into the AAV capsid. The rank is based on gene transfer efficacy assessed by measuring mRNA transcripts in the CNS. The list provides SEQ ID NO of targeting peptide; peptide sequence; and LogFC mean administered by ICM. See Appendix A.

### 7.3. Example 3: Analysis of AAV-^{Lib1} in Non-Human Primate to Identify Capsid Variants with CNS Tropism

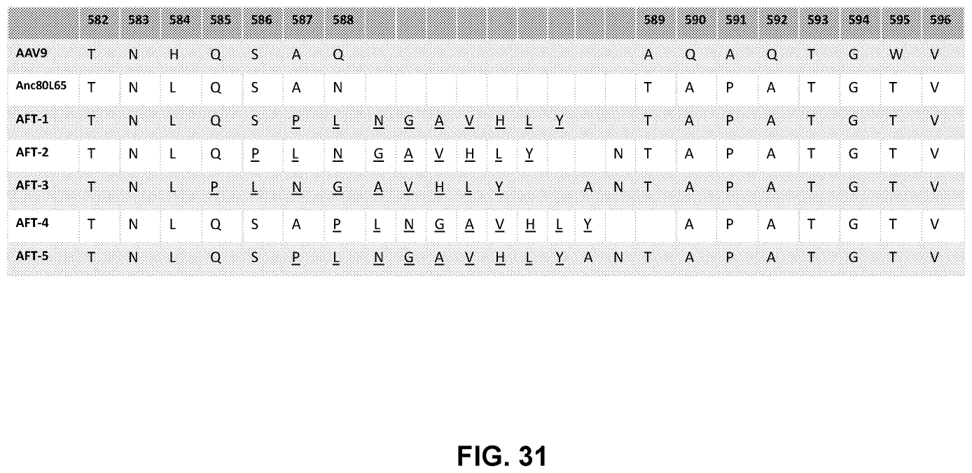

The objective of this study was to assess the modified capsid proteins in the AAV-^{mini} library (i.e., the targeting peptides (and insertion sites) as described, in part, in FIGS. 31-33 and SEQ ID NOs: 3-9, 11-19 or 21-28) in non-human primates (NHP) to identify capsids having targeting peptides (and/or insertion sites) that enhance CNS tropism. Full length modified capsid proteins including the targeting peptides described above are as described in SEQ ID NOs: 34-38 or 55820-55847.

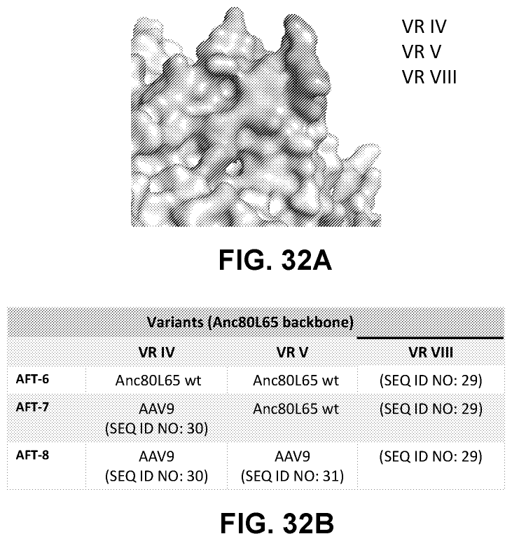

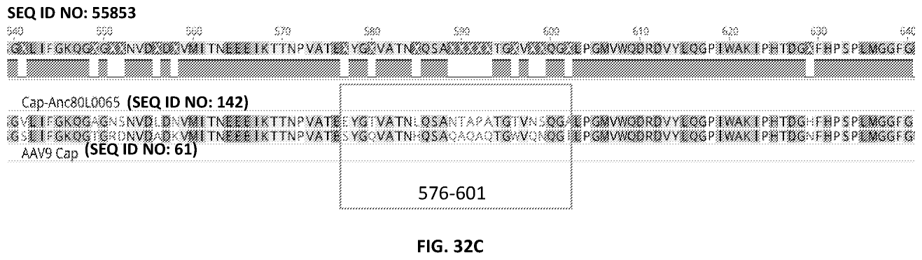

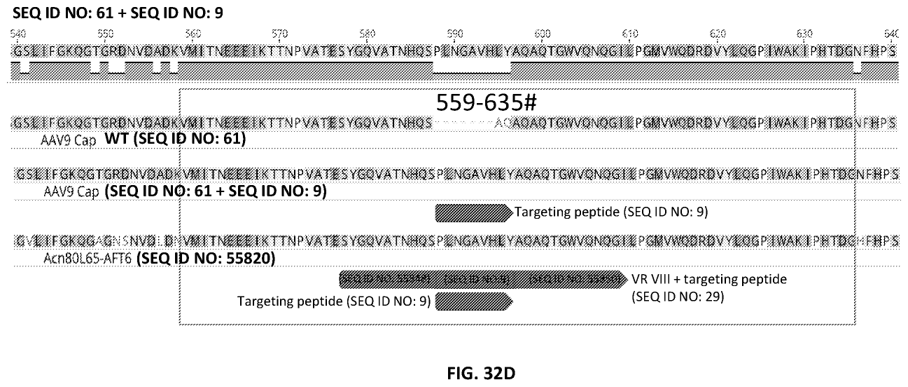

NHPs were administered the AAV-^{mini} library containing rAAVs having the modified capsid proteins sequences produced as described above. The targeting peptides (SEQ ID NOs: 3-9, 11-19 or 21-28) were inserted in VR VIII in either Anc80L65 VP1 capsid proteins or AAV9 VP1 capsid proteins as described, in part, in FIGS. 31-33. Additionally, Anc80L65 capsid proteins comprising one or more additional modifications as described in FIGS. 32A-32D were tested. Specifically, AFT-6, AFT-7 and AFT-8 VP1 capsid proteins were tested. AFT-6 includes Anc80L65 VP1 capsid protein backbone with the Anc80L65 VR VIII region replaced with the AAV9 VR VIII region (SEQ ID NO: 29). AFT-7 includes Anc80L65 VP1 capsid protein backbone with the Anc80L65 VR IV and VIII regions replaced with the AAV9 VR IV region (SEQ ID NO: 30) and AAV9 VIII region (SEQ ID NO: 29), respectively. AFT-8 includes Anc80L65 VP1 capsid protein backbone with the Anc80L65 VR IV, VR V and VIII regions replaced with the AAV9 VR IV region (SEQ ID NO: 30), AAV9 VR V region (SEQ ID NO: 31) and AAV9 VIII region (SEQ ID NO: 29), respectively.

Controls included pLib-AAV9, pLib-AAV9-D1, pLib-Anc80L65 (pLib-AAV9-C1 (AAV9 comprising a targeting peptide having a sequence of SEQ ID NO: 55854 inserted between Q558 and A589), pLib-AAV9-C2 (AAV9 comprising a targeting peptide having a sequence of SEQ ID NO: 55855 inserted between Q558 and A589), pLib-AAV9-C3 (AAV9 comprising a targeting peptide having a sequence of SEQ ID NO: 55856 inserted between Q558 and A589) in library ATP292).

### 7.3.1. Experimental Design

Three animals were treated as summarized in Table 5 below. Immunosuppression of the animals began 7 days prior to vector administration. The animals were administered with the indicated amounts or concentrations of AAV-^{mini} (comprising the 37 modified capsid proteins and controls (see Table 5)) by IV.

TABLE 5
Experimental design for non-human primate study with AAV^{-mini} Library

| | | | | | | Study end |
|---|---|---|---|---|---|---|
| Group | | | Dose | Number of | Dose (vg/animal | point (in- |
| No. | Treatment | Strain | Route | animals | or kg) | life days)) |
| 1 | AAV9/Anc80L65- | Cyno | IV | 3 | 2E+13 (5.4E+11 | 28 |
| | AFT mini library | | | | per variant) | |
| | (37 variants) | | | | | |

### 7.3.2. Analysis

Animals were sacrificed on day 28 after AAV vector administration and their organ samples were collected for analysis. Tissue samples collected included CNS, liver, DRG, peripheral nerves, and spinal cord.

All analytical work was conducted by the Sponsor, using an analytical method developed and qualified by that laboratory.

DNA samples were collected and will be analyzed for biodistribution of vector genomes in the CNS, liver, DRG, peripheral nerves, and spinal cord using NGS.

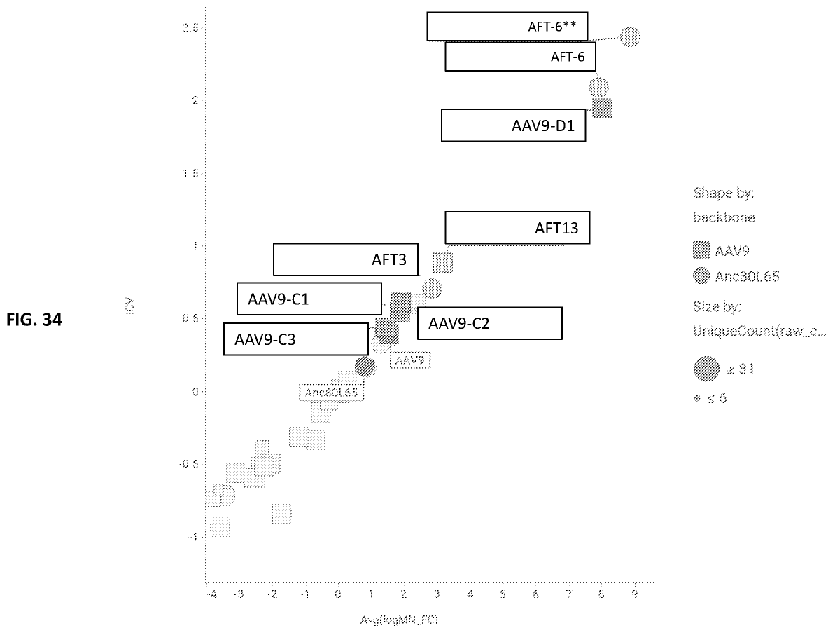

Gene transfer efficacy of each AAV vector was assessed by measuring mRNA transcript in the CNS, liver, DRG, peripheral nerves, and spinal cord using NGS. For these experiments, each AAV comprised a barcode mRNA, which represented the ID of a single AAV. The barcoded mRNA served as a proxy for the presence of the AAV (i.e., an AAV comprising the various capsid protein) and was the entity that was sequenced and used to quantify gene transfer efficacy. To aid quantification, comparison to a test article (TA) was used to normalize for different AAV abundances in the TA occurring due to manufacturing and also was used as a quantitative measurement of expression in the final sample. The sequenced entity in the test article (TA) was the DNA of that same barcode from the packaged capsid. Gene transfer data is shown in FIG. 34 and Table 6.

Table 6 provides a rank order list of the AAV tested where rank is based on LogFC_mean. “LogMN_FC” refers the log 2 fold-change in a sample relative to injection test article (TA). In particular, LogMN_FC refers to log 2(median normalized counts per million (CPM) in a sample for a single AAV/median normalized CPM in a test article (TA) for the same AAV). “CPM” is the count of a single AAV/total count of all AAVs×1E6. “Mean normalized CPM” is the CPM of a single AAV/median (CPM of all AAVs in a sample). Table 6 shows that AFT-6 and AFT-6** (biological replicates of AFT-6 (SEQ ID NO: 55820)) had the highest RNA expression of the tested AAVs.

FIG. 34 shows the inverse coefficient of variation (ICV) for expression levels of single AAVs in brain tissue (“Average LogMN_FC”). In particular, “LogMN_FC” refers to log 2(median normalized counts per million (CPM) in a sample for a single AAV/median normalized CPM in a test article (TA) for the same AAV). “Average LogMN_FC” refers to the mean of logMN_FC values for a single AAV across all samples from all 3 animals in a selected tissue set (i.e., brain tissue). ICV is a measure of variability where a larger value denotes less variability compared to the mean and was calculated as 1/coefficient of variation for each AAV across all samples. As shown in FIG. 34, AFT-6A and AFT-6B (biological replicates of AFT-6 (SEQ ID NO: 55820)) not only had the highest RNA expression of the AAV tested but also had the least amount of variability compared to the mean of all the AAVs tested.

Overall, this data showed that an AAV virion comprising capsid protein AFT-6 (SEQ ID NO: 55820) exhibited the highest RNA expression levels in brain tissue among the capsid proteins tested (see FIG. 34 and Table 6).

TABLE 6

| | | Capsid | | | | |
|---|---|---|---|---|---|---|
| | | protein | | | | |
| | Targeting | (including | | | | |
| | peptide in | targeting | | | | |
| | VR VIII - | peptide) - | | | logFC | scaled |
| variant_{—} | SEQ ID | SEQ ID | Final | logFC | std | logFC |
| name | NO: | NO: | rank | mean | dev | mean |
| AFT-6 | 9 | 55820 | 1 | 8.33 | 3.85 | 0.95 |
| AFT-6** | 9 | 55820 | 2 | 7.92 | 4.09 | 0.92 |
| AAV9_D1 | 9 | * | 3 | 7.82 | 3.95 | 0.92 |
| AFT-13 | 7 | 55829 | 7 | 3.12 | 3.52 | 0.68 |
| AFT-3 | 9 | 36 | 4 | 2.95 | 3.84 | 0.67 |
| AAV9_C3 | 55854 | * | 5 | 1.8 | 3.41 | 0.62 |
| AFT-22 | 18 | 55838 | 5 | 1.8 | 3.98 | 0.61 |
| AAV9_C2 | 55855 | * | 12 | 1.67 | 2.9 | 0.6 |
| AFT-20 | 16 | 55836 | 10 | 1.64 | 3.14 | 0.6 |
| AFT-4 | 9 | 37 | 10 | 1.37 | 3.9 | 0.59 |
| AAV9 | — | 61 | 14 | 1.26 | 4.39 | 0.58 |
| AFT-8 | 9 | 55823 or | 8 | 1.24 | 4.9 | 0.58 |
| | | 55824 | | | | |
| AAV9_C1 | 55856 | * | 16 | 1.24 | 3.38 | 0.58 |
| Anc80L65 | — | 161 | 8 | 1.08 | 4.17 | 0.58 |
| AFT-12 | 6 | 55828 | 16 | 0.45 | 3.95 | 0.54 |
| AFT-17 | 13 | 55833 | 15 | 0.33 | 3.89 | 0.54 |
| AFT-23 | 19 | 55839 | 21 | −0.51 | 2.45 | 0.49 |
| AFT-21 | 17 | 55837 | 18 | −0.51 | 2.59 | 0.49 |
| AFT-15 | 11 | 55831 | 13 | −0.56 | 4.58 | 0.5 |
| AFT-25 | 22 | 55841 | 23 | −0.85 | 3.46 | 0.47 |
| AFT-24 | 21 | 55840 | 22 | −1.55 | 5.6 | 0.44 |
| AFT-19 | 15 | 55835 | 18 | −1.65 | 4.22 | 0.44 |
| AFT-9 | 3 | 55825 | 18 | −1.69 | 5.05 | 0.44 |
| AFT-28 | 25 | 55844 | 24 | −1.96 | 3.66 | 0.42 |
| AFT-31 | 28 | 55847 | 33 | −2.32 | 4.1 | 0.39 |
| AFT-29 | 26 | 55845 | 30 | −2.43 | 5.35 | 0.39 |
| AFT-14 | 8 | 55830 | 24 | −2.49 | 3.87 | 0.39 |
| AFT-5 | 9 | 38 | 29 | −3.23 | 4.97 | 0.35 |
| AFT-30 | 27 | 55846 | 24 | −3.28 | 5.23 | 0.36 |
| AFT-27 | 24 | 55843 | 24 | −3.76 | 5.08 | 0.34 |
| AFT-16 | 12 | 55832 | 30 | −3.76 | 5.7 | 0.33 |
| AFT-18 | 14 | 55834 | 24 | −4.07 | 5.4 | 0.32 |
| AFT-10 | 4 | 55826 | 34 | −4.19 | 5.27 | 0.3 |
| AFT-26 | 23 | 55842 | 36 | −4.72 | 4.88 | 0.28 |
| AFT-11 | 5 | 55827 | 32 | −4.77 | 5.2 | 0.28 |
| AFT-2 | 9 | 35 | 36 | −5.65 | 6.23 | 0.23 |
| AFT-1 | 9 | 34 | 35 | −6.42 | 4.97 | 0.18 |

*denotes an AAV9 backbone (SEQ ID NO: 61) with targeting peptide inserted between Q588 and A589
**denotes technical replicate of AFT-6

Using the sequence data, enrichment scores for each tissue were calculated for each AAV variant containing a unique targeting peptide. In particular, the AAV variants including each targeting peptide were ranked based on a mean log fold-change tissue score (see, e.g., Table 6). After determining a sequence count from sequence read data (FASTQ) following sequencing, AAV variants containing a unique targeting peptide were counted and normalized, followed by performing a tissue enrichment analysis for each tissue. Tissue enrichment analysis included Sequence Activity Relationship (SAR) analysis, Network analysis, and structural modeling. SAR analysis identified particular amino acid at a specific capsid position or sequence motifs (combination of amino acids at several positions) that significantly affects tissue tropism. Network analysis identified modules of variants that shared amino acid or peptide sequences of top performing variants. Structural modeling provided understanding of the structural effect of significant amino acids at specific capsid positions for mechanistic hypothesis formulation.

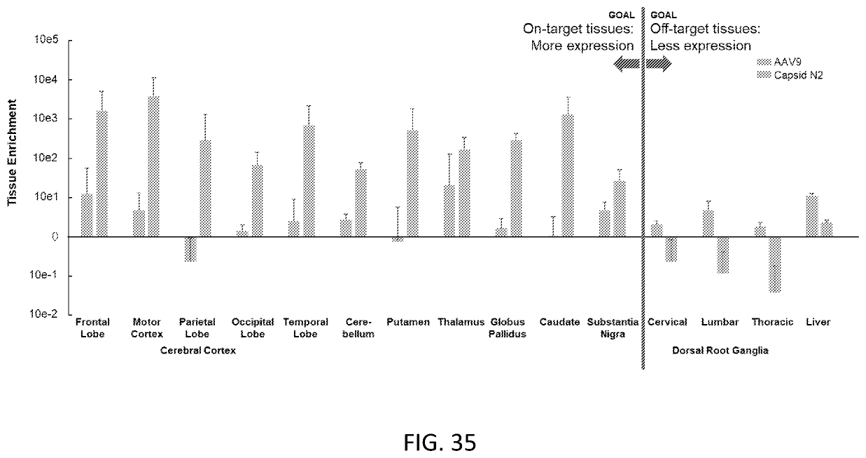

As shown in FIG. 35, SAR analysis identified one modified AAV capsid protein (i.e., an Anc80L65 capsid comprising an N2 targeting peptide (SEQ ID NO: 9; C4) located in VR VIII (full capsid sequence is referred to as AFT-6 (SEQ TD NO: 55820)) that increased tissue enrichment in on-target CNS tissues (as indicated in FIG. 35) as compared to AAV9. In particular, the Anc80L65 modified AAV capsid protein comprising the targeting peptide N2 has 100-1000 increased expression in various brain regions. On-target CNS tissues included frontal lobe, motor cortex, parietal lobe, occipital lobe, temporal lobe, cerebellum, putamen, thalamus, globus pallidus, caudate, and substantia nigra. This Anc80L65 modified AAV capsid protein comprising the N2 targeting peptide also had decreased tissue enrichment in off-target CNS tissues (as indicated in FIG. 35) as compared to AAV9. Off-target CNS tissues included dorsal root ganglion; such as cervical, lumbar, thoracic; and liver.

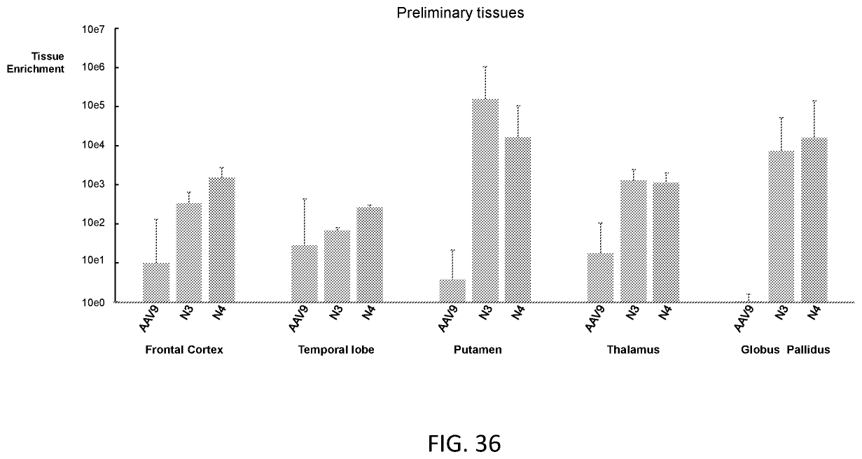

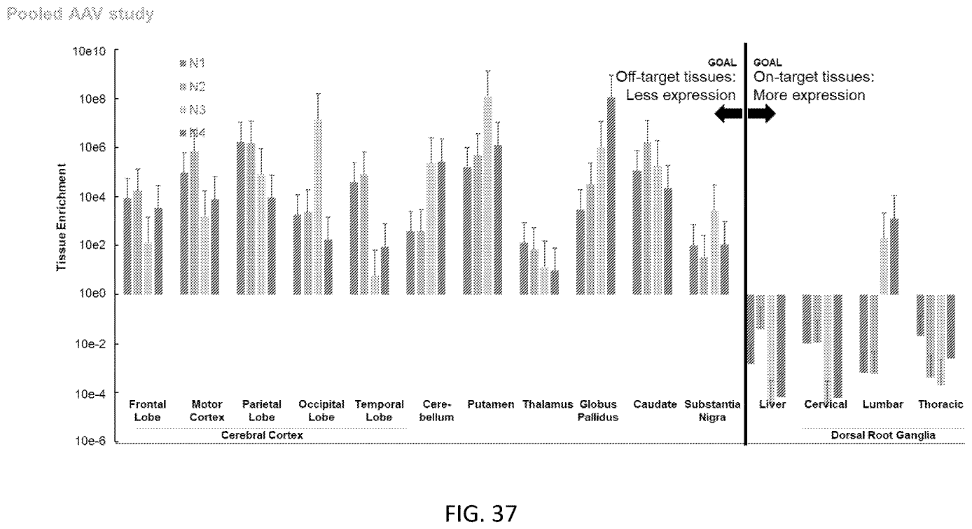

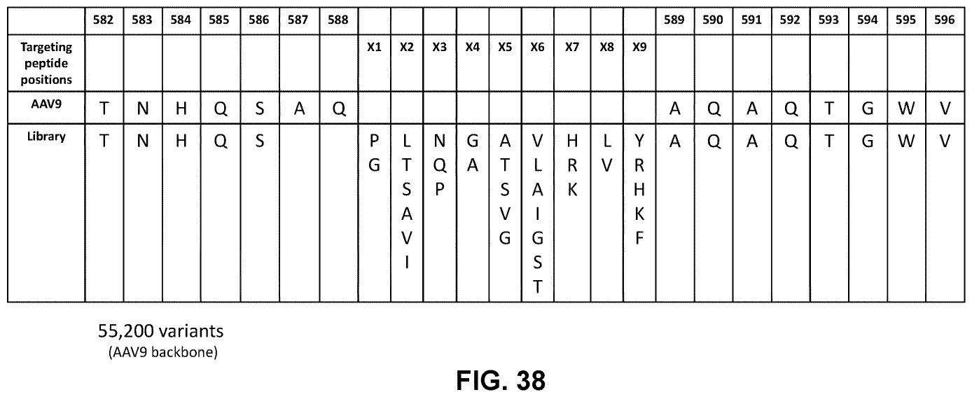

Additional SAR analysis identified three modified AAV capsid proteins (e.g., an Anc80L65 modified AAV capsid protein comprising a targeting peptide N2 (SEQ ID NO: 9) inserted into VR VIII (full capsid sequence is referred to as AFT-6 (SEQ ID NO: 55820)); an AAV9 comprising an N3 targeting peptide (PLNGSVHLY (SEQ ID NO: 3603) positioned in VR VIII between amino acid residues 586 and 589, replacing amino acids A587 and Q588, and an AAV9 comprising an N4 targeting peptide (PLNGTVHLY (SEQ ID NO: 1232) positioned in VR VIII between amino acid residues 586 and 589, replacing amino acids A587 and Q588) that increased tissue enrichment in various CNS tissues compared to controls (see FIG. 36 and FIG. 37).

Overall, this data showed that a subset of the modified AAV capsid proteins described herein have optimal CNS tropism.

### 7.4. Example 4: Analysis of AAV-^{Lib1} in Non-Human Primate to Identify Capsid Variants with CNS Tropism

The objective of this study was to assess the 54,000 modified capsid proteins comprising the AAV-^{Lib} library (i.e., the targeting peptides of Appendix A and inserted between positions Q586 and A589 in AAV, thereby replacing A587 and Q588) in non-human primates (NHP) to identify capsids having targeting peptides that enhance CNS tropism.

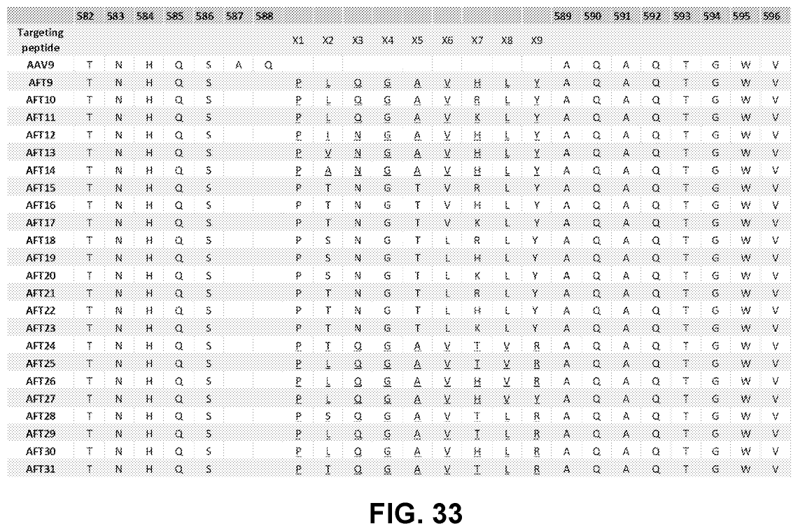

NHPs were administered a library containing about 54,000 unique sequences of modified capsid proteins, which is referred to herein as the AAV-^{Lib} library. The targeting peptides are positioned in VR VIII in AAV9 VP1 capsid proteins between amino acid residues 586 and 589, replacing amino acids A587 and Q588. Targeting peptides includes the sequences as described in SEQ ID NOs:620-55819 and 55857-55859. For example, the amino acid modifications (insertions, deletions, substitutions) in the VR VIII region of AAV9 include those shown in FIG. 33. Controls include pLib-AAV9, pLib-AAV9-C4, pLib-AAV9-C1, pLib-AAV9-C2, pLib-AAV9-C3.

### 7.4.1. Experimental Design

Three animals were treated as summarized in Table 6 below. Immunosuppression of the animals begins 7 days prior to vector administration. The animals are administered with the indicated amounts or concentrations of AAV-Lib (comprising the about 55,200 modified capsid proteins and controls) (see Table 8) by IV. Animals are sacrificed on day 28 after AAV vector administration and their organ samples are collected for analysis.

TABLE 8
Experimental design for non-human
primate study with AAV^{-Lib} Library

| Group | Treat- | Dose | Dose Vol | Dose (vg/ | Study end point |
|---|---|---|---|---|---|
| No. | ment | Route | (ml) | animal or kg) | (necropsy day 28) |
| 1 | AAV^{-Lib} | IV | 2.0 | 2E+13 | 3 |
| | | | | (3.7E+8 per | |
| | | | | variant) | |

### 7.4.2. Analysis

Animals were sacrificed on day 28 after AAV vector administration and their organ samples are collected for analysis. Tissue samples include: Basal Ganglia_Caudate; Basal Ganglia_Globus Pallidus; Basal Ganglia_Substantia Nigra; Cerebellar Cortex; DRG_Cervical; DRG_Lumbar; DRG_Thoracic; Frontal Lobe; Liver; Occipital Lobe; Parietal Lobe; Putamen Sensory, Motor Cortex; Temporal Lobe; and Thalamus.

DNA samples were analyzed for biodistribution of vector genomes in the CNS, liver, DRG, peripheral nerves, and spinal cord using NGS (data not shown).

Gene transfer efficacy of each AAV vector was assessed by measuring mRNA transcript in the tissues described in Appendix B. Tissue enrichment data is also presented in Appendix B, which is hereby incorporated by reference in its entirety. The data represents Average LogMN_FC measured and calculated as described above. Appendix B provides 750 targeting peptides selected among 54,000 modified capsid proteins for their CNS targeting capability.

Overall, this data showed that a subset of the modified AAV capsid proteins, (e.g., capsid proteins provided in Appendix B), have optimal CNS tropism.

Table 7 provides CNS-targeting data (Average LogMN_FC) of some of the AAV vectors, AAV9 or AAV9 with a targeting peptide (N3, N4 or N5).

TABLE 7

| | | | Basal | Basal | | |
|---|---|---|---|---|---|---|
| | Peptide | Basal | Ganglia_{—} | Ganglia_{—} | | |
| | (SEQ ID | Ganglia_{—} | Globus | Substantia | Cerebellar | DRG_{—} |
| | NO) | Caudate | Pallidus | Nigra | Cortex | Cervical |
| AAV9-N3 (PLNGSVHLY) | 3603 | 5.851529775 | 8.06713 | 8.367424 | 8.073194 | 5.790573 |
| AAV9-N4 (PLNGTVHLY) | 1232 | 8.07772938 | 11.44512 | 7.010849 | 9.781078 | 5.13861 |
| AAV9-N5 (PLNGVVHLY) | 5363 | 10.01029843 | 10.1324 | 7.552607 | 7.226381 | 4.15682 |
| AAV9 | Control | 0.858062602 | 0.443734 | 6.192864 | NA | 8.692444 |
| | Peptide | | | | | |
| | (SEQ ID | DRG_{—} | DRG_{—} | Frontal | | Occipital |
| | NO) | Lumbar | Thoracic | Lobe | Liver | Lobe |
| AAV9-N3 (PLNGSVHLY) | 3603 | 6.54552 | 7.087162 | 7.886621 | 4.143932 | 13.85746 |
| AAV9-N4 (PLNGTVHLY) | 1232 | 11.95689 | 7.175497 | 10.41715 | 6.059846 | 7.449502 |
| AAV9-N5 (PLNGVVHLY) | 5363 | 9.225067 | 10.0492 | 9.666616 | 4.089119 | 7.404831 |
| AAV9 | Control | 4.160245 | 10.91645 | 1.463794 | 7.069782 | NA |
| | Peptide | | | Sensory | | |
| | (SEQ ID | Parietal | | Motor | Temporal | |
| | NO) | Lobe | Putamen | Cortex | Lobe | Thalamus |
| AAV9-N3 (PLNGSVHLY) | 3603 | 9.32805 | 20.60432 | 8.240024 | 5.962567 | 10.27073 |
| AAV9-N4 (PLNGTVHLY) | 1232 | 8.248708 | 10.17588 | 7.023651 | 7.868765 | 10.10989 |
| AAV9-N5 (PLNGVVHLY) | 5363 | 9.830613 | 10.34292 | 9.222774 | NA | 10.00561 |
| AAV9 | Control | NA | 0.268243 | 5.332247 | 1.244784 | 8.688741 |

7.4.3. Production of rAAVs Containing AAV-Lib1 Capsids

rAAVs containing various AAV-Lib1 capsids were generated in suspension HEK293 Cells to test their yields and manufacturability. As provided in Table 8, the study showed that rAAV containing AAV9-N3 and AAV9-N4 provide good yields at harvest and are stable during formulation. However, rAAV containing AAV9-N5 capsids provided lower yields at harvest and precipitated during formulation.

TABLE 8

| | AAV-Lib1 capsids | | |
|---|---|---|---|
| | AAV9-N3 | AAV9-N4 | |
| Harvest | PLNGSVHLY | PLNGTVHLY | |
| yields | (SEQ ID | (SEQ ID | |
| (vg/ml) | NO: 3603) | NO: 1232)) | AAV9-N5 |
| Minimum | 2.57E+11 | 2.78E+11 | 9.52E+10 |
| Maximum | 4.2E+11 | 3.54E+11 | 2.03E+11 |

7.4.4. Seroprevalence of rAAVs Containing AAV-Lib1 Capsids

Seroprevalence of various AAV-Lib1 capsids were tested in an in vitro neutralizing antibody assay using donor samples (N=55) that approximately represent demographics according to US Census Bureau, 2019. All samples were run at a 1:5 serum dilution and titers were determined as the reciprocal of the serum dilution reporting 50% relative light units (RLUs) compared to AAV capsid alone. Samples with a titer<5 were defined as seronegative samples and samples with a titer>5 were defined as seropositive samples.

Table 9 shows % of tested donor samples that are seropositive or seronegative. The data show that AAV9-N3, AAV9-N4 have lower seroprevalence than AAV9 and higher seroprevalence than AAV9-C4 and AAV5.

TABLE 9

| | Seropositive | Seronegative |
|---|---|---|
| AAV9-N3 | 34.54545 | 65.45455 |
| PLNGSVHLY | | |
| (SEQ ID | | |
| NO: 3603) | | |
| AAV9-N4 | 34.54545 | 65.45455 |
| PLNGTVHLY | | |
| (SEQ ID | | |
| NO: 1232) | | |
| AAV9 | 36.36364 | 63.63636 |
| AAV9-C4 | 21.81818 | 78.18182 |
| PLNGAVHLY | | |
| (SEQ ID | | |
| NO: 9) | | |
| AAV5 | 30.90909 | 69.09091 |

### 8. SEQUENCES

Many of the nucleotide sequences provided below are obtained from double stranded vectors. Thus, one of skill in the art would appreciate that, unless the references throughout the specification and claims to nucleotide sequences provided herein also include references to the complementary sequences unless the context dictates otherwise. Additional sequences are found in Appendix A and Appendix B each of which are hereby incorporated by reference in their entireties.

| | SEQUENCE (X or Xⁿ can be any of the standard amino acids; for Anc library sequences |
|---|---|
| SEQ ID | (Anc80; Anc81; Anc82; Anc83; Anc84; Anc94; Anc110; Anc113; Anc126; and Anc127), |
| NO | X can be any one of the amino acids listed below for each toggle site) |
| (AAV-Lib | X₁X₂X₃X₄X₅X₆X₇X₈X₉ |
| targeting | X₁, X₂, X₃, X₄, X₅, X₆, X₇, X₈, and X₉ are each independently selected from any |
| peptide) | amino acid residue. |
| SEQ ID | PX₂X₃GAVX₇LY |
| NO: 2 | X₂, X₃, and X₇ are independently selected from any amino acid residue |
| (AAV-mini | |
| consensus | |
| 1) | |
| SEQ ID | PLQGAVHLY |
| NO: 3 | |
| (AFT-9) | |
| SEQ ID | PLQGAVRLY |
| NO: 4 | |
| (AFT-10) | |
| SEQ ID | PLQGAVKLY |
| NO: 5 | |
| (AFT-11) | |
| SEQ ID | PINGAVHLY |
| NO: 6 | |
| (AFT-12) | |
| SEQ ID | PVNGAVHLY |
| NO: 7 | |
| (AFT-13) | |
| SEQ ID | PANGAVHLY |
| NO: 8 | |
| (AFT-14) | |
| SEQ ID | PLNGAVHLY |
| NO: 9 (C4) | |
| SEQ ID | PX₂X₃GX₅X₆X₇LY |
| NO: 10 | X₂, X₃, X₅, X₆, and X₇ are independently selected from any amino acid residue |
| (AAV-mini | |
| consensus | |
| 2) | |
| SEQ ID | PTNGTVRLY |
| NO: 11 | |
| (AFT-15) | |
| SEQ ID | PTNGTVHLY |
| NO: 12 | |
| (AFT-16) | |
| SEQ ID | PTNGTVKLY |
| NO: 13 | |
| (AFT-17) | |
| SEQ ID | PSNGTLRLY |
| NO: 14 | |
| (AFT-18) | |
| SEQ ID | PSNGTLHLY |
| NO: 15 | |
| (AFT-19) | |
| SEQ ID | PSNGTLKLY |
| NO: 16 | |
| (AFT-20) | |
| SEQ ID | PTNGTLRLY |
| NO: 17 | |
| (AFT-21) | |
| SEQ ID | PTNGTLHLY |
| NO: 18 | |
| (AFT-22) | |
| SEQ ID | PTNGTLKLY |
| NO: 19 | |
| (AFT-23) | |
| SEQ ID | PX₂X₃GAVX₇X₈X₉ |
| NO: 20 | X₂, X₃, X₅, X₆, and X₇ are independently selected from any amino acid residue |
| (AAV-mini | |
| consensus | |
| 3) | |
| SEQ ID | PTQGAVTVR |
| NO: 21 | |
| (AFT-24) | |
| SEQ ID | PLQGAVTVR |
| NO: 22 | |
| (AFT-25) | |
| SEQ ID | PLQGAVHVR |
| NO: 23 | |
| (AFT-26) | |
| SEQ ID | PLQGAVHVY |
| NO: 24 | |
| (AFT-27) | |
| SEQ ID | PSQGAVTLR |
| NO: 25 | |
| (AFT-28) | |
| SEQ ID | PLQGAVTLR |
| NO: 26 | |
| (AFT-29) | |
| SEQ ID | PLQGAVHLR |
| NO: 27 | |
| (AFT-30) | |
| SEQ ID | PTQGAVTLR |
| NO: 28 | |
| (AFT-31) | |
| SEQ ID | SYGQVATNHQSPLNGAVHLYAQAQTGWVQNQGI |
| NO: 29 | |
| (AAV9- | |
| VR VIII) | |
| SEQ ID | KTINGSGQNQQTLKFSV |
| NO: 30 | |
| (AAV9- | |
| VR IV) | |
| SEQ ID | TTVTQNNNSEFAWPGASSWA |
| NO: 31 | |
| (AAV9- | |
| VR V) | |
| SEQ ID | PLNGAVHLYN |
| NO: 32 | |
| (AFT-2 | |
| with C- | |
| term) | |
| SEQ ID | PLNGAVHLYAN |
| NO: 33 | |
| (AFT-3 | |
| with C- | |
| term) | |
| SEQ ID | MAADGYLPDWLEDNLSEGIREWWDLKPGAPKPKANQQKQDDGRGLVLPGYKYLGP |
| NO: 34 | FNGLDKGEPVNAADAAALEHDKAYDQQLKAGDNPYLRYNHADAEFQERLQEDTSF |
| (AFT-1 | GGNLGRAVFQAKKRVLEPLGLVEEGAKTAPGKKRPVEQSPQEPDSSSGIGKKGQQPA |
| Capsid + | RKRLNFGQTGDSESVPDPQPLGEPPAAPSGVGSNTMAAGGGAPMADNNEGADGVG |
| targeting | NASGNWHCDSTWLGDRVITTSTRTWALPTYNNHLYKQISSQSGGSTNDNTYFGYSTP |
| peptide) | WGYFDFNRFHCHFSPRDWQRLINNNWGFRPKKLNFKLFNIQVKEVTTNDGTTTIANN |
| | LTSTVQVFTDSEYQLPYVLGSAHQGCLPPFPADVFMIPQYGYLTLNNGSQAVGRSSFY |
| | CLEYFPSQMLRTGNNFQFSYTFEDVPFHSSYAHSQSLDRLMNPLIDQYLYYLSRTQTT |
| | SGTAGNRTLQFSQAGPSSMANQAKNWLPGPCYRQQRVSKTTNQNNNSNFAWTGAT |
| | KYHLNGRDSLVNPGPAMATHKDDEDKFFPMSGVLIFGKQGAGNSNVDLDNVMITNE |
| | EEIKTTNPVATEEYGTVATNLQSPLNGAVHLYTAPATGTVNSQGALPGMVWQDRDV |
| | YLQGPIWAKIPHTDGHFHPSPLMGGFGLKHPPPQILIKNTPVPANPPTTFSPAKFASFIT |
| | QYSTGQVSVEIEWELQKENSKRWNPEIQYTSNYNKSTNVDFAVDINGVYSEPRPIGT |
| | RYLTRNL |
| SEQ ID | MAADGYLPDWLEDNLSEGIREWWDLKPGAPKPKANQQKQDDGRGLVLPGYKYLGP |
| NO: 35 | FNGLDKGEPVNAADAAALEHDKAYDQQLKAGDNPYLRYNHADAEFQERLQEDTSF |
| (AFT-2 | GGNLGRAVFQAKKRVLEPLGLVEEGAKTAPGKKRPVEQSPQEPDSSSGIGKKGQQPA |
| Capsid + | RKRLNFGQTGDSESVPDPQPLGEPPAAPSGVGSNTMAAGGGAPMADNNEGADGVG |
| targeting | NASGNWHCDSTWLGDRVITTSTRTWALPTYNNHLYKQISSQSGGSTNDNTYFGYSTP |
| peptide) | WGYFDFNRFHCHFSPRDWQRLINNNWGFRPKKLNFKLFNIQVKEVTTNDGTTTIANN |
| | LTSTVQVFTDSEYQLPYVLGSAHQGCLPPFPADVFMIPQYGYLTLNNGSQAVGRSSFY |
| | CLEYFPSQMLRTGNNFQFSYTFEDVPFHSSYAHSQSLDRLMNPLIDQYLYYLSRTQTT |
| | SGTAGNRTLQFSQAGPSSMANQAKNWLPGPCYRQQRVSKTTNQNNNSNFAWTGAT |
| | KYHLNGRDSLVNPGPAMATHKDDEDKFFPMSGVLIFGKQGAGNSNVDLDNVMITNE |
| | EEIKTTNPVATEEYGTVATNLQPLNGAVHLYNTAPATGTVNSQGALPGMVWQDRDV |
| | YLQGPIWAKIPHTDGHFHPSPLMGGFGLKHPPPQILIKNTPVPANPPTTFSPAKFASFIT |
| | QYSTGQVSVEIEWELQKENSKRWNPEIQYTSNYNKSTNVDFAVDTNGVYSEPRPIGT |
| | RYLTRNL |
| SEQ ID | MAADGYLPDWLEDNLSEGIREWWDLKPGAPKPKANQQKQDDGRGLVLPGYKYLGP |
| NO: 36 | FNGLDKGEPVNAADAAALEHDKAYDQQLKAGDNPYLRYNHADAEFQERLQEDTSF |
| (AFT-3 | GGNLGRAVFQAKKRVLEPLGLVEEGAKTAPGKKRPVEQSPQEPDSSSGIGKKGQQPA |
| Capsid + | RKRLNFGQTGDSESVPDPQPLGEPPAAPSGVGSNTMAAGGGAPMADNNEGADGVG |
| targeting | NASGNWHCDSTWLGDRVITTSTRTWALPTYNNHLYKQISSQSGGSTNDNTYFGYSTP |
| peptide) | WGYFDFNRFHCHFSPRDWQRLINNNWGFRPKKLNFKLFNIQVKEVTTNDGTTTIANN |
| | LTSTVQVFTDSEYQLPYVLGSAHQGCLPPFPADVFMIPQYGYLTLNNGSQAVGRSSFY |
| | CLEYFPSQMLRTGNNFQFSYTFEDVPFHSSYAHSQSLDRLMNPLIDQYLYYLSRTQTT |
| | SGTAGNRTLQFSQAGPSSMANQAKNWLPGPCYRQQRVSKTTNQNNNSNFAWTGAT |
| | KYHLNGRDSLVNPGPAMATHKDDEDKFFPMSGVLIFGKQGAGNSNVDLDNVMITNE |
| | EEIKTTNPVATEEYGTVATNLPLNGAVHLYANTAPATGTVNSQGALPGMVWQDRDV |
| | YLQGPIWAKIPHTDGHFHPSPLMGGFGLKHPPPQILIKNTPVPANPPTTFSPAKFASFIT |
| | QYSTGQVSVEIEWELQKENSKRWNPEIQYTSNYNKSTNVDFAVDINGVYSEPRPIGT |
| | RYLTRNL |
| SEQ ID | MAADGYLPDWLEDNLSEGIREWWDLKPGAPKPKANQQKQDDGRGLVLPGYKYLGP |
| NO: 37 | FNGLDKGEPVNAADAAALEHDKAYDQQLKAGDNPYLRYNHADAEFQERLQEDTSF |
| (AFT-4 | GGNLGRAVFQAKKRVLEPLGLVEEGAKTAPGKKRPVEQSPQEPDSSSGIGKKGQQPA |
| Capsid + | RKRLNFGQTGDSESVPDPQPLGEPPAAPSGVGSNTMAAGGGAPMADNNEGADGVG |
| targeting | NASGNWHCDSTWLGDRVITTSTRTWALPTYNNHLYKQISSQSGGSTNDNTYFGYSTP |
| peptide) | WGYFDFNRFHCHFSPRDWQRLINNNWGFRPKKLNFKLFNIQVKEVTTNDGTTTIANN |
| | LTSTVQVFTDSEYQLPYVLGSAHQGCLPPFPADVFMIPQYGYLTLNNGSQAVGRSSFY |
| | CLEYFPSQMLRTGNNFQFSYTFEDVPFHSSYAHSQSLDRLMNPLIDQYLYYLSRTQTT |
| | SGTAGNRTLQFSQAGPSSMANQAKNWLPGPCYRQQRVSKTTNQNNNSNFAWTGAT |
| | KYHLNGRDSLVNPGPAMATHKDDEDKFFPMSGVLIFGKQGAGNSNVDLDNVMITNE |
| | EEIKTTNPVATEEYGTVATNLQSAPLNGAVHLYAPATGTVNSQGALPGMVWQDRDV |
| | YLQGPIWAKIPHTDGHFHPSPLMGGFGLKHPPPQILIKNTPVPANPPTTFSPAKFASFIT |
| | QYSTGQVSVEIEWELQKENSKRWNPEIQYTSNYNKSTNVDFAVDTNGVYSEPRPIGT |
| | RYLTRNL |
| SEQ ID | MAADGYLPDWLEDNLSEGIREWWDLKPGAPKPKANQQKQDDGRGLVLPGYKYLGP |
| NO: 38 | FNGLDKGEPVNAADAAALEHDKAYDQQLKAGDNPYLRYNHADAEFQERLQEDTSF |
| (AFT-5 | GGNLGRAVFQAKKRVLEPLGLVEEGAKTAPGKKRPVEQSPQEPDSSSGIGKKGQQPA |
| Capsid + | RKRLNFGQTGDSESVPDPQPLGEPPAAPSGVGSNTMAAGGGAPMADNNEGADGVG |
| targeting | NASGNWHCDSTWLGDRVITTSTRTWALPTYNNHLYKQISSQSGGSTNDNTYFGYSTP |
| peptide) | WGYFDFNRFHCHFSPRDWQRLINNNWGFRPKKLNFKLFNIQVKEVTTNDGTTTIANN |
| | LTSTVQVFTDSEYQLPYVLGSAHQGCLPPFPADVFMIPQYGYLTLNNGSQAVGRSSFY |
| | CLEYFPSQMLRTGNNFQFSYTFEDVPFHSSYAHSQSLDRLMNPLIDQYLYYLSRTQTT |
| | SGTAGNRTLQFSQAGPSSMANQAKNWLPGPCYRQQRVSKTTNQNNNSNFAWTGAT |
| | KYHLNGRDSLVNPGPAMATHKDDEDKFFPMSGVLIFGKQGAGNSNVDLDNVMITNE |
| | EEIKTTNPVATEEYGTVATNLQSPLNGAVHLYANTAPATGTVNSQGALPGMVWQDR |
| | DVYLQGPIWAKIPHTDGHFHPSPLMGGFGLKHPPPQILIKNTPVPANPPTTFSPAKFAS |
| | FITQYSTGQVSVEIEWELQKENSKRWNPEIQYTSNYNKSTNVDFAVDINGVYSEPRPI |
| | GTRYLTRNL |
| SEQ ID | MAADGYLPDWLEDNLSEGIREWWALKPGAPQPKANQQHQDNARGLVLPGYKYLGP |
| NO: 39 | GNGLDKGEPVNAADAAALEHDKAYDQQLKAGDNPYLKYNHADAEFQERLKEDTSF |
| (AAV-retro | GGNLGRAVFQAKKRLLEPLGLVEEAAKTAPGKKRPVEQSPQEPDSSAGIGKSGAQPA |
| Capsid) | KKRLNFGQTGDTESVPDPQPIGEPPAAPSGVGSLTMASGGGAPVADNNEGADGVGSS |
| | SGNWHCDSQWLGDRVITTSTRTWALPTYNNHLYKQISNSTSGGSSNDNAYFGYSTP |
| | WGYFDFNRFHCHFSPRDWQRLINNNWGFRPKRLNFKLFNIQVKEVTDNNGVKTIAN |
| | NLTSTVQVFTDSDYQLPYVLGSAHEGCLPPFPADVFMIPQYGYLTLNDGSQAVGRSSF |
| | YCLEYFPSQMLRTGNNFQFSYEFENVPFHSSYAHSQSLDRLMNPLIDQYLYYLSKTIN |
| | GSGQNQQTLKFSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVDADKVMITNEE |
| | EIKTTNPVATESYGQVATNHQSAQLADQDYTKTAAQAQTGWVQNQGILPGMVWQD |
| | RDVYLQGPIWAKIPHTDGNFHPSPLMGGFGMKHPPPQILIKNTPVPADPPTAFNKDKL |
| | NSFITQYSTGQVSVEIEWELQKENSKRWNPEIQYTSNYYKSNNVEFAVNTEGVYSEPR |
| | PIGTRYLTRNL |
| SEQ ID | MAADGYLPDWLEDTLSEGIRQWWKLKPGPPPPKPAERHKDDSRGLVLPGYRYLGPFNG |
| NO: 78 | LDKGEPVNEADAAALEHDKAYDRQLDSGDNPYLKYNHVDAEFQERLKEDTSFGGNLG |
| (AAVhu.20 | RAVFQAKKRILEPLGLVEEPVKAAPGEKRPVEHSPAEPDSSSGTGKAGQQPARKRLNFG |
| (AAS99271)) | QTGDADSVPDPQPLGQPPAAPSGLGTNTMASGSGAPMADNNEGADGVGNSSGNWHCD |
| | STWMGDRVITTSTRTWALPTYNNHLYKQISSQSGASNDNHYFGYSTPWGHFDFNRFHC |
| | HFSPRDWQRLINNNWGFRPKRLSFKLFNIQVKEVTQNDGTTTIANNLTSTVQVFTDSEYQ |
| | LPYVLGSAHQGCLPPFPADVFMVPQYGYLTLNNGSQAVGRSSFYCLEYFPSQMLRTGNN |
| | FTFSYTFEDVPFHSSYAHSQSLDRLMNPLIDQYLYYLSRTNTPSGTTTMSRLQFSQAGAS |
| | DIRDQSRNWLPGPCYRQQRVSKTAADNNNSDYSWTGATKYHLNGRDSLVNPGPAMAS |
| | HKDDEEKYFPQSGVLIFGKQDSGKTNVDIEKVMITDEEEIRTTNPVATEQYGSVSTNLQS |
| | GNTQAATSDVNTQGVLPGMVWQDRDVYLQGPIWAKIPHTDGHFHPSPPMGGFGLKHPP |
| | PQILIKNTPVPANPSTTFSAAKFASFITQYSTGQVSVEIEWELQKENSKRWNPEIQYTSNY |
| | NKSVNVDFTVDINGVYSEPRPIGARYLTRNL |
| SEQ ID | MAADGYLPDWLEDTLSEGIRQWWKLKPGPPPPKPAERHKDDSRGLVLPGYKYLGPFNG |
| NO: 79 | LDKGEPVNEADAAALEHDKAYDRQLDSGDNPYLKYNHADAEFQERLKEDTSFGGNLG |
| (AAVhu.21 | RAVFQAKKRILEPLGLVEEPVKTAPGKKRPVEHSPAEPDSSSGTGKAGQQPARKRLNFG |
| (AAS99272)) | QTGDADSVPDPRPLGQPPAAPSGLGTNTMASGSGAPMADNNEGADGVGNSSGNWHCD |
| | STWMGDRVITTSTRTWALPTYNNHLYKQISSQSGASNDNHYFGYSTPWGYFDFNRFHC |
| | HFSPRDWQRLINNNWGFRPKRLSFKLFNIQVKEVTQNDGTTTIANNLTSTVQVFTDSEYQ |
| | LPYVLGSAHQGCLPPFPADVFMVPQYGYLTLNNGSQAVGRSSFYCLEYFPSQMLRTGNN |
| | FTFSYTFEDVPFHSSYAHSQSLDRLMNPLIDQYLYYLSRTNTPSGTTTMSRLQFSQAGAS |
| | DIRDQSRNWLPGPCYRQQRVSKTAADNNNSDYSWTGATKYHLNGRDSLVNPGPAMAS |
| | HKDDEEKYFPQSGVLIFGKQDSGKTNVDIEKVMITDEEEIRTTNPVATEQYGSVSTNLQS |
| | GNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNY |
| | NKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNG |
| NO: 80 | LDKGEPVNEADAAALEHDKAYDRQLDSGDNPYLKYNHADAEFQERLKGDTSFGGNLG |
| (AAVhu.22 | RAVFQAKKRILEPLGLVEEPVKTAPGKKRPVEHSPAEPDSSSGTGKAGQQPARKRLNFG |
| (AAS99273)) | QTGDADSVPDPQPLGQPPAAPSGLGTNTMASGSGAPMADNNEGADGVGNSSGNWHCD |
| | STWMGGRVITTSTRTWALPTYNNHLYKQISSQSGASNDNHYFGYSTPWGYFDFNRFHC |
| | HFSPRDWQRLINNNWGFRPKRLSFKLFNIQVKEVTQNDGTTTIANNLTSTVQVFTDSEYQ |
| | LPYVLGSAHQGCLPPFPADVFMVPQYGYLTLNNGSQAVGRSSFYCLEYFPSQTLRTGNN |
| | FTFSYTFEDVPFHSSYAHSQSLDRLMNPLIDQYLYYLSRTNTPSGTTTMSRLQFSQAGAS |
| | DIRDQSRNWLPGPCYRQQRVSKTAADNNNSDYSWTGATKYHLNGRDSLVNPGPAMAS |
| | HKDDEEKYFPQSGVLIFGKQDSGKTNVDIEKVMITDEEEIRTTNPVATEQYGSVSTNLQS |
| | GNTQAATSDVNTQGVLPGMVWQDRDVYLQGPIWAKIPHTDGHFHPSPLMGGFGLKHPP |
| | PQILIKNTPVPANPSTTFSAAKFASFITQYSTGQVSVEIEWELQKENSKRWNPEIQYTSNY |
| | NKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNG |
| NO: 81 | LDKGEPVNEADAAALEHDKAYDRQLDSGDNPYLKYNHADAEFQERLKEDTSFGGNLG |
| (AAVhu.23 | RAVFQAKKRILEPLGLVEEPVKTAPGKKRPVEHSPAEPDSSSGTGKAGQQPARKRLNFG |
| (AAS99274)) | QTGDADSVPDPQPLGQPPAAPSGLGTNTMASGSGAPMADNNEGADGVGNSSGNWHCD |
| | STWMGDRVITTSTRTWALPTCNNHLYKQISSQSGASNDNHYFGYSTPWGYFDFNRFHCH |
| | FSPRDWQRLINNNWGFRPKRLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPSQMLRTGNNF |
| | TFSYTFEDVPFHSSYAHSQSLDRLMNPLIDQYLYYLSRTNTPSGTTTMSRLQFSQAGASDI |
| | RDQSRNWLPGPCYRQQRVSKTAADNNNSDYSWTGATKYHLNGRDSLVNPGPAMASHK |
| | DDEEKYFPQSGVLIFGKQDSGKTNVDIEKVMITDEEEIRTTNPVATEQYGSVSTYLQSGN |
| | TQAATSDVNTQGVLPGMVWQDRDVYLRGPIWAKIPHTDGHFHPSPLMGGFGLKHPPPQI |
| | LIKNTPVPANPSTTFSAAKFASFITQYSTGQVSVEIEWELQKENSKRWNPEIQYTSNYNKS |
| | VNVDFTVDTNGVYSEPRPIGTRYLTRNL |
| SEQ ID | MAADGYLPDWLEDTLSEGIRQWWKLKPGPPPPKPAERHKDGSRGLVLPGYKYLGPFNG |
| NO: 82 | LDKGEPVNEADAAALEHDKAYDRQLNSGDNPYLKYNHADAEFQERLKEDTSFGGNLG |
| (AAVhu.25 | RAVFQAKKRVLEPLGLVEEPVKTAPGKKRPVEHSPAEPDSSSGTGKAGQQPARKRLNFG |
| (AAS99276)) | QTGDADSVPDPQPLGQPPAAPSGLGSTTMATGSGAPMADNNEGADGVGNSSGNWHCD |
| | SQWLGDRVITTSTRTWALPTYNNHLYKQISSQSGASNDNHYFGYSTPWGYFDFNRFHCH |
| | FSPRDWQRLINNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSPFYCLEYFPSQMLRTGNNF |
| | QFSYTFEDVPFHSSYAHSQSLDRLMNPLIDQYLYYLNKTQTNSGTLQQSRLLFSQAGPTN |
| | MSLQAKNWLPGPCYRQQRLSKQANDNNNSNFPWTAATKYHLNGRDSLVNPGPAMASH |
| | KDDEEKFFPMHGTLIFGKQGTNANDADLENVMITDEEEIRTTNPVATEQYGTVSNNLQN |
| | SNTGPTTGTVNHQGALPGMVWQDRDVYLQGPIWAKIPHTDGHFHPSPLMGGFGLKHPP |
| | PQIMIKNTPVPANPPTNFSSAKFASFITQYSTGQVSVEIEWELQKENSKRWNPEIQYTSNY |
| | NKSVNVDFTVDNNGVYSEPRPIGTRYLTRNL |
| SEQ ID | MAADGYLPDWLEDTLSEGIRQWWKLKPGPPPPKPAERHKDDSRGLVLPGYKYLGPFNG |
| NO: 83 | LDKGEPVNEADAAALEHDKAYDRQLDSGDNPYLKYNHADAEFQERLKEDTSFGGNLG |
| (AAVhu.27 | RAVFQAKKRILEPLGLVEEPVKTAPGKKRPVEHSPAEPDSSSGTGKAGQQPARKRLNFG |
| (AAS99277)) | QTGDADSVPDPQPLGQPPAAPSGLGTNTMASGSGAPMADNNEGADGVGNSSGNWHCD |
| | STWMGDRVITTSTRTWALPTYNNHLYKQISSQSGASNDNHYFGYSTPWGYFDFNRFHC |
| | HFSPRDWQRLINNNWGFRPKRLSFKLFNIQVKEVTQNDGTTTIANNLTSTVQVFTDSGYQ |
| | LPYVLGSAHQGCLPPFPADVFMVPQYGYLTLNNGSQAVGRSSFYCLEYFPSQMLRTGNN |
| | FTFSYTFEDVPFHSSYAHGQSLDRLMNPLIDQYLYYLSRTNTPSGTTTMSRLQFSQAGAS |
| | DVRDQSRNWLPGPCYRQQRVSKTAADNNNSDYSWTGATKYHLNGRDSLVNPGPAMAS |
| | HKDDEEKYFPQSGVLVFGKQDSGKTNVDIEKVMITDEEEIRTTNPAATEQYGSVSTNLQS |
| | GNTQAATSDVNTQGVLPGMVWQDRDVYLQGPIWAKIPHTDGHFHPSPLMGGFGLKHPP |
| | PQILIKNTPVPANPSTTFSAAKFVSFITQYSTGQVSVEIEWELQKENSKRWNPEIQYTSNY |
| | NKSVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNG |
| NO: 84 | LDKGEPVNEADAAALEHDKAYDRQLDSGDNPYLKYNHADAEFQERLKEDTSFGGNLG |
| (AAVhu.28 | RAVFQAKKRVLEPLSLVEEPVKTAPGKKRPVEHSPAEPDSSSGTGKSGNQPARKRLNFG |
| (AAS99278)) | QTGDSDSVPDPQPLGQPPAAPSGLGTNTMATGSGAPMADNNEGADGVGNSSGNWHCD |
| | STWMGDRVITTSTRTWALPTYNNHLYKQISSQSGASNDNHYFGYSTPWGYFDFNRFHC |
| | HFSPRDWQRLINNNWGFRPKRLNFKLFNIQVKEVTQNDGTTTIANNLTSTVQVFTDSEY |
| | QLPYVLGSAHQGCLPPFPADVFMVPQYGYLTLNNGSQAVGRSSFYCLEYFPSQMLRTGN |
| | NFTFSYTFEDVPFHSSYAHSQSLDRLMNPLIDQYLYYLSRTNTPSGTTTQSRLQFSQAGAS |
| | DIQDQSRNWLPGPCYRQQRVSKTSADNNNSEYSWTGATKYHLNGRDSLVNPGPAMASH |
| | KDDEEKFFPQSGVLIFGKQGSEKTNVDIEKVMITDEEEIRTTNPVATEQYGSVSTNLQSGN |
| | TQAATADVNTQGVLPGMVGQDRDVYLQGPTWAKIPHTDGHFHPSPLMGGFGLKHPPPQ |
| | ILIKNTPVPANPSTTFSAAKFASFITQYSTGQVSVEIEWELQKENSKRWNPEIQYTSNYNK |
| | SVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPFNG |
| NO: 85 | LDKGEPVNEADAAALEHDKAYDRQLDSGDNPYLKYNHADAEFQERLKEDTSFGGNLG |
| (AAVhu.29 | RAVFQAKKRVLEPLGLVEEPVKTAPGKKRPVEHSPAEPDSSSGTGKSGNQPARKRLNFG |
| (AAS99279)) | QTGDSDSVPDPQPLGQPPAAPSGLGTNTMATGSGAPMADNNEGADGVGNSSGNWHCD |
| | STWMGDRVITTSTRTWALPTYNNHLYKQISSQSGASNDNHYFGYSTPWGYFDFNRFHC |
| | HFSPRDWQRLINNNWGFRPKRLNFKLFNIQVKEVTQNDGTTTIANNLTSTVQVFTDSEY |
| | QLPYVLGSAHQGCLPPFPADVFMVPQYGYLTLNNGSQAVGRSSFYCLGYFPSQMLRTGN |
| | NFTFSYTFEDVPFHSSYAHSQSLDRLMNPLIDQYLYYLSRTNTPSGTTTQSRLQFSQAGAS |
| | DIRDQSRNWLPGPCYRQQRVSKTSADNNNSEYSWTGATKYHLNGRDSLVNPGPAMASH |
| | KDDEEKFFPQSGVLIFGKQGPEKTNVDIEKVMITDEEEIRTTNPVATEQYGSVSTNLQSGN |
| | TQAATADVNTQGVLPGMVWQDRDVYLQGPIWAKIPHTDGHFHPSPLMGGFGLKHPPPQ |
| | ILIKNTPVPANPSTTFSAAKFASFITQYSTGQVSVEIEWELQKENSKRWNPEIQYTSNYNK |
| | SVNVDFTVDTNGVYSEPRPIGTRYLTRNL |
| SEQ ID | MAADGYLPDWLEDTLSEGIRQWWKLKPGPPPPKPAERHKDDSRGLVLPGYKYLGPGNG |
| NO: 86 | LDKGEPVNAADAAALEHDKAYDQQLKAGDNPYLKYNHADAEFQERLKEDTSFGGNLG |
| (AAVhu.31 | RAVFQAKKRLLEPLGLVEEAAKTAPGKKRPVEQSPQEPDSSAGIGKSGSQPAKKKLNFG |
| (AAS99281)) | QTGDTESVPDPQPIGEPPAAPSGVGSLTMASGGGAPVADNNEGADGVGSSSGNWHCDS |
| | QWLGDRVITTSTRTWALPTYNNHLYKQISNSTSGGSSNDNAYFGYSTPWGYFDFNRFHC |
| | HFSPRDWQRLINNNWGFRPKRLNFKLFNIQVKEVTDNNGVKTIANNLTSTVQVFTDSDY |
| | QLPYVLGSAHEGCLPPFPADVFMIPQYGYLTLNDGGQAVGRSSFYCLEYFPSQMLRTGN |
| | NFQFSYEFENVPFHSSYAHSQSLDRLMNPLIDQYLYYLSKTINGSGQNQQTLKFSVAGPS |
| | NMAVQGRNYIPGPSYRQQRVSTTVTQNNNSEFAWPGASSWALNGRNSLMNPGPAMAS |
| | HKEGEDRFFPLSGSLIFGKQGTGRDNVDADKVMITNEEEIKTTNPVATESYGQVATNHQS |
| | AQAQAQTGWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSKRWNPEIQYTSN |
| | YYKSNNVEFAVSTEGVYSEPRPIGTRYLTRNL |
| SEQ ID | MAADGYLPDWLEDTLSEGIRQWWKLKPGPPPPKPAERHKDDSRGLVLPGYKYLGPGNG |
| NO: 87 | LDKGEPVNAADAAALEHDKAYDQQLKAGDNPYLKYNHADAEFQERLKEDTSFGGNLG |
| (AAVhu.32 | RAVFQAKKRLLEPLGLVEEAAKTAPGKKRPVEQSPQEPDSSAGIGKSGSQPAKKKLNFG |
| (AAS99282)) | QTGDTESVPDPQPIGEPPAAPSGVGSLTMASGGGAPVADNNEGADGVGSSSGNWHCDS |
| | QWLGDRVITTSTRTWALPTYNNHLYKQISNSTSGGSSNDNAYFGYSTPWGYFDFNRFHC |
| | HFSPRDWQRLINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSFYCLEYFPSQMLRTGN |
| | NFQFSYEFENVPFHSSYAHSQSLDRLMNPLIDQYLYYLSKTINGSGQNQQTLKFSVAGPS |
| | NMAVQGRNYIPGPSYRQQRVSTTVTQNNNSEFAWPGASSWALNGRNSLMNPGPAMAS |
| | HKEGEDRFFPLSGSLIFGKQGTGRDNVDADKVMITNEEEIKTTNPVATESYGQVATNHQS |
| | AQAQAQTGWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSKRWNPEIQYTSN |
| | YYKSNNVEFAVNTEGVYSEPRPIGTRYLTRNL |
| SEQ ID | MAADGYLPDWLEDTLSEGIRQRWKLKPGPPPPEPAERHKDDSRGLVLPGYKYLGPFNGL |
| NO: 88 | DKGEPVNEADAAALEHDKAYDRQLDSGDNPYLKYNHADAEFQERLKEDTSFGGNLGR |
| (AAVhu.34 | AVFQAKKRVLEPLGLVEEPVKTAPGKKRPVEHSPVEPDSSSGTGKAGQQPARKRLNFGQ |
| (AAS99283)) | TGDADSVPDPQPLGQPPAAPSGLGTNTMATGSGAPMADNNEGADGVGNSSGNWHCDS |
| | TWMGDRVITTSTRTWALPTYNNHLYKQISSQSGASNDNHYFGYSTPWGYFDFNRFHCH |
| | FSPRDWQRLINNNWGFRPKRLNFKLFNIQVKEVTQNDGTTTIANNLTSTVQVFTDSEYQL |
| | PYVLGSAHQGCLPPFPADVFMVPQYGYLTLNNESQAVGRSSFYCLEYFPSQMLRTGNNF |
| | TFSYTFEDVPFHSSYAHSQSLGRLMNPLIDQYLYYLSRTNTPSGTTTQSRLQFSQAGASDI |
| | RDQSRNWLPGPCYRQQRVSKTSADNNNSEYSWTGATKYHLNGRDSLVNPGPAMASHK |
| | DDEEKFFPQSGVLIFGKQGSEKTNVDIEKVMITDEEEIRTTNPVATEQYGSVSTNLQRGNR |
| | QAATADVNTQGVLPGMVWQDRDVYLQGPIWAKIPHTDGHFHPSPLMGGFGLKHPPPQI |
| | LIKNTPVPANPSTTFSAAKFASFITQYSTGQVSVEIEWELQKENSKRWNPEIQYTSNYNKS |
| | VNVDFTVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 89 | GLDKGEPVNAADAAALEHDKAYDQQLKAGDNPYLRYNHADAEFQERLQEDTSFGGNL |
| (AAVhu.37 | GRAVFQAKKRVLEPLGLVEEAAKTAPGKKRPVEPSPQRSPDSSTGIGKKGQQPAKKRLN |
| (AAS99285)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYTFEDVPFHSSYAHSQSLDRLMNPLIDQYLYYLSRTQSTGGTQGTQQLLFSQAG |
| | PANMSAQAKNWLPGPCYRQQRVSTTLSQNNNSNFAWTGATKYHLNGRDSLVNPGVAM |
| | ATHKDDEERFFPSSGVLMFGKQGAGRDNVDYSSVMLTSEEEIKTTNPVATEQYGVVAD |
| | NLQQTNTGPIVGNVNSQGALPGMVWQNRDVYLQGPIWAKIPHTDGNFHPSPLMGGFGL |
| | KHPPPQILIKNTPVPADPPTTFSQAKLASFITQYSTGQVSVEIEWELQKENSKRWNPEIQYT |
| | SNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFN |
| NO: 90 | GLDKGEPVNAADAAALEHDKAYDQQLKAGDNPYLRYNHADAEFQERLQEDTSFGGNL |
| (AAVhu.39 | GRAVFQAKKRVLEPLGLVEEGAKTAPGKKRPVEPSPQRSPDSSTGIGKKGQQPARKRLN |
| (AAS99286)) | FGRTGDSESVPDPQPIGEPPAAPSSVGSGTMAAGGGAPMADNNEGADGVGSSSGNWHC |
| | DSTWLGDRVITTSTRTWALPTYNNHLYKQISNGTSGGSTNDNTYFGYSTPWGYLDFNRF |
| | HCHFSPRDWQRLINNNWGFRPKRLSFKLFNIQVKEVTQNEGTKTIANNLASTIQVFTDSE |
| | YQPPYVLGSAHQGCLPPFPADVFMIPQYGYLTLNNGSQAVGRSSFYCLEYFPSQMLRTG |
| | NNFSFSYTFEDVPFHSSYAHSQSLDRLMNPLIDQYLYYLSRTQSTGGTAGTQQLLFSRAG |
| | PSNMSAQARNWLPGPCYRQQRVSTTLSQNNNSNFAWTGATKYHLNGRDSLVNPGVAM |
| | ATNKDDEDRFFPSSGILMFGKQGAGKDNVDYSNVMLTSEEEIKTTNPVATEQYGVVAD |
| | NLQQQNTAPTVGAVNSQGALPGMVWQNRDVYLQGPIWAKIPHTDGNFHPSPLMGGFG |
| | LKHPPPQILIKNTPVPADPPTAFNQAKLNSFIAQYSTGQVSVEIEWELQKENSKRWNPEIQ |
| | YTSNYYKSTNADFAVNTEGVYSEPRPIGTRYLTRNL |
| SEQ ID | MAADGYLPDWLEDNLSEGIREWWDLKPGAPKPKANQQKQDDGRGLVLPGYKYLGPFN |
| NO: 91 | GLDKGEPVNAADAAALEHDKAYDQQLKAGDNPYLRYNHADAEFQERLQEDTSFGGNL |
| (AAVhu.41 | GRAVFQAKKRVLEPLGPVEEAAKTAPGKKRPVEPPPQRSPDSSTGIGKKGQQPAKKRLN |
| (AAS99289)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLSFKLFNIQVKEVTQNEGTKTVANNLTSTIQVFTDSE |
| | YQLPYVLGSAHQGCLPPFPADVFMIPQYGYLTLNNGSQAVGRSSFYCLEYFPSQMLRTG |
| | NNFEFSYTFEDVPFHSSYAHSQSLDRLMNPLIDQYLYYLSRTQSTGGTQGTQQLLFSQAG |
| | PANMSAQAKNWLPGPCYRQQRVSTTLSQNNNSNFAWTGATKYHLNGRDSLVNPGVAM |
| | ATHKDDEERFFPSSGVLMFGKQGAGRDNVDYSSVMLTSEEEIKTTNPVATEQYGVVAD |
| | NLQQTNTGPIVGNVNSQGALPGMVWQNRDVYLQGPIWAKIPHTDGNFHPSPLMGGFGL |
| | KHPPPQILIKNTPVPADPPTTFSQAKLASFITQYSTGQVSVEIEWELQKENSKRWNPEIQYT |
| | SNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFN |
| NO: 92 | GLDKGEPVNAADAAALEHDKAYDQQLKAGDNPYLRYNHADAEFQERLQEDTSFGGNL |
| (AAVhu.42 | GRAVFQAKKRVLEPLGLVEEAAKTAPGKKRPVEPSPQRSPDSSTGIGKKGQQPAKKRLN |
| (AAS99290)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYTFEDVPFHSSYAHSQSLDRLMNPLIDQYLYYLSRTQSTGGTQGTQQLLFSQAG |
| | PANMSAQAKNWLPGPCYRQQRVSTTLSQSNNSNFAWTGATKYHLNGRDSLVNPGVAM |
| | ATHKDDEERFFPSSGVLMFGKQGAGRDNVDYSSVMLTSEEEIKTTNPVATEQYGVVAD |
| | NLQQTNTGPIVGNVNSQGALPGMVWQNRDVYLQGPIWAKIPHTDGNFHPSPLMGGLGL |
| | KHPPPQILIKNTPVPADPPTTFSQAKLASFITQYSTGQVSVEIEWELQKENSKRWNPEIQYT |
| | SNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFN |
| NO: 93 | GLDKGEPVNAADAAALEHDKAYDQQLKAGDNPYPRYNHADAEFQERLQEDTPFGGNL |
| (AAVhu.43 | GRAVFQAKKRVLEPLGLVEEAAKTAPGKKRPVEPSPQRSPDSSTGIGKKGQQPAKKRLN |
| (AAS99291)) | FGQTGDSESVPDPQPIGEPPAGPSGLGSGTMAAGGGAPMADNNEGADGVGNASGNWHC |
| | DSTWLGDRVITTSTRTWALPTYNNHLYKQISSASTGASNDNHYFGYSTPWGYFDFNRFH |
| | CHFSPRDWQRLINNNWGFRPKRLNFKLFNIQVKEVTTNDGVTTIANNLTSTVQVFSDSEY |
| | QLPYVLGSAHQGCLPPFPADVFMIPQYGYLTLNNGSQAVGRSSFYCLEYFPSQMLRTGN |
| | NFTFSYTFEEVPLHSSYAHSQSLDRLMNPLIVQYLYYLNRTQNQSGSAQNKDLLFSRGSP |
| | AGMSVQPKNWLPGPCYRQQRVSKTKTDNNNSNFTWTGASKYNLNGRESIINPGTAMAS |
| | HKDDEDKFFPMSGVMIFGKESAGASNTALDNVMITDEEEIKATNPVATERFGTVAVNFQ |
| | SSSTDPATGDVHAMGALPGMVWQDRDVYLQGPIWAKIPHTDGHFHPSPLMGGFGLKNP |
| | PPQILIKNTPVPANPPAEFSATKFASFITQYSTGQVSVEIEWELQKENSKRWNPEVQYTSN |
| | YAKSASVDFTVDNNGLYTEPRPIGTRYLTRPL |
| SEQ ID | MAADGYLPDWLEDTLSEGIRQWWKLRPGPPPPKPAERHKDDSRGLVLPGYKYLGPFNG |
| NO: 94 | LDKGEPVNEADAAALEHDKAYDRQLDSGDNPYLKYNHADAEFQERLKEDTSFGGNLG |
| (AAVhu.44 | RAVFQAKKRVLEPLGLVEEGAETAPGKKRPVEQSPQGPDSSSGIGKTGQQPAKKRLNFG |
| (AAS99292)) | QTGDSESVPDPQPLGEPPATPAAVGPTTMASGGGAPMADNNEGADGVGNASGNWHCD |
| | STWLGDRVITTSTRTWALPTYNNHLYKQISSASTGASNDNHYFGYSTPWGYFDFNRFHC |
| | HFSPRDWQRLINNNWGFRPKRLNFKLFNIQVKEVTTNDGVTTIANNLTSTVQVFSDSEYQ |
| | LPYVLGSAHQGCLPPFPADVFMIPQYGYLTLNNGSQAVGRSSFYCLEYFPSQMLRTGNN |
| | FTFSYTFEEVPFHSSYAHSQSLDRLMNPLIDQYLYYPNRTQNQSGSAQNKDLLFSRGSPA |
| | GMSVQPKNWLPGPCYRQQRVSKTKTDNNNSNFTWTGASKYNLNGRESIINPGTAMASH |
| | KDDEDKFFPMSGVMIFGKESAGASNTALDNVMITDEEEIKATNPVATERFGTVAVNFQS |
| | SSTDPATGDVHAMGALPGMVWQGRDVYLQGPIWAKIPHTDGHFHPSPLMGGFGLKNPP |
| | PQILIKNTPVPANPPAEFSATKFASFITQYSTGQVSVEIEWELQKENSKRWNPEVQYTSNY |
| | AKSANVDFTVDNNGLYTEPRPIGTRYLTRPL |
| SEQ ID | MAADGYLPDWLEDNLSEGIREWWDLKPGAPKPKANQQKQDDGRGLVLPGYKYLGPFN |
| NO: 111 | GLDKGEPVNAADAAALEHDKAYDQQLKAGDNPYLRYNHADAEFQERLQEDTSFGGNL |
| (AAVrh.10 | GRAVFQAKKRVLEPLGLVEEGAKTAPGKKRPVEPSPQRSPDSSTGIGKKGQQPAKKRLN |
| (AAO88201)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYQFEDVPFHSSYAHSQSLDRLMNPLIDQYLYYLSRTQSTGGTAGTQQLLFSQAG |
| | PNNMSAQAKNWLPGPCYRQQRVSTTLSQNNNSNFAWTGATKYHLNGRDSLVNPGVAM |
| | ATHKDDEERFFPSSGVLMFGKQGAGKDNVDYSSVMLTSEEEIKTTNPVATEQYGVVAD |
| | NLQQQNAAPIVGAVNSQGALPGMVWQNRDVYLQGPIWAKIPHTDGNFHPSPLMGGFGL |
| | KHPPPQILIKNTPVPADPPTTFSQAKLASFITQYSTGQVSVEIEWELQKENSKRWNPEIQYT |
| | SNYYKSTNVDFAVNTDGTYSEPRPIGTRYLTRNL |
| SEQ ID | MAADGYLPDWLEDNLSEGIREWWDLKPGAPKPKANQQKQDDGRGLVLPGYKYLGPFN |
| NO: 112 | GLDKGEPVNEADAAALEHDKAYDKQLEQGDNPYLKYNHADAEFQERLQEDTSFGGNL |
| (AAVrh.13 | GRAVFQAKKRVLEPLGLVEEGAKTAPGKKRPIESPDSSTGIGKKGQQPAKKKLNFGQTG |
| (AAO88199)) | DSESVPDPQPLGEPPAAPSGLGSGTMAAGGGAPMADNNEGADGVGNASGNWHCDSTW |
| | LGDRVITTSTRTWALPTYNNHLYKQISSQSGATNDNHFFGYSTPWGYFDFNRFHCHFSPR |
| | DWQRLINNNWGFRPRKLRFKLFNIQVKEVTTNDGVTTIANNLTSTIQVFSDSEYQLPYVL |
| | GSAHQGCLPPFPADVFMIPQYGYLTLNNGSQSVGRSSFYCLEYFPSQMLRTGNNFEFSYT |
| | FEEVPFHSSYAHSQSLDRLMNPLIDQYLYYLARTQSTTGSTRELQFHQAGPNTMAEQSK |
| | NWLPGPCYRQQRLSKNIDSNNNSNFAWTGATKYHLNGRNSLTNPGVAMATNKDDEDQ |
| | FFPINGVLVFGETGAANKTTLENVLMTSEEEIKTTNPVATEEYGVVSSNLQSSTAGPQTQ |
| | TVNSQGALPGMVWQNRDVYLQGPIWAKIPHTDGNFHPSPLMGGFGLKHPPPQILIKNTP |
| | VPANPPEVFTPAKFASFITQYSTGQVSVEIEWELQKENSKRWNPEIQYTSNYAKSNNVEF |
| | AVNNEGVYTEPRPIGTRYLTRNL |
| SEQ ID | MAADGYLPDWLEDNLSEGIREWWDLKPGAPKPKANQQKQDDGRGLVLPGYKYLGPFN |
| NO: 113 | GLDKGEPVNEADAAALEHDKAYDKQLEQGDNPYLKYNHADAEFQERLQEDTSFGGNL |
| (AAVrh.19 | GRAVFQAKKRVLEPLGLVEEGAKTAPGKKRPVEPSPQRSPDSSTGIGKTGQQPAKKRLN |
| (AAO88194)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPRKLRFKLFNIQVKEVTTDDGVTTIANNLTSTIQVFSDSE |
| | YQLPYVLGSAHQGCLPPFPADVFMIPQYGYLTLNNGSQSVGRSSFYCLEYFPSQMLRTG |
| | NNFEFSYTFEEVPFHSSYAHSQSLDRLMNPLIDQYLYYLARTQSTTGSTRELQFHQAGPN |
| | TMAEQSKNWLPGPCYRQQRLSKNIDSNNNSNFAWTGATKYHLNGRNSLTNPGVAMAT |
| | NKDDEDQFFPINGVLVFGKTGAANKTTLENVLMTSEEEIKTTNPVATEEYGVVSSNLQSS |
| | TAGPQTQTVNSQGALPGMVWQNRDVYLQGPIWAKIPHTDGNFHPSPLMDGFGLKHPPP |
| | QILIKNTPVPANPPEVFTPAKFASFITQYSTGQVSVEIEWELQKENSKRWNPEIQYTSNYA |
| | KSNNVEFAVNNEGVYTEPRPIGTRYLTRNL |
| SEQ ID | MAADGYLPDWLEDNLSEGIREWWDLKPGAPKPKANQQKQDDGRGLVLPGYKYLGPFN |
| NO: 114 | GLDKGEPVNAADAAALEHDKAYDQQLKAGDNPYLRYNHADAEFQERLQEDTSFGGNL |
| (AAVrh.22 | GRAVFQAKKRVLEPLGLVEEGAKTAPGKKRPIESPDSSTGIGKKGQQPAKKKLNFGQTG |
| (AAO88192)) | DSESVPDPQPIGEPPAGPSGLGSGTMAAGGGAPMADNNEGADGVGNASGNWHCDSTW |
| | LGDRVITTSTRTWALPTYNNHLYKQISSQSGATNDNHFFGYSTPWGYFDFNRFHCHFSPR |
| | DWQRLINNNWGFRPRKLRFKLFNIQVKEVTTNDGVTTIANNLTSTIQVFSDSEYQLPYVL |
| | GSAHQGCLPPFPADVFMIPQYGYLTLNNGSQSVGRSSFYCLEYFPSQMLRTGNNFEFSYT |
| | FEEVPFHSSYAHSQSLDRLMNPLIDQYLYYLARTQSTTGSTRELQFHQAGPNTMAEQSK |
| | NWLPGPCYRRQRLSKDIDSNNNSNFAWTGATKYHLNGRNSLTNPGVAMATNKDDEDQ |
| | FFPINGVLVFGKTGAANKTTLENVLMTSEEEIKTTNPVATEEYGVVSSNLQSSTAGPQTQ |
| | TVNSQGALPGMVWQNRDVYLQGPIWAKIPHTDGNFHPSPLMGGFGLKHPPPQILIKNTP |
| | VPANPPEVFTPAKFASFITQYSTGQVSVEIEWELQKENSKRWNPEIQYTSNYAKSNNVEF |
| | AVNNEGVYTEPRPIGTRYLTRNL |
| SEQ ID | MAADGYLPDWLEDNLSEGIREWWDLKPGAPKPKANQQKQDDGRGLVLPGYKYLGPFN |
| NO: 115 | GLDKGEPVNEADAAALEHDKAYDKQLEQGDNPYLKYNHADAEFQERLQEDTSFGGNL |
| (AAVrh.23 | GRAVFQAKKRVLEPLGLVEEGAKTAPGKKRPVEPSPQRSPDSSTGIGKTGQQPAKKRLN |
| (AAO88191)) | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYQFEDVPFHSSYAHSQSLDRLTNPLIDQYLYYLARTQSTTGSTRGLQFHQAGPN |
| | TMAEQSKNWLPGPCYRQQRLSKNIDSNNNSNFAWTGATKYHLNGRNSLTNPGVAMAT |
| | NKDDEDQFFPINGVLVFGKTGAANKTTLENVLMTSEEEIKTTNPVATEEYGVVSSNLQSS |
| | TAGPQTQTVNSQGALPGMVWQNRDVYLQGPIWAKIPHTDGNFHPSPLMGGFGLKHPPP |
| | QILIKYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGYKYLGPFN |
| NO: 116 | GLDKGEPVNAADAAALEHDKAYDQQLKAGDNPYLRYNHADAEFQERLQEDTSFGGNL |
| (AAVrh.24 | GRAVFQAKKRVLEPLGLVEEGAKTAPGKKRPIESPDSSTGIGKKGQQPAKKKLNFGQTG |
| (AAO88190)) | DSESVPDPQPIGEPPAGPSGLGSGTMAAGGGAPMADNNEGADGVGSSSGNWHCDSTWL |
| | GDRVITTSTRTWALPTYNNHLYKQISNGTSGGSTNDNTYFGYSTPWGYFDFNRFHCHFSP |
| | RDWQRLINNNWGFRPKRLNFKLFNIQVKEVTQNEGTKTIANNLTSTIQVFTDSEYQLPYV |
| | LGSAHQGCLPPFPADVFMIPQYGYLTLNNGSQAVGRSSFYCLEYFPSQMLRTGNNFEFSY |
| | QFEDVPFHSSYAHSQSLDRLMNPLIDQYLYYLSRTQSTGGTAGTQQLLFSQAGPNNMSA |
| | QAKNWLPGPCYRQQRVSTTVSQNNNSNFAWTGATKYHLNGRDSLVNPGVAMATHKG |
| | DEERFFPSSGVLMFGKQGAGKDNVDYSSVMLTSEEEIKTTNPVATEQYGVVADNLQQQ |
| | NAAPIVGAVNSQGALPGMVWQNRDVYLQGPIWAKIPHTDGNFHPSPLMGGFGLKHPPP |
| | QILIKNTPVPADPPTTFSQAKLASFITQYSTGQVSVEIEWELQKENSKRWNPEIQYTSNYY |
| | KSTNVDFAVNTEGTYSEPRPIGTRYLTRSL |
| SEQ ID | MAADGYLPDWLEDNLSEGIREWWDLKPGAPKPKANQQKQDDGRGLVLPGYKYLGPFN |
| NO: 117 | GLDKGEPVNAADAAALEHDKAYDQQLKAGDNPYLRYNHADAEFQERLQEDTSFGGNL |
| (AAVrh.35 | GRAVFQAKKRVLEPLGLVEEGAKTAPGKKRPIDSPDSSTGIGKKGQQPAKKKLNFGQTG |
| (AAO88186)) | DSESVPDPQPLGEPPAAPSSVGSGTMAAGGGAPMADNNEGADGVGNASGNWHCDSTW |
| | LGDRVITTSTRTWALPTYNNHLYKQISSSSSGATNDNHYFGYSTPWGYFDFNRFHCHFSP |
| | RDWQRLINNNWGFRPKKLRFKLFNIQVKEVTTNDGVTTIANNLTSTVQVFSDSEYQLPY |
| | VLGSAHQGCLPPFPADVFMIPQYGYLTLNNGSQSVGRSSFYCLEYFPSQMLRTGNNFEFS |
| | YSFEDVPFHSSYAHSQSLDRLMNPLIDQYLYYLARTQSTTGSTRELQFHQAGPNTMAEQS |
| | KNWLPGPCYRQQGLSKNLDFNNNSNFAWTAATKYHLNGRNSLTNPGIPMATNKDDED |
| | QFFPINGVLVFGKTGAANKTTLENVLMTSEEEIKTTNPVATEEYGVVSSNLQPSTAGPQS |
| | QTINSQGALPGMVWQNRDVYLQGPIWAKIPHTDGNFHPSPLMGGFGLKHPPPQILIKNTP |
| | VPANPPEVFTPAKFASFITQYSTGQVSVEIEWELQKENSKRWNPEIQYTSNYAKSNNVEF |
| | AVNPDGVYTEPRPIGTRYLPRNL |
| SEQ ID | MAADGYLPDWLEDNLSEGIREWWDLKPGAPKPKANQQKQDDGRGLVLPGYKYLGPFN |
| NO: 118 | GLDKGEPVNAADAAALEHDKAYDQQLEAGDNPYLRYNHADAEFQERLQEDTSFGGNL |
| (AAVrh.43 | GRAVFQAKKRVLEPLGLVEEGAKTAPGKKRPVEQSPQEPDSSSGIGKKGQQPARKRLNF |
| (AAS99245)) | GQTGDSESVPDPQPLGEPPAAPSGVGPNTMAAGGGAPMADNNEGADGVGSSSGNWHC |
| | DSTWLGDRVITTSTRTWALPTYNNHLYKQISNGTSGGATNDNTYFGYSTPWGYFDFNRF |
| | HCHFSPRDWQRLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFQFTYTFEDVPFHSSYAHSQSLDRLMNPLIDQYLYYLSRTQTTGGTANTQTLGFSQGG |
| | PNTMANQAKNWLPGPCYRQQRVSTTTGQNNNSNFAWTAGTKYHLNGRNSLANPGIAM |
| | ATHKDDEERFFPVTGSCFWQQNAARDNADYSDVMLTSEEEIKTTNPVATEEYGIVADNL |
| | QQQNTAPQIGTVNSQGALPGMVWQNRDVYLQGPIWAKIPHTDGNFHPSPLMGGFGLKH |
| | PPPQILIKNTPVPADPPTTFNQSKLNSFITQYSTGQVSVEIEWELQKENSKRWNPEIQYTSN |
| | YYKSTSVDFAVNTEGVYSEPRPIGTRYLTRNL |
| SEQ ID | MAADGYLPDWLEDNLSEGIREWWDLKPGAPKPKANQQKQDDGRGLVLPGYKYLGPFN |
| NO: 119 | GLDKGEPVNAADAAALEHDKAYDQQLKAGDNPYLRYNHADAEFQERLQEDTSFGGNL |
| (AAVrh.48 | GRAVFQAKKRVLEPLGLVEEAAKTAPGKKRPVEPSPQRSPDSSTGIGKKGQQPARKRLN |
| (AAS99246)) | FGQTGDSESVPDPQPIGEPPAGPSGLGSGTMAAGGGAPMADNNKGADGVGNASGNWH |
| | CDSTWLGDRVITTSTRTWALPTYNNHLYKQISSQSAGSTNDNVYFGYSTPWGYFDFNRF |
| | HCHFSPRDWQRLINSNWGFRPKKLNFKLFNIQVKEVTTNDGVTTIANNLTSTVQVFSDSE |
| | YQLPYVLGSAHQGCLPPFPADVFMIPQYGYLTLNNGSQSVGRSSFYCLEYFPSQMLRTG |
| | NNFTFSYTFEDVPFHSSYAHSQSLDRLMNPLIDQYLYYLARTQSNAGGTAGNRELQFYQ |
| | GGPTTMAEQAKNWLPGPCFRQQRVSKTLDQNNNSNFAWTGATKYHLNGRNSLVNPGV |
| | AMATHKDDEERFFPSSGVLIFGKTGAANKTTLENVLMTNEEEIRPTNPVATEEYGTVSSN |
| | LQAANTAAQTQVVNNQGALPGMVWQNRDVYLQGPIWAKIPHTDGNFHPSPLMGGFGL |
| | KHPPPQILIKNTPVPANPPEVFTPAKFASFITQYSTGQVSVEIEWELQKENSKRWNPEIQYT |
| | SNFDKQTGVDFAVDSQGVYSEPRPIGTRYLTRNL |
| SEQ ID | MAADGYLPDWLEDNLSEGIREWWDLKPGAPKPKANQQKQDDGRGLVLPGYKYLGPFN |
| NO: 120 | GLDKGEPVNAADAAALEHDKAYDQQLKAGDNPHLRYNHADAEFQERLQEDTSFGGNL |
| (AAVrh.49 | GRAVFQAKKRVLEPLGLVEEGAKTAPGKKRPVEPSPQRSPDSSTGIGKKGQQPARKRLN |
| (AAS99247)) | FGQTGDSESVPDPQLIGEPPAAPSSVGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLSFKLFNIQVKEVTQNEGTKTIANNLTSTIQVFTDSE |
| | YQLPYVLGSAHQGCLPPFPADVFMIPQYGNLTLNNGSQAVGRSSFYCLEYFPSQMLRTG |
| | NNFSFSYTFEDVPFHSSYAHSQSLDRLMNPLIDQYLYYLSRTQSTGGTAGTQQLLFSQAG |
| | PSNMSAQARNWLPGPCYRQQRVSTTLSQNNNSNFAWTGATKYHLNGRDSLVNPGVAM |
| | ATNKDDEDRFFPSSGILMFGKQGAGKDNMGYSNVMLTSEEEIKTTNPVATEQYGVVAD |
| | NLQQQNTAPIVGAVNSQGALPGMVWQNRDVYLQGPIWAKIPHTDGNFHPSPLMGGFGL |
| | KHPPPQILIKNTPVPADPPTAFNQAKLNSFITQYGTGQVSVEIEWELQKENSKRWNPEIQY |
| | TSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFN |
| NO: 121 | GLDKGEPVNAADAAALEHDKAYDQQLKAGDNPYLRYNHADAEFQERLQEDTSFGGNL |
| (AAVrh.50 | GRAVFQAKKRVLEPLGLVEEGAKTAPGKKRPVEPSPQRSPDSSTGIGKKGQQPAGKRLN |
| (AAS99248)) | FGQTGDSESVPDPQPIGEPPAAPSSVGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFSFSYTFEDVPFHSSYAHSQSLDRLMNPLVDQYLYYLSRTQSTGGTAGTQQLLFSQA |
| | GPSNMSAQARNWLPGPCYRQQRVSTTLSQNNNSNFAWTGATKYHLNGRDSLVNPGVA |
| | MATNKDDEDRFFPSSGILMFGKQGAGKDNVDYSNVMLTSEEEIKTTNPVATEQYGVVA |
| | DNLQQQNTAPIVGAVNSQGALPGMVWQNRDVYLQGPIWAKIPHTDGNFHPSPLMGGFG |
| | LKHPPPQILIKNTPVPADPPTAFNQAKLNSFITQYSTGQVSVEIEWELQKENSKRWSPEIQ |
| | YTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGYKYLGPFN |
| NO: 132 | GLDKGEPVNAADAAALEHDKAYDQQLKAGDNPYLRYNHADAEFQERLQEDTSFGGNL |
| (Anc80) | GRAVFQAKKRVLEPLGLVEEGAKTAPGKKRPVEQSPQEPDSSSGIGKKGQQPAXKRLNF |
| | GQTGDSESVPDPQPLGEPPAAPSGVGSNTMAXGGGAPMADNNEGADGVGNASGNWHC |
| | DSTWLGDRVITTSTRTWALPTYNNHLYKQISSQSGXSTNDNTYFGYSTPWGYFDFNRFH |
| | CHFSPRDWQRLINNNWGFRPKXLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFXFSYTFEDVPFHSSYAHSQSLDRLMNPLIDQYLYYLSRTQTTSGTAGNRXLQFSQAGP |
| | SSMANQAKNWLPGPCYRQQRVSKTXNQNNNSNFAWTGATKYHLNGRDSLVNPGPAM |
| | ATHKDDEDKFFPMSGVLIFGKQGAGNSNVDLDNVMITXEEEIKTTNPVATEXYGTVATN |
| | LQSXNTAPATGTVNSQGALPGMVWQXRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDINGVYSEPRPIGTRYLTRNL |
| | (168)..(168) Lys or Arg |
| | (205)..(205) Ala or Ser |
| | (266)..(266) Ala or Gly |
| | (311)..(311) Arg or Lys |
| | (411)..(411) Glu or Gln |
| | (460)..(460) Thr or Glu |
| | (493)..(493) Ala or Thr |
| | (562)..(562) Ser or Asn |
| | (576)..(576) Gln or Glu |
| | (587)..(587) Ser or Ala |
| | (609)..(609) Asn or Asp |
| SEQ ID | MAADGYLPDWLEDNLSEGIREWWDLKPGAPKPKANQQKQDDGRGLVLPGYKYLGPFN |
| NO: 133 | GLDKGEPVNAADAAALEHDKAYDQQLKAGDNPYLRYNHADAEFQERLQEDTSFGGNL |
| (Anc81 | GRAVFQAKKRVLEPLGLVEEGAKTAPGKKRPVEQSPQEPDSSXGIGKKGQQPAXKRLNF |
| (AKU89596)) | GQTGDSESVPDPQPLGEPPAAPSGVGSNTMAAGGGAPMADNNEGADGVGNASGNWHC |
| | DSTWLGDRVITTSTRTWALPTYNNHLYKQISXXQSGGSTNDNTYFGYSTPWGYFDFNRF |
| | HCHFSPRDWQRLINNNWGFRPKXLNFKLFNIQVKEVTINDGTTTIANNLTSTVQVFTDSE |
| | YQLPYVLGSAHQGCLPPFPADVFMIPQYGYLTLNNGSQAVGRSSFYCLEYFPSQMLRTG |
| | NNFXFSYTFEDVPFHSSYAHSQSLDRLMNPLIDQYLYYLSRTQTTGGTAGNXXLQFSQA |
| | GPSSMANQAKNWLPGPCYRQQRVSKTTNQNNNSNFAWTGATKYHLNGRDSLVNPGVA |
| | MATHKDDEDRFFPSSGVLIFGKQGAGNXNVDXXNVMITXEEEIKTTNPVATEEYGXVAT |
| | NLQSXNTAPQTGTVNSQGALPGMVWQNRDVYLQGPIWAKIPHTDGNFHPSPLMGGFGL |
| | KHPPPQILIKNTPVPANPPTTFXPAKFASFITQYSTGQVSVEIEWELQKENSKRWNPEIQYT |
| | SNYNKSTNVDFAVDTEGVYSEPRPIGTRYLTRNL |
| | (157)..(157) Thr or Ser |
| | (168)..(168) Lys or Arg |
| | (262)..(262) Asn or Ser |
| | (263)..(263) Ser or His |
| | (312)..(312) Arg or Lys |
| | (412)..(412) Glu or Gln |
| | (460)..(460) Arg or Gln |
| | (461)..(461) Thr or Glu |
| | (552)..(552) Asp or Ser |
| | (556)..(556) Leu or Tyr |
| | (557)..(557) Asp or Ser |
| | (563)..(563) Ser or Asn |
| | (580)..(580) Val or Ile |
| | (588)..(588) Asn or Ser |
| | (664)..(664) Ser or Thr |
| SEQ ID | MAADGYLPDWLEDNLSEGIREWWDLKPGAPKPKANQQKQDDGRGLVLPGYKYLGPFN |
| NO: 134 | GLDKGEPVNAADAAALEHDKAYDQQLKAGDNPYLRYNHADAEFQERLQEDTSFGGNL |
| (Anc82 | GRAVFQAKKRVLEPLGLVEEGAKTAPGKKRPVEQSPQREPDSSXGIGKKGQQPAXKRLN |
| (AKU89597)) | FGQTGDSESVPDPQPLGEPPAAPSGVGSNTMAAGGGAPMADNNEGADGVGNSSGNWH |
| | CDSTWLGDRVITTSTRTWALPTYNNHLYKQISNGTSGGSTNDNTYFGYSTPWGYFDFNR |
| | FHCHFSPRDWQRLINNNWGFRPKRLNFKLFNIQVKEVTTNEGTKTIANNLTSTVQVFTDS |
| | EYQLPYVLGSAHQGCLPPFPADVFMIPQYGYLTLNNGSQAVGRSSFYCLEYFPSQMLRT |
| | GNNFQFSYTFEDVPFHSSYAHSQSLDRLMNPLIDQYLYYLSRTQTTGGTAGTQTLQFSQA |
| | GPSSMANQAKNWLPGPCYRQQRVSTTTNQNNNSNFAWTGATKYHLNGRDSLVNPGVA |
| | MATHKDDEDRFFPSSGVLIFGKQGAGNDNVDYSNVMITXEEEIKTTNPVATEEYGVVAT |
| | NLQSANTAPQTGTVNSQGALPGMVWQNRDVYLQGPIWAKIPHTDGNFHPSPLMGGFGL |
| | KHPPPQILIKNTPVPADPPTTFNQAKLNSFITQYSTGQVSVEIEWELQKENSKRWNPEIQY |
| | TSNYYKSTNVDFAVNTEGVYSEPRPIGTRYLTRNL |
| | (158)..(158) Thr or Ser |
| | (169)..(169) Lys or Arg |
| | (564)..(564) Ser or Asn |
| SEQ ID | MAADGYLPDWLEDNLSEGIREWWDLKPGAPKPKANQQKQDDGRGLVLPGYKYLGPFN |
| NO: 135 | GLDKGEPVNAADAAALEHDKAYDQQLKAGDNPYLRYNHADAEFQERLQEDTSFGGNL |
| (Anc83 | GRAVFQAKKRVLEPLGLVEEGAKTAPGKKRPVEQSPQREPDSSXGIGKKGQQPAXKRLN |
| (AKU89598)) | FGQTGDSESVPDPQPLGEPPAAPSGVGSNTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLXFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFXFSYTFEDVPFHSSYAHSQSLDRLMNPLIDQYLYYLSRTQTTGGTAGTQTLQFSQAG |
| | PSXMANQAKNWLPGPCYRQQRVSTTTSQNNNSNFAWTGATKYHLNGRDSLVNPGVAM |
| | ATHKDDEXRFFPSS |
| | GXLIFGKQGAGKDNVDYSNVMLTSEEEIKTTNPVATEEYGVVADNLQQQNTAPQXGTV |
| | NSQGALPGMVWQNRDVYLQGPIWAKIPHTDGNFHPSPLMGGFGLKHPPPQILIKNTPVP |
| | ADPPTTFNQAKLNSFITQYSTGQVSVEIEWELQKENSKRWNPEIQYTSNYYKSTNVDFAV |
| | NTEGVYSEPRPIGTRYLTRNL |
| | (158)..(158) Thr or Ser |
| | (169)..(169) Arg or Lys |
| | (315)..(315) Asn or Ser |
| | (413)..(413) Gln or Glu |
| | (472)..(472) Asn, Thr or Ser |
| | (534)..(534) Asp or Glu |
| | (542)..(542) Ile or Val |
| | (595)..(595) Ile or Val |
| SEQ ID | MAADGYLPDWLEDNLSEGIREWWDLKPGAPKPKANQQKQDDGRGLVLPGYKYLGPFN |
| NO: 136 | GLDKGEPVNAADAAALEHDKAYDQQLKAGDNPYLRYNHADAEFQERLQEDTSFGGNL |
| (Anc84 | GRAVFQAKKRVLEPLGLVEEGAKTAPGKKRPVEPSPQRSPDSSTGIGKKGQQPAXKRLN |
| (AKU89599)) | FGQTGDSESVPDPQPIGEPPAAPSGVGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLXFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYTFEDVPFHSSYAHSQSLDRLMNPLIDQYLYYLSRTQSTGGTAGTQQLLFSQAG |
| | PSNMSAQAKNWLPGPCYRQQRVSTTLSQNNNSNFAWTGATKYHLNGRDSLVNPGVAM |
| | ATHKDDEXRFFPSSGXLMFGKQGAGKDNVDYSNVMLTSEEEIKTTNPVATEQYGVVAD |
| | NLQQQNTAPIVGAVNSQGALPGMVWQNRDVYLQGPIWAKIPHTDGNFHPSPLMGGFGL |
| | KHPPPQILIKNTPVPADPPTTFNQAKLNSFITQYSTGQVSVEIEWELQKENSKRWNPEIQY |
| | TSNYYKSTNVDFAVNTEGVYSEPRPIGTRYLTRNL |
| | (169)..(169) Arg or Lys |
| | (315)..(315) Asn or Ser |
| | (534)..(534) Asp or Glu |
| | (542)..(542) Ile or Val |
| SEQ ID | MAADGYLPDWLEDNLSEGIREWWDLKPGAPKPKANQQKQDDGRGLVLPGYKYLGPFN |
| NO: 137 | GLDKGEPVNAADAAALEHDKAYDQQLKAGDNPYLRYNHADAEFQERLQEDTSFGGNL |
| (Anc94) | GRAVFQAKKRVLEPLGLVEEGAKTAPGKKRPVEPSPQRSPDSSTGIGKKGQQPAKKRLN |
| | FGQTGDSESVPDPQPIGEPPAGPSGLGSGTMAAGGGAPMADNNEGADGVGSSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEYFPSQMLRTG |
| | NNFEFSYTFEDVPFHSSYAHSQSLDRLMNPLIDQYLYYLSRTQSTGGTAGTQQLLFSQAG |
| | PXNMSAQAKNWLPGPCYRQQRVSTTLSQNNNSNFAWTGATKYHLNGRDSLVNPGVAM |
| | ATHKDDEERFFPSSGVLMFGKQGAGKDNVDYSSVMLTSEEEIKTTNPVATEQYGVVAD |
| | NLQQQNTAPIVGAVNSQGALPGMVWQNRDVYLQGPIWAKIPHTDGNFHPSPLMGGFGL |
| | KHPPPQILIKNTPVPADPPTTFSQAKLASFITQYSTGQVSVEIEWELQKENSKRWNPEIQYT |
| | SNYYKSTNVDFAVNTEGTYSEPRPIGTRYLTRNL |
| | (471)..(471) Ser or Asn |
| SEQ ID | MAADGYLPDWLEDNLSEGIREWWDLKPGAPKPKANQQKQDDGRGLVLPGYKYLGPFN |
| NO: 138 | GLDKGEPVNAADAAALEHDKAYDQQLKAGDNPYLRYNHADAEFQERLQEDTSFGGNL |
| (Anc110 | GRAVFQAKKRVLEPLGLVEEGAKTAPGKKRPVEQSPQEPDSSXLGIGKTGQQPAXKRLN |
| (AKU89600)) | FGQTGDSESVPDPQPLGEPPAAPSGVGSNTMASGGGAPMADNNEGADGVGNSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLNFKLFNIQVKEVTTNEGTKTIANNLTSTVQVFTDSE |
| | YQLPYVLGSAHQGCLPPFPADVFMIPQYGYLTLNNGSQAVGRSSFYCLEYFPSQMLRTG |
| | NNFQFSYTFEDVPFHSSYAHSQSLDRLMNPLIDQYLYYLSRTQTTGTXGTQTLXFSQAGP |
| | SSMANQARNWVPGPCYRQQRVSTTTNQNNNSNFAWTGAXKXXLNGRDSLMNPGVAM |
| | ASHKDDEDRFFPSSGVLIFGKQGAGNDNVDYSXVMITNEEEIKTTNPVATEEYGAVATN |
| | XQXLANTQAQTGLVHNQGVLPGMVWQNRDVYLQGPIWAKIPHTDGNFHPSPLMGGFG |
| | LKHPPPQILIKNTPVPADPPTTFNQAKLNSFITQYSTGQVSVEIEWELQKENSKRWNPEIQ |
| | YTSNYYKSTNVDFAVNTEGVYSEPRPIGTRYLTRNL |
| | (157)..(157) Ser or Thr |
| | (169)..(169) Lys or Arg |
| | (457)..(457) Ala or Gly |
| | (463)..(463) Gln or Ala |
| | (508)..(508) Thr or Ala |
| | (510)..(510) Tyr or Phe |
| | (511)..(511) His or Lys |
| | (558)..(558) Gln or Asn |
| | (585)..(585) Asn or His |
| | (587)..(587) Ser or Ala |
| SEQ ID | MAADGYLPDWLEDNLSEGIREWWDLKPGAPKPKANQQKQDDGRGLVLPGYKYLGPFN |
| NO: 139 | GLDKGEPVNAADAAALEHDKAYDQQLKAGDNPYLRYNHADAEFQERLQEDTSFGGNL |
| (Anc113 | GRAVFQAKKRVLEPLGLVEEGAKTAPGKKRPVEXSPQRSPDSSTGIGKKGQQPAXKRLN |
| (AKU89601)) | FGQTGDSESVPDPQPLGEPPAAPSGVGSGTMAAGGGAPMADNNEGADGVGNASGNWH |
| | CDSTWLGDRVITTSTRTWALPTYNNHLYKQISSQSAGSTNDNTYFGYSTPWGYFDFNRF |
| | HCHFSPRDWQRLINNNWGFRPKKLXFKLFNIQVKEVTTNDGVTTIANNLTSTVQVFSDSE |
| | YQLPYVLGSAHQGCLPPFPADVFMIPQYGYLTLNNGSQSVGRSSFYCLEYFPSQMLRTG |
| | NNFEFSYTFEDVPFHSSYAHSQSLDRLMNPLIDQYLYYLARTQSTTGGTAGNRELQFXQA |
| | GPSTMAEQAKNWLPGPCYRQQRVSKTLDQNNNSNFAWTGATKYHLNGRNSLVNPGVA |
| | MATHKDDEDRFFPSSGVLIFGKTGAANKTTLENVLMTXEEEIKTTNPVATEEYGXVSSNL |
| | QSXNTAPQTQTVNSQGALPGMVWQNRDVYLQGPIWAKIPHTDGNFHPSPLMGGFGLKH |
| | PPPQILIKNTPVPANPPEVFTPAKFASFITQYSTGQVSVEIEWELQKENSKRWNPEIQYTSN |
| | YDKSTNVDFAVDSEGVYSEPRPIGTRYLTRNL |
| | (148)..(148) Pro or Gln |
| | (169)..(169) Lys or Arg |
| | (314)..(314) Arg or Asn |
| | (466)..(466) Tyr or His |
| | (563)..(563) Asn or Ser |
| | (580)..(580) Val or Ile |
| | (588)..(588) Ala or Ser |
| SEQ ID | MAADGYLPDWLEDNLSEGIREWWDLKPGAPKPKANQQKQDDGRGLVLPGYKYLGPFN |
| NO: 140 | GLDKGEPVNAADAAALEHDKAYDQQLKAGDNPYLRYNHADAEFQERLQEDTSFGGNL |
| (Anc126 | GRAVFQAKKRVLEPLGLVEEGAKTAPGKKRPVEQSPQEPDSSSGIGKXGQQPAXKRLNF |
| (AKU89602)) | GQTGDSESVPDPQPLGEPPAAPSGVGSNTMASGGGAPMADNNEGADGVGNXSGNWHC |
| | DSTWLGDRVITTSTRTWALPTYNNHLYKQISSQSGASNDNHYFGYSTPWGYFDFNRFHC |
| | HFSPRDWQRLINNNWGFRPKXLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFPSQMLRTGNN |
| | FXFSYTFEDVPFHSSYAHSQSLDRLMNPLIDQYLYYLXRTQTTSGTAQNRELXFSQAGPS |
| | SMXNQAKNWLPGPCYRQQRVSKTANDNNNSNFAWTGATKYHLNGRDSLVNPGPAMA |
| | SHKDDEDKFFPMSGVLIFGKQGAGASNVDLDNVMITDEEEIKTTNPVATEQYGTVATNL |
| | QSSNTAPATGTVNSQGALPGMVWQDRDVYLQGPIWAKIPHTDGHFHPSPLMGGFGLKH |
| | PPPQILIKNTPVPANPPTTFSPAKFASFITQYSTGQVSVEIEWELQKENSKRWNPEIQYTSN |
| | YNKSXNVDFTVDINGVYSEPRPIGTRYLTRNL |
| | (162)..(162) Ser or Thr |
| | (168)..(168) Lys or Arg |
| | (224)..(224) Ala or Ser |
| | (310)..(310) Arg or Lys |
| | (410)..(410) Thr or Gln |
| | (446)..(446) Ser or Asn |
| | (461)..(461) Gln or Leu |
| | (471)..(471) Ala or Ser |
| | (708)..(708) Ala or Thr |
| SEQ ID | MAADGYLPDWLEDNLSEGIREWWDLKPGAPQPKANQQHQDDXRGLVLPGYKYLGPFN |
| NO: 141 | GLDKGEPVNEADAAALEHDKAYDQQLKAGDNPYLKYNHADAEFQERLQEDTSFGGNL |
| Anc127 | GRAVFQAKKRVLEPLGLVEEAAKTAPGKKRPVEQSPQEPDSSSGIGKSGQQPAXKRLNF |
| (AKU89603) | GQTGDSESVPDPQPLGEPPAAPSGVGSNTMASGGGAPMADNNEGADGVGNSSGNWHC |
| | DSTWLGDRVITTSTRTWALPTYNNHLYKQISSQSGASNDNHYFGYSTPWGYFDFNRFHC |
| | HFSPRDWQRLINNNWGFRPKXLNFKLFNIQVKEVTQNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFXFSYTFEDVPFHSSYAHSQSLDRLMNPLIDQYLYYLXRTQTTSGTTQQSRLXFSQAGP |
| | SSMXQQAXNWLPGPCYRQQRVSKTANDNNNSNFAWTXATKYHLNGRDSLVNPGPAM |
| | ASHKDDEEKFFPMHGXLIFGKQGTGASNVDLDNVMITDEEEIRTTNPVATEQYGTVATN |
| | LQSSNTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSVNVDFTVDTNGVYSEPRPIGTRYLTRNL |
| | (42)..(42) Gly or Ser |
| | (168)..(168) Arg or Lys |
| | (310)..(310) Lys or Arg |
| | (410)..(410) Thr or Gln |
| | (446)..(446) Ser or Arg |
| | (461)..(461) Gln or Leu |
| | (471)..(471) Ala or Ser |
| | (475)..(475) Lys or Arg |
| | (504)..(504) Gly or Ala |
| | (539)..(539) Val or Asn |
| SEQ ID | MAADGYLPDWLEDNLSEGIREWWDLKPGAPKPKANQQKQDDGRGLVLPGYKYLGPFN |
| NO: 142 | GLDKGEPVNAADAAALEHDKAYDQQLKAGDNPYLRYNHADAEFQERLQEDTSFGGNL |
| (Anc80L65 | GRAVFQAKKRVLEPLGLVEEGAKTAPGKKRPVEQSPQEPDSSSGIGKKGQQPARKRLNF |
| (AKU89595)) | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRTLQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITNEEEIKTTNPVATEEYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDINGVYSEPRPIGTRYLTRNL |
| SEQ ID | MAADGYLPDWLEDNLSEGIREWWDLKPGAPKPKANQQKQDDGRGLVLPGYKYLGPFN |
| NO: 143 | GLDKGEPVNAADAAALEHDKAYDQQLKAGDNPYLRYNHADAEFQERLQEDTSFGGNL |
| (Anc80L1) | GRAVFQAKKRVLEPLGLVEEGAKTAPGKKRPVEQSPQEPDSSSGIGKKGQQPAKKRLNF |
| | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRTLQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITSEEEIKTTNPVATEQYGTVATN |
| | LQSSNTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDINGVYSEPRPIGTRYLTRNL |
| SEQ ID | MAADGYLPDWLEDNLSEGIREWWDLKPGAPKPKANQQKQDDGRGLVLPGYKYLGPFN |
| NO: 144 | GLDKGEPVNAADAAALEHDKAYDQQLKAGDNPYLRYNHADAEFQERLQEDTSFGGNL |
| (Anc80L27) | GRAVFQAKKRVLEPLGLVEEGAKTAPGKKRPVEQSPQEPDSSSGIGKKGQQPARKRLNF |
| | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRTLQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITNEEEIKTTNPVATEQYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDINGVYSEPRPIGTRYLTRNL |
| SEQ ID | MAADGYLPDWLEDNLSEGIREWWDLKPGAPKPKANQQKQDDGRGLVLPGYKYLGPFN |
| NO: 145 | GLDKGEPVNAADAAALEHDKAYDQQLKAGDNPYLRYNHADAEFQERLQEDTSFGGNL |
| (Anc80L33) | GRAVFQAKKRVLEPLGLVEEGAKTAPGKKRPVEQSPQEPDSSSGIGKKGQQPAKKRLNF |
| | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRTLQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITSEEEIKTTNPVATEQYGTVATN |
| | LQSSNTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 146 | GLDKGEPVNAADAAALEHDKAYDQQLKAGDNPYLRYNHADAEFQERLQEDTSFGGNL |
| (Anc80L36) | GRAVFQAKKRVLEPLGLVEEGAKTAPGKKRPVEQSPQEPDSSSGIGKKGQQPAKKRLNF |
| | GQTGDSESVPDPQPLGEPPAAPSGVGSNTMASGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRTLQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITSEEEIKTTNPVATEEYGTVATN |
| | LQSSNTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 147 | GLDKGEPVNAADAAALEHDKAYDQQLKAGDNPYLRYNHADAEFQERLQEDTSFGGNL |
| (Anc80L44) | GRAVFQAKKRVLEPLGLVEEGAKTAPGKKRPVEQSPQEPDSSSGIGKKGQQPAKKRLNF |
| | GQTGDSESVPDPQPLGEPPAAPSGVGSNTMASGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRELQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITNEEEIKTTNPVATEQYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 148 | GLDKGEPVNAADAAALEHDKAYDQQLKAGDNPYLRYNHADAEFQERLQEDTSFGGNL |
| (Anc80L59) | GRAVFQAKKRVLEPLGLVEEGAKTAPGKKRPVEQSPQEPDSSSGIGKKGQQPAKKRLNF |
| | GQTGDSESVPDPQPLGEPPAAPSGVGSNTMASGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRELQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITNEEEIKTTNPVATEEYGTVATN |
| | LQSANTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 149 | GLDKGEPVNAADAAALEHDKAYDQQLKAGDNPYLRYNHADAEFQERLQEDTSFGGNL |
| (Anc80L60) | GRAVFQAKKRVLEPLGLVEEGAKTAPGKKRPVEQSPQEPDSSSGIGKKGQQPARKRLNF |
| | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRELQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITSEEEIKTTNPVATEEYGTVATN |
| | LQSSNTAPATGTVNSQGALPGMVWQERDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 150 | GLDKGEPVNAADAAALEHDKAYDQQLKAGDNPYLRYNHADAEFQERLQEDTSFGGNL |
| (Anc80L62) | GRAVFQAKKRVLEPLGLVEEGAKTAPGKKRPVEQSPQEPDSSSGIGKKGQQPARKRLNF |
| | GQTGDSESVPDPQPLGEPPAAPSGVGSNTMASGGGAPMADNNEGADGVGNASGNWHC |
| | DSTWLGDRVITTSTRTWALPTYNNHLYKQISSQSGGSTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRELQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITSEEEIKTTNPVATEEYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWDLKPGAPKPKANQQKQDDGRGLVLPGYKYLGPFN |
| NO: 151 | GLDKGEPVNAADAAALEHDKAYDQQLKAGDNPYLRYNHADAEFQERLQEDTSFGGNL |
| (Anc82DI) | GRAVFQAKKRVLEPLGLVEEGAKTAPGKKRPVEQSPQREPDSSTGIGKSGQQPAKKRLN |
| | FGQTGDSESVPDPQPLGEPPAAPSGVGSNTMASGGGAPMADNNEGADGVGNSSGNWHC |
| | DSTWLGDRVITTSTRTWALPTYNNHLYKQISNGTSGGSTNDNTYFGYSTPWGYFDFNRF |
| | HCHFSPRDWQRLINNNWGFRPKRLNFKLFNIQVKEVTTNEGTKTIANNLTSTVQVFTDSE |
| | YQLPYVLGSAHQGCLPPFPADVFMIPQYGYLTLNNGSQAVGRSSFYCLEYFPSQMLRTG |
| | NNFQFSYTFEDVPFHSSYAHSQSLDRLMNPLIDQYLYYLSRTQTTGGTAGTQTLQFSQAG |
| | PSSMANQARNWVPGPCYRQQRVSTTTNQNNNSNFAWTGATKYHLNGRDSLMNPGVA |
| | MASHKDDEDRFFPSSGVLIFGKQGAGNDNVDYSNVMITSEEEIKTTNPVATEEYGVVAT |
| | NHQSANTQAQTGTVQNQGILPGMVWQNRDVYLQGPIWAKIPHTDGNFHPSPLMGGFGL |
| | KHPPPQILIKNTPVPADPPTTFNQAKLNSFITQYSTGQVSVEIEWELQKENSKRWNPEIQY |
| | TSNYYKSTNVDFAVNTEGVYSEPRPIGTRYLTRNL |
| SEQ ID | MAADGYLPDWLEDNLSEGIREWWDLKPGAPKPKANQQKQDNGRGLVLPGYKYLGPFN |
| NO: 152 | GLDKGEPVNAADAAALEHDKAYDQQLQAGDNPYLRYNHADAEFQERLQEDTSFGGNL |
| (AAVrh.74) | GRAVFQAKKRVLEPLGLVESPVKTAPGKKRPVEPSPQRSPDSSTGIGKKGQQPAKKRLNF |
| | GQTGDSESVPDPQPIGEPPAGPSGLGSGTMAAGGGAPMADNNEGADGVGSSSGNWHCD |
| | STWLGDRVITTSTRTWALPTYNNHLYKQISNGTSGGSTNDNTYFGYSTPWGYFDFNRFH |
| | CHFSPRDWQRLINNNWGFRPKRLNFKLFNIQVKEVTQNEGTKTIANNLTSTIQVFTDSEY |
| | QLPYVLGSAHQGCLPPFPADVFMIPQYGYLTLNNGSQAVGRSSFYCLEYFPSQMLRTGN |
| | NFEFSYNFEDVPFHSSYAHSQSLDRLMNPLIDQYLYYLSRTQSTGGTAGTQQLLFSQAGP |
| | NNMSAQAKNWLPGPCYRQQRVSTTLSQNNNSNFAWTGATKYHLNGRDSLVNPGVAM |
| | ATHKDDEERFFPSSGVLMFGKQGAGKDNVDYSSVMLTSEEEIKTTNPVATEQYGVVAD |
| | NLQQQNAAPIVGAVNSQGALPGMVWQNRDVYLQGPIWAKIPHTDGNFHPSPLMGGFGL |
| | KHPPPQILIKNTPVPADPPTTFNQAKLASFITQYSTGQVSVEIEWELQKENSKRWNPEIQYT |
| | SNYYKSTNVDFAVNTEGTYSEPRPIGTRYLTRNL |
| Anc80- | MAADGYLPDWLEDNLSEGIREWWDLKPGAPKPKANQQKQDDGRGLVLPGYKYLGPFN |
| 55 | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPAKKRLNF |
| NO: 153 | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRELQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITNEEEIKTTNPVATEEYGTVATN |
| | LQSSNTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| Anc80- | MAADGYLPDWLEDNLSEGIREWWDLKPGAPKPKANQQKQDDGRGLVLPGYKYLGPFN |
| 129 | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPAKKRLNF |
| NO: 154 | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRTLQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITNEEEIKTTNPVATEQYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| Anc80- | MAADGYLPDWLEDNLSEGIREWWDLKPGAPKPKANQQKQDDGRGLVLPGYKYLGPFN |
| 156 | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPAKKRLNF |
| NO: 155 | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRTLQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITSEEEIKTTNPVATEEYGTVATN |
| | LQSANTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| Anc80- | MAADGYLPDWLEDNLSEGIREWWDLKPGAPKPKANQQKQDDGRGLVLPGYKYLGPFN |
| 751- | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPAKKRLNF |
| NO: 156 | GQTGDSESVPDPQPLGEPPAAPSGVGSNTMASGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFEFSYTFEDVPFHSSYAHSQSLDRLMNPLIDQYLYYLSRTQTTSGTAGNRELQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITSEEEIKTTNPVATEEYGTVATN |
| | LQSSNTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| Anc80- | MAADGYLPDWLEDNLSEGIREWWDLKPGAPKPKANQQKQDDGRGLVLPGYKYLGPFN |
| 1029 | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPARKRLNF |
| NO: 157 | GQTGDSESVPDPQPLGEPPAAPSGVGSNTMAAGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKKLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRTLQFSQAGP |
| | SSMANQAKNWLPGPCYRQQRVSKTTNQNNNSNFAWTGATKYHLNGRDSLVNPGPAM |
| | ATHKDDEDKFFPMSGVLIFGKQGAGNSNVDLDNVMITNEEEIKTTNPVATEEYGTVATN |
| | LQSANTAPATGTVNSQGALPGMVWQDRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| Anc80- | MAADGYLPDWLEDNLSEGIREWWDLKPGAPKPKANQQKQDDGRGLVLPGYKYLGPFN |
| 1712 | GLDKGEPVNAADAAALEHDKAYDQQLKAGDNPYLRYNHADAEFQERLQEDTSFGGNL |
| SEQ ID | GRAVFQAKKRVLEPLGLVEEGAKTAPGKKRPVEQSPQEPDSSSGIGKKGQQPARKRLNF |
| NO: 158 | GQTGDSESVPDPQPLGEPPAAPSGVGSNTMASGGGAPMADNNEGADGVGNASGNWHC |
| | DSTWLGDRVITTSTRTWALPTYNNHLYKQISSQSGASTNDNTYFGYSTPWGYFDFNRFH |
| | CHFSPRDWQRLINNNWGFRPKRLNFKLFNIQVKEVTTNDGTTTIANNLTSTVQVFTDSEY |
| | QLPYVLGSAHQGCLPPFPADVFMIPQYGYLTLNNGSQAVGRSSFYCLEYFPSQMLRTGN |
| | NFQFSYTFEDVPFHSSYAHSQSLDRLMNPLIDQYLYYLSRTQTTSGTAGNRELQFSQAGP |
| | SSMANQAKNWLPGPCYRQQRVSKTANQNNNSNFAWTGATKYHLNGRDSLVNPGPAM |
| | ATHKDDEDKFFPMSGVLIFGKQGAGNSNVDLDNVMITNEEEIKTTNPVATEQYGTVATN |
| | LQSANTAPATGTVNSQGALPGMVWQNRDVYLQGPIWAKIPHTDGHFHPSPLMGGFGLK |
| | HPPPQILIKNTPVPANPPTTFSPAKFASFITQYSTGQVSVEIEWELQKENSKRWNPEIQYTS |
| | NYNKSTNVDFAVDTNGVYSEPRPIGTRYLTRNL |
| SEQ ID | MAADGYLPDWLEDNLSEGIREWWALKPGAPQPKANQQHQDNARGLVLPGYKYLGPGN |
| NO: 159 | GLDKGEPVNAADAAALEHDKAYDQQLKAGDNPYLKYNHADAEFQERLKEDTSFGGNL |
| AAV9- | GRAVFQAKKRLLEPLGLVEEAAKTAPGKKRPVEQSPQEPDSSAGIGKSGAQPAKKRLNF |
| retro | GQTGDTESVPDPQPIGEPPAAPSGVGSLTMASGGGAPVADNNEGADGVGSSSGNWHCD |
| | SQWLGDRVITTSTRTWALPTYNNHLYKQISNSTSGGSSNDNAYFGYSTPWGYFDFNRFH |
| | CHFSPRDWQRLINNNWGFRPKRLNFKLFNIQVKEVTDNNGVKTIANNLTSTVQVFTDSD |
| | YQLPYVLGSAHEGCLPPFPADVFMIPQYGYLTLNDGSQAVGRSSFYCLEYFPSQMLRTG |
| | NNFQFSYEFENVPFHSSYAHSQSLDRLMNPLIDQYLYYLSKTINGSGQNQQTLKFSVAGP |
| | SNMAVQGRNYIPGPSYRQQRVSTTVTQNNNSEFAWPGASSWALNGRNSLMNPGPAMAS |
| | HKEGEDRFFPLSGSLIFGKQGTGRDNVDADKVMITNEEEIKTTNPVATESYGQVATNHQS |
| | AQLADQDYTKTAAQAQTGWVQNQGILPGMVWQDRDVYLQGPIWAKIPHTDGNFHPSP |
| | LMGGFGMKHPPPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AFT-6 | MAADGYLPDWLEDNLSEGIREWWDLKPGAPKPKANQQKQDDGRGLVLP |
| Capsid + | GYKYLGPFNGLDKGEPVNAADAAALEHDKAYDQQLKAGDNPYLRYNHA |
| targeting | DAEFQERLQEDTSFGGNLGRAVFQAKKRVLEPLGLVEEGAKTAPGKKRPV |
| peptide | EQSPQEPDSSSGIGKKGQQPARKRLNFGQTGDSESVPDPQPLGEPPAAPSGV |
| SEQ ID | GSNTMAAGGGAPMADNNEGADGVGNASGNWHCDSTWLGDRVITTSTRT |
| NO: 55820 | WALPTYNNHLYKQISSQSGGSTNDNTYFGYSTPWGYFDFNRFHCHFSPRD |
| | WQRLINNNWGFRPKKLNFKLFNIQVKEVTTNDGTTTIANNLTSTVQVFTDS |
| | EYQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSRT |
| | QTTSGTAGNRTLQFSQAGPSSMANQAKNWLPGPCYRQQRVSKTTNQNNN |
| | SNFAWTGATKYHLNGRDSLVNPGPAMATHKDDEDKFFPMSGVLIFGKQG |
| | AGNSNVDLDNVMITNEEEIKTTNPVATESYGQVATNHQSPLNGAVHLYAQ |
| | AQTGWVQNQGILPGMVWQDRDVYLQGPIWAKIPHTDGHFHPSPLMGGFG |
| | LKHPPPQILIKNTPVPANPPTTFSPAKFASFITQYSTGQVSVEIEWELQKENS |
| | KRWNPEIQYTSNYNKSTNVDFAVDTNGVYSEPRPIGTRYLTRNL |
| AFT-7(1) | MAADGYLPDWLEDNLSEGIREWWDLKPGAPKPKANQQKQDDGRGLVLP |
| Capsid + | GYKYLGPFNGLDKGEPVNAADAAALEHDKAYDQQLKAGDNPYLRYNHA |
| targeting | DAEFQERLQEDTSFGGNLGRAVFQAKKRVLEPLGLVEEGAKTAPGKKRPV |
| peptide | EQSPQEPDSSSGIGKKGQQPARKRLNFGQTGDSESVPDPQPLGEPPAAPSGV |
| SEQ ID | GSNTMAAGGGAPMADNNEGADGVGNASGNWHCDSTWLGDRVITTSTRT |
| NO: 55821 | WALPTYNNHLYKQISSQSGGSTNDNTYFGYSTPWGYFDFNRFHCHFSPRD |
| | WQRLINNNWGFRPKKLNFKLFNIQVKEVTTNDGTTTIANNLTSTVQVFTDS |
| | EYQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSKTI |
| | NGSGQNQQTLKFSVAGPSSMANQAKNWLPGPCYRQQRVSKTTNQNNNSN |
| | FAWTGATKYHLNGRDSLVNPGPAMATHKDDEDKFFPMSGVLIFGKQGAG |
| | NSNVDLDNVMITNEEEIKTTNPVATESYGQVATNHQSPLNGAVHLYAQAQ |
| | TGWVQNQGILPGMVWQDRDVYLQGPIWAKIPHTDGHFHPSPLMGGFGLK |
| | HPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| AFT-7(2) | MAADGYLPDWLEDNLSEGIREWWDLKPGAPKPKANQQKQDDGRGLVLP |
| Capsid + | GYKYLGPFNGLDKGEPVNAADAAALEHDKAYDQQLKAGDNPYLRYNHA |
| targeting | DAEFQERLQEDTSFGGNLGRAVFQAKKRVLEPLGLVEEGAKTAPGKKRPV |
| peptide | EQSPQEPDSSSGIGKKGQQPARKRLNFGQTGDSESVPDPQPLGEPPAAPSGV |
| SEQ ID | GSNTMAAGGGAPMADNNEGADGVGNASGNWHCDSTWLGDRVITTSTRT |
| NO: 55822 | WALPTYNNHLYKQISSQSGGSTNDNTYFGYSTPWGYFDFNRFHCHFSPRD |
| | WQRLINNNWGFRPKKLNFKLFNIQVKEVTTNDGTTTIANNLTSTVQVFTDS |
| | EYQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSRT |
| | QTTSGTAGNRTLQFSQAGPSSMANQAKNWLPGPCYRQQRVSKTTNQNNN |
| | SNFAWTGATKYHLNGRDSLVNPGPAMATHKDDEDKFFPMSGVLIFGKQG |
| | AGNSNVDLDNVMITNEEEIKTTNPVATESYGQVATNHQSPLNGAVHLYAQ |
| | AQTGWVQNQGILPGMVWQDRDVYLQGPIWAKIPHTDGHFHPSPLMGGFG |
| | LKHPPPQILIKNTPVPANPPTTFSPAKFASFITQYSTGQVSVEIEWELQKENS |
| | KRWNPEIQYTSNYNKSTNVDFAVDTNGVYSEPRPIGTRYLTRNL |
| AFT-8(1) | MAADGYLPDWLEDNLSEGIREWWDLKPGAPKPKANQQKQDDGRGLVLP |
| Capsid + | GYKYLGPFNGLDKGEPVNAADAAALEHDKAYDQQLKAGDNPYLRYNHA |
| targeting | DAEFQERLQEDTSFGGNLGRAVFQAKKRVLEPLGLVEEGAKTAPGKKRPV |
| peptide | EQSPQEPDSSSGIGKKGQQPARKRLNFGQTGDSESVPDPQPLGEPPAAPSGV |
| SEQ ID | GSNTMAAGGGAPMADNNEGADGVGNASGNWHCDSTWLGDRVITTSTRT |
| NO: 55823 | WALPTYNNHLYKQISSQSGGSTNDNTYFGYSTPWGYFDFNRFHCHFSPRD |
| | WQRLINNNWGFRPKKLNFKLFNIQVKEVTTNDGTTTIANNLTSTVQVFTDS |
| | EYQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSKTI |
| | NGSGQNQQTLKFSVAGPSSMANQAKNWLPGPCYRQQRVSTTVTQNNNSE |
| | FAWPGASSWALNGRDSLVNPGPAMATHKDDEDKFFPMSGVLIFGKQGAG |
| | NSNVDLDNVMITNEEEIKTTNPVATESYGQVATNHQSPLNGAVHLYAQAQ |
| | TGWVQNQGILPGMVWQDRDVYLQGPIWAKIPHTDGHFHPSPLMGGFGLK |
| | HPPPQILIKNTPVPANPPTTFSPAKFASFITQYSTGQVSVEIEWELQKENSKR |
| | WNPEIQYTSNYNKSTNVDFAVDTNGVYSEPRPIGTRYLTRNL |
| AFT-8(2) | MAADGYLPDWLEDNLSEGIREWWDLKPGAPKPKANQQKQDDGRGLVLP |
| Capsid + | GYKYLGPFNGLDKGEPVNAADAAALEHDKAYDQQLKAGDNPYLRYNHA |
| targeting | DAEFQERLQEDTSFGGNLGRAVFQAKKRVLEPLGLVEEGAKTAPGKKRPV |
| peptide | EQSPQEPDSSSGIGKKGQQPARKRLNFGQTGDSESVPDPQPLGEPPAAPSGV |
| SEQ ID | GSNTMAAGGGAPMADNNEGADGVGNASGNWHCDSTWLGDRVITTSTRT |
| NO: 55824 | WALPTYNNHLYKQISSQSGGSTNDNTYFGYSTPWGYFDFNRFHCHFSPRD |
| | WQRLINNNWGFRPKKLNFKLFNIQVKEVTTNDGTTTIANNLTSTVQVFTDS |
| | EYQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSRT |
| | QTTSGTAGNRTLQFSQAGPSSMANQAKNWLPGPCYRQQRVSTTVTQNNNS |
| | EFAWPGASSWALNGRDSLVNPGPAMATHKDDEDKFFPMSGVLIFGKQGA |
| | GNSNVDLDNVMITNEEEIKTTNPVATESYGQVATNHQSPLNGAVHLYAQA |
| | QTGWVQNQGILPGMVWQDRDVYLQGPIWAKIPHTDGHFHPSPLMGGFGL |
| | KHPPPQILIKNTPVPANPPTTFSPAKFASFITQYSTGQVSVEIEWELQKENSK |
| | RWNPEIQYTSNYNKSTNVDFAVDTNGVYSEPRPIGTRYLTRNL |
| AFT-9 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55825 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-10 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55826 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVRLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-11 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55827 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVKLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-12 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55828 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPINGAVHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-13 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55829 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPVNGAVHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-14 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55830 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPANGAVHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-15 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55831 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTVRLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT16 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55832 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTVHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-17 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55833 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTVKLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-18 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55834 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPSNGTLRLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-19 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55835 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPSNGTLHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-20 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55836 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPSNGTLKLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-21 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55837 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTLRLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-22 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55838 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTLHLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-23 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55839 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTNGTLKLYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-24 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55840 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTQGAVTVRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-25 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55841 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVTVRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-26 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55842 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVHVRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-27 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55843 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVHVYAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-28 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55844 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPSQGAVTLRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-29 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55845 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVTLRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-30 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55846 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPLQGAVHLRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AFT-31 | MAADGYLPDWLEDNLSEGIREWWALKPGAPQPKANQQHQDNARGLVLP |
| Capsid + | GYKYLGPGNGLDKGEPVNAADAAALEHDKAYDQQLKAGDNPYLKYNHA |
| targeting | DAEFQERLKEDTSFGGNLGRAVFQAKKRLLEPLGLVEEAAKTAPGKKRPV |
| peptide | EQSPQEPDSSAGIGKSGAQPAKKRLNFGQTGDTESVPDPQPIGEPPAAPSGV |
| SEQ ID | GSLTMASGGGAPVADNNEGADGVGSSSGNWHCDSQWLGDRVITTSTRTW |
| NO: 55847 | ALPTYNNHLYKQISNSTSGGSSNDNAYFGYSTPWGYFDFNRFHCHFSPRD |
| | WQRLINNNWGFRPKRLNFKLFNIQVKEVTDNNGVKTIANNLTSTVQVFTD |
| | SDYQLPYVLGSAHEGCLPPFPADVFMIPQYGYLTLNDGSQAVGRSSFYCLE |
| | YFPSQMLRTGNNFQFSYEFENVPFHSSYAHSQSLDRLMNPLIDQYLYYLSK |
| | TINGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSPTQGAVTLRAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKENSKR |
| | WNPEIQYTSNYYKSNNVEFAVNTEGVYSEPRPIGTRYLTRNL |
| AAV9 N- | SYGQVATNHQS |
| terminal | |
| flanking | |
| region | |
| SEQ ID | |
| NO: 55848 | |
| AAV9 N- | B₁YGB₂VATNB₃QS |
| terminal | |
| flanking | |
| region- | |
| consensus | |
| SEQ ID | |
| NO: 55849 | |
| AAV9 C- | AQAQTGWVQNQGI |
| terminal | |
| flanking | |
| region | |
| SEQ ID | |
| NO: 55850 | |
| AAV9 C- | AQAQTGZ₁VZ₂Z₃QGZ4 |
| terminal | |
| flanking | |
| region- | |
| consensus | |
| SEQ ID | |
| NO: 55851 | |
| SEQ ID | B₁YGB₂VATNB₃QSPLMGAVHLYAQAQTGZ₁VZ₂Z₃QGZ₄ |
| NO: 55852 | |
| SEQ ID | GXLIFGKQGXGXXNVDXDXVMITNEEEIKTTNPVATEXYGXVATNXQSAX |
| NO: 55853 | XXXXTGXVXXQGXLPGMVWQDRDVYLQGPIWAKIPHTDGXFHPSPLMGG |
| | FG |
| SEQ ID | PTQGTVR |
| NO: 55854 | |
| SEQ ID | PSQGTLR |
| NO: 55855 | |
| SEQ ID | PTQGTLR |
| NO: 55856 | |

### 9. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

APPENDIX A

| SEQ | ICM | logFC |
|---|---|---|
| ID NO | peptide sequence | mean |
| 518 | TDFRSPQ | 1.83 |
| 396 | MPGRAPI | 1.23 |
| 393 | LTVSALK | 1.07 |
| 186 | AQTNRTIQTAQ | 1.07 |
| 368 | LGRLTAN | 1.01 |
| 493 | SGSLAVPFKAQ | 0.99 |
| 179 | AQSVSKPFLAQ | 0.79 |
| 217 | CTDIKGKC | 0.7 |
| 614 | YGSRSVD | 0.59 |
| 475 | RQPTTIP | 0.58 |
| 523 | TGAFSST | 0.56 |
| 411 | NIPKAYG | 0.52 |
| 443 | PYASITG | 0.51 |
| 192 | ATTLATPFKAQ | 0.51 |
| 403 | MTLTRQE | 0.45 |
| 266 | FGQKASS | 0.43 |
| 416 | NQTLAVPFKAQ | 0.4 |
| 579 | VQSGRRGDMSMLNS | 0.35 |
| 619 | YTIWMPENPRPGTP | 0.35 |
| 589 | VSNPLNQ | 0.35 |
| 247 | EGTLAVPFKAQ | 0.31 |
| 367 | LGNGKMTVQP | 0.3 |
| 263 | EVRGGPS | 0.26 |
| 410 | NEARVRE | 0.23 |
| 615 | YLPDQQLTWFPS | 0.17 |
| 597 | VTKSEWQQGFV | 0.13 |
| 184 | AQTLSTPFKAQ | −0.05 |
| 268 | FNSPVIQ | −0.05 |
| 586 | VRSEHRGDLSALNT | −0.05 |
| 259 | ESTLAVPFKAQ | −0.07 |
| 322 | HSPGKO | −0.08 |
| 273 | FTVSALK | −0.11 |
| 333 | ISDTRIS | −0.14 |
| 181 | AQTLATPFKAQ | −0.19 |
| 616 | YPGRNPD | −0.2 |
| 539 | TQSERRGDLAALNT | −0.2 |
| 565 | VGARLSA | −0.24 |
| 471 | RPLTAND | −0.27 |
| 257 | ERVGFAQ | −0.28 |
| 445 | QAVRTSL | −0.33 |
| 587 | VSALK | −0.38 |
| 185 | AQTLTMPFKAQ | −0.41 |
| 293 | GLNLPQNKVSFS | −0.41 |
| 354 | KVDISSQISSMNQS | −0.43 |
| 255 | EQFRNLA | −0.45 |
| 504 | SRAGTVP | −0.45 |
| 335 | ISTHSPP | −0.45 |
| 190 | ATDIKGKEV | −0.48 |
| 341 | KDSTAFG | −0.53 |
| 173 | AKEGANVAG | −0.56 |
| 568 | VISDRSS | −0.59 |
| 236 | DVGIRPS | −0.61 |
| 206 | CLSSRLDAC | −0.61 |
| 212 | CPQVKPGIKLEC | −0.66 |
| 557 | TVSALK | −0.68 |
| 492 | SGNLAVPFKAQ | −0.71 |
| 440 | PVGQPSHAPSVHLA | −0.73 |
| 379 | LPVGQPSHAPSVHL | −0.76 |
| 572 | VPALR | −0.77 |
| 172 | AHSLKSITNHGL | −0.78 |
| 425 | PAIKTYS | −0.78 |
| 512 | SYGTEWSSYKT | −0.81 |
| 464 | RGDLGLS | −0.81 |

APPENDIX B

| targeting | SEQ | CNS | DRG | Liver |
|---|---|---|---|---|
| peptide | ID NO | median | median | median |
| GINGAVKVK | 41077 | 16.12 | 2.48 | −1.75 |
| PAQGGLKLK | 46272 | 15.79 | #N/A | 4.18 |
| GLQGVLHVF | 3563 | 15.28 | 3.02 | 3.15 |
| GAQGVLRVR | 46143 | 14.97 | #N/A | −0.16 |
| PTNGSVHVH | 12416 | 14.31 | 6.46 | 3.53 |
| GTNGAIRLH | 37190 | 14.18 | 1.30 | −0.25 |
| PSPGTARLY | 19617 | 14.18 | 3.79 | 3.42 |
| GSQGSIRLR | 46035 | 14.16 | #N/A | 0.41 |
| GINGAVRVK | 46161 | 14.09 | 0.66 | 0.42 |
| GIPATLRLR | 6566 | 14.02 | #N/A | 1.27 |
| PVNAGLRVF | 36248 | 13.93 | 4.47 | 3.27 |
| PTQAVGKLR | 13308 | 13.60 | #N/A | −1.43 |
| GVPASGKVH | 9054 | 13.55 | 3.51 | 4.82 |
| PSPGTLKLH | 13781 | 13.43 | 3.58 | −0.18 |
| PSPGSIRVK | 46268 | 13.37 | #N/A | 1.86 |
| PVQGVVRVR | 46004 | 13.27 | #N/A | −0.23 |
| PLPGTLRLK | 41473 | 13.24 | #N/A | −0.05 |
| PVPGVIHVY | 2457 | 13.23 | 7.26 | 4.16 |
| GVQGVGKLH | 8980 | 13.18 | 4.86 | 3.86 |
| PLQAVLHVF | 41448 | 13.17 | #N/A | −1.11 |
| GAPATGRVR | 8848 | 12.89 | #N/A | −0.35 |
| GAPASLHVY | 38330 | 12.87 | #N/A | 3.14 |
| GVQAGVHLK | 31460 | 12.80 | 3.18 | −0.51 |
| GLQATAHLE | 8417 | 12.77 | #N/A | 0.73 |
| PSNAAVKVK | 35475 | 12.76 | 3.01 | 0.69 |
| PLNAGLKLF | 9763 | 12.69 | 1.61 | −1.67 |
| GSQGVVKLK | 43744 | 12.68 | #N/A | 2.55 |
| PSPGTLRLK | 41419 | 12.64 | #N/A | #N/A |
| GAPGAARLR | 46237 | 12.60 | #N/A | #N/A |
| GAPGAVHLF | 30029 | 12.60 | 4.50 | 1.81 |
| GINAVLRVY | 46293 | 12.47 | 0.59 | 2.15 |
| PSQGGVHVY | 35644 | 12.46 | 1.43 | −0.68 |
| PTNGVLRLY | 38876 | 12.44 | 1.17 | −0.70 |
| GTPGGGRVF | 27391 | 12.39 | 5.38 | 4.71 |
| PIQAGVHLY | 24062 | 12.33 | 4.86 | 1.59 |
| PSPGAVKVK | 46084 | 12.31 | #N/A | 1.68 |
| PIQGSARVF | 40066 | 12.30 | 5.08 | 3.41 |
| GSPGAIRLH | 37823 | 12.29 | 0.49 | 0.63 |
| GSQATLRVF | 28464 | 12.28 | 3.37 | −1.17 |
| GLQGALRIF | 3524 | 12.24 | #N/A | 1.04 |
| PLPGGLRLY | 46124 | 12.23 | #N/A | #N/A |
| PAPGVLRLY | 41427 | 12.18 | #N/A | 2.72 |
| PSQATVRLY | 46083 | 12.18 | #N/A | 2.07 |
| PSPGAGKVH | 19589 | 12.13 | 8.57 | 2.25 |
| GVQGVLRŁY | 41516 | 12.05 | #N/A | 1.60 |
| PTQAGVRVH | 7090 | 12.05 | 3.68 | 0.10 |
| PAQGAVRVK | 39802 | 11.98 | 2.66 | 3.70 |
| GTQGAGRVK | 26706 | 11.97 | 2.82 | 2.82 |
| PAQAGVKVK | 21019 | 11.94 | 3.93 | 2.89 |
| PSPGSLKLF | 46181 | 11.92 | #N/A | #N/A |
| GAPGVVKLY | 30183 | 11.88 | 2.49 | 0.25 |
| GVPGAGKVR | 45916 | 11.82 | #N/A | 2.88 |
| GINASGHVR | 46248 | 11.80 | 4.30 | 0.93 |
| GLPGAARLR | 46218 | 11.78 | #N/A | −0.53 |
| PSPGTLHLR | 46089 | 11.78 | #N/A | 1.41 |
| GLNAGIKLH | 8390 | 11.75 | 3.22 | 3.64 |
| PTQGVGRVK | 45692 | 11.73 | #N/A | 1.12 |
| PSPGAGHVF | 749 | 11.71 | 3.20 | 0.42 |
| PSPATGRVR | 7686 | 11.70 | #N/A | 1.80 |
| GTNAGGKVF | 26636 | 11.68 | 4.59 | 4.05 |
| GIQGSGHLK | 45223 | 11.67 | 0.95 | 1.09 |
| GVPATIKVF | 9042 | 11.66 | 1.47 | −0.01 |
| PTNGSLRVH | 31643 | 11.64 | 3.51 | 0.94 |
| PSPAAIRLF | 7669 | 11.61 | 5.56 | 2.35 |
| GINGVVKLY | 38890 | 11.54 | 4.80 | 3.80 |
| GANGVIKLK | 44051 | 11.53 | #N/A | 7.86 |
| PSPGTVKLK | 46087 | 11.53 | #N/A | 0.08 |
| PSPAVVKVR | 35840 | 11.50 | 3.49 | 2.13 |
| PVNGAVRVK | 46098 | 11.49 | 2.77 | 0.28 |
| PANGAVKLK | 35863 | 11.48 | 4.59 | 2.56 |
| GSPAGVHVY | 29139 | 11.46 | 2.70 | −2.78 |
| PSPAVVRVR | 45987 | 11.43 | #N/A | −1.70 |
| PSPGALHVR | 19546 | 11.41 | 0.85 | −3.41 |
| PANGTIKVR | 13978 | 11.39 | 1.49 | −0.97 |
| PVQGAIKVR | 36268 | 11.35 | 1.08 | 1.43 |
| GLQGVAKLY | 25326 | 11.33 | 2.31 | −0.63 |
| GTNATIKVR | 46022 | 11.32 | #N/A | #N/A |
| GLQAAIHVY | 25427 | 11.32 | 3.35 | 1.84 |
| PIPAGIRVR | 3352 | 11.30 | 1.65 | −3.28 |
| PINATIKVR | 42509 | 11.30 | #N/A | 4.93 |
| GTQGTIRVH | 37317 | 11.29 | 5.46 | 2.09 |
| GLNAALKLR | 24910 | 11.29 | 5.67 | 1.84 |
| PAPGTLKVY | 1512 | 11.29 | 5.53 | 3.86 |
| PLQGALKLF | 1310 | 11.26 | 2.27 | 1.16 |
| PLPGSLHLY | 8564 | 11.26 | 2.53 | 2.51 |
| GUNGVARLY | 45890 | 11.25 | #N/A | #N/A |
| PANGTLRLK | 39765 | 11.23 | #N/A | 0.28 |
| GSNGAVRVK | 27692 | 11.22 | 1.80 | 0.48 |
| PANAVIKVF | 35959 | 11.22 | 2.46 | −0.73 |
| GSPGTLKLH | 16894 | 11.19 | 1.98 | 1.15 |
| PLPGVLRVK | 27289 | 11.19 | 2.98 | #N/A |
| GINGVIHLH | 32185 | 11.18 | 0.47 | −1.59 |
| PLQGGLKLH | 22779 | 11.18 | 1.28 | −0.12 |
| PAPGAARLR | 36082 | 11.15 | #N/A | #N/A |
| PLNGGARVR | 18583 | 11.14 | #N/A | −0.18 |
| GVPAGARVK | 6014 | 11.11 | 9.00 | 3.41 |
| GVPGGARVK | 9030 | 11.10 | #N/A | #N/A |
| PSQGVGHVF | 46082 | 11.09 | 2.24 | 1.20 |
| PSQASIKVY | 46180 | 11.08 | #N/A | 1.48 |
| PTNGGIRLR | 18174 | 11.07 | 4.44 | 5.23 |
| GVNGSARLH | 8881 | 11.07 | #N/A | 2.07 |
| GTQASAHVY | 26965 | 11.06 | 5.70 | 2.26 |
| GAPATIRVF | 30384 | 11.06 | #N/A | 6.73 |
| PANGAVRVR | 39758 | 11.05 | #N/A | −0.08 |
| GTNASVRLK | 11390 | 11.04 | 4.36 | 1.06 |
| GTQGGIKVR | 4002 | 11.03 | 5.17 | 2.50 |
| GVNAVVHLK | 38489 | 11.03 | #N/A | 1.16 |
| PLNGGAHVK | 9242 | 11.02 | 5.38 | 3.02 |
| PANAVGRVK | 46185 | 11.01 | #N/A | 0.01 |
| PLNGSIRVE | 4074 | 10.99 | 4.32 | 1.19 |
| PSPAAVHLY | 46269 | 10.98 | 2.33 | −0.14 |
| GTPGAGHLK | 4083 | 10.97 | 5.45 | 3.13 |
| GINGAVHVH | 38834 | 10.96 | 5.77 | 2.27 |
| PSQAAIKVY | 35681 | 10.95 | 6.56 | 1.67 |
| GSQGVLHLH | 28308 | 10.93 | 3.97 | 0.54 |
| PTQGSIRLR | 18519 | 10.92 | #N/A | −1.84 |
| GVNAGLHVY | 30959 | 10.92 | 3.46 | −2.95 |
| GLNGTLKLK | 46111 | 10.91 | 1.21 | 0.32 |
| PAQAGIKLH | 7859 | 10.91 | 1.96 | 0.31 |
| GSNASVHVR | 37632 | 10.90 | 2.07 | 1.75 |
| PVPATLHLR | 22935 | 10.90 | 1.72 | −1.37 |
| GSNATLHLR | 4415 | 10.87 | 3.55 | 2.03 |
| GANGAAHVE | 37941 | 10.84 | 5.85 | 3.42 |
| GVNASGHLK | 30887 | 10.73 | 0.77 | −0.57 |
| PSPATARLY | 19893 | 10.72 | 1.13 | 1.89 |
| GAPGTARLR | 46147 | 10.71 | #N/A | 2.08 |
| PANASARLR | 46094 | 10.67 | #N/A | 1.31 |
| PTQAVARLY | 45928 | 10.63 | #N/A | 1.09 |
| GAQATARLF | 5195 | 10.63 | 3.29 | 1.42 |
| PTQAVAHLR | 13274 | 10.62 | 2.83 | −2.74 |
| PINGSGKLH | 10690 | 10.62 | 6.44 | 2.90 |
| PSPGAGHLY | 746 | 10.62 | 4.49 | 0.12 |
| PSPAGIHVY | 7695 | 10.62 | 2.30 | −0.64 |
| PLNGVGRVK | 46255 | 10.61 | #N/A | 0.99 |
| PLPAGLKVR | 46046 | 10.60 | #N/A | #N/A |
| GINAVVHLK | 32450 | 10.58 | 4.14 | 2.50 |
| PLPATLRVF | 41504 | 10.57 | 3.41 | 7.66 |
| GSPGTARLK | 46129 | 10.57 | #N/A | 1.34 |
| GIPGAARVR | 33147 | 10.56 | #N/A | 0.95 |
| PSPGALHLR | 46085 | 10.55 | 1.35 | 1.38 |
| PAQGTLHLY | 35978 | 10.55 | 1.83 | −1.49 |
| PIQGVVKLK | 46207 | 10.55 | #N/A | 1.00 |
| GTQATVKVR | 11496 | 10.54 | #N/A | 1.37 |
| PIPATLRLR | 3315 | 10.51 | #N/A | −1.07 |
| PAQGSIRLK | 41889 | 10.50 | #N/A | 6.73 |
| PLNGSLHVY | 26771 | 10.49 | 2.97 | −0.02 |
| GSNGVVRLY | 46030 | 10.48 | −1.10 | −0.24 |
| PLNGVLHLF | 31625 | 10.47 | 2.04 | −3.53 |
| GSQGVLRVK | 46126 | 10.46 | #N/A | 1.52 |
| GINAGGHLY | 6232 | 10.45 | 3.72 | 1.90 |
| PIQAVVRVR | 4601 | 10.43 | #N/A | #N/A |
| GIPGTVRLK | 41526 | 10.41 | #N/A | 2.16 |
| PVQGVGRLK | 46196 | 10.40 | 3.43 | 0.36 |
| PINGALKVH | 2624 | 10.39 | 4.20 | 1.85 |
| GAPGTAKLF | 30105 | 10.39 | 2.87 | 1.76 |
| PSPAVVRLY | 13912 | 10.38 | #N/A | −1.61 |
| PSPAAVRVY | 35809 | 10.38 | 5.57 | 2.02 |
| PLNGAVKLK | 8179 | 10.38 | 1.90 | 0.29 |
| PINAVARVF | 2871 | 10.38 | 4.58 | 2.10 |
| GAPATIKVR | 46048 | 10.36 | #N/A | 0.95 |
| PAPAVVKVY | 10174 | 10.36 | 5.54 | −0.12 |
| GIPGTLRLK | 6458 | 10.35 | 9.51 | 8.25 |
| PLNAAVHVF | 7119 | 10.35 | 2.42 | 2.21 |
| GAPGSLRVY | 30146 | 10.35 | 4.01 | 1.17 |
| PINAGLHVK | 23518 | 10.34 | 3.13 | −1.78 |
| GANGVLRVK | 46138 | 10.33 | #N/A | −1.86 |
| GVQAGGHVH | 46152 | 10.33 | 3.82 | 1.61 |
| GAQGTLRLR | 5074 | 10.33 | #N/A | #N/A |
| PVNGTVRVK | 42099 | 10.33 | #N/A | 2.85 |
| PLQGGLKLF | 22789 | 10.32 | 2.54 | 0.13 |
| GANGAVHVR | 43987 | 10.30 | 2.51 | 3.54 |
| GAPAGIRLF | 8855 | 10.30 | 4.42 | 2.85 |
| PSPGAGHVY | 19581 | 10.29 | 5.81 | 1.97 |
| GVNGTLHLF | 38384 | 10.25 | #N/A | −0.77 |
| PANAGLKLH | 1192 | 10.24 | 3.79 | 1.27 |
| GTNAVLHVF | 41477 | 10.24 | #N/A | −1.14 |
| GAPAGGKLY | 30577 | 10.23 | 4.56 | 4.39 |
| GTQATVHLF | 46225 | 10.23 | 1.71 | −0.31 |
| PTPATLKVY | 13488 | 10.22 | #N/A | 6.32 |
| GSPATARLK | 40701 | 10.21 | #N/A | 1.93 |
| PANGTAKVR | 45994 | 10.21 | #N/A | 0.38 |
| PAPATVKLK | 10132 | 10.21 | #N/A | 2.58 |
| PTQGVLRLY | 34452 | 10.20 | #N/A | 0.88 |
| GIPASIKVH | 6608 | 10.20 | 5.98 | 8.96 |
| PAQGVGRLR | 36012 | 10.18 | #N/A | 2.71 |
| GINGAVKLK | 38835 | 10.18 | 6.14 | −1.06 |
| PTQGAARLR | 45660 | 10.18 | 2.89 | 2.54 |
| GSQASARVK | 16836 | 10.17 | #N/A | −0.41 |
| PSQAGARVE | 19501 | 10.17 | 5.93 | 3.80 |
| GSNAVAHVY | 4478 | 10.17 | 0.09 | 4.07 |
| PTNGAVKLK | 8874 | 10.17 | 6.07 | 1.97 |
| GINGALKLH | 31991 | 10.17 | 2.91 | 1.47 |
| GSPGTAKVK | 11892 | 10.16 | 3.27 | −1.17 |
| PAPATVKVK | 10134 | 10.15 | −1.46 | −0.24 |
| GIPGAGRLR | 46251 | 10.15 | #N/A | 0.84 |
| PSPATIRVF | 19906 | 10.15 | 5.27 | 1.21 |
| PTNAVVRVR | 46073 | 10.15 | #N/A | #N/A |
| GVQATVKLF | 5778 | 10.13 | 6.78 | 4.80 |
| GSQAGVRLY | 28568 | 10.12 | 5.11 | 6.51 |
| PIQAGIRLY | 46012 | 10.12 | #N/A | 4.25 |
| GVQGTVHVF | 31051 | 10.12 | 5.17 | 2.91 |
| PVQAGVRLY | 2362 | 10.09 | 5.62 | 3.78 |
| GTPASIRVK | 46283 | 10.09 | #N/A | 1.42 |
| GANAGLHVH | 5030 | 10.09 | #N/A | 0.85 |
| GANAGGHVR | 5045 | 10.08 | 2.52 | 1.07 |
| GANGGIHLF | 29341 | 10.07 | 1.99 | −1.16 |
| GVNGVVHLF | 30675 | 10.06 | 1.15 | −3.85 |
| GTNGGIKVR | 40382 | 10.06 | #N/A | 3.06 |
| GTNGTARVR | 43117 | 10.05 | 2.15 | 2.22 |
| GSPAAVHVH | 4788 | 10.05 | 4.26 | 8.95 |
| GANGGLHLY | 46139 | 10.05 | #N/A | −2.16 |
| GAQGAVKVY | 29573 | 10.04 | 3.18 | 0.72 |
| PTNGVAKVK | 32355 | 10.03 | #N/A | #N/A |
| GLPAVGRVF | 26089 | 10.02 | 3.19 | 3.84 |
| PVPGSIRVK | 46275 | 10.01 | #N/A | 1.64 |
| GSNGSGKVY | 27847 | 10.00 | 3.70 | 3.37 |
| PSNAGIRLE | 7637 | 10.00 | 2.20 | 0.07 |
| PTQGSGHLK | 34434 | 9.99 | 3.75 | 1.15 |
| PTNATIKVR | 41298 | 9.99 | 4.41 | −0.08 |
| PIPGVLKLY | 11022 | 9.97 | 2.39 | 1.94 |
| PLQGGIRVY | 22979 | 9.97 | 4.82 | 6.69 |
| PTQGSAKLH | 9225 | 9.97 | 4.47 | 1.77 |
| GVQGGAHVK | 8987 | 9.97 | 5.75 | 4.65 |
| PSQATGKVR | 41417 | 9.96 | #N/A | 0.92 |
| PTQGGLRLH | 34495 | 9.95 | 2.65 | 0.94 |
| PSNGGLHVR | 9419 | 9.94 | 2.75 | −1.58 |
| PSQATIHLK | 19389 | 9.93 | 6.76 | 3.52 |
| PANGGGKVR | 39781 | 9.93 | #N/A | 1.00 |
| PVPGAVKLK | 8090 | 9.89 | 3.43 | 1.04 |
| GAPAVIHLK | 17492 | 9.89 | 3.25 | 1.21 |
| PAPGVIRVF | 36104 | 9.89 | 2.86 | 1.20 |
| GTPAALHLK | 27407 | 9.89 | 2.27 | #N/A |
| PVNGGLHLK | 7984 | 9.88 | 5.09 | 1.45 |
| PTPATLRVR | 7338 | 9.88 | #N/A | 2.72 |
| PAQAGLHVH | 1447 | 9.87 | 2.62 | 2.48 |
| PINGSIHLR | 2718 | 9.86 | 3.43 | −0.82 |
| GSNAGGKVF | 16664 | 9.86 | 4.60 | 5.12 |
| GVNGSVKVR | 30646 | 9.86 | 3.56 | 1.46 |
| GSQAGIRLY | 11862 | 9.86 | −0.21 | −0.58 |
| GSNGGGHVF | 16553 | 9.85 | 1.00 | 1.36 |
| GINASVHLK | 18001 | 9.85 | 3.23 | 2.89 |
| PLNGATRVY | 41241 | 9.84 | 2.92 | 5.24 |
| PLPGTAHLF | 16045 | 9.84 | 4.35 | 0.36 |
| GAQAGIRLR | 30005 | 9.84 | #N/A | #N/A |
| GTNAALRIK | 43164 | 9.82 | #N/A | 1.50 |
| GVPATLRLR | 5965 | 9.82 | #N/A | −1.20 |
| GAPGGGHVF | 17439 | 9.80 | 3.33 | 0.71 |
| GSPAVVHLH | 8768 | 9.80 | #N/A | 1.62 |
| GIQGSAKLY | 41156 | 9.79 | 5.10 | 3.23 |
| PVPASIRLK | 23011 | 9.78 | #N/A | 0.85 |
| PLQGALKLY | 45874 | 9.78 | 4.65 | 2.89 |
| PLQGTLRLY | 21189 | 9.77 | 4.72 | 9.00 |
| GVQGTLHLK | 38538 | 9.77 | 3.19 | −1.22 |
| PAPASIRVY | 1700 | 9.76 | 4.32 | 1.78 |
| GIQGGLKVF | 18114 | 9.75 | 4.23 | 3.32 |
| PANAVAKVH | 20443 | 9.75 | 4.38 | 3.20 |
| GVQGAIHVY | 40975 | 9.74 | −0.14 | 1.21 |
| PINGTGRVH | 10674 | 9.73 | 4.12 | 2.27 |
| GLNAVIRVF | 46215 | 9.73 | #N/A | −2.86 |
| PINGAVRLY | 5380 | 9.72 | 3.97 | −0.56 |
| PSPAVVRLR | 45986 | 9.71 | #N/A | 1.22 |
| PSQGTLHLH | 39672 | 9.70 | #N/A | −1.27 |
| PVPGAVRVK | 46102 | 9.70 | #N/A | −0.12 |
| PVQATLRVY | 39947 | 9.70 | 3.59 | 2.01 |
| PVNAALRLF | 36215 | 9.69 | 1.16 | −0.11 |
| GLQATAHVF | 46115 | 9.69 | #N/A | 4.68 |
| PVNATGRVR | 8024 | 9.69 | 3.26 | −0.28 |
| GVPAVIRVF | 46245 | 9.68 | #N/A | −1.30 |
| PSQGGVRVF | 39701 | 9.68 | 4.78 | 4.61 |
| GTQGVLRVK | 43308 | 9.68 | #N/A | 2.27 |
| GSPATAKLK | 40702 | 9.67 | #N/A | 0.84 |
| PLNATIKVR | 45977 | 9.66 | #N/A | #N/A |
| GTNAVLKVY | 26520 | 9.65 | 1.16 | 0.02 |
| PLNGVVHLY | 5363 | 9.64 | 7.85 | 2.11 |
| PSQGTVHLF | 46174 | 9.64 | #N/A | #N/A |
| PSNGSLKVH | 46076 | 9.64 | 5.00 | 1.97 |
| PTNGVIHLH | 32369 | 9.64 | 0.92 | 0.18 |
| PLQGVLKVY | 22233 | 9.63 | 4.84 | 2.60 |
| GIPGAARLR | 6434 | 9.63 | #N/A | 2.49 |
| PVNGVIHVF | 21733 | 9.63 | 3.82 | 0.26 |
| PIQGGGHLR | 23825 | 9.62 | 5.53 | 3.74 |
| GIQASVHLF | 18184 | 9.62 | 4.61 | 2.18 |
| GANGAVRVK | 46136 | 9.61 | #N/A | 1.00 |
| PVNAALHLK | 39898 | 9.61 | 7.06 | 0.39 |
| PVQGSARVK | 46195 | 9.60 | #N/A | −1.25 |
| PINASGKLR | 46107 | 9.59 | #N/A | 0.86 |
| PSPASARVF | 19946 | 9.58 | 2.98 | 0.55 |
| GTNAAGHLK | 3832 | 9.58 | 7.47 | 4.05 |
| PLPAVVRVR | 44226 | 9.58 | #N/A | 1.34 |
| PANAVIHLY | 1157 | 9.57 | 3.78 | 1.63 |
| PSNAGVKVK | 19081 | 9.56 | 3.50 | 2.29 |
| GLQAAGRVY | 25448 | 9.56 | 2.17 | 2.86 |
| PLNASIRLK | 46264 | 9.55 | 0.18 | 0.79 |
| GLQGSARVH | 41458 | 9.55 | #N/A | 1.65 |
| GSQGVGRVK | 43759 | 9.55 | #N/A | 4.19 |
| PTQGTIKLR | 45927 | 9.55 | 1.54 | 1.14 |
| PSNGVLHLY | 45933 | 9.54 | #N/A | −0.21 |
| GANATVRLF | 44099 | 9.54 | #N/A | 0.97 |
| PLNASLRLF | 9543 | 9.54 | #N/A | −0.55 |
| PTPATLKLR | 7340 | 9.54 | #N/A | 2.55 |
| GVNAGGHLK | 5649 | 9.53 | 4.23 | 3.73 |
| GSNAAIRLH | 27997 | 9.53 | 2.68 | 2.22 |
| GUNGTARLK | 46112 | 9.52 | #N/A | 0.76 |
| PLNGAGRVF | 946 | 9.51 | 3.30 | 4.00 |
| GIPAGVKVR | 46166 | 9.51 | #N/A | #N/A |
| PTQGVVRLK | 41316 | 9.51 | #N/A | 0.56 |
| PLNASIRLE | 46265 | 9.51 | 0.26 | −1.56 |
| PTQGTVRLR | 34374 | 9.51 | 3.19 | 1.43 |
| GVQATIHVE | 38625 | 9.50 | 5.48 | 2.53 |
| PVPASIRVK | 42410 | 9.49 | #N/A | 1.91 |
| GTNGVAHLR | 8479 | 9.49 | 4.57 | 2.84 |
| PTNGAAKVK | 17646 | 9.49 | 3.34 | 1.34 |
| GIPAAIKVR | 6555 | 9.49 | 2.06 | 0.62 |
| GIPGTLRVK | 33208 | 9.49 | #N/A | −0.06 |
| PINGAVRVY | 23157 | 9.49 | 1.82 | 0.01 |
| PLNAVIHVF | 9663 | 9.48 | 6.00 | −0.41 |
| GTPAAIRVF | 37516 | 9.48 | 2.06 | 0.54 |
| GVPGVIHLF | 38754 | 9.48 | 2.42 | 0.67 |
| GVQGAGHVK | 8965 | 9.47 | 4.83 | 3.09 |
| GVNGVGKVY | 5487 | 9.47 | 1.75 | 1.12 |
| PAQATIHVF | 14208 | 9.47 | 3.78 | 3.59 |
| GIPGAAKLR | 33151 | 9.46 | 1.86 | 0.24 |
| PTQGALKLH | 34335 | 9.46 | 1.83 | −1.12 |
| GANGVLRVF | 40752 | 9.45 | 1.42 | 4.37 |
| PANASIRLR | 45872 | 9.45 | #N/A | −0.76 |
| GSQATLHVR | 40632 | 9.45 | 4.40 | 3.43 |
| GLNAVVRLH | 46214 | 9.45 | 2.31 | −2.24 |
| PANGTGKVR | 41424 | 9.44 | #N/A | 1.30 |
| GAQAGAHLF | 44410 | 9.44 | #N/A | 3.38 |
| PLNAALKVY | 34931 | 9.43 | 0.09 | −1.54 |
| PLPGTLRVF | 46223 | 9.43 | #N/A | #N/A |
| PAPGTLKLY | 1509 | 9.43 | 2.80 | 8.95 |
| PLNGTVRLK | 7846 | 9.43 | −0.47 | 2.33 |
| GLQAGAHLF | 8421 | 9.41 | #N/A | −0.08 |
| GINATLHLY | 32374 | 9.40 | 1.91 | −2.66 |
| GIPAAIHVK | 6551 | 9.40 | 1.17 | −1.17 |
| PIQATLHLH | 40079 | 9.40 | 1.59 | 0.04 |
| PVNGGVHLK | 1927 | 9.39 | 2.22 | 0.03 |
| GSPGVVHLH | 28791 | 9.39 | 4.29 | #N/A |
| PINGGIKLH | 15021 | 9.39 | 2.04 | 0.55 |
| GAPGAARLK | 46145 | 9.38 | #N/A | 3.26 |
| PLQASGHVF | 15461 | 9.37 | 3.87 | −1.67 |
| GSNGALRVY | 27712 | 9.37 | 4.36 | 0.83 |
| GVPAAVRLR | 5931 | 9.36 | #N/A | 1.80 |
| PINAVIRVF | 46205 | 9.35 | #N/A | −0.05 |
| PAPGSVRLY | 10078 | 9.34 | 3.42 | 0.98 |
| PVPGTGKVR | 42349 | 9.32 | #N/A | 1.85 |
| PTPATIRVY | 7353 | 9.32 | 7.16 | 2.54 |
| GSNAAVKVK | 27974 | 9.32 | 2.03 | 0.28 |
| GVNGSGHVK | 38413 | 9.31 | 2.06 | 5.75 |
| PLQGAARLF | 20775 | 9.31 | 4.98 | 2.84 |
| GTQASIRLK | 45959 | 9.31 | #N/A | 0.32 |
| PSQGGAHVH | 46262 | 9.30 | 3.40 | 3.33 |
| PLNGSGKVR | 41512 | 9.30 | #N/A | −0.14 |
| GANGVIRLH | 11997 | 9.29 | 1.34 | 1.47 |
| GTQAVVKVH | 27011 | 9.29 | 3.70 | 1.46 |
| PAQAGIKLF | 7860 | 9.29 | 3.75 | 1.72 |
| GSPGVLRLK | 28811 | 9.29 | 1.93 | −3.12 |
| GTQGAVHLF | 26640 | 9.29 | 2.98 | 1.42 |
| PLQGVGRVK | 42337 | 9.28 | #N/A | 3.25 |
| PTQGVVHVY | 6979 | 9.28 | 3.93 | 2.59 |
| PLNASIRVK | 45980 | 9.27 | #N/A | −2.00 |
| GSNAVIRLY | 46032 | 9.25 | #N/A | 2.36 |
| GVQAVIRLF | 38662 | 9.24 | #N/A | 3.80 |
| PLNATGKLK | 45979 | 9.24 | #N/A | 3.15 |
| PLPGGVKVF | 4304 | 9.23 | 2.25 | 3.19 |
| PLNGTVHLY | 1232 | 9.23 | 4.70 | 3.08 |
| GSPGGGHVH | 40692 | 9.22 | 5.12 | 3.12 |
| GVPAAGKLR | 38783 | 9.22 | 4.60 | 1.96 |
| PTNASARLR | 46172 | 9.22 | #N/A | #N/A |
| PVQGVVRVK | 46101 | 9.22 | #N/A | 1.14 |
| PTQGVGKLK | 13146 | 9.21 | #N/A | 1.44 |
| PLQGSGRLH | 10321 | 9.21 | 3.57 | 4.59 |
| PSQAAVHVY | 46263 | 9.20 | 4.38 | 2.28 |
| GSNGTIKVY | 16504 | 9.19 | 3.36 | 2.19 |
| PTQATVRVR | 13215 | 9.19 | #N/A | 3.94 |
| GSPGGIKVR | 4783 | 9.19 | 3.54 | 2.26 |
| GSNGSIHLE | 43556 | 9.19 | #N/A | 0.20 |
| GANGAVHLH | 37930 | 9.18 | 2.90 | −2.38 |
| PLPAGVRLY | 30064 | 9.18 | 4.37 | 1.18 |
| PANGVLRLY | 41425 | 9.18 | 2.20 | −0.28 |
| PINASLHLR | 46105 | 9.18 | 0.87 | −1.91 |
| GVNGAVRVK | 40908 | 9.17 | #N/A | −2.34 |
| PVNGSGKVH | 1872 | 9.17 | 1.49 | 0.22 |
| GTPGGGHLR | 27378 | 9.16 | 3.07 | 1.02 |
| PSNGSGHLR | 18883 | 9.15 | 3.80 | 3.11 |
| GAQGTLRVE | 46142 | 9.15 | 1.07 | 1.20 |
| GAPGAIKLK | 45966 | 9.14 | #N/A | #N/A |
| PANGTVRVK | 41734 | 9.14 | #N/A | 2.39 |
| GTPATAHVY | 16420 | 9.14 | 5.97 | 1.72 |
| GIQGALHLF | 38994 | 9.14 | 1.24 | −2.17 |
| GLQGSGKLY | 25298 | 9.14 | #N/A | 5.37 |
| PVPAALRVF | 2516 | 9.13 | 3.58 | 1.43 |
| GVPGAGKLF | 5832 | 9.13 | 4.94 | 3.88 |
| GTQGAVRLH | 26646 | 9.13 | 4.59 | 2.21 |
| GVNAVLRLY | 40958 | 9.12 | 1.78 | #N/A |
| GSQATGRLH | 4652 | 9.12 | 3.69 | 4.53 |
| GLNGVAHVK | 3434 | 9.11 | 1.57 | 3.39 |
| PANGVGRVK | 20249 | 9.11 | #N/A | −1.48 |
| PLPAAVHLR | 28186 | 9.11 | 3.59 | −0.11 |
| PLQATIKLH | 10900 | 9.10 | 2.26 | 1.54 |
| PVQAGLKLY | 2369 | 9.10 | 6.11 | 1.07 |
| GVQGAGHLH | 5676 | 9.10 | 2.28 | 1.68 |
| PSPAALRVF | 804 | 9.09 | #N/A | 0.70 |
| GTQGAVHVR | 26641 | 9.09 | 3.28 | 1.58 |
| PAPASIRLF | 1699 | 9.08 | 3.24 | 0.98 |
| GIQAVAKVF | 9120 | 9.08 | 3.99 | 0.70 |
| PLNATIRVK | 46259 | 9.08 | #N/A | −0.45 |
| PSPGALRVF | 738 | 9.07 | 5.12 | 0.74 |
| PAPAGAHVK | 1728 | 9.06 | 7.13 | 0.07 |
| GVQAVARVE | 38656 | 9.06 | #N/A | 3.73 |
| PSPGAAHLR | 19555 | 9.06 | 5.86 | 3.74 |
| PTPGAGKLY | 13390 | 9.05 | 2.85 | 2.76 |
| PIPAGIKVR | 3356 | 9.05 | 1.46 | −1.16 |
| PVQGVGKVK | 42258 | 9.05 | #N/A | 0.24 |
| GIPAVLKLH | 6622 | 9.05 | 0.79 | #N/A |
| GLNATLKLK | 46017 | 9.05 | #N/A | −0.65 |
| GSPGVVRVK | 28799 | 9.04 | 2.00 | −1.37 |
| PLNGVTRLY | 46068 | 9.04 | #N/A | −0.59 |
| PTQAGLHVH | 7091 | 9.04 | 2.29 | 0.53 |
| PTNGVGHLR | 32454 | 9.03 | 5.43 | 1.64 |
| PVNAGVRVH | 10324 | 9.03 | 5.77 | 5.29 |
| PSNASLHVH | 35538 | 9.03 | 3.97 | −0.83 |
| GVNGTARLK | 46151 | 9.03 | #N/A | 1.67 |
| GTPGAAKLY | 27122 | 9.02 | 2.89 | 0.88 |
| GINATIHLF | 38947 | 9.01 | 0.90 | −4.84 |
| PINAGARVR | 23526 | 9.01 | 3.59 | 0.74 |
| GANGGAKVH | 38039 | 9.00 | 5.88 | 3.67 |
| PLNATGRLR | 19049 | 9.00 | #N/A | 0.97 |
| PLPGGIKVY | 28088 | 9.00 | 3.69 | 0.45 |
| GANGTIHLF | 37960 | 8.99 | 4.60 | 2.83 |
| GTPAGGRLY | 27676 | 8.99 | 5.25 | 4.93 |
| PVNAGVHVH | 2097 | 8.98 | #N/A | 2.35 |
| GVNAAVHLY | 44633 | 8.98 | 2.13 | 0.02 |
| GSPGSLKVY | 11900 | 8.98 | 4.72 | 3.49 |
| PVNGVGHVK | 1917 | 8.98 | 4.76 | 8.37 |
| PVQASAKVK | 39957 | 8.97 | #N/A | −1.33 |
| PSNGGAKLY | 9428 | 8.97 | 6.48 | 2.95 |
| PLPGVVHLH | 4064 | 8.97 | 1.33 | −4.92 |
| PLPGGVRLY | 11661 | 8.97 | 1.82 | 0.81 |
| GAPGTAKLY | 30104 | 8.97 | 2.07 | 1.40 |
| PIQATIHLF | 10888 | 8.96 | 3.45 | 1.69 |
| GTNATLKLK | 45899 | 8.96 | 3.11 | 0.79 |
| GIQASVRLR | 12702 | 8.96 | #N/A | −0.48 |
| PSQGGVHVR | 19260 | 8.96 | 3.38 | 2.20 |
| PLNGGARVH | 13178 | 8.96 | 3.44 | 2.28 |
| GVQGALKVF | 5667 | 8.95 | 2.93 | 3.86 |
| PTNGGVKLY | 6285 | 8.95 | 5.73 | 3.38 |
| GAQGGAKVF | 5158 | 8.95 | 4.17 | 3.64 |
| GTPGAVKLK | 40440 | 8.95 | 2.71 | −0.16 |
| GIPATARVY | 6571 | 8.95 | 1.82 | 0.23 |
| PTQGVGRLR | 13142 | 8.95 | #N/A | −1.11 |
| GANATVHVK | 12015 | 8.94 | 1.78 | 2.10 |
| GTNGTLHLY | 3732 | 8.94 | 4.04 | 3.46 |
| PTPATLKVH | 7341 | 8.93 | #N/A | 5.38 |
| GTPGTARLK | 43429 | 8.93 | #N/A | 6.55 |
| GTNASIRVK | 43216 | 8.93 | #N/A | 3.47 |
| PVQASIRLK | 22443 | 8.93 | #N/A | 4.14 |
| PSQASGRVH | 19456 | 8.92 | 3.32 | 1.52 |
| GAPGAVRVF | 40841 | 8.92 | #N/A | 2.49 |
| PIQAGVRLY | 24069 | 8.92 | 3.02 | 3.74 |
| GTNAGAHLK | 26575 | 8.91 | 2.52 | 1.10 |
| PVQGTAHLH | 2157 | 8.91 | 5.10 | 1.31 |
| GANASIHVY | 4983 | 8.91 | 2.80 | 0.30 |
| PVNAAGRVR | 8007 | 8.91 | 2.09 | 1.69 |
| PTPGGVHLR | 9291 | 8.91 | 4.29 | 0.74 |
| GANAGAKLE | 5038 | 8.90 | 2.92 | 2.12 |
| PIPGSVRVY | 10994 | 8.90 | 1.87 | 1.88 |
| GVQAVIHVH | 31437 | 8.89 | 7.62 | 1.18 |
| PVNASIRVK | 42166 | 8.88 | #N/A | 2.66 |
| GSNGAVRVH | 8639 | 8.88 | 1.06 | −1.49 |
| GTPGALHLH | 404 | 8.88 | 2.33 | 2.54 |
| PVNAAVRVK | 21797 | 8.88 | #N/A | 2.60 |
| PLNASLKLF | 9549 | 8.88 | 5.58 | −2.03 |
| PAPATIKLF | 42049 | 8.88 | 0.68 | 1.47 |
| PLNGSVHLY | 3603 | 8.87 | 3.89 | −0.60 |
| PINGSARVK | 46204 | 8.87 | #N/A | 0.23 |
| PSPAGIRLF | 851 | 8.86 | 2.63 | 8.76 |
| GANAALKLK | 38068 | 8.86 | 1.84 | −0.01 |
| PINGSLRLF | 46009 | 8.85 | #N/A | 2.39 |
| GAQAGARVY | 29997 | 8.85 | 3.00 | 2.75 |
| GTPGAVKVK | 40441 | 8.85 | −0.50 | 0.87 |
| PAPGVARLR | 46190 | 8.84 | 4.71 | 2.39 |
| GAPGTARLY | 30099 | 8.83 | 0.74 | −0.77 |
| GINAGVHLR | 32515 | 8.83 | #N/A | 1.38 |
| PLNGVIKLK | 41303 | 8.83 | #N/A | −1.66 |
| PTQATIKVR | 45716 | 8.83 | #N/A | 2.43 |
| PTPGGLHLH | 13456 | 8.82 | 5.64 | −0.98 |
| PSPGAGHLR | 19580 | 8.82 | 5.59 | −0.62 |
| GVQGVLRVY | 41517 | 8.81 | #N/A | 4.11 |
| GSQATLHLF | 37750 | 8.81 | 1.79 | 0.21 |
| PLNGSLKLF | 11593 | 8.80 | 3.09 | −0.44 |
| PIPAGVHVY | 3342 | 8.80 | 5.13 | #N/A |
| GLNAALHLK | 24907 | 8.80 | 4.84 | #N/A |
| GANATIHLF | 46038 | 8.80 | #N/A | 0.10 |
| GLNAAGKVR | 41453 | 8.79 | #N/A | 2.26 |
| PANGGAHVH | 999 | 8.79 | 4.07 | 2.54 |
| GVQGGARLH | 8988 | 8.79 | #N/A | 0.44 |
| GLNGAVHVR | 42758 | 8.79 | #N/A | 3.87 |
| GVPAVAKLK | 46160 | 8.79 | #N/A | 1.76 |
| GVPATVRLY | 5963 | 8.79 | 1.44 | 0.47 |
| GVQGTAHLY | 5697 | 8.78 | 4.66 | 2.34 |
| GTPATLRLK | 41489 | 8.78 | 1.44 | −0.86 |
| GAPGAVKVR | 45913 | 8.78 | #N/A | −1.22 |
| PSNGVLRVH | 46258 | 8.78 | 2.22 | 1.03 |
| GANGAIRLK | 4865 | 8.78 | #N/A | 3.96 |
| PIQGVGKVR | 2989 | 8.78 | #N/A | 0.64 |
| PSPGAAHVY | 19558 | 8.78 | 6.02 | 3.08 |
| GIPAGGHVH | 12873 | 8.77 | 1.69 | 2.90 |
| GINAVVHLH | 32449 | 8.77 | 4.09 | 0.70 |
| PLQATLHLH | 15189 | 8.77 | 2.26 | −2.25 |
| GVPGTGKVK | 41045 | 8.76 | #N/A | 1.47 |
| GANGTIKVR | 46037 | 8.76 | 2.57 | #N/A |
| GTPGVGHLH | 11557 | 8.76 | 2.92 | 2.54 |
| PAQGGVHLH | 1316 | 8.76 | 6.55 | 2.59 |
| PVNAGLKVK | 10328 | 8.76 | #N/A | −0.68 |
| GSNAVLHLY | 46031 | 8.75 | #N/A | −0.14 |
| PLQGALHLK | 20632 | 8.75 | 2.68 | −0.92 |
| GSQGVIKVH | 4591 | 8.75 | 2.20 | 6.86 |
| PLPAVLKLH | 5134 | 8.75 | 2.39 | −0.34 |
| PSPAVAHVR | 13915 | 8.75 | 2.21 | 6.14 |
| PVQAGVHLH | 46005 | 8.75 | #N/A | #N/A |
| PTQAGAHVY | 13336 | 8.75 | 3.15 | 1.83 |
| PTNAVIRLH | 34224 | 8.74 | #N/A | 1.02 |
| PAQGVGKLK | 41908 | 8.74 | #N/A | 2.14 |
| PAQGSARVK | 46187 | 8.74 | #N/A | 2.73 |
| GLNGTVHVK | 3382 | 8.74 | 1.46 | −0.16 |
| PTNGVIHVY | 6164 | 8.74 | 3.88 | 3.49 |
| GTQASIKVR | 46027 | 8.74 | #N/A | 0.41 |
| GANGTVKVK | 40727 | 8.73 | 2.68 | 2.41 |
| GAQGALHVK | 29580 | 8.73 | 5.68 | 2.73 |
| GLQGTGRVH | 15687 | 8.73 | 2.50 | 1.71 |
| PTQAGGRVY | 13367 | 8.73 | #N/A | 5.48 |
| GVNAVLRVF | 30910 | 8.72 | 6.41 | 2.34 |
| GIPGSLHLY | 33269 | 8.72 | 0.19 | −2.23 |
| GANGVVRLF | 29301 | 8.72 | 1.38 | 0.22 |
| GLNAAVKLK | 36902 | 8.72 | #N/A | 0.06 |
| PLQGSAHVK | 10261 | 8.72 | 3.94 | 0.72 |
| GIQASARVK | 46250 | 8.71 | #N/A | −0.27 |
| GSNATGKVR | 41499 | 8.71 | #N/A | 0.67 |
| GVQAGARVY | 38675 | 8.71 | 1.17 | 2.14 |
| GTPAAVHLK | 11574 | 8.71 | 3.86 | 1.42 |
| GTNAVGKLF | 26548 | 8.71 | 4.28 | 4.33 |
| PLQGVGRLF | 22520 | 8.70 | 4.80 | 3.28 |
| PAPGTGKVR | 21160 | 8.69 | #N/A | 0.15 |
| PLQGASKVH | 12903 | 8.69 | #N/A | 11.52 |
| PSQATVKVY | 19364 | 8.69 | 3.50 | 3.27 |
| PSQATAHLY | 19374 | 8.68 | 4.34 | 1.45 |
| PTPATVRLR | 7329 | 8.68 | #N/A | 1.54 |
| PVQGVGRVK | 2226 | 8.68 | 2.06 | 1.33 |
| PTNATIKVK | 41299 | 8.68 | #N/A | 1.10 |
| GIQATVRLY | 32907 | 8.67 | 1.81 | 1.34 |
| GAQGVIRLF | 5143 | 8.66 | 14.24 | 1.58 |
| GLPGVVHVF | 41470 | 8.66 | 0.49 | 0.59 |
| PTQGVIKER | 45690 | 8.66 | #N/A | 3.03 |
| GVQGGVHLY | 31196 | 8.65 | 0.55 | −0.74 |
| PSNGGIHVR | 9435 | 8.65 | 3.01 | 3.77 |
| PVNGAVKLK | 36170 | 8.64 | 4.79 | 3.71 |
| GANAGAHLY | 29536 | 8.64 | 5.15 | 3.38 |
| PVQAGVKLR | 36391 | 8.64 | #N/A | 1.85 |
| GSPAGVRLY | 29142 | 8.64 | 6.32 | 3.40 |
| PVNAAVKVK | 21801 | 8.63 | 3.14 | 0.29 |
| GANGSIRLF | 4900 | 8.62 | 1.81 | 0.38 |
| GIQGSGKLK | 46163 | 8.62 | #N/A | 0.74 |
| GTQAVAHLH | 16309 | 8.62 | 4.38 | 0.25 |
| PSPGGLKVF | 13860 | 8.62 | 2.58 | −0.59 |
| GSPATVRLY | 29000 | 8.61 | 1.88 | 0.78 |
| GIPGGARLR | 33438 | 8.61 | 2.64 | −1.87 |
| PSPAAGRVR | 7677 | 8.61 | #N/A | 1.53 |
| GANGVVHVK | 37989 | 8.60 | 3.44 | 1.65 |
| PSQATVHLF | 46179 | 8.60 | 0.81 | #N/A |
| GTPGGLHVF | 37492 | 8.59 | 3.90 | 1.02 |
| GANGSVRLF | 37963 | 8.59 | 2.59 | 1.74 |
| PVQASGHLY | 2331 | 8.59 | 0.23 | 4.97 |
| GSQGVAKVR | 4582 | 8.59 | #N/A | 0.27 |
| PLPAALHLY | 37682 | 8.59 | #N/A | 1.14 |
| PLNGSAHLY | 4253 | 8.58 | 1.99 | −2.81 |
| PSPAVIHLR | 45991 | 8.58 | 4.02 | −0.05 |
| PSPGSLHVR | 35782 | 8.57 | 1.80 | 0.33 |
| GTNGTARVK | 46117 | 8.57 | #N/A | 1.48 |
| GTPGVARLF | 27304 | 8.57 | 1.52 | 1.94 |
| PSNGGIHVH | 7554 | 8.56 | 2.30 | −0.07 |
| PIQGGIRLK | 41438 | 8.56 | 1.72 | −0.67 |
| GTPAVVRVR | 46029 | 8.56 | #N/A | 0.37 |
| GLNATLHLR | 3470 | 8.56 | 1.27 | −0.40 |
| PLPGAIRVH | 25998 | 8.55 | 2.40 | −1.42 |
| PANGGIKLK | 45938 | 8.55 | 1.81 | −0.02 |
| PLNAVVKLF | 9616 | 8.55 | 1.43 | 7.11 |
| PTQAAVHVY | 7020 | 8.54 | 5.97 | −0.11 |
| PVNASIRVH | 36237 | 8.54 | 0.45 | 0.21 |
| PSPGVGHVH | 13855 | 8.54 | 2.40 | −0.04 |
| GLNAVIKVF | 11180 | 8.54 | 1.56 | 0.90 |
| GAQGVGHVH | 5150 | 8.53 | 0.77 | −1.08 |
| PAPGTVKVK | 36088 | 8.53 | 2.37 | 0.54 |
| GAPATGRLF | 30394 | 8.53 | 3.93 | 3.20 |
| GAQGTARLF | 5082 | 8.53 | 2.61 | 1.72 |
| GTPAVVRVF | 27595 | 8.52 | 1.05 | 0.83 |
| PLQGVVKLK | 42223 | 8.51 | #N/A | 5.90 |
| GLNGALKLH | 15495 | 8.51 | 4.51 | 1.96 |
| PAQATLHLY | 9975 | 8.51 | 1.15 | −4.51 |
| PSPAVVHVY | 19983 | 8.51 | #N/A | −0.03 |
| GIQAVVHLR | 9112 | 8.51 | 2.40 | 5.13 |
| PSQGGVKVF | 697 | 8.50 | 2.93 | 4.27 |
| PTNGVSKLK | 46171 | 8.50 | #N/A | −1.37 |
| GIPAGGKLK | 46168 | 8.50 | #N/A | −0.77 |
| PIQGTLHLF | 46206 | 8.49 | 1.30 | −1.34 |
| GVNGSIHVH | 5440 | 8.49 | 6.59 | 1.23 |
| GLQAVVHVF | 8419 | 8.49 | #N/A | 0.44 |
| PLNGVTHVR | 33837 | 8.49 | 1.94 | −0.38 |
| GSQAVAKVF | 40650 | 8.49 | 1.33 | −0.30 |
| GVNAAVKVK | 30736 | 8.48 | 3.45 | 0.20 |
| GVQAVIKLH | 44853 | 8.48 | 2.18 | 2.14 |
| GSQASIRVY | 28512 | 8.48 | #N/A | 1.32 |
| GLNAVARVF | 3509 | 8.48 | #N/A | −0.53 |
| PINGTLRLR | 2661 | 8.48 | #N/A | 0.21 |
| PTPAAAHLH | 7298 | 8.48 | 3.50 | 0.62 |
| PIPAGGRVF | 11124 | 8.47 | 4.74 | 3.53 |
| GVPGGGHVK | 12460 | 8.47 | 5.85 | 2.03 |
| GLPASGHLK | 26038 | 8.47 | 2.49 | 1.43 |
| PSNASGKVR | 41414 | 8.47 | #N/A | −0.14 |
| GINASAKVH | 32433 | 8.46 | 3.41 | 1.84 |
| GLNAVVHLF | 40213 | 8.46 | 2.74 | −1.07 |
| PSQGSVHLK | 35606 | 8.46 | 5.09 | #N/A |
| PTPGTLRLR | 7137 | 8.45 | #N/A | −0.17 |
| GIPGTARLR | 46252 | 8.45 | #N/A | 2.00 |
| PLPGAGHLR | 15930 | 8.45 | 5.26 | 1.84 |
| PSQGVIKLK | 19251 | 8.45 | 3.16 | 2.13 |
| GTPAGVKLY | 40506 | 8.45 | 4.16 | 4.93 |
| PSNGSARLY | 7516 | 8.45 | 3.14 | 2.30 |
| GIPAALRER | 6540 | 8.45 | #N/A | 1.26 |
| PVNATIKVR | 46000 | 8.45 | #N/A | −0.62 |
| PLNGSVKLK | 8512 | 8.45 | #N/A | 0.92 |
| PSQGAGRVY | 19150 | 8.44 | 4.34 | 3.49 |
| PIQGAIKLY | 2928 | 8.44 | 3.21 | −0.61 |
| GVPAVAKVY | 45013 | 8.44 | −1.96 | 0.25 |
| GTQGALHLR | 37297 | 8.43 | 3.47 | 0.05 |
| PAPAVIRVF | 1720 | 8.43 | #N/A | 7.73 |
| PVQAGIHLY | 2377 | 8.42 | 1.81 | 2.13 |
| PAPGGVRVY | 21298 | 8.42 | 1.08 | 2.46 |
| PSNAAGRVR | 9464 | 8.42 | 3.18 | 2.30 |
| PINATARLH | 8230 | 8.42 | 1.06 | 0.03 |
| GAQGVLHLF | 5130 | 8.42 | 11.36 | 2.43 |
| GAPGVAKLR | 8828 | 8.41 | 5.49 | 1.79 |
| GAPGVAKLF | 12175 | 8.41 | 1.67 | 2.18 |
| GIQAVLHVF | 9117 | 8.40 | #N/A | 4.74 |
| GLQATAHVR | 37007 | 8.39 | 4.43 | 2.94 |
| GSPGGLKLF | 37863 | 8.38 | 7.32 | 1.07 |
| PIQATVRLY | 42617 | 8.38 | 1.18 | 1.25 |
| PTQGAIRLR | 45926 | 8.38 | 2.85 | 0.38 |
| PLPASARLY | 29342 | 8.38 | 3.16 | 0.84 |
| GLNGALRVF | 46209 | 8.37 | 0.07 | −2.65 |
| GIQGVGKLR | 12671 | 8.37 | 0.74 | −0.24 |
| PIPAAIKVR | 3299 | 8.36 | 1.44 | 0.13 |
| GVNGGVHVY | 5490 | 8.35 | 3.25 | 0.50 |
| PVQATARVK | 46198 | 8.35 | 4.32 | 2.92 |
| PLNAVVRLH | 19579 | 8.35 | 6.04 | 2.43 |
| GVQGSARLF | 44749 | 8.34 | #N/A | 3.54 |
| PLPAVLKVY | 12087 | 8.34 | 3.30 | 0.05 |
| PSNATGKVR | 41412 | 8.34 | #N/A | 0.67 |
| GVNGSVKLK | 38401 | 8.34 | #N/A | 1.87 |
| GTNGTIKVR | 46021 | 8.33 | 1.05 | −0.30 |
| PLPGSGHVY | 27014 | 8.33 | 1.38 | −1.14 |
| PLNGSVRLF | 40352 | 8.33 | 2.93 | −1.29 |
| GTQGGIHVH | 3998 | 8.33 | 1.71 | 0.15 |
| PAQAGIRVK | 45940 | 8.33 | #N/A | 1.30 |
| GLQGALRVF | 3525 | 8.32 | 2.44 | 3.12 |
| PSPATVKLK | 9657 | 8.32 | #N/A | 4.42 |
| PLNAGGHLY | 1133 | 8.32 | 3.08 | 3.00 |
| GINAVLHVH | 46059 | 8.32 | 1.94 | −0.44 |
| GIPAGGHLY | 6654 | 8.32 | 5.31 | 1.55 |
| PTPATGHVY | 13496 | 8.31 | 4.16 | 7.25 |
| GINGSIKLF | 6080 | 8.31 | 1.68 | −0.79 |
| PVQGSLHVY | 46003 | 8.31 | 3.39 | #N/A |
| GVNGTGHVR | 44588 | 8.31 | 4.60 | 3.52 |
| GLNGGVKLY | 3444 | 8.31 | 3.13 | 1.07 |
| GVQAVLHVK | 31419 | 8.31 | 7.70 | 2.41 |
| GTNGTVRLF | 26210 | 8.31 | 3.25 | 1.14 |
| GINAVGKVY | 32511 | 8.30 | 1.04 | −0.21 |
| PANGGVRLY | 988 | 8.30 | 2.83 | 2.59 |
| PANGGGHVH | 7763 | 8.30 | 4.17 | 0.79 |
| GANGAIKLK | 43994 | 8.29 | #N/A | 12.37 |
| GVNGSIRVE | 5441 | 8.29 | 1.79 | 0.94 |
| GINGTVRLF | 32051 | 8.29 | 0.51 | −1.97 |
| GIQGSGRVK | 18084 | 8.29 | #N/A | 4.75 |
| GTQAAIRVR | 4017 | 8.29 | 6.29 | 2.76 |
| PSQGGIRVY | 41620 | 8.28 | #N/A | 2.11 |
| GVPGAARLR | 38703 | 8.28 | #N/A | 0.05 |
| GVPGGGKLR | 12464 | 8.28 | #N/A | 0.13 |
| GSQGVLKVY | 11781 | 8.28 | 0.84 | −0.95 |
| GLNAAAKVR | 46279 | 8.28 | 3.12 | −0.60 |
| PVQGAAKVR | 42208 | 8.28 | 3.15 | 3.16 |
| GLQAAIKLH | 25434 | 8.27 | 2.74 | 0.07 |
| GSNGVIRLY | 11705 | 8.27 | 2.62 | 1.22 |
| GANGSVHLH | 29266 | 8.27 | 2.36 | 5.90 |
| GIQAGIKVY | 33079 | 8.27 | 3.90 | 3.61 |
| GTPATVRLK | 41488 | 8.27 | #N/A | 1.28 |
| GLPATVHLY | 15887 | 8.27 | 5.01 | 2.51 |
| GTQGTVKVR | 11422 | 8.26 | #N/A | 0.89 |
| PTNGSLHLR | 5859 | 8.26 | 2.23 | 6.14 |
| PSPAGIKLF | 7704 | 8.26 | 0.98 | 3.72 |
| GANGAIRLH | 37947 | 8.26 | #N/A | 3.04 |
| PTPAVVRVR | 46074 | 8.26 | #N/A | −1.69 |
| PLQASIRVK | 46277 | 8.26 | #N/A | −1.89 |
| GLQGALKVY | 11187 | 8.25 | 1.39 | 0.22 |
| PVNGSIHVK | 1860 | 8.25 | 3.82 | 2.88 |
| PLPGSARVF | 26847 | 8.25 | 1.18 | 0.36 |
| PSNGTGHLF | 7501 | 8.25 | 1.85 | −1.31 |
| GVNGSVRVH | 30644 | 8.24 | 5.87 | 2.99 |
| PTQGGVHLR | 9236 | 8.24 | 7.43 | 1.52 |
| GLQATGHLF | 8418 | 8.24 | #N/A | −2.18 |
| GAQGGGHVY | 44332 | 8.24 | #N/A | 2.96 |
| GVPAGIKVF | 9064 | 8.23 | 2.77 | 0.34 |
| GLNAGVRLY | 25125 | 8.23 | 2.14 | 7.74 |
| PVNAAGKVR | 21839 | 8.23 | 0.98 | 0.74 |
| PIQGVARLH | 36647 | 8.23 | 2.92 | 0.83 |
| GSQGTVHLF | 16696 | 8.23 | #N/A | −2.02 |
| GVNGGIKLY | 12289 | 8.22 | 3.69 | 0.13 |
| PVNGGVRVK | 21757 | 8.22 | #N/A | 4.33 |
| PLNASARVK | 46178 | 8.22 | 2.24 | 0.41 |
| PINGVAKLK | 32328 | 8.22 | #N/A | −1.43 |
| GANASIRVK | 44139 | 8.21 | #N/A | 1.87 |
| GLNASIHVH | 3495 | 8.21 | 3.71 | 0.82 |
| GANAGGHVY | 5044 | 8.20 | 1.53 | −0.60 |
| PSNGTIKVR | 39593 | 8.20 | #N/A | 0.02 |
| GLQGSIHVY | 46113 | 8.20 | −0.43 | −1.81 |
| GANASIRLF | 4987 | 8.20 | #N/A | 2.15 |
| GSNGVLRLY | 27870 | 8.20 | 2.18 | 1.88 |
| GTQGSIRLK | 41480 | 8.20 | #N/A | 0.92 |
| PTPATLRLR | 7335 | 8.20 | #N/A | 9.83 |
| GSNAGIHVF | 37654 | 8.19 | 1.16 | 1.23 |
| PANAALRVF | 1023 | 8.19 | 2.90 | 2.81 |
| GLNGAVRLH | 24666 | 8.19 | 2.03 | −2.01 |
| GAQAVIHVH | 29948 | 8.19 | 0.24 | −2.12 |
| PLNAVARVF | 802 | 8.18 | 3.79 | −0.07 |
| PINGASHLR | 33954 | 8.17 | 1.54 | −1.39 |
| PANASIRLY | 7772 | 8.17 | #N/A | −0.41 |
| GVQAVIHLY | 46242 | 8.17 | #N/A | 0.60 |
| PSNGGARLY | 13607 | 8.16 | 5.30 | 4.17 |
| PLNAGVRVR | 20134 | 8.16 | 2.20 | 0.86 |
| PVNASGKVR | 41431 | 8.15 | #N/A | 0.64 |
| PLQASAHLY | 3233 | 8.15 | −1.12 | −1.97 |
| PVQASLHVH | 2319 | 8.15 | 4.04 | #N/A |
| PLNGGAKVY | 13201 | 8.15 | 5.29 | 1.90 |
| GVPGSIRLY | 46158 | 8.15 | 1.97 | 0.39 |
| GIQGALHVF | 32572 | 8.15 | #N/A | 2.54 |
| PINAGGKLK | 36614 | 8.14 | 2.45 | 0.49 |
| GLPGTIRLY | 15832 | 8.14 | #N/A | −0.91 |
| PLNGAVHLY | 7599 | 8.14 | 1.52 | 1.54 |
| PLQGAVHLH | 1238 | 8.13 | 2.96 | −0.21 |

What is claimed is:

1. A modified adeno-associated virus (AAV) capsid protein, comprising a targeting peptide within variable region VIII (VR VIII),
wherein the targeting peptide has a sequence of X₁X₂X₃X₄X₅X₆X₇X₈X₉ and X₁, X₂, X₃, X₄, X₅, X₆, X₇, X₈, and X₉ are each independently selected from any amino acid residue.

2. The modified AAV capsid protein of claim 1, wherein
(i) X₁ is independently selected from a proline (P) and a glycine (G);
(ii) X₂ is independently selected from a lysine (L), a threonine (T), a serine (S), an alanine (A), a valine (V), and an isoleucine (I);
(iii) X₃ is independently selected from an asparagine (N), a glutamine (Q), and a proline (P);
(iv) X₄ is independently selected from a glycine (G) and an alanine (A);
(v) X₅ is independently selected from an alanine (A), a threonine (T), a serine (S), a valine (V), and a glycine (G);
(vi) X₆ is independently selected from a valine (V), a leucine (L), an alanine (A), an isoleucine (I), a glycine (G), a serine (S), and a threonine (T);
(vii) X₇ is independently selected from a histidine (H), an arginine (R), and a lysine (K);
(viii) X₈ is independently selected from a leucine (L) and a valine (V); and
(ix) X₉ is independently selected from a tyrosine (Y), an arginine (R), a histidine (H), a lysine (K), and a phenylalanine (F).

3. The modified AAV capsid protein of claim 1 or 2, wherein a targeting peptide within VR VIII has a sequence selected from SEQ ID NO: 160-55819 and 55857-55859.

4. The modified AAV capsid protein of any one of claims 1-3, wherein the targeting peptide has a sequence of PX₂X₃GAVX₇LY (SEQ ID NO: 2) and X₂, X₃, and X₇ are independently selected from any amino acid residue.

5. The modified AAV capsid protein of claim 3, wherein
(i) X₂ is independently selected from a lysine (L), an isoleucine (I), a valine (V), and an alanine (A);
(ii) X₃ is an asparagine (N) or a glutamine (Q); and
(iii) X₇ is independently selected from a histidine (H), an arginine (R) and a lysine (K).

6. The modified AAV capsid protein of claim 4 or 5, wherein the targeting peptide is:
(i)
(SEQ ID NO: 3)
PLQGAVHLY;
(ii)
(SEQ ID NO: 4)
PLQGAVRLY;
(iii)
(SEQ ID NO: 5)
PLQGAVKLY;
(iv)
(SEQ ID NO: 6)
PINGAVHLY;
(v)
(SEQ ID NO: 7)
PVNGAVHLY;
(vi)
(SEQ ID NO: 8)
PANGAVHLY;
or
(vii)
(SEQ ID NO: 9)
PLNGAVHLY.

7. The modified AAV capsid protein of any one of claims 1-6, wherein the targeting peptide is inserted between S586 and A589 of an AAV9 capsid protein, thereby replacing A587 and Q588 of the AAV9 capsid protein.

8. The modified AAV capsid protein of any one of claims 1-7, having at least 90%, 95%, 96%, 97%, 98% or 99% sequence identity to an AAV9 capsid protein.

9. The modified AAV capsid protein of any one of claims 1-6, wherein the targeting peptide is inserted
(i) between S586 and T589 of an Anc80L65 capsid protein, thereby replacing A587 and N588 of the Anc80L65 capsid protein;
(ii) between Q585 and N588 of an Anc80L65 capsid protein, thereby replacing S586 and A587 of the Anc80L65 capsid protein;
(iii) between L584 and A587 of an Anc80L65 capsid protein, thereby replacing Q585 and S586 of the Anc80L65 capsid protein;
(iv) between A587 and A590 of an Anc80L65 capsid protein, thereby replacing N588 and T589 of the Anc80L65 capsid protein; or
(v) between S586 and A587 of an Anc80L65 capsid protein.

10. The modified AAV capsid protein of claim 9, wherein the targeting peptide comprises: PLNGAVHLY (SEQ ID NO: 9).

11. The modified AAV capsid protein of claim 9 or 10, having at least 90%, 95%, 96%, 97%, 98% or 99% sequence identity to an Anc80L65 capsid protein.

12. The modified AAV capsid protein of claim 1 or 2, wherein the targeting peptide has a sequence of PX₂X₃GX₅X₆X₇LY (SEQ ID NO: 10) and X₂, X₃, X₅, X₆, and X₇ are independently selected from any amino acid residue.

13. The modified AAV capsid protein of claim 12, wherein
(i) X₂ is independently selected from a leucine (L), a threonine (T), or a serine (S);
(ii) X₃ is independently selected from an asparagine (N) and a glutamine (Q);
(iii) X₅ is independently selected from an alanine (A) and a threonine (T);
(iv) X₆ is independently selected from a valine (V) and a leucine (L); and
(v) X₇ is independently selected from a histidine (H), an arginine (R), and a lysine (K).

14. The modified AAV capsid protein of claim 12 or 13, wherein the targeting peptide is:
(i)
(SEQ ID NO: 11)
PTNGTVRLY;
(ii)
(SEQ ID NO: 12)
PTNGTVHLY;
(iii)
(SEQ ID NO: 13)
PTNGTVKLY;
(iv)
(SEQ ID NO: 14)
PSNGTLRLY;
(v)
(SEQ ID NO: 15)
PSNGTLHLY;
(vi)
(SEQ ID NO: 16)
PSNGTLKLY
(vii)
(SEQ ID NO: 17)
PTNGTLRLY;
(viii)
(SEQ ID NO: 18)
PTNGTLHLY;
or
(ix)
(SEQ ID NO: 19)
PTNGTLKLY.

15. The modified AAV capsid protein of claim 1 or 2, wherein the targeting peptide has a sequence of PX₂X₃GAVX₇X₈X₉ (SEQ ID NO: 20) and X₂, X₃, X₅, X₆, and X₇ are independently selected from any amino acid residue.

16. The modified AAV capsid protein of claim 15, wherein
(i) X₂ is independently selected from a leucine (L), a threonine (T), or a serine (S);
(ii) X₃ is independently selected from an asparagine (N) and a glutamine (Q);
(iii) X₇ is independently selected from a histidine (H) and a threonine (T);
(iv) X₈ is independently selected from a valine (V) and a leucine (L); and
(v) X₉ is independently selected from a tyrosine (Y) and an arginine (R).

17. The modified AAV capsid protein of claim 15 or 16, wherein the targeting peptide is:
(i)
(SEQ ID NO: 21)
PTQGAVTVR;
(ii)
(SEQ ID NO: 22)
PLQGAVTVR;
(iii)
(SEQ ID NO: 23)
PLQGAVHVR;
(iv)
(SEQ ID NO: 24)
PLQGAVHVY;
(v)
(SEQ ID NO: 25)
PSQGAVTLR;
(vi)
(SEQ ID NO: 26)
PLQGAVTLR;
(vii)
(SEQ ID NO: 27)
PLQGAVHLR;
or
(viii)
(SEQ ID NO: 28)
PTQGAVTLR.

18. The modified AAV capsid protein of any one of claims 1-17, wherein the targeting peptide does not comprise PLNGAVHLY (SEQ ID NO: 9).

19. The modified AAV capsid protein of any one of claims 1-17, wherein the targeting peptide comprises PLNGSVHLY (SEQ ID NO: 3603) or PLNGTVHLY (SEQ ID NO: 1232).

20. The modified AAV capsid protein of any one of claims 12-19, wherein the targeting peptide is inserted between S586 and A589 of an AAV9 capsid protein, thereby replacing A587 and Q588 of the AAV9 capsid protein.

21. A modified adeno-associated virus (AAV) capsid protein, comprising:
a targeting peptide within VR VIII wherein the targeting peptide has a sequence selected from SEQ ID NO: 160-619.

22. The modified AAV capsid protein of any one of claims 1-21, having at least 90%, 91%, 92%, 93%, 94%, 95%, 96%, 97%, 98%, 99% or 99.5% sequence identity to a sequence of a reference AAV capsid protein.

23. The modified AAV capsid protein of claim 22, wherein the reference AAV capsid protein is selected from VP1, VP2 and VP3.

24. The modified AAV capsid protein of claim 22 or 23, wherein the reference AAV capsid protein is a capsid protein of an AAV selected from the group consisting of: AAV9; Anc80L65; Anc80-55, Anc80-129, Anc80-156, Anc80-751, Anc80-1029, Anc80-1712, AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu. 9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

25. The modified AAV capsid protein of any one of claims 22-24, wherein the reference AAV capsid protein is a capsid protein having a sequence selected from SEQ ID Nos: 54-158, or a fragment thereof.

26. The modified AAV capsid protein of any one of claims 22-25, wherein the reference AAV capsid protein is a capsid protein having a sequence of SEQ ID NO: 61 or a fragment thereof.

27. The modified AAV capsid protein of any one of claims 22-26, wherein the reference AAV capsid protein is a capsid protein having a sequence of SEQ ID NO: 142 or a fragment thereof.

28. The modified AAV capsid protein of any one of claims 22-27, wherein the targeting peptide is positioned between 576 and 601 within VR VIII of the modified AAV capsid protein.

29. The modified AAV capsid protein of any one of claims 1-28, further comprising an N-terminal flanking region on the N-terminal end of the targeting peptide.

30. The modified AAV capsid protein of claim 29, wherein the N-terminal flanking region comprises at least four consecutive amino acids from amino acid residues 576-585 of the reference AAV capsid protein, wherein the amino acid residues are numbered according to the amino acid sequence of the reference AAV capsid protein.

31. The modified AAV capsid protein of claim 29 or 30, wherein the N-terminal flanking region has the sequence of B₁YGB₂VATNB₃QS (SEQ ID NO: 55849), and B₁, B₂, and B₃ are each independently selected from any amino acid residue.

32. The modified AAV capsid protein of claim 31, wherein B₁ is selected from a glutamate (E) or a serine (S), B₂ is selected from a threonine (T) or a glutamine (Q), and B₃ is selected from a leucine (L) or a histidine (H).

33. The modified AAV capsid protein of any one of claims 29-32, wherein the N-terminal flanking region has the sequence of SYGQVATNHQS (SEQ ID NO: 55848).

34. The modified AAV capsid protein of any one of claims 29-33, wherein the N-terminal flanking region replaces N-terminal reference sequence of the reference AAV capsid protein, wherein the N-terminal reference sequence has at least 60% sequence identity to the N-terminal flanking region and positioned at N-terminal end of an insertion site of the targeting peptide within the reference AAV capsid protein.

35. The modified AAV capsid protein of claim 34, wherein the N-terminal reference sequence has the sequence of B₁YGB₂VATNB₃QS (SEQ ID NO: 55849).

36. The modified AAV capsid protein of any one of claims 1-35, further comprising a C-terminal flanking region on the C-terminal end of the targeting peptide.

37. The modified AAV capsid protein of claim 36, wherein the C-terminal flanking region comprises at least four consecutive amino acids from amino acid residues 589-602 of the reference AAV capsid protein, wherein the amino acid residues are numbered according to the amino acid sequence of the reference AAV capsid protein.

38. The modified AAV capsid protein of claim 36 or 37, wherein the C-terminal flanking region has the sequence of AQAQTGZ₁VZ₂Z₃QGZ₄ (SEQ ID NO: 55851), and Z₁, Z₂, Z₃, and Z₄ are each independently selected from any amino acid residue.

39. The modified AAV capsid protein of claim 38, wherein Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

40. The modified AAV capsid protein of any one of claims 36-39, wherein the C-terminal flanking region has the sequence of AQAQTGWVQNQGI (SEQ ID NO: 55850).

41. The modified AAV capsid protein of any one of claims 36-40, wherein the C-terminal flanking region replaces C-terminal reference sequence of the reference AAV capsid protein, wherein the C-terminal reference sequence has at least 60% sequence identity to the C-terminal flanking region and positioned at C-terminal end of an insertion site of the targeting peptide within the reference AAV capsid protein.

42. The modified AAV capsid protein of claim 41, wherein the C-terminal reference sequence has the sequence of AQAQTGZ₁VZ₂Z₃QGZ₄ (SEQ ID NO: 55851).

43. The modified AAV capsid protein of any one of claims 1-28, further comprising:
(i) a N-terminal flanking region having the sequence of B₁YGB₂VATNB₃QS (SEQ ID NO: 55849, where B₁, B₂, and B₃ are each independently selected from any amino acid residue; and
(ii) a C-terminal flanking region having the sequence of AQAQTGZ₁VZ₂Z₃QGZ₄ (SEQ ID NO: 55851), where Z₁, Z₂, Z₃, and Z₄ are each independently selected from any amino acid residue.

44. The modified AAV capsid protein of any one of claims 1-28, further comprising:
(i) a N-terminal flanking region having the sequence of B₁YGB₂VATNB₃QS (SEQ ID NO: 55860) and B₁ is selected from a glutamate (E) or a serine (S), B₂ is selected from a threonine (T) or a glutamine (Q), and B₃ is selected from a leucine (L) or a histidine (H); and
(ii) a C-terminal flanking region having the sequence of AQAQTGZ₁VZ₂Z₃QGZ₄ (SEQ ID NO: 55861) and Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

45. The modified AAV capsid protein of any one of claims 1-28, further comprising:
(i) a N-terminal flanking region having the sequence of SYGQVATNHQS (SEQ ID NO: 55848), and
(ii) a C-terminal flanking region having the sequence of AQAQTGWVQNQGI (SEQ ID NO: 55850).

46. The modified AAV capsid protein of any one of claims 1-28, comprising an amino acid sequence of B₁YGB₂VATNB₃QSPLMGAVHLYAQAQTGZ₁VZ₂Z₃QGZ₄ (SEQ ID NO: 55852), where B₁, B₂, B₃, Z₁, Z₂, Z₃, and Z₄ are each independently selected from any amino acid residue.

47. The modified AAV capsid protein of claim 46, wherein B₁ is selected from a glutamate (E) or a serine (S), B₂ is selected from a threonine (T) or a glutamine (Q), B₃ is selected from a leucine (L) or a histidine (H), Z₁ is selected from a threonine (T) or a tryptophan (W), Z₂ is selected from an asparagine (N) or a glutamine (Q), Z₃ is selected from a serine (S) or an asparagine (N), and Z₄ is selected from an alanine (A) or an isoleucine (I).

48. The modified capsid protein of any one of claims 1-28, wherein the targeting peptide is a peptide having 1, 2, 3, 4, 5, 6, 7, 8, 9, 10, or more amino acid residues different to a sequence of SEQ LD NO: 29, wherein the different amino acid comprise an insertion, a deletion, or a substitution compared to the sequence of SEQ ID NO: 29.

49. The modified AAV capsid protein of claim 48, wherein the targeting peptide has the sequence of SEQ ID NO: 29.

50. The modified AAV capsid protein of any one of claims 22-49, wherein the modified AAV capsid protein has one or more modifications comprising amino acid insertions, deletions, substitutions or combinations thereof as compared to a reference AAV capsid protein.

51. The modified AAV capsid protein of any one of claims 22-50, wherein the one or more modifications comprises an amino acid insertion, deletion, substitution, or a combination thereof to introduce the targeting peptide into VR VIII of the reference AAV capsid protein.

52. The modified AAV capsid protein of any one of claim 22-50, wherein the one or more modifications comprises an amino acid modification outside of VR VIII of the reference AAV capsid protein.

53. The modified AAV capsid protein of claim 52, wherein the one or more modifications outside of VR VIII of the reference AAV capsid protein comprise one or more modifications in VR I, VR II, VR III, VR IV, VR V, VR VI, or VR VII.

54. The modified AAV capsid protein of claim 53, wherein the one or more modifications outside of VR VIII of the reference AAV capsid protein comprise one or more modifications in VR IV and VR V.

55. The modified AAV capsid protein of any one of claims 52-54, wherein the one or more modifications in VR IV result in introduction of a sequence of SEQ ID NO: 30.

56. The modified AAV capsid protein of any one of claims 52-55, wherein the one or more modifications in VR V result in introduction of a sequence of SEQ ID NO: 31.

57. The modified AAV capsid protein of any one of claims 1-52, wherein the one or more modifications comprises one or more amino acid deletions within VR VIII.

58. The modified AAV capsid protein of claim 57, wherein the one or more amino acid deletions comprise a deletion of one, two, three, four, or five or more amino acid residues immediately adjacent to the N terminal end of the targeting peptide within VR VIII.

59. The modified AAV capsid protein of claim 57 or 58, wherein the one or more amino acid deletions comprise a deletion of at least one amino acid residue at a position selected from 584, 585, 586, 587, or 588, or a combination thereof, relative to a reference sequence numbered according to the amino acid sequence of the reference AAV capsid protein.

60. The modified AAV capsid protein of claim 59, wherein the one or more amino acid deletions comprises a deletion of the amino acid residue at position 587, relative to a reference sequence numbered according to the amino acid sequence of the reference AAV capsid protein.

61. The modified AAV capsid protein of claim 59, wherein the one or more amino acid deletions comprises a deletion of the amino acid residue at position 588, relative to a reference sequence numbered according to the amino acid sequence of the reference AAV capsid protein.

62. The modified AAV capsid protein of any one of claims 57-61, wherein the amino acid deletion comprises deletion of one, two, three, four, or five or more amino acid residues immediately adjacent to the C terminal end of the targeting peptide within VR VIII.

63. The modified AAV capsid protein of claim 62, wherein the amino acid deletion comprises deletion of an amino acid residue at position 589, 590, or 591, or a combination thereof, relative to a reference sequence numbered according to the amino acid sequence of the reference AAV capsid protein.

64. The modified AAV capsid protein of any one of claims 50-63, wherein the amino acid insertion comprises insertion of one, two, three, four, or five or more amino acid residues immediately adjacent to C terminal end of the targeting peptide within VR VIII.

65. The modified AAV capsid protein of claim 64, wherein the inserted amino acid residue is independently selected from any amino acid residue.

66. The modified AAV capsid protein of claim 64 or 65, wherein the inserted amino acid residue(s) is identical to the amino acid residue deleted adjacent to the N terminal end or C terminal end of the targeting peptide within VR VIII.

67. The modified AAV capsid protein of any one of claims 64-66, wherein the inserted amino acid residue is an alanine (A) or an asparagine (N).

68. The modified AAV capsid protein of claim 65, wherein the inserted amino acids are an alanine (A) at the position immediately adjacent to the C terminal end of the targeting peptide and an asparagine (N) at the next subsequent position, thereby having an amino acid sequence of AN immediately adjacent to the C terminal end of the targeting peptide.

69. The modified AAV capsid protein of any one of claims 22-61, wherein the targeting peptide is:
(i)
(SEQ ID NO: 32)
PLNGAVHLYN;
or
(ii)
(SEQ ID NO: 33)
PLNGAVHLYAN.

70. The modified AAV capsid protein of any one of claims 1-69, wherein
(i) the reference AAV capsid protein is a capsid protein of AAV1 or a modification thereof and the targeting peptide is between S586 and T589 of the reference AAV capsid protein, thereby replacing residues S587 and S588 of the reference capsid protein;
(ii) the reference AAV capsid protein is a capsid protein of AAV2 or a modification thereof and the targeting peptide is between R585 and R588 of the reference AAV capsid protein, thereby replacing residues G586 and N587 of the reference capsid protein;
(iii) the reference AAV capsid protein is a capsid protein of AAV3 or a modification thereof and the targeting peptide is between S586 and T589 of the reference AAV capsid protein, thereby replacing residues S587 and N588 of the reference capsid protein;
(iv) the reference AAV capsid protein is a capsid protein of AAV4 or a modification thereof and the targeting peptide is between D582 and N585 of the reference AAV capsid protein, thereby replacing residues Q583 and S584 of the reference capsid protein;
(v) the reference AAV capsid protein is a capsid protein of AAV5 or a modification thereof and the targeting peptide is between S575 and T578 of the reference AAV capsid protein, thereby replacing residues S576 and S577 of the reference capsid protein;
(vi) the reference AAV capsid protein is a capsid protein of AAV6 or a modification thereof and the targeting peptide is between S586 and T589 of the reference AAV capsid protein, thereby replacing residues S587 and S588 of the reference capsid protein;
(vii) the reference AAV capsid protein is a capsid protein of AAV7 or a modification thereof and the targeting peptide is between A587 and T590 of the reference AAV capsid protein, thereby replacing residues A588 and N589 of the reference capsid protein;
(viii) the reference AAV capsid protein is a capsid protein of AAV8 or a modification thereof and the targeting peptide is between Q588 and A591 of the modified AAV capsid protein thereby replacing residues Q589 and N590 of the reference capsid protein;
(ix) the reference AAV capsid protein is a capsid protein of AAV9 or a modification thereof and the targeting peptide is between S586 and A589 of the reference AAV capsid protein thereby replacing residues A587 and Q588 of the reference capsid protein;
(x) the reference AAV capsid protein is a capsid protein of AAVrh10 or a modification thereof and the targeting peptide is between Q588 and A591 of the reference AAV capsid protein thereby replacing residues Q589 and N590 of the reference capsid protein;
(xi) the reference AAV capsid protein is a capsid protein of AAVpo.1 or a modification thereof and the targeting peptide is between N565 and S568 of the reference AAV capsid protein thereby replacing residues Q566 and N567 of the reference capsid protein;
(xii) the reference AAV capsid protein is a capsid protein of AAV12 or a modification thereof and the targeting peptide is between N590 and A593 of the reference AAV capsid protein thereby replacing residues Q591 and N592 of the reference capsid protein;
(xiii) the reference AAV capsid protein is a capsid protein of Anc80 or a modification thereof and the targeting peptide is between S586 and T589 of the reference AAV capsid protein thereby replacing residues S587 and N588 of the reference capsid protein;
(xiv) the reference AAV capsid protein is a capsid protein of Anc80-55 or a modification thereof and the targeting peptide is between S586 and T589 of the reference AAV capsid protein thereby replacing residues S587 and N588 of the reference capsid protein;
(xv) the reference AAV capsid protein is a capsid protein of Anc80-129 or a modification thereof and the targeting peptide is between S586 and T589 of the reference AAV capsid protein thereby replacing residues A587 and N588 of the reference capsid protein;
(xvi) the reference AAV capsid protein is a capsid protein of Anc80-156 or a modification thereof and the targeting peptide is between S586 and T589 of the reference AAV capsid protein thereby replacing residues A587 and N588 of the reference capsid protein;
(xvii) the reference AAV capsid protein is a capsid protein of Anc80-751 or a modification thereof and the targeting peptide is between S586 and T589 of the reference AAV capsid protein thereby replacing residues A587 and N588 of the reference capsid protein;
(xviii) the reference AAV capsid protein is a capsid protein of Anc80-1029 or a modification thereof and the targeting peptide is between S586 and T589 of the reference AAV capsid protein thereby replacing residues A587 and N588 of the reference capsid protein; or
(xix) the reference AAV capsid protein is a capsid protein of Anc80-1712 or a modification thereof and the targeting peptide is between S586 and T589 of the reference AAV capsid protein thereby replacing residues A587 and N588 of the reference capsid protein.

71. The modified AAV capsid protein of any one of claims 1-69, wherein:
(i) the reference AAV capsid protein is a capsid protein of AAV1 or a modification thereof and the targeting peptide is between D590 and P591 or between S588 and T589 of the reference AAV capsid protein;
(ii) the reference AAV capsid protein is a capsid protein of AAV2 or a modification thereof and the targeting peptide is between R588 and Q589 or between N587 and R588 of the reference AAV capsid protein;
(iii) the reference AAV capsid protein is a capsid protein of AAV3 or a modification thereof and the targeting peptide is between S586 and S587 or between N588 and T589 of the reference AAV capsid protein;
(iv) the reference AAV capsid protein is a capsid protein of AAV4 or a modification thereof and the targeting peptide is between S584 and N585 or between S586 and N587 of the reference AAV capsid protein;
(v) the reference AAV capsid protein is a capsid protein of AAV5 or a modification thereof and the targeting peptide is between S575 and S576 or between T577 and T578 of the reference AAV capsid protein;
(vi) the reference AAV capsid protein is a capsid protein of AAV6 or a modification thereof and the targeting peptide is between D590 and P591 or S588 and T589 of the reference AAV capsid protein;
(vii) the reference AAV capsid protein is a capsid protein of AAV7 or a modification thereof and the targeting peptide is between N589 and T590 of the reference AAV capsid protein;
(viii) the reference AAV capsid protein is a capsid protein of AAV8 or a modification thereof and the targeting peptide is between N590 and T591 of the modified AAV capsid protein;
(ix) the reference AAV capsid protein is a capsid protein of AAV9 or a modification thereof and the targeting peptide is between Q588 and A589 of the reference AAV capsid protein;
(x) the reference AAV capsid protein is a capsid protein of AAVrh10 or a modification thereof and the targeting peptide is between N590 and A591 of the reference AAV capsid protein;
(xi) the reference AAV capsid protein is a capsid protein of AAVpo.1 or a modification thereof and the targeting peptide is between N567 and S568 or between N569 and T570 of the reference AAV capsid protein;
(xii) the reference AAV capsid protein is a capsid protein of AAV12 or a modification thereof and the targeting peptide is between N592 and A593 or between T594 and T595 of the reference AAV capsid protein;
(xiii) the reference AAV capsid protein is a capsid protein of Anc80 or a modification thereof and the targeting peptide is between T589 and A590, between N588 and T589, or between N587 and T588 of the reference AAV capsid protein;
(xiv) the reference AAV capsid protein is a capsid protein of Anc80-55 or a modification thereof and the targeting peptide is between T589 and A590 or between N587 and T588 of the reference AAV capsid protein;
(xv) the reference AAV capsid protein is a capsid protein of Anc80-129 or a modification thereof and the targeting peptide is between T589 and A590 or between N587 and T588 of the reference AAV capsid protein;
(xvi) the reference AAV capsid protein is a capsid protein of Anc80-156 or a modification thereof and the targeting peptide is between T589 and A590 or between N587 and T588 of the reference AAV capsid protein;
(xvii) the reference AAV capsid protein is a capsid protein of Anc80-751 or a modification thereof and the targeting peptide is between T589 and A590 or between N587 and T588 of the reference AAV capsid protein;
(xviii) the reference AAV capsid protein is a capsid protein of Anc80-1029 or a modification thereof and the targeting peptide is between T589 and A590 or between N587 and T588 of the reference AAV capsid protein; or
(xix) the reference AAV capsid protein is a capsid protein of Anc80-1712 or a modification thereof and the targeting peptide is between T589 and A590 or between N587 and T588 of the reference AAV capsid protein.

72. A modified AAV capsid protein having a sequence selected from SEQ ID NO: 34-39.

73. A polynucleotide encoding the modified AAV capsid protein of any one of claims 1-72.

74. A vector comprising the polynucleotide of claim 72.

75. The vector of claim 74, further comprising a promoter operably linked to the polynucleotide.

76. A host cell comprising the modified AAV capsid protein of any one of claims 1-71, the polynucleotide specified in claim 72, or the vector of claim 74 or 75.

77. A recombinant AAV virion (rAAV) comprising the modified AAV capsid protein of any one of claims 1-71.

78. The AAV virion of claim 77, further comprising an exogenous polynucleotide.

79. The AAV virion of claim 78, wherein the exogenous polynucleotide comprises a template for homology directed repair.

80. The AAV virion of claim 78, wherein the exogenous polynucleotide comprises an expressible polynucleotide encoding a therapeutic tRNA, miRNA, gene editing guide RNA, or RNA-editing guide RNA.

81. The AAV virion of claim 80, wherein the exogenous polynucleotide comprises an expressible polynucleotide encoding a therapeutic protein.

82. The AAV virion of claim 81, wherein the therapeutic protein is used for treating and/or preventing a disease of the central nervous system (CNS).

83. The AAV virion of any one of claims 77-82, wherein the AAV virion, when administered in a therapeutically effective amount to a subject, has increased specificity to a central nervous system (CNS) tissue, relative to a reference AAV virion comprising a reference AAV capsid protein without the targeting peptide.

84. The AAV virion of any one of claims 77-83, wherein the AAV virion, when administered in a therapeutically effective amount to a subject, has increased transduction efficiency in the CNS, relative to a reference AAV virion comprising a reference AAV capsid protein without the targeting peptide.

85. The AAV virion of any one of claims 77-84, wherein the AAV virion, when administered in a therapeutically effective amount to a subject, has increased blood brain barrier penetration in the subject, relative to a reference AAV virion comprising a reference capsid protein without the targeting peptide.

86. A pharmaceutical composition comprising the AAV virion of any one of claims 77-85.

87. A method for treating or ameliorating or preventing a disease or condition in a subject, comprising administering a therapeutically effective amount of the AAV virion of any one of claims 77-85 or the pharmaceutical composition of claim 86.

88. The method of treating or ameliorating or preventing a disease according to claim 87, wherein the disease is a disease of the central nervous system (CNS).

89. The method of treating or ameliorating or preventing a disease according to claim 88, the CNS disease is a lysosomal storage disease (LSD).

90. The method of treating or ameliorating or preventing a disease according to claim 88, wherein the CNS disease is a leukodystrophy.

91. The method of treating or ameliorating or preventing a disease according to claim 88, wherein the CNS disease is metachromatic leukodystrophy (MILD).

92. The method of treating or ameliorating or preventing a disease according to claim 88, wherein the CNS disease is Krabbe.

93. The method of treating or ameliorating or preventing a disease according to claim 88, wherein the CNS disease is cancer.

94. The method of treating or ameliorating or preventing a disease according to claim 88, wherein the CNS disease is metastatic breast cancer.

95. The modified adeno-associated virus (AAV) capsid protein of any one of claims 1-71, for use in treating and/or preventing a disease of the central nervous system (CNS).

96. An AAV virion comprising the modified AAV capsid protein of any one of claims 1-71 or an AAV virion of any one of claims 77-85 for use in treating and/or preventing a disease of the central nervous system (CNS).

97. A pharmaceutical composition comprising the modified AAV capsid protein of any one of claims 1-71, and/or the AAV virion specified in any one of claims 77-85 for use in treating and/or preventing a disease of the central nervous system (CNS).

98. A method of transferring an exogenous polynucleotide to the central nervous system (CNS), comprising the step of administering the AAV virion specified in any one of claims 77-85 to a subject.

99. The method of claim 98, wherein the administration results in transfer of the exogenous polynucleotide in the CNS, at a CNS:liver infection ratio of greater than 1 when measured by genome copies of the AAV virion.

100. The method of claim 98 or 99, wherein the administration results in expression of the exogenous polynucleotide in CNS, at a CNS:liver expression ratio of greater than 10.

101. The method of any one of claims 98-100, wherein the CNS:liver expression ratio of greater than 10 when measured by protein expression.

102. Use of the modified AAV capsid protein of any one of claims 1-71, and/or the AAV virion specified in any one of claims 77-85 for transferring an exogenous polynucleotide to the central nervous system.

103. The use according to claim 102, wherein said use is a non-therapeutic use.
